(19) United States
(12) Patent Application Publication
(10) Pub. No.: US 2026/0083863 A1
(43) Pub. Date: Mar. 26, 2026

(54) CAPSID POLYPEPTIDES AND METHODS OF USE THEREOF

(71) Applicant: DYNO THERAPEUTICS, INC., Watertown, MA (US)

(72) Inventors: Samuel WOLOCK, Brookline, MA (US); Stephen MALINA, New York, NY (US); Demarcus BRIERS, Somerville, MA (US)

(73) Assignee: DYNO THERAPEUTICS, INC., Watertown, MA (US)

(21) Appl. No.: 19/342,097

(22) Filed: Sep. 26, 2025

Related U.S. Application Data
(63) Continuation of application No. PCT/US2024/044794, filed on Aug. 30, 2024.
(60) Provisional application No. 63/535,698, filed on Aug. 31, 2023.

(51) Int. Cl. A61K 48/00; C07K 14/005; C12N 15/86

(52) U.S. Cl. CPC A61K 48/0058; A61K 48/0091; C07K 14/005; C12N 15/86; C12N 2750/14122; C12N 2750/14143; C12N 2750/14145; C12N 2750/14152

(57) ABSTRACT

The disclosure is directed in part to dependoparvovirus capsid polypeptides that can be used to deliver payloads.

### 1. CROSS-REFERENCE TO RELATED APPLICATIONS

This application is a continuation of international application no. PCT/US2024/044794, filed Aug. 30, 2024, which claims the priority benefit of U.S. provisional application No. 63/535,698, filed Aug. 31, 2023, the contents of each of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Sep. 26, 2025, is named DYN-004C1_SL.xml and is 157,308 bytes in size.

### 3. BACKGROUND

Dependoparvoviruses, e.g., adeno-associated dependoparvoviruses, e.g., adeno-associated viruses (AAVs), are of interest as vectors for delivering various payloads to cells, including in human subjects.

### 4. SUMMARY

The present disclosure relates, in part, to improved dependoparvovirus capsid polypeptides, such as VP1, VP2 and/or VP3 capsid polypeptides, methods of producing a dependoparvovirus comprising capsid polypeptides, compositions for use in the same, as well as viral particles produced by the same. In certain aspects, the present disclosure relates to viral particles comprising the improved dependoparvovirus capsid polypeptides, with increased central nervous system (CNS) biodistribution and/or transduction as compared to viral particles, e.g., without the mutations in the improved dependoparvovirus capsid polypeptides.

Accordingly, the present disclosure provides a capsid polypeptide described herein.

In some embodiments, the capsid polypeptide comprises an amino acid sequence having at least 70%, 75%, 80%, 85%, 90%, 91%, 92%, 93%, 94%, 95%, 96%, 97%, 98%, 99% or 100% identity to a VP1 polypeptide of SEQ ID NO:12, 26, 28, 30, 32, 34, 36 or 38, or to a VP2 or VP3 portion thereof. Typically, a capsid polypeptide of the disclosure comprises a mutation at the position corresponding to A472 (e.g., A472S), V473 (e.g., V473A), S483 (e.g., S483F), and/or T492 (e.g., T492S) as compared to a capsid polypeptide of SEQ ID NO:1. A capsid polypeptide of the disclosure optionally further comprises a mutation at one or more of Q579 (e.g., Q579V), Q592 (e.g., Q592I), T593 (e.g., T593V), W595 (e.g., W595A), V596 (e.g., V596L), N598 (e.g., N598S), and I601 (e.g., I601A) as compared to a capsid polypeptide of SEQ ID NO:1. In some embodiments, the capsid polypeptide comprises a mutation at A472 as compared to a capsid polypeptide of SEQ ID NO:1. In some embodiments, the capsid polypeptide comprises a mutation at V473 as compared to a capsid polypeptide of SEQ ID NO:1. In some embodiments, the capsid polypeptide comprises a mutation at S483 as compared to a capsid polypeptide of SEQ ID NO:1. In some embodiments, the capsid polypeptide comprises a mutation at T492 as compared to a capsid polypeptide of SEQ ID NO:1. In some embodiments, the capsid polypeptide comprises a mutation at A472, V473, S483, and T492 as compared to a capsid polypeptide of SEQ ID NO:1.

In some embodiments, a capsid polypeptide of the disclosure comprises one, two, three, or all four mutations selected from: (a) a serine at a position corresponding to A472 of the VP1 capsid polypeptide of SEQ ID NO:1, (b) an alanine at a position corresponding to V473 of the VP1 capsid polypeptide of SEQ ID NO:1, (c) a phenylalanine at a position corresponding to S483 of the VP1 capsid polypeptide of SEQ ID NO:1, and (d) a serine at a position corresponding to T492 of the VP1 capsid polypeptide of SEQ ID NO:1. In some embodiments, a capsid polypeptide of the disclosure comprises a serine at a position corresponding to A472 of the VP1 capsid polypeptide of SEQ ID NO:1, an alanine at a position corresponding to V473 of the VP1 capsid polypeptide of SEQ ID NO:1, a phenylalanine at a position corresponding to S483 of the VP1 capsid polypeptide of SEQ ID NO:1, and a serine at a position corresponding to T492 of the VP1 capsid polypeptide of SEQ ID NO:1.

In further embodiments, the capsid polypeptide comprises a mutation at any of the foregoing combinations of positions as compared to a capsid polypeptide of SEQ ID NO:1, together with a mutation at Q579, Q592, T593, W595, V596, N598, 1601, or any combination of two, three, four, five, six, or all seven of Q579, Q592, T593, W595, V596, N598, and I601. In some embodiments, the capsid polypeptide comprises a mutation at any combination of A472 (e.g., A472S), V473 (e.g., V473A), S483 (e.g., S483F), and T492 (e.g., T492S) as compared to a capsid polypeptide of SEQ ID NO:1, and further comprises one, two, three, four, five, six, or all seven mutations selected from (a) a valine at a position corresponding to Q579 of the VP1 capsid polypeptide of SEQ ID NO:1, (b) an isoleucine at a position corresponding to Q592 of the VP1 capsid polypeptide of SEQ ID NO:1, (c) a valine at a position corresponding to T593 of the VP1 capsid polypeptide of SEQ ID NO:1, (d) an alanine at a position corresponding to W595 of the VP1 capsid polypeptide of SEQ ID NO:1, (e) a leucine at a position corresponding to V596 of the VP1 capsid polypeptide of SEQ ID NO:1, (f) a serine at a position corresponding to N598 of the VP1 capsid polypeptide of SEQ ID NO:1, and (g) an alanine at a position corresponding to I601 of the VP1 capsid polypeptide of SEQ ID NO:1.

In some embodiments, the capsid polypeptide comprises a mutation at S483 (e.g., S483F) and further comprises one, two, three, four, five, six, or all seven mutations selected from (a) a valine at a position corresponding to Q579 of the VP1 capsid polypeptide of SEQ ID NO:1, (b) an isoleucine at a position corresponding to Q592 of the VP1 capsid polypeptide of SEQ ID NO:1, (c) a valine at a position corresponding to T593 of the VP1 capsid polypeptide of SEQ ID NO:1, (d) an alanine at a position corresponding to W595 of the VP1 capsid polypeptide of SEQ ID NO:1, (e) a leucine at a position corresponding to V596 of the VP1 capsid polypeptide of SEQ ID NO:1, (f) a serine at a position corresponding to N598 of the VP1 capsid polypeptide of SEQ ID NO:1, and (g) an alanine at a position corresponding to I601 of the VP1 capsid polypeptide of SEQ ID NO:1. In some embodiments, the capsid polypeptide comprises a mutation at S483 (e.g., S483F) and further comprises a serine at a position corresponding to N598 of the VP1 capsid polypeptide of SEQ ID NO:1.

In some embodiments, the percentage sequence identity is calculated excluding any targeting peptide sequence insertion(s) in the capsid polypeptide sequence. In other embodiments, the percentage sequence identity is calculated including any targeting peptide sequence insertion(s) in the capsid polypeptide sequence.

Additional exemplary capsid polypeptides are disclosed in Section 6.2 and numbered embodiments 1 to 189.

The present disclosure further provides a nucleic acid comprising a nucleotide sequence encoding a capsid polypeptide as provided for herein, e.g., a capsid polypeptide disclosed in Section 6.2 or any one of numbered embodiments 1 to 189. In some embodiments, the nucleic acid molecule comprises a nucleotide sequence of SEQ ID NO:13, 27, 29, 31, 33, 35, 37 or 39, a fragment thereof (e.g., a fragment thereof encoding a VP2 or VP3 polypeptide), or a variant of any of the foregoing having at least 70%, 75%, 80%, 85%, 90%, 91%, 92%, 93%, 94%, 95%, 96%, 97%, 98%, 99%, or 100% sequence identity thereto. In some embodiments, the percentage sequence identity is calculated excluding any nucleotide sequence(s) encoding targeting peptide sequence insertion(s). In some embodiments, the percentage sequence identity is calculated including any nucleotide sequence(s) encoding targeting peptide sequence insertion(s). In some embodiments, the nucleic acid is a vector, e.g., a plasmid. Exemplary nucleic acids are disclosed in Section 6.2 and numbered embodiments 190 to 192.

The present disclosure further provides a dependoparvovirus particle comprising a capsid polypeptide, a capsid polypeptide disclosed in Section 6.2 or any one of numbered embodiments 1 to 189 and/or a nucleic acid described herein, e.g., a nucleic acid disclosed in Section 6.2 or any one of numbered embodiments 190 to 192 or a nucleic acid comprising a transgene as disclosed in Section 6.6.1. In some embodiments, the dependoparvovirus is an adeno-associated dependoparvovirus (AAV). In some embodiments, the AAV is AAV9, e.g., a variant AAV9. Exemplary virus particles are disclosed in Section 6.3 and numbered embodiments 193 to 221. In some embodiments, the virus particles have one or more characteristics disclosed in Section 6.4 and numbered embodiments 193 to 221.

In some embodiments, the disclosure is directed, in part, to a cell, cell-free system, or other translation system comprising a nucleic acid or vector described herein, e.g., comprising a sequence encoding a capsid polypeptide having one or more mutations described herein, for example a capsid polypeptide disclosed in Section 6.2 or any one of numbered embodiments 1 to 189. In some embodiments, the cell, cell-free system, or other translation system comprises a dependoparvovirus particle described herein, e.g., wherein the particle comprises a nucleic acid comprising a sequence encoding a capsid polypeptide, e.g., a capsid polypeptide disclosed in Section 6.2 or any one of numbered embodiments 1 to 189 and/or a nucleic acid described herein, e.g., a nucleic acid disclosed in Section 6.2 or any one of numbered embodiments 190 to 192 or a nucleic acid comprising a transgene as disclosed in Section 6.6.1. Exemplary cells, cell-free and other translation systems and their use to produce dependoparvovirus particles are disclosed in Section 6.5 and in numbered embodiments 1172 to 1181.

The present disclosure further provides methods of using a dependoparvovirus disclosed herein, e.g., for delivering a payload to a cell or treating a disease or condition in a subject. The methods typically comprise contacting the cell or administering to the subject a dependoparvovirus particle described herein in an amount effective to treat the disease or condition. Exemplary methods are disclosed in Section 6.6 and numbered embodiments 222 to 1171. The dependoparvovirus particles may be in the form of a composition, e.g., a pharmaceutical composition comprising the dependoparvovirus particles and a pharmaceutically acceptable carrier or excipient, for example as described in Section 6.6.2 and numbered embodiment 1182.

Additional features, advantages and applications of the capsid polypeptides, nucleic acids, dependoparvovirus particles of the disclosure and methods of their production and use are more particularly described below.

### 5. BRIEF DESCRIPTION OF THE DRAWINGS

FIGS. 1A-1C. Illustration of exemplary AAV serotype alignments. Amino acids that are present only in VP1 polypeptides are in normal text; amino acids that are present only in VP1 and VP2 polypeptides are in bold; amino acids that are present in VP1, VP2 and VP3 polypeptides are underlined. Figures disclose SEQ ID NOS 5, 3, 1, 7, and 9, respectively, in order of appearance.

FIGS. 2A-2D. AAV9 capsid protein structure visualization (pdb structure 7MT0). FIG. 2A shows a full view of the AAV9 capsid. FIG. 2B shows a side view of a portion of the AAV9 capsid at pH 7.4, highlighting a luminal pocket directly under the 3-fold symmetry axis (dark color). FIG. 2C shows an alternate side view of this luminal pocket, highlighting the locations of the S483 and N598 positions of the AAV9 capsid. FIG. 2D shows a top view of this luminal pocket, highlighting the locations of the S483 and N598 positions of the AAV9 capsid. In all of FIGS. 2A-2D, the scale bar=50 angstroms.

### 6. DETAILED DESCRIPTION

### 6.1. Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

A, An, The: As used herein, the singular forms “a,” “an” and “the” include plural referents unless the context clearly dictates otherwise.

About, Approximately: As used herein, the terms “about” and “approximately” shall generally mean an acceptable degree of error for the quantity measured given the nature or precision of the measurements. Exemplary degrees of error are within 15 percent (%), typically, within 10%, and more typically, within 5% of a given value or range of values. Any disclosure herein of a value preceded by the term “about” or “approximately” is also a disclosure of the value per se. For example, disclosure of “about 10 μg/ml” is a disclosure of the value “10 μg/ml.”

CNS: As used herein, “CNS” means one or more regions of the central nervous system. In embodiments, the CNS includes one or more of: brain and spinal cord.

Corresponds to: As used herein, the term “corresponds to” as used in reference to a position in a sequence, such as an amino acid or nucleic acid sequence, can be used in reference to an entire capsid polypeptide or polynucleotide sequence, such as the full-length sequence of the capsid polypeptide that comprises a VP1, VP2, and VP3 polypeptide, or a nucleic acid molecule encoding the same. In some embodiments, the term “corresponds to” can be used in reference to a region or domain of the capsid polypeptide. For example, a position that corresponds to a position in the VP1 section of the reference capsid polypeptide can correspond to the VP1 portion of the polypeptide of the variant capsid polypeptide. Thus, when aligning the two sequences to determine whether a position corresponds to another position the full-length polypeptide can be used or domains (regions) can be used to determine whether a position corresponds to a specific position. In some embodiments, the region is the VP1 polypeptide. In some embodiments, the region is the VP2 polypeptide. In some embodiments, the region is the VP3 polypeptide. In some embodiments, when the reference polypeptide is the wild-type sequence (e.g., full-length or region) of a certain serotype of AAV, the variant polypeptide can be of the same serotype with a mutation made at such corresponding position as compared to the reference sequence (e.g., full-length or region). In some embodiments, the variant capsid polypeptide is a different serotype as compared to the reference sequence.

Dependoparvovirus capsid: As used herein, the term “dependoparvovirus capsid” refers to an assembled viral capsid comprising dependoparvovirus polypeptides. In some embodiments, a dependoparvovirus capsid is a functional dependoparvovirus capsid, e.g., is fully folded and/or assembled, is competent to infect a target cell, or remains stable (e.g., folded/assembled and/or competent to infect a target cell) for at least a threshold time.

Dependoparvovirus particle: As used herein, the term “dependoparvovirus particle” refers to an assembled viral capsid comprising dependoparvovirus polypeptides and a packaged nucleic acid, e.g., comprising a payload, one or more components of a dependoparvovirus genome (e.g., a whole dependoparvovirus genome), or both. In some embodiments, a dependoparvovirus particle is a functional dependoparvovirus particle, e.g., comprises a desired payload, is fully folded and/or assembled, is competent to infect a target cell, or remains stable (e.g., folded/assembled and/or competent to infect a target cell) for at least a threshold time.

Dependoparvovirus X particle/capsid: As used herein, the term “dependoparvovirus X particle/capsid” refers to a dependoparvovirus particle/capsid comprising at least one polypeptide or polypeptide encoding nucleic acid sequence derived from a naturally occurring dependoparvovirus X species or serotype. For example, a dependoparvovirus B particle refers to a dependoparvovirus particle comprising at least one polypeptide or polypeptide encoding nucleic acid sequence derived from a naturally occurring dependoparvovirus B sequence. Derived from, as used in this context, means having at least 70, 75, 80, 85, 90, 91, 92, 93, 94, 95, 96, 97, 98, 99, or 100% identity to the sequence in question. Correspondingly, an AAVX particle/capsid, as used herein, refers to an AAV particle/capsid comprising at least one polypeptide or polypeptide encoding nucleic acid sequence derived from a naturally occurring AAV X serotype. For example, an AAV9 particle refers to an AAV particle comprising at least one polypeptide or polypeptide encoding nucleic acid sequence derived from a naturally occurring AAV9 sequence. Sometimes, a dependoparvovirus X capsid is referred to as “Wild Type” or “wt” when such capsid comprises capsid polypeptides from a specified sequence identifier associated with such dependoparvovirus X capsid. Thus, for example, the terms wild-type AAV9 capsid or wtAAV9 capsid (or simply wtAAV9) are used interchangeably and refer to a capsid that comprises capsid polypeptides of SEQ ID NO:1 (e.g., a VP1 capsid of SEQ ID NO:1 and VP2 and VP3 portions thereof).

Edit Distance: Sequences disclosed herein may be described in terms of “edit distance.” The minimum number of sequence edits, i.e., additions, substitutions, or deletions of a single amino acid (for amino acid sequence) or a single nucleotide (for nucleotide sequences), which change one sequence into another sequence is the edit distance between the two sequences. The term “edit distance” is often used interchangeably with the term “Levenshtein distance.”

Exogenous: As used herein, the term “exogenous” refers to a feature, sequence, or component present in a circumstance (e.g., in a nucleic acid, polypeptide, or cell) that does not naturally occur in said circumstance. For example, a nucleic acid sequence encoding a polypeptide can comprise an exogenous codon (e.g., codon encoding for an amino acid that does not naturally occur in that position, for example in a reference sequence), such as provided for herein. Use of the term exogenous in this fashion means that the codon in question at this position does not occur naturally, e.g., is not present in AAV9, e.g., is not present in SEQ ID NO:1. In some embodiments, the codon replaces an endogenous codon. In some embodiments, the exogenous codon is inserted into the nucleic acid sequence, for example, relative to a reference sequence. A person of skill will readily understand that a sequence (e.g., a codon) can be exogenous when provided in a particular sequence (e.g., that does not naturally comprise the codon at the site in question) but may not be exogenous in a second sequence (e.g., that does naturally comprise that particular codon at the site in question).

Functional: As used herein in reference to a polypeptide component of a dependoparvovirus capsid (e.g., Cap (e.g., VP1, VP2, and/or VP3) or Rep), the term “functional” refers to a polypeptide which provides at least 50, 60, 70, 80, 90, or 100% of the activity of a naturally occurring version of that polypeptide component (e.g., when present in a host cell). For example, a functional VP1 polypeptide can stably fold and assemble into a dependoparvovirus capsid (e.g., that is competent for packaging and/or secretion). As used herein in reference to a dependoparvovirus capsid or particle, “functional” refers to a capsid or particle comprising one or more of the following production characteristics: comprises a desired payload, is fully folded and/or assembled, is competent to infect a target cell, or remains stable (e.g., folded/assembled and/or competent to infect a target cell) for at least a threshold time.

Mutation Difference: As used herein with respect to a polypeptide sequence, means a single amino acid mutation (e.g., substitution, insertion or deletion) present in a subject polypeptide sequence, relative to a reference polypeptide sequence. In various embodiments, the reference polypeptide sequence is a polypeptide of any one of SEQ ID NO:1, SEQ ID NO:3, SEQ ID NO:7, SEQ ID NO:9, SEQ ID NO:11, SEQ ID NO:12, and SEQ ID NO:14 or a VP2 or VP3 portion thereof. In some embodiments, the reference polypeptide is any one of SEQ ID NO:1, SEQ ID NO:12, SEQ ID NO:14, SEQ ID NO:26, SEQ ID NO:28, SEQ ID NO:30, SEQ ID NO:32, SEQ ID NO:34, SEQ ID NO:36, or SEQ ID NO:38, or a VP2 or VP3 portion thereof. In a preferred embodiment, the reference polypeptide is a polypeptide of SEQ ID NO:1. In various embodiments, the subject polypeptide is SEQ ID NO:12, or a VP2 or VP3 portion thereof. In various embodiments, the subject polypeptide is SEQ ID NO:26, or a VP2 or VP3 portion thereof.

Mutation Set: As used herein, the term “mutation set” refers to the complete set of single amino acid mutations (substitutions, deletions and/or insertions) in a variant capsid polypeptide sequence (e.g., a polypeptide sequence of SEQ ID NO:12, SEQ ID NO:26, SEQ ID NO:28, SEQ ID NO:30, SEQ ID NO:32, SEQ ID NO:34, SEQ ID NO:36, or SEQ ID NO:38 or a VP2 or VP3 portion thereof) relative to a reference sequence (e.g., a wild-type reference sequence). In some embodiments, the reference sequence is wild-type AAV9 VP1 capsid polypeptide (SEQ ID NO:1) or a VP2 or VP3 portion thereof. In some cases, part of the mutation set (i.e., more than one single amino acid mutation) is notated collectively, however, it will be understood that even when referred to in this way, the mutation set is a collection of single amino acid mutations. For example, an insertion of amino acid 1, 2, and 3 between amino acid N at position nn and amino acid W at position ww of a reference sequence may be notated as “Nnn_3aa_Www_123,” and it will be understood that each of amino acids 1, 2 and 3 represent separate single amino acid mutations within the mutation set. In some embodiments, a variant capsid polypeptide of the disclosure comprises a mutation set not consisting solely of a mutation set present in a capsid polypeptide of SEQ ID NO:12.

Nucleic Acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid monomer (e.g., a nucleotide and/or nucleoside); in some embodiments, “nucleic acid” refers to an oligonucleotide chain comprising individual nucleic acid monomers or a longer polynucleotide chain comprising many individual nucleic acid monomers. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid is, comprises, or consists of one or more modified, synthetic, or non-naturally occurring nucleotides. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid has a nucleotide sequence that encodes a functional gene product such as an RNA or protein. In some embodiments, a nucleic acid is partly or wholly single stranded; in some embodiments, a nucleic acid is partly or wholly double stranded.

Or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Percent Identity: Sequences disclosed herein may be described in terms of “percent identity” (% identity). For calculating percent identity between two amino acid sequences or two nucleic acid sequences, the two sequences to be compared are aligned using the EMBOSS Needle Pairwise Sequence Alignment software tool based on the Needleman and Wunsch algorithm (Needleman & Wunsch, 1970, J. Mol. Biol. 48(3):443-53) (available at www.ebi.ac.uk/Tools/psa/emboss_needle/) using the following parameters: Matrix: BLOSUM62 (for amino acid sequences) or DNAfull (for DNA sequences); Gap Open: 10; Gap Extend: 0.5; End Gap Penalty: false; End Gap Open: 10; and End Gap Extend: 0.5. Percent identity is determined by dividing the number of amino acid or nucleotide matches in the alignment by the length of the alignment and multiplying by 100. For example, if an alignment of two amino acid sequences has 95 matching amino acids and an alignment length of 100 amino acids, the two sequences have 95% identity.

When calculating percent identity of two capsid polypeptides, one or both of which contain(s) one or more targeting peptide insertions, percent identity can be determined without removing the targeting peptide insertion sequence(s) from the capsid polypeptide sequence(s) or, alternatively, percent identity can be determined after removing the targeting peptide insertion sequence(s) from the capsid polypeptide sequence(s). For example, if a first capsid polypeptide has an identical sequence to a second capsid polypeptide, except that the first capsid polypeptide has a 7-mer targeting peptide insertion, the two capsid polypeptides have less than 100% sequence identity when percent identity is determined without removal of the targeting peptide insertion sequence from the first capsid polypeptide sequence, whereas the two capsid polypeptides have 100% sequence identity when the targeting peptide insertion sequence is removed from the first capsid polypeptide sequence prior to calculating percent identity. References herein to percent identity of capsid polypeptides without mention of a targeting peptide refer to percent identity of the capsid polypeptides determined following removal of targeting polypeptide insertion sequence(s), if any, present in both capsid polypeptides, unless required otherwise by context. References herein to percent identity calculated “taking targeting peptide insertions into account” means that the percent identity is calculated without removal of targeting polypeptide insertion sequence(s), if any, present in both capsid polypeptides. References herein to percent identity calculated “without taking targeting peptide insertions into account” means that the percent identity is calculated following removal of targeting polypeptide insertion sequence(s), if any, present in both capsid polypeptides.

PNS: as used herein, “PNS” means one or more regions of the peripheral nervous system that does not include the CNS. In embodiments, the PNS includes dorsal root ganglia. In embodiments, the PNS includes sensory neurons and motor neurons.

Polypeptide, peptide, and protein: The terms “polypeptide,” “peptide” and “protein” are used interchangeably herein to refer to polymers of amino acids of any length. The polymer may be linear or branched, it may comprise modified amino acids, and it may be interrupted by non-amino acids.

Targeting Peptide: As used herein, a “targeting peptide” refers to a peptide inserted into, or attached to, a capsid polypeptide to alter the tropism of the capsid polypeptide. A targeting peptide can be inserted into an AAV capsid sequence for enhanced targeting to a desired cell-type, tissue, or organ, for example for enhanced targeting to the CNS. A targeting peptide is typically 3 to 20 amino acids in length, for example, 3 to 12 amino acids, 5 to 12 amino acids, 5 to 10 amino acids, or 7 to 10 amino acids in length.

Treating: As used herein, the term “treating a disease or condition” refers to treating a manifest disease or condition, for example, where the subject is already suffering from one or more symptoms of the disease or condition, or refers to treating a pre-manifest disease or condition, for example, where the subject is identified as having a disease or condition but is not yet exhibiting one or more symptoms of the disease or condition. Pre-manifest conditions may be identified by, for example, genetic testing.

Variant: As used herein, a “variant capsid polypeptide” refers to a polypeptide that differs from a reference sequence (e.g., SEQ ID NO:1, SEQ ID NO:3, SEQ ID NO:7, SEQ ID NO:9, or SEQ ID NO:11, preferably SEQ ID NO:1, or sequence subunit thereof such as a VP2 or VP3 portion thereof). The variant capsid polypeptide can, for example, comprise a mutation (e.g., substitution, deletion, or insertion). In some embodiments, the variant is about, or at least, 70%, 75%, 80%, 85%, 86%, 87%, 88%, 89%, 90%, 91%, 92%, 93%, 94%, 95%, 96%, 97%, 98%, or 99% identical to the reference sequence. It will be clear to the skilled artisan from this disclosure that any capsid polypeptide, for example any capsid polypeptide disclosed herein, for example, a capsid polypeptide of SEQ ID NO:12, is a variant capsid polypeptide with respect to another capsid polypeptide having a different amino acid sequence, e.g., another capsid polypeptide with a reference sequence as set forth above. Thus, the term “variant capsid polypeptide” herein means, and is used interchangeably with, “capsid polypeptide,” and does not require any comparison to a specific reference sequence. In some embodiments, the reference sequence is a polypeptide comprising SEQ ID NO:1. In some embodiments, the reference sequence comprises or consists of a VP1, VP2 or VP3 polypeptide, e.g., of SEQ ID NO:1. In some contexts used herein, the term “variant” refers to a virus particle that includes a variant capsid polypeptide, e.g., described herein.

### 6.2. Capsid Polypeptides and Nucleic Acids Encoding the Same

The disclosure is directed, in part, to a variant capsid polypeptide, and to a nucleic acid comprising a sequence encoding the variant capsid polypeptide, wherein the variant capsid polypeptide comprises a mutation (insertion, deletion, or substitution) as compared to the wild-type sequence. In some embodiments, the wild-type sequence is SEQ ID NO:1. The disclosure is directed, in part, to a variant capsid polypeptide comprising SEQ ID NO:1 with one or more mutations as compared to SEQ ID NO:1, and nucleic acid molecules encoding the variant capsid polypeptide. The mutation can be, for example, an insertion, deletion, or substitution as compared to the wild-type sequence. In some embodiments, the wild-type sequence is SEQ ID NO:1.

In some embodiments, the variant capsid polypeptide comprises a mutation that corresponds to a position of a mutation present in SEQ ID NO:12, 26, 28, 30, 32, 34, 36 or 38 as compared to SEQ ID NO:1.

In some embodiments, the disclosure provides a variant capsid polypeptide (and nucleic acids encoding said variant capsid polypeptide) that comprises at least 1 of the mutation differences associated with any variant capsid polypeptide of SEQ ID NO:12, 26, 28, 30, 32, 34, 36 or 38 or comprises at least 1 mutation which corresponds to a mutation difference associated with the variant capsid polypeptide of SEQ ID NO:12, 26, 28, 30, 32, 34, 36 or 38. In some embodiments, the disclosure provides a variant capsid polypeptide (and nucleic acids encoding said capsid polypeptide) that comprises at least 2 mutation differences associated with the variant capsid polypeptide of SEQ ID NO:12, 26, 28, 30, 32, 34, 36 or 38 or comprises at least 2 mutations which corresponds to 2 mutation differences associated with the variant capsid polypeptide of SEQ ID NO:12, 26, 28, 30, 32, 34, 36 or 38. In some embodiments, the disclosure provides a variant capsid polypeptide (and nucleic acids encoding said capsid polypeptide) that comprises at least 3 mutation differences associated with the variant capsid polypeptide of SEQ ID NO:12, 26, 28, 30, 32, 34, 36 or 38 or comprises at least 3 mutations which corresponds to 3 mutation differences associated with the variant capsid polypeptide of SEQ ID NO:12, 26, 28, 30, 32, 34, 36 or 38. In some embodiments, the disclosure provides a variant capsid polypeptide (and nucleic acids encoding said capsid polypeptide) that comprises at least 4 mutation differences associated with the variant capsid polypeptide of SEQ ID NO:12, 26, 28, 30, 32, 34, 36 or 38 or comprises at least 4 mutations which corresponds to 4 mutation differences associated with the variant capsid polypeptide of SEQ ID NO:12, 26, 28, 30, 32, 34, 36 or 38. In some embodiments, the disclosure provides a variant capsid polypeptide (and nucleic acids encoding said capsid polypeptide) that comprises at least 5 mutation differences associated with the variant capsid polypeptide of SEQ ID NO:12, 26, 28, 30, 32, 34, 36 or 38 or comprises at least 5 mutations which corresponds to 5 mutation differences associated with the variant capsid polypeptide of SEQ ID NO:12, 26, 28, 30, 32, 34, 36 or 38. In some embodiments, the disclosure provides a variant capsid polypeptide (and nucleic acids encoding said capsid polypeptide) that comprises at least 6 mutation differences associated with the variant capsid polypeptide of SEQ ID NO:12, 26, 28, 30, 32, 34, 36 or 38 or comprises at least 6 mutations which corresponds to 6 mutation differences associated with the variant capsid polypeptide of SEQ ID NO:12, 26, 28, 30, 32, 34, 36 or 38. In some embodiments, the disclosure provides a variant capsid polypeptide (and nucleic acids encoding said capsid polypeptide) that comprises at least 7 mutation differences associated with the variant capsid polypeptide of SEQ ID NO:12, 26, 28, 30, 32, 34, 36 or 38 or comprises at least 7 mutations which corresponds to 7 mutation differences associated with the variant capsid polypeptide of SEQ ID NO:12, 26, 28, 30, 32, 34, 36 or 38. In some embodiments, the disclosure provides a variant capsid polypeptide (and nucleic acids encoding said capsid polypeptide) that comprises at least 8 mutation differences associated with the variant capsid polypeptide of SEQ ID NO:12, 26, 28, 30, 32, 34, 36 or 38 or comprises at least 8 mutations which corresponds to 8 mutation differences associated with the variant capsid polypeptide of SEQ ID NO:12, 26, 28, 30, 32, 34, 36 or 38. In some embodiments, the disclosure provides a variant capsid polypeptide (and nucleic acids encoding said capsid polypeptide) that comprises at least 9 mutation differences associated with the variant capsid polypeptide of SEQ ID NO:12, 28, 32, or 34, or comprises at least 9 mutations which corresponds to 9 mutation differences associated with the variant capsid polypeptide of SEQ ID NO:12, 28, 32, or 34. In some embodiments, the disclosure provides a variant capsid polypeptide (and nucleic acids encoding said capsid polypeptide) that comprises at least 10 mutation differences associated with the variant capsid polypeptide of SEQ ID NO:12 or SEQ ID NO:32 or comprises at least 10 mutations which corresponds to 10 mutation differences associated with the variant capsid polypeptide of SEQ ID NO:12 or SEQ ID NO:32.

Mutations associated with V1-V8 (corresponding to capsid polypeptides of SEQ ID NOs:12, 26, 28, 30, 32, 34, 36 and 38, respectively) are shown in Table 1 in relation to a VP1 polypeptide of SEQ ID NO:1.

TABLE 1

| | | | | | | | | | | | | Edit |
|---|---|---|---|---|---|---|---|---|---|---|---|---|
| | | | | | | | | | | | | distance |
| Capsid | | | | | | | | | | | | to SEQ |
| Polypeptide | A472 | V473 | S483 | T492 | Q579 | Q592 | T593 | W595 | V596 | N598 | I601 | ID NO: 1 |
| V1 | S | A | F | S | V | I | V | A | L | S | A | 11 |
| V2 | | | F | | V | I | V | A | L | S | A | 8 |
| V3 | | A | | S | V | I | V | A | L | S | A | 9 |
| V4 | | A | | | V | I | V | A | L | S | A | 8 |
| V5 | S | A | | S | V | I | V | A | L | S | A | 10 |
| V6 | S | A | | | V | I | V | A | L | S | A | 9 |
| V7 | | | | S | V | I | V | A | L | S | A | 8 |
| V8 | S | | | | V | I | V | A | L | S | A | 8 |

In some embodiments, the disclosure provides a variant capsid polypeptide (and nucleic acids encoding said capsid polypeptide) that comprises all of the mutation differences associated with the variant capsid polypeptide of SEQ ID NO:12, 26, 28, 30, 32, 34, 36 or 38 or comprises mutations which correspond to all of the mutation differences associated with the variant capsid polypeptide of SEQ ID NO:12, 26, 28, 30, 32, 34, 36 or 38.

In any of the above aspects it will be understood that in variant capsid polypeptides described above where a number of mutation differences associated with or corresponding to the mutation differences of the variant capsid polypeptide of SEQ ID NO:12, 26, 28, 30, 32, 34, 36 or 38 is specified, the mutations may be chosen from any of the mutation differences associated with that variant capsid polypeptide. Thus, for example, with respect to the mutation differences of a variant having mutation differences #1, #2, #3, and #4 relative to a reference polypeptide, where a variant capsid polypeptide comprises 1 of the mutation differences, it is #1 or #2 or #3 or #4; likewise, where a variant capsid comprises 2 of the mutation differences, those two are #1 and #2, #1 and #3, #1 and #4, #2 and #3, #2 and #4, or #3 and #4; likewise, where the variant comprises 3 of the mutation differences, those 3 are #1 and #2 and #3, #1 and #2 and #4, #1 and #3 and #4, or #2 and #3 and #4; likewise, where the variant comprises all 4 of the mutation differences, those four are #1, #2, #3 and #4. It will be understood by the skilled artisan that all the possible combinations of numbers of mutation differences for the variant capsid polypeptide of SEQ ID NO:12, 26, 28, 30, 32, 34, 36 or 38 (up to the total number of mutation differences for that variant capsid polypeptide) can be generated using routine skill and such a table for SEQ ID NO:12, 26, 28, 30, 32, 34, 36 or 38 is incorporated herein in its entirety. Such tables can be generated, for example, using the “combinations” method from the “itertools” package in Python, such method is hereby incorporated by reference in its entirety.

In some embodiments, the variant capsid polypeptide comprises one or more mutation differences associated with a capsid polypeptide described herein, e.g., associated with SEQ ID NO:12, 26, 28, 30, 32, 34, 36 or 38, and has at least 80%, at least 85%, at least 90%, at least 91%, at least 92%, at least 93%, at least 94%, at least 95%, at least 96%, at least 97%, at least 98%, or at least 99% sequence identity to a reference AAV serotype, e.g., as described herein, e.g., to SEQ ID NO:1. In embodiments, the variant capsid polypeptide comprises one or more mutation differences associated with a capsid polypeptide described herein, e.g., associated with SEQ ID NO:12, 26, 28, 30, 32, 34, 36 or 38 or which correspond to one or more mutation differences associated with a capsid polypeptide described herein, e.g., associated with SEQ ID NO:12, 26, 28, 30, 32, 34, 36 or 38. In embodiments, the variant capsid polypeptide is, but for the mutation differences described in or corresponding to the mutation differences associated with a capsid polypeptide described herein, e.g., associated with SEQ ID NO:12, 26, 28, 30, 32, 34, 36 or 38, at least 90%, at least 95%, 96%, 97%, 98%, 99%, or 100% identical to a reference AAV serotype described herein. In embodiments, the variant capsid polypeptide described herein is, but for the mutation differences associated with a capsid polypeptide described herein, e.g., associated with SEQ ID NO:12, 26, 28, 30, 32, 34, 36 or 38, or which correspond to the mutation differences associated with a capsid polypeptide described herein, e.g., associated with SEQ ID NO:12, 26, 28, 30, 32, 34, 36 or 38, comprised within such variant capsid polypeptide, at least 90%, at least 95%, 96%, 97%, 98%, 99%, or 100% identical to a capsid polypeptide of SEQ ID NO:1 (e.g., a VP1, VP2 or VP3 sequence of SEQ ID NO:1). In embodiments, the variant capsid polypeptide described herein is, but for the mutation differences associated with a capsid polypeptide described herein, e.g., associated with SEQ ID NO:12, 26, 28, 30, 32, 34, 36 or 38, or which correspond to the mutation differences associated with a capsid polypeptide described herein, e.g., associated with SEQ ID NO:12, 26, 28, 30, 32, 34, 36 or 38 comprised within such variant capsid polypeptide, at least 90%, at least 95%, 96%, 97%, 98%, 99%, or 100% identical to a capsid polypeptide of SEQ ID NO:3 (e.g., a VP1, VP2 or VP3 sequence of SEQ ID NO:3). In embodiments, the variant capsid polypeptide described herein is, but for the mutation differences associated with SEQ ID NO:12, 26, 28, 30, 32, 34, 36 or 38, or which correspond to the mutation differences associated with SEQ ID NO:12, 26, 28, 30, 32, 34, 36 or 38, comprised within such variant capsid polypeptide, at least 90%, at least 95%, 96%, 97%, 98%, 99%, or 100% identical to a capsid polypeptide of SEQ ID NO:5 (e.g., a VP1, VP2 or VP3 sequence of SEQ ID NO:5). In embodiments, the variant capsid polypeptide described herein is, but for the mutation differences associated with SEQ ID NO:12, 26, 28, 30, 32, 34, 36 or 38, or which correspond to the mutation differences associated with SEQ ID NO:12, 26, 28, 30, 32, 34, 36 or 38, comprised within such variant capsid polypeptide, at least 90%, at least 95%, 96%, 97%, 98%, 99%, or 100% identical to a capsid polypeptide of SEQ ID NO:7 (e.g., a VP1, VP2 or VP3 sequence of SEQ ID NO:7). In embodiments, the variant capsid polypeptide described herein is, but for the mutation differences associated with SEQ ID NO:12, 26, 28, 30, 32, 34, 36 or 38, or which correspond to the mutation differences associated with SEQ ID NO:12, 26, 28, 30, 32, 34, 36 or 38, comprised within such variant capsid polypeptide, at least 90%, at least 95%, 96%, 97%, 98%, 99%, or 100% identical to a capsid polypeptide of SEQ ID NO:9 (e.g., a VP1, VP2 or VP3 sequence of SEQ ID NO:9). In embodiments, the variant capsid polypeptide described herein is, but for the mutation differences associated with SEQ ID NO:12, 26, 28, 30, 32, 34, 36 or 38, or which correspond to the mutation differences associated with SEQ ID NO:12, 26, 28, 30, 32, 34, 36 or 38, comprised within such variant capsid polypeptide, at least 90%, at least 95%, 96%, 97%, 98%, 99%, or 100% identical to a capsid polypeptide of SEQ ID NO:11 (e.g., a VP1, VP2 or VP3 sequence of SEQ ID NO:11).

In some embodiments, a variant capsid polypeptide is provided that comprises a variant capsid polypeptide that is at least 70%, 75%, 80%, 85%, 90%, 91%, 92%, 93%, 94%, 95%, 96%, 97%, 98%, 99%, or 100% identical to a variant capsid polypeptide as provided herein.

In some embodiments, a variant capsid polypeptide is provided that comprises a variant capsid polypeptide that is at least 70%, 75%, 80%, 85%, 90%, 91%, 92%, 93%, 94%, 95%, 96%, 97%, 98%, 99%, or 100% identical (not including targeting peptide insertions) to a variant capsid polypeptide as provided herein.

In some embodiments, the variant capsid polypeptide comprises a VP1, VP2 VP3, or any combination thereof, that is each at least, or about, 95%, 96%, 97%, 98% or 99% identical to a polypeptide of SEQ ID NO:12, 26, 28, 30, 32, 34, 36 or 38, and optionally comprises at least one of, e.g., all of, the mutation differences of such SEQ ID NO:12, 26, 28, 30, 32, 34, 36 or 38.

In some embodiments, the variant capsid polypeptide comprises a VP1, VP2 VP3, or any combination thereof, that is each at least, or about, 95%, 96%, 97%, 98% or 99% identical (not including target peptide insertions) to a polypeptide of SEQ ID NO:12, 26, 28, 30, 32, 34, 36 or 38, and optionally comprises at least one of, e.g., all of, the mutation differences of such SEQ ID NO:12, 26, 28, 30, 32, 34, 36 or 38.

In some embodiments, the variant capsid polypeptide comprises a VP1, VP2, VP3, or any combination thereof, that each has about 1 to about 20 mutations as compared to a polypeptide of SEQ ID NO:12, 26, 28, 30, 32, 34, 36 or 38, and optionally comprises at least one of, e.g., all of, the mutation differences of such SEQ ID NO:12, 26, 28, 30, 32, 34, 36 or 38.

In some embodiments, the variant capsid polypeptide comprises a VP1, VP2, VP3, or any combination thereof, that each has about 1 to about 10 mutations as compared to a polypeptide of SEQ ID NO:12, 26, 28, 30, 32, 34, 36 or 38, and optionally comprises at least one of, e.g., all of, the mutation differences of such SEQ ID NO:12, 26, 28, 30, 32, 34, 36 or 38.

In some embodiments, the variant capsid polypeptide comprises a VP1, VP2, VP3, or any combination thereof, that each has 1 to 5 mutations as compared to a polypeptide of SEQ ID NO:12, 26, 28, 30, 32, 34, 36 or 38, and optionally comprises at least one of, e.g., all of, the mutation differences of such SEQ ID NO:12, 26, 28, 30, 32, 34, 36 or 38.

In aspects, provided herein are nucleic acid molecules encoding a variant capsid polypeptide as provided herein. In aspects, the nucleic acid molecule comprises sequence encoding a variant capsid polypeptide (e.g., a VP1, VP2 or VP3 capsid polypeptide) of SEQ ID NO:12, 26, 28, 30, 32, 34, 36 or 38, or fragment thereof. In aspects, the nucleic acid molecule comprises SEQ ID NO:13, 27, 29, 31, 33, 35, 37 or 39, or fragment thereof (e.g., a VP1-encoding, VP2-encoding or VP3-encoding fragment thereof).

In some embodiments, the nucleic acid molecule encodes a variant capsid polypeptide that is at least 70%, 75%, 80%, 85%, 90%, 91%, 92%, 93%, 94%, 95%, 96%, 97%, 98%, 99%, or 100% identical to a variant capsid polypeptide as provided herein.

In some embodiments, the nucleic acid molecule or the nucleic acid molecule encoding the reference polypeptide for purposes of % identity, comprises a nucleotide sequence of SEQ ID NO:13. In some embodiments, the nucleic acid molecule or the nucleic acid molecule encoding the reference polypeptide for purposes of % identity, comprises a nucleotide sequence of SEQ ID NO:27. In some embodiments, the nucleic acid molecule or the nucleic acid molecule encoding the reference polypeptide for purposes of % identity, comprises a nucleotide sequence of SEQ ID NO:29. In some embodiments, the nucleic acid molecule or the nucleic acid molecule encoding the reference polypeptide for purposes of % identity, comprises a nucleotide sequence of SEQ ID NO:31. In some embodiments, the nucleic acid molecule or the nucleic acid molecule encoding the reference polypeptide for purposes of % identity, comprises a nucleotide sequence of SEQ ID NO:33. In some embodiments, the nucleic acid molecule or the nucleic acid molecule encoding the reference polypeptide for purposes of % identity, comprises a nucleotide sequence of SEQ ID NO:35. In some embodiments, the nucleic acid molecule or the nucleic acid molecule encoding the reference polypeptide for purposes of % identity, comprises a nucleotide sequence of SEQ ID NO:37. In some embodiments, the nucleic acid molecule or the nucleic acid molecule encoding the reference polypeptide for purposes of % identity, comprises a nucleotide sequence of SEQ ID NO:39.

In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes SEQ ID NO:12. In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes SEQ ID NO:26. In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes SEQ ID NO:28. In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes SEQ ID NO:30. In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes SEQ ID NO:32. In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes SEQ ID NO:34. In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes SEQ ID NO:36. In some embodiments, the nucleic acid molecule or the nucleic acid molecule encoding the reference polypeptide for purposes of % identity, comprises a nucleotide sequence that encodes a sequence of a variant capsid polypeptide, e.g., as described herein, e.g., encodes SEQ ID NO:38.

In some embodiments, the variant capsid polypeptide, or the reference polypeptide for purposes of % identity, comprises a sequence of SEQ ID NO:12, that is encoded by a nucleotide sequence of SEQ ID NO:13, respectively. In some embodiments, the variant capsid polypeptide, or the reference polypeptide for purposes of % identity, comprises a sequence of SEQ ID NO:26, that is encoded by a nucleotide sequence of SEQ ID NO:27, respectively. In some embodiments, the variant capsid polypeptide, or the reference polypeptide for purposes of % identity, comprises a sequence of SEQ ID NO:28, that is encoded by a nucleotide sequence of SEQ ID NO:29, respectively. In some embodiments, the variant capsid polypeptide, or the reference polypeptide for purposes of % identity, comprises a sequence of SEQ ID NO:30, that is encoded by a nucleotide sequence of SEQ ID NO:31, respectively. In some embodiments, the variant capsid polypeptide, or the reference polypeptide for purposes of % identity, comprises a sequence of SEQ ID NO:32, that is encoded by a nucleotide sequence of SEQ ID NO:33, respectively. In some embodiments, the variant capsid polypeptide, or the reference polypeptide for purposes of % identity, comprises a sequence of SEQ ID NO:34, that is encoded by a nucleotide sequence of SEQ ID NO:35, respectively. In some embodiments, the variant capsid polypeptide, or the reference polypeptide for purposes of % identity, comprises a sequence of SEQ ID NO:36, that is encoded by a nucleotide sequence of SEQ ID NO:37, respectively. In some embodiments, the variant capsid polypeptide, or the reference polypeptide for purposes of % identity, comprises a sequence of SEQ ID NO:38, that is encoded by a nucleotide sequence of SEQ ID NO:39, respectively.

In some embodiments, the variant capsid polypeptide comprises a sequence that includes all of the mutation differences associated with SEQ ID NO:12, 26, 28, 30, 32, 34, 36 or 38, e.g., relative to SEQ ID NO:1.

In some embodiments, the variant capsid polypeptide is a VP1 capsid polypeptide. In some embodiments, the variant capsid polypeptide is a VP2 capsid polypeptide. In some embodiments, the variant capsid polypeptide is a VP3 capsid polypeptide. With respect to reference sequence SEQ ID NO:1, a VP1 capsid polypeptide comprises amino acids 1-737 of SEQ ID NO:1. With respect to reference sequence SEQ ID NO:1, a VP2 capsid polypeptide comprises amino acids 138-737 of SEQ ID NO:1. With respect to reference sequence SEQ ID NO:1, a VP3 capsid polypeptide comprises amino acids 203-737 of SEQ ID NO:1.

With respect to a variant capsid polypeptide sequence of SEQ ID NO:12, 26, 28, 30, 32, 34, 36 or 38, a VP1 capsid polypeptide comprises all of the amnio acids of SEQ ID NO:12, 26, 28, 30, 32, 34, 36 or 38. With respect to a sequence of SEQ ID NO:12, 26, 28, 30, 32, 34, 36 or 38, a VP2 capsid polypeptide comprises, e.g., consists of, a sequence that begins with the threonine corresponding to the threonine at position 138 of SEQ ID NO:1, and continuing to the C-terminus of SEQ ID NO:12, 26, 28, 30, 32, 34, 36 or 38. With respect to a sequence of SEQ ID NO:12, 26, 28, 30, 32, 34, 36 or 38, a VP3 capsid polypeptide comprises, e.g., consists of, a sequence that begins with the methionine corresponding to methionine at position 203 of SEQ ID NO:1 and continuing to the C-terminus of SEQ ID NO:12, 26, 28, 30, 32, 34, 36 or 38.

Exemplary sequences of variant capsid polypeptides are provided in SEQ ID NO:12, 26, 28, 30, 32, 34, 36 and 38 and exemplary nucleic acid molecules encoding the same are provided in SEQ ID NO:13, 27, 29, 31, 33, 35, 37 and 39.

In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity to a VP1, VP2, or VP3 sequence of SEQ ID NO:12. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity to a VP1, VP2, or VP3 sequence of SEQ ID NO:26. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity to a VP1, VP2, or VP3 sequence of SEQ ID NO:28. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity to a VP1, VP2, or VP3 sequence of SEQ ID NO:30. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity to a VP1, VP2, or VP3 sequence of SEQ ID NO:32. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity to a VP1, VP2, or VP3 sequence of SEQ ID NO:34. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity to a VP1, VP2, or VP3 sequence of SEQ ID NO:36. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity to a VP1, VP2, or VP3 sequence of SEQ ID NO:38.

In some embodiments, the nucleic acid molecule encodes a variant capsid polypeptide that has at least 70%, 75%, 80%, 85%, 90%, 91%, 92%, 93%, 94%, 95%, 96%, 97%, 98%, 98, 99%, or 100% identity (not including targeting peptide insertions) to a VP1, VP2, or VP3 sequence of SEQ ID NO:12. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity (not including targeting peptide insertions) to a VP1, VP2, or VP3 sequence of SEQ ID NO:26. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity (not including targeting peptide insertions) to a VP1, VP2, or VP3 sequence of SEQ ID NO:28. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity (not including targeting peptide insertions) to a VP1, VP2, or VP3 sequence of SEQ ID NO:30. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity (not including targeting peptide insertions) to a VP1, VP2, or VP3 sequence of SEQ ID NO:32. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity (not including targeting peptide insertions) to a VP1, VP2, or VP3 sequence of SEQ ID NO:34. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity (not including targeting peptide insertions) to a VP1, VP2, or VP3 sequence of SEQ ID NO:36. In some embodiments, the nucleic acid molecule encodes a variant capsid polypeptide that has at least 70%, 75%, 80%, 85%, 90%, 91%, 92%, 93%, 94%, 95%, 96%, 97%, 98%, 99%, or 100% identity (not including targeting peptide insertions) to a VP1, VP2, or VP3 sequence of SEQ ID NO:38.

### 6.2.1. Variant Capsid Polypeptides (Corresponding Positions)

The mutations to capsid polypeptide sequences described herein are described in relation to a position and/or amino acid at a position within a reference sequence, e.g., SEQ ID NO:1. Thus, in some embodiments, the capsid polypeptides described herein are variant capsid polypeptides of the reference sequence, e.g., SEQ ID NO:1, e.g., include capsid polypeptides comprising at least 80%, at least 85%, at least 90%, at least 91%, at least 92%, at least 93%, at least 94%, at least 95%, at least 96%, at least 97%, at least 98%, or at least 99% identity to the reference capsid polypeptide sequence (e.g., reference capsid polypeptide VP1, VP2 and/or VP3 sequence), e.g., SEQ ID NO:1 (or VP2 or VP3 sequence comprised therein) and include one or more mutations described herein.

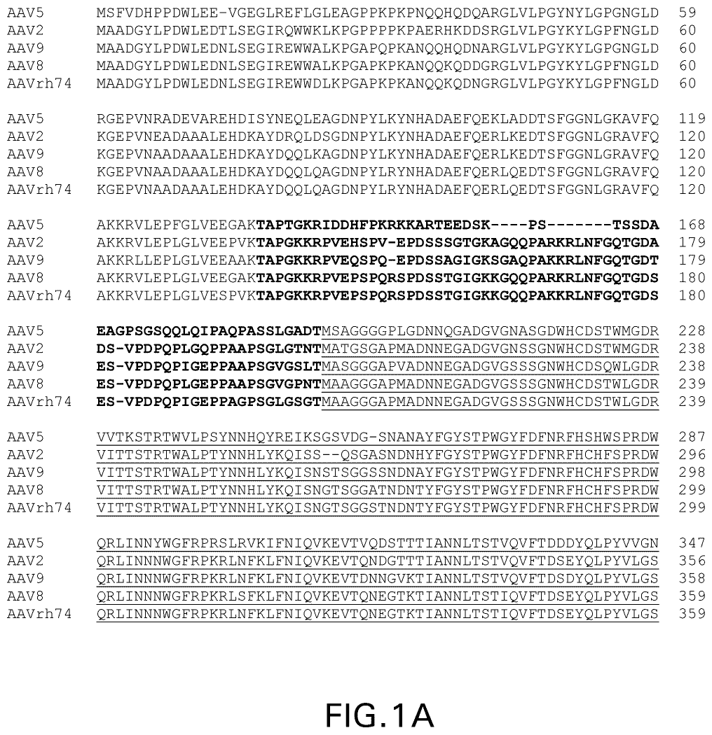

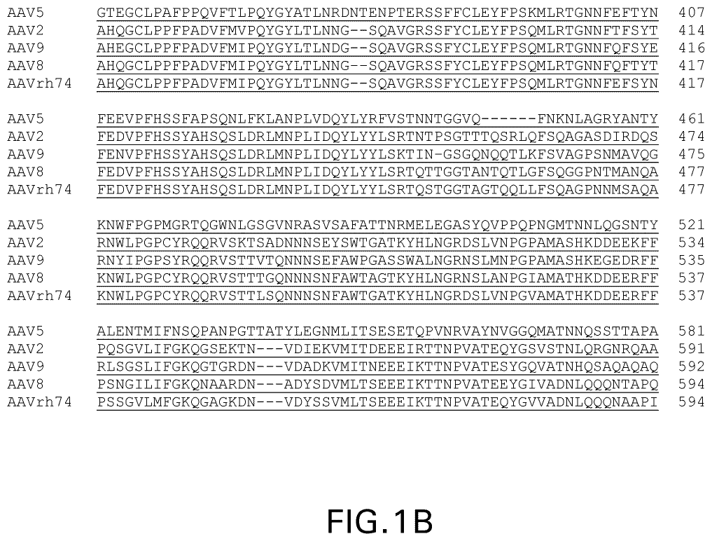

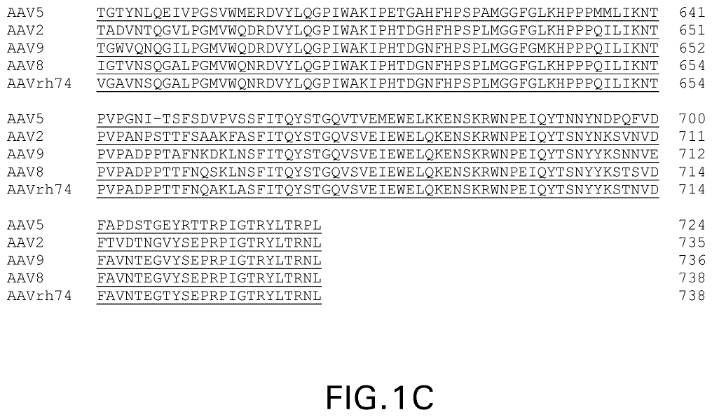

It will be understood by the skilled artisan, and without being bound by theory, that each amino acid position within a reference sequence corresponds to a position within the sequence of other reference capsid polypeptides such as capsid polypeptides derived from dependoparvoviruses with different serotypes. Such corresponding positions are identified using sequence alignment tools known in the art. A particularly preferred sequence alignment tool is EMBOSS Needle Pairwise Sequence Alignment software tool based on the Needleman and Wunsch algorithm (Needleman & Wunsch, 1970, J. Mol. Biol. 48(3):443-53) (available on the World Wide Web at ebi.ac.uk/Tools/psa/emboss_needle/). An alignment of exemplary reference capsid polypeptides is shown in FIGS. 1A-1C. Thus, in some embodiments, the variant capsid polypeptides of the invention include variants of reference capsid polypeptides that include one or more mutations described herein in such reference capsid polypeptides at positions corresponding to the position of the mutation described herein in relation to a different reference capsid polypeptide. Thus, for example, a mutation described as XnnnY relative to SEQ ID NO:1 (where X is the amino acid present at position nnn in SEQ ID NO:1 and Y is the amino acid mutation at that position, e.g., described herein), the disclosure provides variant capsid polypeptides comprising at least 70%, at least 75%, at least 80%, at least 85%, at least 90%, at least 91%, at least 92%, at least 93%, at least 94%, at least 95%, at least 96%, at least 97%, at least 98%, or at least 99% identity to a reference capsid polypeptide sequence (e.g., reference capsid polypeptide VP1, VP2 and/or VP3 sequence) other than SEQ ID NO:1 (or VP2 or VP3 sequence comprised therein) and further comprising the disclosed mutation at a position corresponding to position nnn of SEQ ID NO:1 (e.g., comprising Y at the position in the new variant capsid polypeptide sequence that corresponds to position nnn of SEQ ID NO:1). As described above, such corresponding position is determined using a sequence alignment tool, such as, for example, the clustal omega tool described above. Examples of corresponding amino acid positions of exemplary known AAV serotypes is provided in FIG. 1A-1C. In some embodiments, the variant is a variant of the AAV9 capsid polypeptide, which can be referred to as a “AAV9 variant capsid polypeptide” or “variant AAV9 capsid polypeptide.”

Thus, in embodiments, the disclosure provides variant capsid polypeptide sequences that are variants of a reference sequence other than SEQ ID NO:1, e.g., a reference sequence other than SEQ ID NO:1 as described herein, which include one or more mutation corresponding to the mutations described herein. In embodiments, such variants include mutations corresponding to all of the mutations associated with SEQ ID NO:12, 26, 28, 30, 32, 34, 36 or 38.

The variant capsid polypeptides described herein are optionally variants of reference capsids serotypes known in the art. Non-limiting examples of such reference AAV serotypes include AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.eB, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9K449R (or K449R AAV9), AAV9.16, AAV9.24, AAV9.45, AAbiodisV9.47, AAV9.61, AAV9.68, AAV9.84, AAV9.9, AAV10, AAV11, AAV12, AAV16.3, AAV24.1, AAV27.3, AAV42.12, AAV42-1 b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LKO2, AAV-LK03, AAV-LKO4, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof.

In some embodiments, the reference AAV capsid sequence comprises an AAV2 sequence. In some embodiments, the reference AAV capsid sequence comprises an AAV5 sequence. In some embodiments, the reference AAV capsid sequence comprises an AAV8 sequence. In some embodiments, the reference AAV capsid sequence comprises an AAV9 sequence. In some embodiments, the reference AAV capsid sequence comprises an AAVrh74 sequence. While not wishing to be bound by theory, it is understood that a reference AAV capsid sequence comprises a VP1 region. In certain embodiments, a reference AAV capsid sequence comprises a VP1, VP2 and/or VP3 region, or any combination thereof. A reference VP1 sequence may be considered synonymous with a reference AAV capsid sequence.

The wild-type reference sequence ofAAV9, SEQ ID NO:1 is as follows:

(SEQ ID NO: 1)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL

Unless otherwise noted, SEQ ID NO:1 is the reference sequence. In the sequence above, the sequence found in VP1, VP2 and VP3 is underlined (e.g., a VP3 capsid polypeptide includes, e.g., consists of, amino acids corresponding to amino acids 203-737 of SEQ ID NO:1), the sequence found in both VP1 and VP2 is in bold (e.g., a VP2 capsid polypeptide includes, e.g., consists of, the sequence corresponding to amino acids 138-737 of SEQ ID NO:1) and the sequence that is not underlined or bold is found only in VP1 (e.g., a VP1 capsid polypeptide includes, e.g., consists of, amino acids corresponding to amino acids 1-737 of SEQ ID NO:1).

The wild-type reference sequence of SEQ ID NO:1 can be encoded by a reference nucleic acid molecule sequence of SEQ ID NO:2.

An exemplary reference sequence of wild-type AAV2, SEQ ID NO:3 (wild-type AAV2) is as follows:

(SEQ ID NO: 3)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDINGVYSEPRPIGTRYLTRNL.

In the sequence above, the sequence found in VP1, VP2 and VP3 is underlined (e.g., a VP3 capsid polypeptide includes, e.g., consists of, amino acids corresponding to amino acids 203-735 of SEQ ID NO:3), the sequence found in both VP1 and VP2 is in bold (e.g., a VP2 capsid polypeptide includes, e.g., consists of, the sequence corresponding to amino acids 138-735 of SEQ ID NO:3) and the sequence that is not underlined or bold is found only in VP1 (e.g., a VP1 capsid polypeptide includes, e.g., consists of, amino acids corresponding to amino acids 1-735 of SEQ ID NO:3).

An example nucleic acid sequence encoding SEQ ID NO:3 is SEQ ID NO:4.

An exemplary reference sequence of wild type AAV5, SEQ ID NO:5 (wild-type AAV5), is as follows:

(SEQ ID NO: 5)
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDFNRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGTYNLQEIVPGSVWMERD
VYLQGPIWAKIPETGAHFHPSPAMGGFGLKHPPPMMLIKNTPVPGNITSF
SDVPVSSFITQYSTGQVTVEMEWELKKENSKRWNPEIQYTNNYNDPQFVD
FAPDSTGEYRTTRPIGTRYLTRPL

In the sequence above, the sequence found in VP1, VP2 and VP3 is underlined (e.g., a VP3 capsid polypeptide includes, e.g., consists of, amino acids corresponding to amino acids 193-725 of SEQ ID NO:5), the sequence found in both VP1 and VP2 is in bold (e.g., a VP2 capsid polypeptide includes, e.g., consists of, the sequence corresponding to amino acids 137-725 of SEQ ID NO:5) and the sequence that is not underlined or bold is found only in VP1 (e.g., a VP1 capsid polypeptide includes, e.g., consists of, amino acids corresponding to amino acids 1-725 of SEQ ID NO:5).

An example nucleic acid sequence encoding SEQ ID NO:5 is SEQ ID NO:6.

An exemplary reference sequence of wild-type AAV8, SEQ ID NO:7 (wild-type AAV8), is as follows:

(SEQ ID NO: 7)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGLVEEGAKTAPGKKRPVEPSP
QRSPDSSTGIGKKGQQPARKRLNFGQTGDSESVPDPQPLGEPPAAPSGVG
PNTMAAGGGAPMADNNEGADGVGSSSGNWHCDSTWLGDRVITTSTRTWAL
PTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFQFTYTFEDVPFHSSYAHSQSLDRLMNPLIDQYLYYLSR
TQTTGGTANTQTLGFSQGGPNTMANQAKNWLPGPCYRQQRVSTTTGQNNN
SNFAWTAGTKYHLNGRNSLANPGIAMATHKDDEERFFPSNGILIFGKQNA
ARDNADYSDVMLTSEEEIKTTNPVATEEYGIVADNLQQQNTAPQIGTVNS
QGALPGMVWQNRDVYLQGPIWAKIPHTDGNFHPSPLMGGFGLKHPPPQIL
IKNTPVPADPPTTFNQSKLNSFITQYSTGQVSVEIEWELQKENSKRWNPE
IQYTSNYYKSTSVDFAVNTEGVYSEPRPIGTRYLTRNL

In the sequence above, the sequence found in VP1, VP2 and VP3 is underlined (e.g., a VP3 capsid polypeptide includes, e.g., consists of, amino acids corresponding to amino acids 204-739 of SEQ ID NO:7), the sequence found in both VP1 and VP2 is in bold (e.g., a VP2 capsid polypeptide includes, e.g., consists of, the sequence corresponding to amino acids 138-735 of SEQ ID NO:7) and the sequence that is not underlined or bold is found only in VP1 (e.g., a VP1 capsid polypeptide includes, e.g., consists of, amino acids corresponding to amino acids 1-739 of SEQ ID NO:7).

An example nucleic acid sequence encoding SEQ ID NO:7 is SEQ ID NO:8.

An exemplary reference sequence of wild-type AAVrh74, SEQ ID NO:9 (wild-type AAVrh74), is as follows:

(SEQ ID NO: 9)
MAADGYLPDWLEDNLSEGIREWWDLKPGAPKPKANQQKQDNGRGLVLPGY
KYLGPFNGLDKGEPVNAADAAALEHDKAYDQQLQAGDNPYLRYNHADAEF
QERLQEDTSFGGNLGRAVFQAKKRVLEPLGLVESPVKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYNFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFNQAKLASFITQYSTGQVSVEIEWELQKENSKRWNPE
IQYTSNYYKSTNVDFAVNTEGTYSEPRPIGTRYLTRNL

An alternative exemplary reference sequence of SEQ ID NO:11 (alternate wild-type AAVrh74) is as follows:

(SEQ ID NO: 11)
MAADGYLPDWLEDNLSEGIREWWDLKPGAPKPKANQQKQDNGRGLVLPGY
KYLGPFNGLDKGEPVNAADAAALEHDKAYDQQLQAGDNPYLRYNHADAEF
QERLQEDTSFGGNLGRAVFQAKKRVLEPLGLVESPVKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYNFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFTKAKLASFITQYSTGQVSVEIEWELQKENSKRWNPE
IQYTSNYYKSTNVDFAVNTEGTYSEPRPIGTRYLTRNL

In the sequences above (SEQ ID NO:9 or SEQ ID NO:11), the sequence found in VP1, VP2 and VP3 is underlined (e.g., a VP3 capsid polypeptide includes, e.g., consists of, amino acids corresponding to amino acids 204-739 of SEQ ID NO:9), the sequence found in both VP1 and VP2 is in bold (e.g., a VP2 capsid polypeptide includes, e.g., consists of, the sequence corresponding to amino acids 137-739 of SEQ ID NO:9) and the sequence that is not underlined or bold is found only in VP1 (e.g., a VP1 capsid polypeptide includes, e.g., consists of, amino acids corresponding to amino acids 1-739 of SEQ ID NO:9).

An example nucleic acid sequence encoding SEQ ID NO:9 is SEQ ID NO:10.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins. Where the Met/AA-clipping is incomplete, a mixture of one or more (one, two or three) VP capsid proteins comprising the viral capsid can be produced, some of which include a Met1/AA1 amino acid (Met+/AA+) and some of which lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods.2017 Oct.28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety. According to the present disclosure, references to capsid polypeptides is not limited to either clipped (Met−/AA−) or unclipped (Met+/AA+) and, in context, also refer to independent capsid polypeptides, viral capsids comprised of a mixture of capsid proteins, and/or polynucleotide sequences (or fragments thereof) which encode, describe, produce or result in capsid polypeptides of the present disclosure. A direct reference to a “capsid polypeptide” (such as VP1, VP2 or VP3) also comprise VP capsid proteins which include a Met1/AA1 amino acid (Met+/AA+) as well as corresponding VP capsid polypeptide which lack the Met1/AA1 amino acid as a result of Met/AA-clipping (Met−/AA−). Further according to the present disclosure, a reference to a specific SEQ ID NO: (whether a protein or nucleic acid) which comprises or encodes, respectively, one or more capsid polypeptides which include a Met1/AA1 amino acid (Met+/AA+) should be understood to teach the VP capsid polypeptides which lack the Met1/AA1 amino acid as upon review of the sequence, it is readily apparent any sequence which merely lacks the first listed amino acid (whether or not Met1/AA1). As a non-limiting example, reference to a VP1 polypeptide sequence which is 736 amino acids in length and which includes a “Met1” amino acid (Met+) encoded by the AUG/ATG start codon is also understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon can also be understood to teach a VP1 polypeptide sequence which is 735 amino acids in length and which does not include the “AA1” amino acid (AA1−) of the 736 amino acid AA1+ sequence. References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−). As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In some embodiments, the reference AAV capsid sequence comprises an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above.

In some embodiments, the reference AAV capsid sequence is encoded by a nucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described above. In certain embodiments, the reference sequence is not an AAV capsid sequence and is instead a different vector (e.g., lentivirus, plasmid, etc.).

In some embodiments, a nucleic acid of the disclosure (e.g., encoding an AAV9 variant capsid protein) comprises conventional control elements or sequences which are operably linked to the nucleic acid molecule in a manner which permits transcription, translation and/or expression in a cell transfected with the nucleic acid (e.g., a plasmid vector comprising said nucleic acid) or infected with a virus comprising said nucleic acid. As used herein, “operably linked” sequences include both expression control sequences that are contiguous with the gene of interest and expression control sequences that act in trans or at a distance to control the gene of interest.

Expression control sequences include efficient RNA processing signals such as splicing and polyadenylation (polyA) signals; appropriate transcription initiation, termination, promoter and enhancer sequences; sequences that stabilize cytoplasmic mRNA; sequences that enhance protein stability; sequences that enhance translation efficiency (e.g., Kozak consensus sequence); and in some embodiments, sequences that enhance secretion of the encoded transgene product. Expression control sequences, including promoters which are native, constitutive, inducible and/or tissue-specific, are known in the art and can be utilized with the compositions and methods disclosed herein.

In some embodiments, the native promoter for the transgene is used. Without wishing to be bound by theory, the native promoter can mimic native expression of the transgene, or provide temporal, developmental, or tissue-specific expression, or expression in response to specific transcriptional stimuli. In some embodiments, the transgene is operably linked to other native expression control elements, such as enhancer elements, polyadenylation sites or Kozak consensus sequences, e.g., to mimic the native expression.

In some embodiments, the transgene is operably linked to a tissue-specific promoter, e.g., a promoter active specifically in one or more CNS cell types.

In some embodiments, a vector, e.g., a plasmid, carrying a transgene includes a selectable marker or a reporter gene. Such selectable reporters or marker genes can be used to signal the presence of the vector, e.g., plasmid, in bacterial cells. Other components of the vector, e.g., plasmid, include an origin of replication. Selection of these and other promoters and vector elements are conventional and many such sequences are available (see, e.g., Sambrook et al, and references cited therein).

In some embodiments, the viral particle comprising a variant capsid polypeptide, e.g., a variant capsid polypeptide described herein, exhibits increased CNS transduction as compared to a viral particle with the wild-type capsid polypeptide (SEQ ID NO:1).

In some embodiments, the capsid polypeptide is an isolated or purified polypeptide (e.g., isolated or purified from a cell, other biological component, or contaminant). In some embodiments, the variant polypeptide is present in a dependoparvovirus particle, e.g., described herein. In some embodiments, the variant capsid polypeptide is present in a cell, cell-free system, or translation system, e.g., described herein.

In some embodiments, the capsid polypeptide is present in a dependoparvovirus B (e.g., AAV9) particle. In some embodiments, the capsid particle has increased CNS transduction.

In some embodiments, a dependoparvovirus particle comprises an amino acid sequence that has at least 80, 85, 90, 91, 92, 93, 94, 95, 96, 97, 98, 99, or 100% identity to the amino acid sequences provided for herein (e.g., SEQ ID NO:12, 26, 28, 30, 32, 34, 36 or 38). In some embodiments, the variant capsid polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from the amino acid sequence of a variant capsid polypeptide provided for herein.

In some embodiments, the additional alteration improves a production characteristic of a dependoparvovirus particle or method of making the same. In some embodiments, the additional alteration improves or alters another characteristic of a dependoparvovirus particle, e.g., tropism.

### 6.2.1. Targeting Peptides

The capsid polypeptides of the disclosure can include (but do not necessarily include) a targeting peptide to alter the tropism of the capsid polypeptides, for example to enhance targeting to the CNS. Thus, in some embodiments, a capsid polypeptide of the disclosure includes a targeting peptide. In other embodiments, a capsid polypeptide of the disclosure does not include a targeting peptide.

Various targeting peptides for enhancing CNS tropism, and which can be included in capsid polypeptides of the disclosure, are described in the art, for example in WO 2017/197355, WO 2019/006182, WO 2019/060454, WO 2012/145601, WO 2018/022905, WO 2021/243085, WO 2019/076856, WO2015/038958, WO 2015/191508, WO 2020/068990, WO 2020/210655, WO 2020/198737, WO 2020/028751, WO 2019/028306, WO 2017/100671 A1, WO 2020/028751 A2, WO 2020/072683 A1, WO 2020/160337 A1, WO 2020/223280 A1, WO 2021/025995 A1, WO 2021/202651 A1, WO 2021/230987 A1, WO 2022/235702 A1, WO 2020/014471, WO 2018/189244, WO 2019/141765, WO 2019/207132, WO 2019/210267, WO 2018/156654, WO 2010/093784, WO 2015/048534, WO 2017/058892, WO 2019/169132, WO 2021/108468, WO 2021/102234, WO 2022/173847, WO 2021/077000, WO 2020/160337, WO 2021/050974, WO 2021/222831, WO 2022/020616, WO 2020/193799, WO 2021/072197, WO 2022/126188, WO 2022/126189, WO 2021/165544, WO 2021/084133, WO 2022/040527, WO 2022/221400, WO 2022/221404, WO 2022/221420, WO 2021/216456, WO 2021/009684, WO 2021/242909, WO 2019/158619, WO 2021/226267, WO 2023/283962, WO 2021/219762, WO 2022/226374, WO 2022/226375, WO 2022/229703, and WO 2022/229702, the contents of which are incorporated herein by reference in their entireties. Targeting peptides are typically 3 to 20 amino acids in length. In some embodiments, a targeting peptide is 3 to 12 amino acids in length. In other embodiments, a targeting peptide is 5 to 12 amino acids in length. In other embodiments, a targeting peptide is 5 to 10 amino acids in length. In other embodiments, a targeting peptide is 7 to 10 amino acids in length. In some embodiments, a targeting peptide is 7 amino acids in length. In other embodiments, a targeting peptide is 9 amino acids in length.

In some embodiments, the targeting peptide comprises at least 3, 4, 5, 6, 7, 8, or 9 consecutive amino acids from the amino acid sequence of PLNGAVHLY (SEQ ID NO:16). In some embodiments, the targeting peptide comprises the amino acid sequence PLNGAVHLY (SEQ ID NO:16). In some embodiments, the targeting peptide comprises at least 3, 4, 5, 6, or 7 consecutive amino acids from the amino acid sequence of IVMNSLK (SEQ ID NO:17). In some embodiments, the targeting peptide comprises the amino acid sequence IVMNSLK (SEQ ID NO:17). In some embodiments, the targeting peptide comprises at least 3, 4, 5, 6, or 7 consecutive amino acids from the amino acid sequence of RDSPKGW (SEQ ID NO:18). In some embodiments, the targeting peptide comprises the amino acid sequence RDSPKGW (SEQ ID NO:18). In some embodiments, the targeting peptide comprises at least 3, 4, 5, 6, or 7 consecutive amino acids from the amino acid sequence of YSTDVRM (SEQ ID NO:19). In some embodiments, the targeting peptide comprises the amino acid sequence YSTDVRM (SEQ ID NO:19). In some embodiments, the targeting peptide comprises at least 3, 4, 5, 6, or 7 consecutive amino acids from the amino acid sequence of RESPRGL (SEQ ID NO:20). In some embodiments, the targeting peptide comprises the amino acid sequence RESPRGL (SEQ ID NO:20). In some embodiments, the targeting peptide comprises 4, 5, 6, or 7 consecutive amino acids from GNNTRSV (SEQ ID NO:21), GNNTRDT (SEQ ID NO:22) or TNSTRPV (SEQ ID NO:23). In some embodiments, the targeting peptide comprises the amino acid sequence GNNTRSV (SEQ ID NO:21). In some embodiments, the targeting peptide comprises the amino acid sequence GNNTRDT (SEQ ID NO:22). In some embodiments, the targeting peptide comprises the amino acid sequence TNSTRPV (SEQ ID NO:23).

In some embodiments, the targeting peptide is present in, e.g., inserted into, loop VIII of the capsid polypeptide. In some embodiments, the targeting peptide is inserted at any amino acid position corresponding to positions 586-592, inclusive, of the wild-type capsid polypeptide (SEQ ID NO:1). For example, the targeting peptide can be inserted between amino acids 588-589 (positions corresponding to the wild-type capsid polypeptide (SEQ ID NO:1)). In some embodiments, the targeting peptide is present, e.g., inserted, immediately subsequent to the position corresponding to 586, 588, or 589 of the wild-type capsid polypeptide (SEQ ID NO:1). In some embodiments, the capsid polypeptide further comprises a deletion at the position corresponding to 587 and/or a deletion at the position corresponding to 588 of the wild-type capsid polypeptide (SEQ ID NO:1).

### 6.2.2. Nucleic Acids and Polypeptides

The disclosure is further directed, in part, to a nucleic acid comprising a sequence encoding a variant capsid polypeptide as provided for herein. In embodiments the nucleic acid encodes a VP1 variant capsid polypeptide, e.g., as described herein. In embodiments, the nucleic acid encodes a VP2 variant capsid polypeptide, e.g., as described herein. In embodiments, the nucleic acid encodes a VP3 variant capsid polypeptide, e.g., as described herein. In embodiments, the nucleic acid encodes a VP1, VP2 and VP3 variant capsid polypeptide, e.g., as described herein. In some embodiments, the variant capsid polypeptide comprises a sequence of SEQ ID NO:12. In some embodiments, the variant capsid polypeptide comprises a sequence of SEQ ID NO:26. In some embodiments, the variant capsid polypeptide comprises a sequence of SEQ ID NO:28. In some embodiments, the variant capsid polypeptide comprises a sequence of SEQ ID NO:30. In some embodiments, the variant capsid polypeptide comprises a sequence of SEQ ID NO:32. In some embodiments, the variant capsid polypeptide comprises a sequence of SEQ ID NO:34. In some embodiments, the variant capsid polypeptide comprises a sequence of SEQ ID NO:36. In some embodiments, the variant capsid polypeptide comprises a sequence of SEQ ID NO:38. In some embodiments, the nucleic acid comprises a sequence of SEQ ID NO:13. In some embodiments, the nucleic acid comprises a sequence of SEQ ID NO:27. In some embodiments, the nucleic acid comprises a sequence of SEQ ID NO:29. In some embodiments, the nucleic acid comprises a sequence of SEQ ID NO:31. In some embodiments, the nucleic acid comprises a sequence of SEQ ID NO:33. In some embodiments, the nucleic acid comprises a sequence of SEQ ID NO:35. In some embodiments, the nucleic acid comprises a sequence of SEQ ID NO:37. In some embodiments, the nucleic acid comprises a sequence of SEQ ID NO:39.

### 6.3. Dependoparvovirus Particles

The disclosure is also directed, in part, to a dependoparvovirus particle (e.g., a functional dependoparvovirus particle) comprising a nucleic acid or polypeptide described herein or produced by a method described herein.

Dependoparvovirus is a single-stranded DNA parvovirus that grows only in cells in which certain functions are provided, e.g., by a co-infecting helper virus. Several species of dependoparvovirus are known, including dependoparvovirus A and dependoparvovirus B, which include serotypes known in the art as adeno-associated viruses (AAV). At least thirteen serotypes ofAAV that have been characterized. General information and reviews of AAV can be found in, for example, Carter, Handbook of Parvoviruses, Vol. 1, pp. 169-228 (1989), and Berns, Virology, pp. 1743-1764, Raven Press, (New York, 1990). AAV serotypes, and to a degree, dependoparvovirus species, are significantly interrelated structurally and functionally. (See, for example, Blacklowe, pp. 165-174 of Parvoviruses and Human Disease, J. R. Pattison, ed. (1988); and Rose, Comprehensive Virology 3:1-61 (1974)). For example, all AAV serotypes apparently exhibit very similar replication properties mediated by homologous rep genes; and all bear three related capsid proteins. In addition, heteroduplex analysis reveals extensive cross-hybridization between serotypes along the length of the genome, further suggesting interrelatedness. Dependoparvoviruses genomes also comprise self-annealing segments at the termini that correspond to “inverted terminal repeat sequences” (ITRs).

The genomic organization of naturally occurring dependoparvoviruses, e.g., AAV serotypes, is very similar. For example, the genome of AAV is a linear, single-stranded DNA molecule that is approximately 5,000 nucleotides (nt) in length or less. Inverted terminal repeats (ITRs) flank the unique coding nucleotide sequences for the non-structural replication (Rep) proteins and the structural capsid (Cap) proteins. Three different viral particle (VP) proteins form the capsid. The terminal approximately 145 nt of the genome are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins: Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In some embodiments, a dependoparvovirus particle of the disclosure comprises a nucleic acid comprising a variant capsid polypeptide provided for herein. In some embodiments, the particle comprises a polypeptide as provided for herein.

In some embodiments, the dependoparvovirus particle of the disclosure is an AAV9 particle. In some embodiments, the AAV9 particle comprises a variant capsid polypeptide as provided for herein or a nucleic acid molecule encoding the same.

In some embodiments the dependoparvovirus particle comprises a variant capsid comprising a variant capsid polypeptide described herein. In embodiments, the dependoparvovirus particle comprises variant capsid polypeptide described herein and a nucleic acid molecule. In embodiments, the dependoparvovirus particle comprises variant capsid polypeptide described herein and a nucleic acid molecule comprising one or more inverted terminal repeat sequences (ITRs), for example, ITRs derived from an AAV9 dependoparvovirus or an AAV2 dependoparvovirus, one or more regulatory elements (for example, a promoter), and a payload (e.g., as described herein, e.g., a heterologous transgene). In embodiments, at least one of the ITRs is modified. In embodiments, the nucleic acid molecule is single-stranded. In embodiments, the nucleic acid molecule is double stranded, for example, self-complementary. Various ITRs and their use in self-complementary AAV vectors are recognized in the art and include those described in U.S. Pat. Nos. 7,465,583, 8,298,818 and 9,150,882; in U.S. Patent Application Publication Nos. 2004/0029106 A1, 2023/0139985 A1, 2022/0175887 A1; and in Wilmott et al., 2019, Hum. Gene. Ther. Methods, 30(6):206-213 and McCarty, 2008, Mol. Ther., 16(10):1648-1656, each incorporated herein by reference.

### 6.4. Improved Biodistribution and Transduction Characteristics

The disclosure is directed, in part, to nucleic acids, polypeptides, cells, cell free systems, translation systems, viral particles, and methods associated with using and making the same to produce viral particles that have increased distribution to tissues and cells of the CNS and/or CNS transduction as compared to a viral particle comprising a reference sequence that does not otherwise comprise the mutations described herein (or mutations corresponding thereto), for example, as compared with a viral particle comprising a capsid polypeptide sequence of SEQ ID NO:1. In some embodiments, a use of a viral particle comprising the variant capsid polypeptides as described in Section 6.2 or any one of numbered embodiments 1 to 189 (e.g., a viral particle as described in Section 6.3 or any one of numbered embodiments 193 to 221) leads to increased CNS biodistribution of the viral particle and/or increased transduction of a transgene virus particle in the cells of the CNS broadly or brain specifically, and, therefore, increased expression of the payload (transgene) in the CNS or brain. In some embodiments, use of a viral particle comprising the variant capsid polypeptides as described in Section 6.2 or any one of numbered embodiments 1 to 189 (e.g., a viral particle as described in Section 6.3 or any one of numbered embodiments 193 to 221) further leads to reduced (or non-increased) biodistribution of the viral particle and/or reduced (or non-increased) transduction of the transgene in one or more peripheral tissues, e.g., liver, spleen, dorsal root ganglia, or any combination of two or more of the foregoing peripheral tissue types.

In some embodiments, biodistribution and transduction (e.g., of the tissue types described in this section) are measured as described herein, for example as described in Section 8 (e.g., Example 1, Example 2, and/or Example 3). Biodistribution and/or transduction of a virus particle having a variant capsid polypeptide can be measured using virus particles having a transgene operably linked to a ubiquitous promoter or a CNS-specific promoter. For example, biodistribution can be measured using virus particles having a transgene operably linked to CBh promoter or a hSYN promoter. In various embodiments, the transgene is a transgene encoding a capsid polypeptide or any other suitable heterologous transgene, for example a nucleic acid sequence encoding a synthetic, mammalian or human therapeutic protein or nucleic acid (e.g., mRNA or RNAi) or reporter gene such as, for example a nucleic acid encoding a GFP or mCherry reporter.

In embodiments, the virus particle, e.g., as described herein, e.g., comprising a variant capsid polypeptide described herein, is capable of crossing the blood-brain barrier. In embodiments the virus particle, e.g., as described herein, e.g., comprising a variant capsid polypeptide described herein, exhibits increased crossing of the blood-brain barrier relative to a virus particle comprising a reference capsid polypeptide, e.g., a reference capsid polypeptide of SEQ ID NO:1. In embodiments, the virus particle, e.g., as described herein, e.g., comprising a variant capsid polypeptide described herein, exhibits increased transduction of neurons, astrocytes, glial cells, or combinations thereof, relative to a virus particle comprising a reference capsid polypeptide, e.g., a reference capsid polypeptide of SEQ ID NO:1.

In some embodiments, a viral particle comprising the variant capsid polypeptide, e.g., the variant capsid polypeptide described herein, exhibits improved properties, e.g., improved biodistribution, transduction and/or production. Unless indicated otherwise, improvement rates are presented as fold-improvement over the rates exhibited by a virus particle comprising capsid polypeptides of SEQ ID NO:1. In some embodiments, improvement means an increase, e.g., in the case of CNS biodistribution or CNS transduction. In other embodiments, improvement means a decrease, e.g., in the case of liver biodistribution or liver transduction. A virus particle having increased biodistribution or transduction in target cells or target tissue types, e.g., CNS, and/or decreased biodistribution or transduction in off-target cells or off-target tissue types, e.g., liver, may have improved specificity for a target cell or target tissue type. This improvement may be beneficial in the use of the viral particle to deliver a therapeutic transgene to the target cell or target tissue type in a subject afflicted with a disease affecting the target cell or target tissue type, for example.

In some embodiments, one or more improved properties (e.g., increased or decreased biodistribution and/or transduction) is exhibited in a mammal, e.g., a primate, e.g., a human. In embodiments, the increased or decreased biodistribution and/or transduction is exhibited upon administration of the virus particle or pharmaceutical composition comprising the virus particle, e.g., as described herein, by systemic administration, e.g., intravenous administration.

In some embodiments, a viral particle comprising the variant capsid polypeptide exhibits an improvement in one or more categories as defined below. In some embodiments, the viral particle exhibits an improvement in Category A (CNS biodistribution) and Category B (CNS transduction). These improvements may be beneficial in the use of the viral particle to deliver a therapeutic transgene to the CNS in a subject afflicted with a disease affecting the CNS, for example. In some embodiments, the viral particle exhibits an improvement in Category A (CNS biodistribution) and Category B (CNS transduction), and one or more of Categories C (PNS biodistribution and/or transduction), D (liver biodistribution and/or transduction) and E (spleen biodistribution and/or transduction). In some embodiments, the viral particle exhibits an improvement in Category A (CNS biodistribution), Category B (CNS transduction), and Category D (liver biodistribution and/or transduction), and optionally exhibits an improvement in Category C (PNS biodistribution and/or transduction) and/or E (spleen biodistribution and/or transduction).

Category A (CNS Biodistribution): In some aspects of the disclosure, a viral particle comprising the variant capsid polypeptide, e.g., the variant capsid polypeptide described herein, exhibits increased CNS biodistribution as compared to a viral particle with the wild-type capsid polypeptide (SEQ ID NO:1). In some embodiments, the increased CNS biodistribution comprises increased brain biodistribution and/or spinal cord biodistribution. In some embodiments, increased brain biodistribution is aggregated brain biodistribution, or biodistribution in a particular brain tissue such as brain stem, basal ganglia, cerebellum, forebrain, hippocampus, midbrain, or temporal cortex, in each case measured with a CNS-specific promoter such as hSyn, with a constitutive promoter such as Cbh, or as an aggregate of a CNS-specific promoter such as hSyn and a constitutive promoter such as Cbh. In some embodiments, the increased biodistribution is increased aggregated brain biodistribution using a Cbh or hSyn promoter and/or is as defined in any one of embodiments A-1 through A-43.
Embodiment A-1: In an embodiment of Category A, the biodistribution is about (or at least about) 15 times better relative to a virus particle comprising a variant capsid polypeptide having a reference sequence, e.g., having the wild-type capsid protein, e.g., having capsid polypeptides of SEQ ID NO:1.
Embodiment A-2: In an embodiment of Category A, the biodistribution is about (or at least about) 20 times better relative to a virus particle comprising a variant capsid polypeptide having a reference sequence, e.g., having the wild-type capsid protein, e.g., having capsid polypeptides of SEQ ID NO:1.
Embodiment A-3: In an embodiment of Category A, the biodistribution is about (or at least about) 25 times better relative to a virus particle comprising a variant capsid polypeptide having a reference sequence, e.g., having the wild-type capsid protein, e.g., having capsid polypeptides of SEQ ID NO:1.
Embodiment A-4: In an embodiment of Category A, the biodistribution is about (or at least about) 30 times better relative to a virus particle comprising a variant capsid polypeptide having a reference sequence, e.g., having the wild-type capsid protein, e.g., having capsid polypeptides of SEQ ID NO:1.
Embodiment A-5: In an embodiment of Category A, the biodistribution is about (or at least about) 35 times better relative to a virus particle comprising a variant capsid polypeptide having a reference sequence, e.g., having the wild-type capsid protein, e.g., having capsid polypeptides of SEQ ID NO:1.
Embodiment A-6: In an embodiment of Category A, the biodistribution is about (or at least about) 40 times better relative to a virus particle comprising a variant capsid polypeptide having a reference sequence, e.g., having the wild-type capsid protein, e.g., having capsid polypeptides of SEQ ID NO:1.
Embodiment A-7: In an embodiment of Category A, the biodistribution is about (or at least about) 50 times better relative to a virus particle comprising a variant capsid polypeptide having a reference sequence, e.g., having the wild-type capsid protein, e.g., having capsid polypeptides of SEQ ID NO:1.
Embodiment A-8: In an embodiment of Category A, the biodistribution is about (or at least about) 60 times better relative to a virus particle comprising a variant capsid polypeptide having a reference sequence, e.g., having the wild-type capsid protein, e.g., having capsid polypeptides of SEQ ID NO:1.
Embodiment A-9: In an embodiment of Category A, the biodistribution is about (or at least about) 70 times better relative to a virus particle comprising a variant capsid polypeptide having a reference sequence, e.g., having the wild-type capsid protein, e.g., having capsid polypeptides of SEQ ID NO:1.
Embodiment A-10: In an embodiment of Category A, the biodistribution is about (or at least about) 80 times better relative to a virus particle comprising a variant capsid polypeptide having a reference sequence, e.g., having the wild-type capsid protein, e.g., having capsid polypeptides of SEQ ID NO:1.
Embodiment A-11: In an embodiment of Category A, the biodistribution is about (or at least about) 90 times better relative to a virus particle comprising a variant capsid polypeptide having a reference sequence, e.g., having the wild-type capsid protein, e.g., having capsid polypeptides of SEQ ID NO:1.
Embodiment A-12: In an embodiment of Category A, the biodistribution is about (or at least about) 100 times better relative to a virus particle comprising a variant capsid polypeptide having a reference sequence, e.g., having the wild-type capsid protein, e.g., having capsid polypeptides of SEQ ID NO:1.
Embodiment A-13: In an embodiment of Category A, the biodistribution is about (or at least about) 150 times better relative to a virus particle comprising a variant capsid polypeptide having a reference sequence, e.g., having the wild-type capsid protein, e.g., having capsid polypeptides of SEQ ID NO:1.
Embodiment A-14: In an embodiment of Category A, the biodistribution is about (or at least about) 200 times better relative to a virus particle comprising a variant capsid polypeptide having a reference sequence, e.g., having the wild-type capsid protein, e.g., having capsid polypeptides of SEQ ID NO:1.
Embodiment A-15: In an embodiment of Category A, the biodistribution is about (or at least about) 250 times better relative to a virus particle comprising a variant capsid polypeptide having a reference sequence, e.g., having the wild-type capsid protein, e.g., having capsid polypeptides of SEQ ID NO:1.
Embodiment A-16: In an embodiment of Category A, the biodistribution is about (or at least about) 300 times better relative to a virus particle comprising a variant capsid polypeptide having a reference sequence, e.g., having the wild-type capsid protein, e.g., having capsid polypeptides of SEQ ID NO:1.
Embodiment A-17: In an embodiment of Category A, the biodistribution is about (or at least about) 350 times better relative to a virus particle comprising a variant capsid polypeptide having a reference sequence, e.g., having the wild-type capsid protein, e.g., having capsid polypeptides of SEQ ID NO:1.
Embodiment A-18: In an embodiment of Category A, the biodistribution is about (or at least about) 400 times better relative to a virus particle comprising a variant capsid polypeptide having a reference sequence, e.g., having the wild-type capsid protein, e.g., having capsid polypeptides of SEQ ID NO:1.
Embodiment A-19: In an embodiment of Category A, the biodistribution is about (or at least about) 450 times better relative to a virus particle comprising a variant capsid polypeptide having a reference sequence, e.g., having the wild-type capsid protein, e.g., having capsid polypeptides of SEQ ID NO:1.
Embodiment A-20: In an embodiment of Category A, the biodistribution is about (or at least about) 500 times better relative to a virus particle comprising a variant capsid polypeptide having a reference sequence, e.g., having the wild-type capsid protein, e.g., having capsid polypeptides of SEQ ID NO:1.

In some embodiments, the improved biodistribution is in a range bounded by any two values set forth in embodiments A-1 to A-20. Exemplary ranges are set forth in embodiments A-21 to A-43 below.
Embodiment A-21: In an embodiment of Category A, the biodistribution ranges between about 15 and about 600 times better relative to a virus particle comprising a variant capsid polypeptide having a reference sequence, e.g., having the wild-type capsid protein, e.g., having capsid polypeptides of SEQ ID NO:1.
Embodiment A-22: In an embodiment of Category A, the biodistribution ranges between about 15 and about 500 times better relative to a virus particle comprising a variant capsid polypeptide having a reference sequence, e.g., having the wild-type capsid protein, e.g., having capsid polypeptides of SEQ ID NO:1.
Embodiment A-23: In an embodiment of Category A, the biodistribution ranges between about 15 and about 400 times better relative to a virus particle comprising a variant capsid polypeptide having a reference sequence, e.g., having the wild-type capsid protein, e.g., having capsid polypeptides of SEQ ID NO:1.
Embodiment A-24: In an embodiment of Category A, the biodistribution ranges between about 15 and about 300 times better relative to a virus particle comprising a variant capsid polypeptide having a reference sequence, e.g., having the wild-type capsid protein, e.g., having capsid polypeptides of SEQ ID NO:1.
Embodiment A-25: In an embodiment of Category A, the biodistribution ranges between about 15 and about 200 times better relative to a virus particle comprising a variant capsid polypeptide having a reference sequence, e.g., having the wild-type capsid protein, e.g., having capsid polypeptides of SEQ ID NO:1.
Embodiment A-26: In an embodiment of Category A, the biodistribution ranges between about 15 and about 100 times better relative to a virus particle comprising a variant capsid polypeptide having a reference sequence, e.g., having the wild-type capsid protein, e.g., having capsid polypeptides of SEQ ID NO:1.
Embodiment A-27: In an embodiment of Category A, the biodistribution ranges between about 15 and about 50 times better relative to a virus particle comprising a variant capsid polypeptide having a reference sequence, e.g., having the wild-type capsid protein, e.g., having capsid polypeptides of SEQ ID NO:1.
Embodiment A-28: In an embodiment of Category A, the biodistribution ranges between about 50 and about 600 times better relative to a virus particle comprising a variant capsid polypeptide having a reference sequence, e.g., having the wild-type capsid protein, e.g., having capsid polypeptides of SEQ ID NO:1.
Embodiment A-29: In an embodiment of Category A, the biodistribution ranges between about 50 and about 500 times better relative to a virus particle comprising a variant capsid polypeptide having a reference sequence, e.g., having the wild-type capsid protein, e.g., having capsid polypeptides of SEQ ID NO:1.
Embodiment A-30: In an embodiment of Category A, the biodistribution ranges between about 50 and about 400 times better relative to a virus particle comprising a variant capsid polypeptide having a reference sequence, e.g., having the wild-type capsid protein, e.g., having capsid polypeptides of SEQ ID NO:1.
Embodiment A-31: In an embodiment of Category A, the biodistribution ranges between about 50 and about 300 times better relative to a virus particle comprising a variant capsid polypeptide having a reference sequence, e.g., having the wild-type capsid protein, e.g., having capsid polypeptides of SEQ ID NO:1.
Embodiment A-32: In an embodiment of Category A, the biodistribution ranges between about 50 and about 200 times better relative to a virus particle comprising a variant capsid polypeptide having a reference sequence, e.g., having the wild-type capsid protein, e.g., having capsid polypeptides of SEQ ID NO:1.
Embodiment A-33: In an embodiment of Category A, the biodistribution ranges between about 50 and about 100 times better relative to a virus particle comprising a variant capsid polypeptide having a reference sequence, e.g., having the wild-type capsid protein, e.g., having capsid polypeptides of SEQ ID NO:1.
Embodiment A-34: In an embodiment of Category A, the biodistribution ranges between about 100 and about 600 times better relative to a virus particle comprising a variant capsid polypeptide having a reference sequence, e.g., having the wild-type capsid protein, e.g., having capsid polypeptides of SEQ ID NO:1.
Embodiment A-35: In an embodiment of Category A, the biodistribution ranges between about 100 and about 500 times better relative to a virus particle comprising a variant capsid polypeptide having a reference sequence, e.g., having the wild-type capsid protein, e.g., having capsid polypeptides of SEQ ID NO:1.
Embodiment A-36: In an embodiment of Category A, the biodistribution ranges between about 100 and about 400 times better relative to a virus particle comprising a variant capsid polypeptide having a reference sequence, e.g., having the wild-type capsid protein, e.g., having capsid polypeptides of SEQ ID NO:1.
Embodiment A-37: In an embodiment of Category A, the biodistribution ranges between about 100 and about 300 times better relative to a virus particle comprising a variant capsid polypeptide having a reference sequence, e.g., having the wild-type capsid protein, e.g., having capsid polypeptides of SEQ ID NO:1.
Embodiment A-38: In an embodiment of Category A, the biodistribution ranges between about 100 and about 200 times better relative to a virus particle comprising a variant capsid polypeptide having a reference sequence, e.g., having the wild-type capsid protein, e.g., having capsid polypeptides of SEQ ID NO:1.
Embodiment A-39: In an embodiment of Category A, the biodistribution ranges between about 15 and about 50 times better relative to a virus particle comprising a variant capsid polypeptide having a reference sequence, e.g., having the wild-type capsid protein, e.g., having capsid polypeptides of SEQ ID NO:1.
Embodiment A-40: In an embodiment of Category A, the biodistribution ranges between about 15 and about 40 times better relative to a virus particle comprising a variant capsid polypeptide having a reference sequence, e.g., having the wild-type capsid protein, e.g., having capsid polypeptides of SEQ ID NO:1.
Embodiment A-41: In an embodiment of Category A, the biodistribution ranges between about 15 and about 30 times better relative to a virus particle comprising a variant capsid polypeptide having a reference sequence, e.g., having the wild-type capsid protein, e.g., having capsid polypeptides of SEQ ID NO:1.
Embodiment A-42: In an embodiment of Category A, the biodistribution ranges between about 15 and about 25 times better relative to a virus particle comprising a variant capsid polypeptide having a reference sequence, e.g., having the wild-type capsid protein, e.g., having capsid polypeptides of SEQ ID NO:1.
Embodiment A-43: In an embodiment of Category A, the biodistribution ranges between about 15 and about 20 times better relative to a virus particle comprising a variant capsid polypeptide having a reference sequence, e.g., having the wild-type capsid protein, e.g., having capsid polypeptides of SEQ ID NO:1.
Category B (CNS Transduction): In some aspects of the disclosure, a viral particle comprising the variant capsid polypeptide, e.g., the variant capsid polypeptide described herein, exhibits increased CNS transduction as compared to a viral particle with the wild-type capsid polypeptide (SEQ ID NO:1). In some embodiments, the increased CNS transduction comprises increased brain transduction and/or spinal cord transduction. In some embodiments, increased brain transduction is aggregated brain transduction, or transduction in a particular brain tissue such as brain stem, basal ganglia, cerebellum, forebrain, hippocampus, midbrain, or temporal cortex, in each case measured with a CNS-specific promoter such as hSyn, with a constitutive promoter such as Cbh, or as an aggregate of a CNS-specific promoter such as hSyn and a constitutive promoter such as Cbh. In some embodiments, the increased transduction is increased aggregated brain transduction using a Cbh or hSyn promoter and/or is as defined in any one of embodiments B-1 through B-43.
Embodiment B-1: In an embodiment of Category B, the biodistribution is about (or at least about) 15 times better relative to a virus particle comprising a variant capsid polypeptide having a reference sequence, e.g., having the wild-type capsid protein, e.g., having capsid polypeptides of SEQ ID NO:1.
Embodiment B-2: In an embodiment of Category B, the biodistribution is about (or at least about) 20 times better relative to a virus particle comprising a variant capsid polypeptide having a reference sequence, e.g., having the wild-type capsid protein, e.g., having capsid polypeptides of SEQ ID NO:1.
Embodiment B-3: In an embodiment of Category B, the biodistribution is about (or at least about) 25 times better relative to a virus particle comprising a variant capsid polypeptide having a reference sequence, e.g., having the wild-type capsid protein, e.g., having capsid polypeptides of SEQ ID NO:1.
Embodiment B-4: In an embodiment of Category B, the biodistribution is about (or at least about) 30 times better relative to a virus particle comprising a variant capsid polypeptide having a reference sequence, e.g., having the wild-type capsid protein, e.g., having capsid polypeptides of SEQ ID NO:1.
Embodiment B-5: In an embodiment of Category B, the biodistribution is about (or at least about) 35 times better relative to a virus particle comprising a variant capsid polypeptide having a reference sequence, e.g., having the wild-type capsid protein, e.g., having capsid polypeptides of SEQ ID NO:1.
Embodiment B-6: In an embodiment of Category B, the biodistribution is about (or at least about) 40 times better relative to a virus particle comprising a variant capsid polypeptide having a reference sequence, e.g., having the wild-type capsid protein, e.g., having capsid polypeptides of SEQ ID NO:1.
Embodiment B-7: In an embodiment of Category B, the biodistribution is about (or at least about) 50 times better relative to a virus particle comprising a variant capsid polypeptide having a reference sequence, e.g., having the wild-type capsid protein, e.g., having capsid polypeptides of SEQ ID NO:1.
Embodiment B-8: In an embodiment of Category B, the biodistribution is about (or at least about) 60 times better relative to a virus particle comprising a variant capsid polypeptide having a reference sequence, e.g., having the wild-type capsid protein, e.g., having capsid polypeptides of SEQ ID NO:1.
Embodiment B-9: In an embodiment of Category B, the biodistribution is about (or at least about) 70 times better relative to a virus particle comprising a variant capsid polypeptide having a reference sequence, e.g., having the wild-type capsid protein, e.g., having capsid polypeptides of SEQ ID NO:1.
Embodiment B-10: In an embodiment of Category B, the biodistribution is about (or at least about) 80 times better relative to a virus particle comprising a variant capsid polypeptide having a reference sequence, e.g., having the wild-type capsid protein, e.g., having capsid polypeptides of SEQ ID NO:1.
Embodiment B-11: In an embodiment of Category B, the biodistribution is about (or at least about) 90 times better relative to a virus particle comprising a variant capsid polypeptide having a reference sequence, e.g., having the wild-type capsid protein, e.g., having capsid polypeptides of SEQ ID NO:1.
Embodiment B-12: In an embodiment of Category B, the biodistribution is about (or at least about) 100 times better relative to a virus particle comprising a variant capsid polypeptide having a reference sequence, e.g., having the wild-type capsid protein, e.g., having capsid polypeptides of SEQ ID NO:1.
Embodiment B-13: In an embodiment of Category B, the biodistribution is about (or at least about) 150 times better relative to a virus particle comprising a variant capsid polypeptide having a reference sequence, e.g., having the wild-type capsid protein, e.g., having capsid polypeptides of SEQ ID NO:1.
Embodiment B-14: In an embodiment of Category B, the biodistribution is about (or at least about) 200 times better relative to a virus particle comprising a variant capsid polypeptide having a reference sequence, e.g., having the wild-type capsid protein, e.g., having capsid polypeptides of SEQ ID NO:1.
Embodiment B-15: In an embodiment of Category B, the biodistribution is about (or at least about) 250 times better relative to a virus particle comprising a variant capsid polypeptide having a reference sequence, e.g., having the wild-type capsid protein, e.g., having capsid polypeptides of SEQ ID NO:1.
Embodiment B-16: In an embodiment of Category B, the biodistribution is about (or at least about) 300 times better relative to a virus particle comprising a variant capsid polypeptide having a reference sequence, e.g., having the wild-type capsid protein, e.g., having capsid polypeptides of SEQ ID NO:1.
Embodiment B-17: In an embodiment of Category B, the biodistribution is about (or at least about) 350 times better relative to a virus particle comprising a variant capsid polypeptide having a reference sequence, e.g., having the wild-type capsid protein, e.g., having capsid polypeptides of SEQ ID NO:1.
Embodiment B-18: In an embodiment of Category B, the biodistribution is about (or at least about) 400 times better relative to a virus particle comprising a variant capsid polypeptide having a reference sequence, e.g., having the wild-type capsid protein, e.g., having capsid polypeptides of SEQ ID NO:1.
Embodiment B-19: In an embodiment of Category B, the biodistribution is about (or at least about) 450 times better relative to a virus particle comprising a variant capsid polypeptide having a reference sequence, e.g., having the wild-type capsid protein, e.g., having capsid polypeptides of SEQ ID NO:1.
Embodiment B-20: In an embodiment of Category B, the biodistribution is about (or at least about) 500 times better relative to a virus particle comprising a variant capsid polypeptide having a reference sequence, e.g., having the wild-type capsid protein, e.g., having capsid polypeptides of SEQ ID NO:1.

In some embodiments, the improved biodistribution is in a range bounded by any two values set forth in embodiments B-1 to B-20. Exemplary ranges are set forth in embodiments B-21 to B-43 below.
Embodiment B-21: In an embodiment of Category B, the biodistribution ranges between about 15 and about 600 times better relative to a virus particle comprising a variant capsid polypeptide having a reference sequence, e.g., having the wild-type capsid protein, e.g., having capsid polypeptides of SEQ ID NO:1.
Embodiment B-22: In an embodiment of Category B, the biodistribution ranges between about 15 and about 500 times better relative to a virus particle comprising a variant capsid polypeptide having a reference sequence, e.g., having the wild-type capsid protein, e.g., having capsid polypeptides of SEQ ID NO:1.
Embodiment B-23: In an embodiment of Category B, the biodistribution ranges between about 15 and about 400 times better relative to a virus particle comprising a variant capsid polypeptide having a reference sequence, e.g., having the wild-type capsid protein, e.g., having capsid polypeptides of SEQ ID NO:1.
Embodiment B-24: In an embodiment of Category B, the biodistribution ranges between about 15 and about 300 times better relative to a virus particle comprising a variant capsid polypeptide having a reference sequence, e.g., having the wild-type capsid protein, e.g., having capsid polypeptides of SEQ ID NO:1.
Embodiment B-25: In an embodiment of Category B, the biodistribution ranges between about 15 and about 200 times better relative to a virus particle comprising a variant capsid polypeptide having a reference sequence, e.g., having the wild-type capsid protein, e.g., having capsid polypeptides of SEQ ID NO:1.
Embodiment B-26: In an embodiment of Category B, the biodistribution ranges between about 15 and about 100 times better relative to a virus particle comprising a variant capsid polypeptide having a reference sequence, e.g., having the wild-type capsid protein, e.g., having capsid polypeptides of SEQ ID NO:1.
Embodiment B-27: In an embodiment of Category B, the biodistribution ranges between about 15 and about 50 times better relative to a virus particle comprising a variant capsid polypeptide having a reference sequence, e.g., having the wild-type capsid protein, e.g., having capsid polypeptides of SEQ ID NO:1.
Embodiment B-28: In an embodiment of Category B, the biodistribution ranges between about 50 and about 600 times better relative to a virus particle comprising a variant capsid polypeptide having a reference sequence, e.g., having the wild-type capsid protein, e.g., having capsid polypeptides of SEQ ID NO:1.
Embodiment B-29: In an embodiment of Category B, the biodistribution ranges between about 50 and about 500 times better relative to a virus particle comprising a variant capsid polypeptide having a reference sequence, e.g., having the wild-type capsid protein, e.g., having capsid polypeptides of SEQ ID NO:1.
Embodiment B-30: In an embodiment of Category B, the biodistribution ranges between about 50 and about 400 times better relative to a virus particle comprising a variant capsid polypeptide having a reference sequence, e.g., having the wild-type capsid protein, e.g., having capsid polypeptides of SEQ ID NO:1.
Embodiment B-31: In an embodiment of Category B, the biodistribution ranges between about 50 and about 300 times better relative to a virus particle comprising a variant capsid polypeptide having a reference sequence, e.g., having the wild-type capsid protein, e.g., having capsid polypeptides of SEQ ID NO:1.
Embodiment B-32: In an embodiment of Category B, the biodistribution ranges between about 50 and about 200 times better relative to a virus particle comprising a variant capsid polypeptide having a reference sequence, e.g., having the wild-type capsid protein, e.g., having capsid polypeptides of SEQ ID NO:1.
Embodiment B-33: In an embodiment of Category B, the biodistribution ranges between about 50 and about 100 times better relative to a virus particle comprising a variant capsid polypeptide having a reference sequence, e.g., having the wild-type capsid protein, e.g., having capsid polypeptides of SEQ ID NO:1.
Embodiment B-34: In an embodiment of Category B, the biodistribution ranges between about 100 and about 600 times better relative to a virus particle comprising a variant capsid polypeptide having a reference sequence, e.g., having the wild-type capsid protein, e.g., having capsid polypeptides of SEQ ID NO:1.
Embodiment B-35: In an embodiment of Category B, the biodistribution ranges between about 100 and about 500 times better relative to a virus particle comprising a variant capsid polypeptide having a reference sequence, e.g., having the wild-type capsid protein, e.g., having capsid polypeptides of SEQ ID NO:1.
Embodiment B-36: In an embodiment of Category B, the biodistribution ranges between about 100 and about 400 times better relative to a virus particle comprising a variant capsid polypeptide having a reference sequence, e.g., having the wild-type capsid protein, e.g., having capsid polypeptides of SEQ ID NO:1.
Embodiment B-37: In an embodiment of Category B, the biodistribution ranges between about 100 and about 300 times better relative to a virus particle comprising a variant capsid polypeptide having a reference sequence, e.g., having the wild-type capsid protein, e.g., having capsid polypeptides of SEQ ID NO:1.
Embodiment B-38: In an embodiment of Category B, the biodistribution ranges between about 100 and about 200 times better relative to a virus particle comprising a variant capsid polypeptide having a reference sequence, e.g., having the wild-type capsid protein, e.g., having capsid polypeptides of SEQ ID NO:1.
Embodiment B-39: In an embodiment of Category B, the biodistribution ranges between about 15 and about 50 times better relative to a virus particle comprising a variant capsid polypeptide having a reference sequence, e.g., having the wild-type capsid protein, e.g., having capsid polypeptides of SEQ ID NO:1.
Embodiment B-40: In an embodiment of Category B, the biodistribution ranges between about 15 and about 40 times better relative to a virus particle comprising a variant capsid polypeptide having a reference sequence, e.g., having the wild-type capsid protein, e.g., having capsid polypeptides of SEQ ID NO:1.
Embodiment B-41: In an embodiment of Category B, the biodistribution ranges between about 15 and about 30 times better relative to a virus particle comprising a variant capsid polypeptide having a reference sequence, e.g., having the wild-type capsid protein, e.g., having capsid polypeptides of SEQ ID NO:1.
Embodiment B-42: In an embodiment of Category B, the biodistribution ranges between about 15 and about 25 times better relative to a virus particle comprising a variant capsid polypeptide having a reference sequence, e.g., having the wild-type capsid protein, e.g., having capsid polypeptides of SEQ ID NO:1.
Embodiment B-43: In an embodiment of Category B, the biodistribution ranges between about 15 and about 20 times better relative to a virus particle comprising a variant capsid polypeptide having a reference sequence, e.g., having the wild-type capsid protein, e.g., having capsid polypeptides of SEQ ID NO:1.
Category C (Peripheral Nervous System (“PNS”) Biodistribution and/or Transduction): In some aspects of the disclosure, a viral particle comprising the variant capsid polypeptide, e.g., the variant capsid polypeptide described herein, exhibits similar or reduced PNS biodistribution and/or transduction as compared to a viral particle with the wild-type capsid polypeptide (SEQ ID NO:1). In some embodiments, the PNS biodistribution and/or transduction is measured with a CNS-specific promoter such as hSyn, with a constitutive promoter such as Cbh, or as an aggregate of a CNS-specific promoter such as hSyn and a constitutive promoter such as Cbh. In some embodiments, the PNS biodistribution and/or transduction is DRG biodistribution and/or transduction and/or is as defined in any one of embodiments C-1 through C-22.
Embodiment C-1: In an embodiment of Category C, the PNS biodistribution and/or transduction is about (or no more than about) 10 times increased relative to a virus particle comprising a variant capsid polypeptide having a reference sequence, e.g., having the wild-type capsid protein, e.g., having capsid polypeptides of SEQ ID NO:1.
Embodiment C-2: In an embodiment of Category C, the PNS biodistribution and/or transduction is about (or no more than about) 5 times increased relative to a virus particle comprising a variant capsid polypeptide having a reference sequence, e.g., having the wild-type capsid protein, e.g., having capsid polypeptides of SEQ ID NO:1.
Embodiment C-3: In an embodiment of Category C, the PNS biodistribution and/or transduction is about (or no more than about) 3 times increased relative to a virus particle comprising a variant capsid polypeptide having a reference sequence, e.g., having the wild-type capsid protein, e.g., having capsid polypeptides of SEQ ID NO:1.
Embodiment C-4: In an embodiment of Category C, the PNS biodistribution and/or transduction is about (or no more than about) 1.5 times increased relative to a virus particle comprising a variant capsid polypeptide having a reference sequence, e.g., having the wild-type capsid protein, e.g., having capsid polypeptides of SEQ ID NO:1.
Embodiment C-5: In an embodiment of Category C, the PNS biodistribution and/or transduction is about (or no more than about) 1 times a virus particle comprising a variant capsid polypeptide having a reference sequence, e.g., having the wild-type capsid protein, e.g., having capsid polypeptides of SEQ ID NO:1.
Embodiment C-6: In an embodiment of Category C, the PNS biodistribution and/or transduction is about (or no more than about) 0.9 times the PNS biodistribution and/or transduction of a virus particle comprising a variant capsid polypeptide having a reference sequence, e.g., having the wild-type capsid protein, e.g., having capsid polypeptides of SEQ ID NO:1.
Embodiment C-7: In an embodiment of Category C, the PNS biodistribution and/or transduction is about (or no more than about) 0.8 times the PNS biodistribution and/or transduction of a virus particle comprising a variant capsid polypeptide having a reference sequence, e.g., having the wild-type capsid protein, e.g., having capsid polypeptides of SEQ ID NO:1.
Embodiment C-8: In an embodiment of Category C, the PNS biodistribution and/or transduction is about (or no more than) 0.7 times the PNS biodistribution and/or transduction of a virus particle comprising a variant capsid polypeptide having a reference sequence, e.g., having the wild-type capsid protein, e.g., having capsid polypeptides of SEQ ID NO:1.
Embodiment C-9: In an embodiment of Category C, the PNS biodistribution and/or transduction is about (or no more than) 0.6 times the PNS biodistribution and/or transduction of a virus particle comprising a variant capsid polypeptide having a reference sequence, e.g., having the wild-type capsid protein, e.g., having capsid polypeptides of SEQ ID NO:1.
Embodiment C-10: In an embodiment of Category C, the PNS biodistribution and/or transduction is about (or no more than) 0.6 times the PNS biodistribution and/or transduction of a virus particle comprising a variant capsid polypeptide having a reference sequence, e.g., having the wild-type capsid protein, e.g., having capsid polypeptides of SEQ ID NO:1.

In some embodiments, the improved PNS biodistribution and/or transduction is in a range bounded by any two values set forth in embodiments C-1 to C-13. Exemplary ranges are set forth in embodiments C-11 to C-22 below.
Embodiment C-11: In an embodiment of Category C, the PNS biodistribution and/or transduction ranges between about 0.5 and about 10 times the PNS biodistribution and/or transduction of capsid polypeptide having a reference sequence, e.g., having the wild-type capsid protein, e.g., having capsid polypeptides of SEQ ID NO:1.
Embodiment C-12: In an embodiment of Category C, the PNS biodistribution and/or transduction ranges between about 0.5 and about 5 times the PNS biodistribution and/or transduction of capsid polypeptide having a reference sequence, e.g., having the wild-type capsid protein, e.g., having capsid polypeptides of SEQ ID NO:1.
Embodiment C-13: In an embodiment of Category C, the PNS biodistribution and/or transduction ranges between about 0.6 and about 10 times the PNS biodistribution and/or transduction of capsid polypeptide having a reference sequence, e.g., having the wild-type capsid protein, e.g., having capsid polypeptides of SEQ ID NO:1.
Embodiment C-14: In an embodiment of Category C, the PNS biodistribution and/or transduction ranges between about 0.6 and about 5 times the PNS biodistribution and/or transduction of capsid polypeptide having a reference sequence, e.g., having the wild-type capsid protein, e.g., having capsid polypeptides of SEQ ID NO:1.
Embodiment C-15: In an embodiment of Category C, the PNS biodistribution and/or transduction ranges between about 0.7 and about 10 times the PNS biodistribution and/or transduction of capsid polypeptide having a reference sequence, e.g., having the wild-type capsid protein, e.g., having capsid polypeptides of SEQ ID NO:1.
Embodiment C-16: In an embodiment of Category C, the PNS biodistribution and/or transduction ranges between about 0.7 and about 5 times the PNS biodistribution and/or transduction of capsid polypeptide having a reference sequence, e.g., having the wild-type capsid protein, e.g., having capsid polypeptides of SEQ ID NO:1.
Embodiment C-17: In an embodiment of Category C, the PNS biodistribution and/or transduction ranges between about 0.8 and about 10 times the PNS biodistribution and/or transduction of capsid polypeptide having a reference sequence, e.g., having the wild-type capsid protein, e.g., having capsid polypeptides of SEQ ID NO:1.
Embodiment C-18: In an embodiment of Category C, the PNS biodistribution and/or transduction ranges between about 0.8 and about 5 times the PNS biodistribution and/or transduction of capsid polypeptide having a reference sequence, e.g., having the wild-type capsid protein, e.g., having capsid polypeptides of SEQ ID NO:1.
Embodiment C-19: In an embodiment of Category C, the PNS biodistribution and/or transduction ranges between about 0.9 and about 10 times the PNS biodistribution and/or transduction of capsid polypeptide having a reference sequence, e.g., having the wild-type capsid protein, e.g., having capsid polypeptides of SEQ ID NO:1.
Embodiment C-20: In an embodiment of Category C, the PNS biodistribution and/or transduction ranges between about 0.9 and about 5 times the PNS biodistribution and/or transduction of capsid polypeptide having a reference sequence, e.g., having the wild-type capsid protein, e.g., having capsid polypeptides of SEQ ID NO:1.
Embodiment C-21: In an embodiment of Category C, the PNS biodistribution and/or transduction ranges between about 1 and about 10 times the PNS biodistribution and/or transduction of capsid polypeptide having a reference sequence, e.g., having the wild-type capsid protein, e.g., having capsid polypeptides of SEQ ID NO:1.
Embodiment C-22: In an embodiment of Category C, the PNS biodistribution and/or transduction ranges between about 1 and about 5 times the PNS biodistribution and/or transduction of capsid polypeptide having a reference sequence, e.g., having the wild-type capsid protein, e.g., having capsid polypeptides of SEQ ID NO:1.
Category D (Liver Biodistribution and/or Transduction): In some aspects of the disclosure, a viral particle comprising the variant capsid polypeptide, e.g., the variant capsid polypeptide described herein, exhibits similar or reduced liver biodistribution and/or transduction as compared to a viral particle with the wild-type capsid polypeptide (SEQ ID NO:1). In some embodiments, the liver biodistribution and/or transduction is measured with a CNS-specific promoter such as hSyn, with a constitutive promoter such as Cbh, or as an aggregate of a CNS-specific promoter such as hSyn and a constitutive promoter such as Cbh. In some embodiments, the liver biodistribution and/or transduction is as defined in any one of embodiments D-1 through D-30.
Embodiment D-1: In an embodiment of Category D, the liver biodistribution and/or transduction is about (or no more than about) 1-times the liver biodistribution and/or transduction of a virus particle comprising a variant capsid polypeptide having a reference sequence, e.g., having the wild-type capsid protein, e.g., having capsid polypeptides of SEQ ID NO:1.
Embodiment D-2: In an embodiment of Category D, the liver biodistribution and/or transduction is about (or no more than about) 0.9-times the liver biodistribution and/or transduction of a virus particle comprising a variant capsid polypeptide having a reference sequence, e.g., having the wild-type capsid protein, e.g., having capsid polypeptides of SEQ ID NO:1.
Embodiment D-3: In an embodiment of Category D, the liver biodistribution and/or transduction is about (or no more than about) 0.7-times the liver biodistribution and/or transduction of a virus particle comprising a variant capsid polypeptide having a reference sequence, e.g., having the wild-type capsid protein, e.g., having capsid polypeptides of SEQ ID NO:1.
Embodiment D-4: In an embodiment of Category D, the liver biodistribution and/or transduction is about (or no more than about) 0.6-times the liver biodistribution and/or transduction of a virus particle comprising a variant capsid polypeptide having a reference sequence, e.g., having the wild-type capsid protein, e.g., having capsid polypeptides of SEQ ID NO:1.
Embodiment D-5: In an embodiment of Category D, the liver biodistribution and/or transduction is about (or no more than about) 0.5-times the liver biodistribution and/or transduction of a virus particle comprising a variant capsid polypeptide having a reference sequence, e.g., having the wild-type capsid protein, e.g., having capsid polypeptides of SEQ ID NO:1.
Embodiment D-6: In an embodiment of Category D, the liver biodistribution and/or transduction is about (or no more than about) 0.4-times the liver biodistribution and/or transduction of a virus particle comprising a variant capsid polypeptide having a reference sequence, e.g., having the wild-type capsid protein, e.g., having capsid polypeptides of SEQ ID NO:1.
Embodiment D-7: In an embodiment of Category D, the liver biodistribution and/or transduction is about (or no more than about) 0.3-times the liver biodistribution and/or transduction of a virus particle comprising a variant capsid polypeptide having a reference sequence, e.g., having the wild-type capsid protein, e.g., having capsid polypeptides of SEQ ID NO:1.
Embodiment D-8: In an embodiment of Category D, the liver biodistribution and/or transduction is about (or no more than about) 0.2-times the liver biodistribution and/or transduction of a virus particle comprising a variant capsid polypeptide having a reference sequence, e.g., having the wild-type capsid protein, e.g., having capsid polypeptides of SEQ ID NO:1.
Embodiment D-9: In an embodiment of Category D, the liver biodistribution and/or transduction is about (or no more than about) 0.1-times the liver biodistribution and/or transduction of a virus particle comprising a variant capsid polypeptide having a reference sequence, e.g., having the wild-type capsid protein, e.g., having capsid polypeptides of SEQ ID NO:1.

In some embodiments, the improved liver biodistribution and/or transduction is in a range bounded by any two values set forth in embodiments D-1 to D-9. Exemplary ranges are set forth in embodiments D-10 to D-23 below.
Embodiment D-10: In an embodiment of Category D, the liver biodistribution and/or transduction ranges between about 0.1 and about 1 times the liver biodistribution and/or transduction of capsid polypeptide having a reference sequence, e.g., having the wild-type capsid protein, e.g., having capsid polypeptides of SEQ ID NO:1.
Embodiment D-11: In an embodiment of Category D, the liver biodistribution and/or transduction ranges between about 0.2 and about 1 times the liver biodistribution and/or transduction of capsid polypeptide having a reference sequence, e.g., having the wild-type capsid protein, e.g., having capsid polypeptides of SEQ ID NO:1.
Embodiment D-12: In an embodiment of Category D, the liver biodistribution and/or transduction ranges between about 0.1 and about 0.9 times the liver biodistribution and/or transduction of capsid polypeptide having a reference sequence, e.g., having the wild-type capsid protein, e.g., having capsid polypeptides of SEQ ID NO:1.
Embodiment D-13: In an embodiment of Category D, the liver biodistribution and/or transduction ranges between about 0.2 and about 0.9 times the liver biodistribution and/or transduction of capsid polypeptide having a reference sequence, e.g., having the wild-type capsid protein, e.g., having capsid polypeptides of SEQ ID NO:1.
Embodiment D-14: In an embodiment of Category D, the liver biodistribution and/or transduction ranges between about 0.1 and about 0.8 times the liver biodistribution and/or transduction of capsid polypeptide having a reference sequence, e.g., having the wild-type capsid protein, e.g., having capsid polypeptides of SEQ ID NO:1.
Embodiment D-15: In an embodiment of Category D, the liver biodistribution and/or transduction ranges between about 0.2 and about 0.8 times the liver biodistribution and/or transduction of capsid polypeptide having a reference sequence, e.g., having the wild-type capsid protein, e.g., having capsid polypeptides of SEQ ID NO:1.
Embodiment D-16: In an embodiment of Category D, the liver biodistribution and/or transduction ranges between about 0.1 and about 0.7 times the liver biodistribution and/or transduction of capsid polypeptide having a reference sequence, e.g., having the wild-type capsid protein, e.g., having capsid polypeptides of SEQ ID NO:1.
Embodiment D-17: In an embodiment of Category D, the liver biodistribution and/or transduction ranges between about 0.2 and about 0.7 times the liver biodistribution and/or transduction of capsid polypeptide having a reference sequence, e.g., having the wild-type capsid protein, e.g., having capsid polypeptides of SEQ ID NO:1.
Embodiment D-18: In an embodiment of Category D, the liver biodistribution and/or transduction ranges between about 0.1 and about 0.6 times the liver biodistribution and/or transduction of capsid polypeptide having a reference sequence, e.g., having the wild-type capsid protein, e.g., having capsid polypeptides of SEQ ID NO:1.
Embodiment D-19: In an embodiment of Category D, the liver biodistribution and/or transduction ranges between about 0.2 and about 0.6 times the liver biodistribution and/or transduction of capsid polypeptide having a reference sequence, e.g., having the wild-type capsid protein, e.g., having capsid polypeptides of SEQ ID NO:1.
Embodiment D-20: In an embodiment of Category D, the liver biodistribution and/or transduction ranges between about 0.1 and about 0.5 times the liver biodistribution and/or transduction of capsid polypeptide having a reference sequence, e.g., having the wild-type capsid protein, e.g., having capsid polypeptides of SEQ ID NO:1.
Embodiment D-21: In an embodiment of Category D, the liver biodistribution and/or transduction ranges between about 0.2 and about 0.5 times the liver biodistribution and/or transduction of capsid polypeptide having a reference sequence, e.g., having the wild-type capsid protein, e.g., having capsid polypeptides of SEQ ID NO:1.
Embodiment D-22: In an embodiment of Category D, the liver biodistribution and/or transduction ranges between about 0.1 and about 0.4 times the liver biodistribution and/or transduction of capsid polypeptide having a reference sequence, e.g., having the wild-type capsid protein, e.g., having capsid polypeptides of SEQ ID NO:1.
Embodiment D-23: In an embodiment of Category D, the liver biodistribution and/or transduction ranges between about 0.2 and about 0.4 times the liver biodistribution and/or transduction of capsid polypeptide having a reference sequence, e.g., having the wild-type capsid protein, e.g., having capsid polypeptides of SEQ ID NO:1.
Category E (Spleen Biodistribution and/or Transduction): In some aspects of the disclosure, a viral particle comprising the variant capsid polypeptide, e.g., the variant capsid polypeptide described herein, exhibits similar or reduced spleen biodistribution and/or transduction as compared to a viral particle with the wild-type capsid polypeptide (SEQ ID NO:1). In some embodiments, the spleen biodistribution and/ortransduction is measured with a CNS-specific promoter such as hSyn, with a constitutive promoter such as Cbh, or as an aggregate of a CNS-specific promoter such as hSyn and a constitutive promoter such as Cbh. In some embodiments, the spleen biodistribution and/or transduction is as defined in any one of embodiments E-1 through E-30.
Embodiment E-1: In an embodiment of Category E, the spleen biodistribution and/or transduction is about (or no more than about) 10-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-2: In an embodiment of Category E, the spleen biodistribution and/or transduction is about (or no more than about) 5-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-3: In an embodiment of Category E, the spleen biodistribution and/or transduction is about (or no more than about) 3-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-4: In an embodiment of Category E, the spleen biodistribution and/or transduction is about (or no more than about) 2-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-5: In an embodiment of Category E, the spleen biodistribution and/or transduction is about (or no more than about) 1.5-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-6: In an embodiment of Category E, the spleen biodistribution and/or transduction is about (or no more than about) 1-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-7: In an embodiment of Category E, the spleen biodistribution and/or transduction is about (or no more than about) 0.8-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-8: In an embodiment of Category E, the spleen biodistribution and/or transduction is about (or no more than about) 0.6-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-9: In an embodiment of Category E, the spleen biodistribution and/or transduction is about (or no more than about) 0.4-times the spleen biodistribution and/or transduction of a virus particle comprising a variant capsid polypeptide having a reference sequence, e.g., having the wild-type capsid protein, e.g., having capsid polypeptides of SEQ ID NO:1.
Embodiment E-10: In an embodiment of Category E, the spleen biodistribution and/or transduction is about (or no more than about) 0.2-times the spleen biodistribution and/or transduction of a virus particle comprising a variant capsid polypeptide having a reference sequence, e.g., having the wild-type capsid protein, e.g., having capsid polypeptides of SEQ ID NO:1.

In some embodiments, the improved spleen biodistribution and/or transduction is in a range bounded by any two values set forth in embodiments E-1 to E-10. Exemplary ranges are set forth in embodiments E-11 to E-22 below.
Embodiment E-11: In an embodiment of Category E, the spleen biodistribution and/or transduction ranges between about 0.2 and about 10 times the spleen biodistribution and/or transduction of capsid polypeptide having a reference sequence, e.g., having the wild-type capsid protein, e.g., having capsid polypeptides of SEQ ID NO:1.
Embodiment E-12: In an embodiment of Category E, the spleen biodistribution and/or transduction ranges between about 0.2 and about 5 times the spleen biodistribution and/or transduction of capsid polypeptide having a reference sequence, e.g., having the wild-type capsid protein, e.g., having capsid polypeptides of SEQ ID NO:1.
Embodiment E-13: In an embodiment of Category E, the spleen biodistribution and/or transduction ranges between about 0.2 and about 3 times the spleen biodistribution and/or transduction of capsid polypeptide having a reference sequence, e.g., having the wild-type capsid protein, e.g., having capsid polypeptides of SEQ ID NO:1.
Embodiment E-14: In an embodiment of Category E, the spleen biodistribution and/or transduction ranges between about 0.2 and about 2 times the spleen biodistribution and/or transduction of capsid polypeptide having a reference sequence, e.g., having the wild-type capsid protein, e.g., having capsid polypeptides of SEQ ID NO:1.
Embodiment E-15: In an embodiment of Category E, the spleen biodistribution and/or transduction ranges between about 0.2 and about 1.5 times the spleen biodistribution and/or transduction of capsid polypeptide having a reference sequence, e.g., having the wild-type capsid protein, e.g., having capsid polypeptides of SEQ ID NO:1.
Embodiment E-16: In an embodiment of Category E, the spleen biodistribution and/or transduction ranges between about 0.2 and about 1 times the spleen biodistribution and/or transduction of capsid polypeptide having a reference sequence, e.g., having the wild-type capsid protein, e.g., having capsid polypeptides of SEQ ID NO:1.
Embodiment E-17: In an embodiment of Category E, the spleen biodistribution and/or transduction ranges between about 0.4 and about 10 times the spleen biodistribution and/or transduction of capsid polypeptide having a reference sequence, e.g., having the wild-type capsid protein, e.g., having capsid polypeptides of SEQ ID NO:1.
Embodiment E-18: In an embodiment of Category E, the spleen biodistribution and/or transduction ranges between about 0.4 and about 5 times the spleen biodistribution and/or transduction of capsid polypeptide having a reference sequence, e.g., having the wild-type capsid protein, e.g., having capsid polypeptides of SEQ ID NO:1.
Embodiment E-19: In an embodiment of Category E, the spleen biodistribution and/or transduction ranges between about 0.4 and about 3 times the spleen biodistribution and/or transduction of capsid polypeptide having a reference sequence, e.g., having the wild-type capsid protein, e.g., having capsid polypeptides of SEQ ID NO:1.
Embodiment E-20: In an embodiment of Category E, the spleen biodistribution and/or transduction ranges between about 0.4 and about 2 times the spleen biodistribution and/or transduction of capsid polypeptide having a reference sequence, e.g., having the wild-type capsid protein, e.g., having capsid polypeptides of SEQ ID NO:1.
Embodiment E-21: In an embodiment of Category E, the spleen biodistribution and/or transduction ranges between about 0.4 and about 1.5 times the spleen biodistribution and/or transduction of capsid polypeptide having a reference sequence, e.g., having the wild-type capsid protein, e.g., having capsid polypeptides of SEQ ID NO:1.
Embodiment E-22: In an embodiment of Category E, the spleen biodistribution and/or transduction ranges between about 0.4 and about 1 times the spleen biodistribution and/or transduction of capsid polypeptide having a reference sequence, e.g., having the wild-type capsid protein, e.g., having capsid polypeptides of SEQ ID NO:1.

### 6.5. Methods of Making Compositions Described Herein

The disclosure is directed, in part, to a method of making a dependoparvovirus particle, e.g., a dependoparvovirus particle described herein. In some embodiments, a method of making a dependoparvovirus particle comprises providing a cell, cell-free system, or other translation system, comprising a nucleic acid described encoding a variant capsid polypeptide provided for herein, or a polypeptide provided for herein (e.g., a variant capsid polypeptide); and cultivating the cell, cell-free system, or other translation system under conditions suitable for the production of the dependoparvovirus particle, thereby making the dependoparvovirus particle.

In some embodiments, a nucleic acid or polypeptide described herein is produced by a method known to one of skill in the art. In embodiments, the nucleic acids, polypeptides, and fragments thereof of the disclosure are produced by any suitable means, including recombinant production, chemical synthesis, or other synthetic means. Such production methods are within the knowledge of those of skill in the art and are not a limitation of the present invention.

### 6.5.1. Host Cells

Aspects of the disclosure are directed to a host cell comprising a nucleic acid of the disclosure (e.g., encoding a variant capsid polypeptide as described in Section 6.2).

A host cell of the disclosure, e.g., a host cell useful to general AAV virus particles comprising a variant AAV capsid as described herein, generally comprises one or more nucleic acids comprising a coding sequence encoding a variant capsid polypeptide of the disclosure (e.g., as described in Section 6.2), together with a payload (e.g., transgene) and one or more coding sequences encoding additional components useful for promoting packaging of the payload into a dependoparvovirus capsid. Additional components include, for example, coding sequences for a rep protein and dependoparvovirus inverted terminal repeats (ITRs), as well as helper sequences which promote dependoparvovirus particle production and/or secretion. Examples of helper sequences include E1a, E1 b, E2a, E4, and VA. Such helper sequences may be included endogenously within the host cell (e.g., the host cell may be engineered to express such helper sequences, e.g., integrated into the host cell genome) or may be provided exogenously (e.g., transduced on the same or a different nucleic acid as the variant capsid polypeptide, payload, rep, and/or ITRs).

In some embodiments, the helper sequences include AdV5 helper sequences. An exemplary AdV5 genome is disclosed in the art as NCBI Reference Sequence AC_000008.1 (disclosed as SEQ ID NO:1 of PCT Patent Application Publication No. WO/2022/079429 A1). In some embodiments, a host cell disclosed herein comprises a portion of an AdV5 genome encoding for one or more helper protein sequences (e.g., E1a, E1b, E2a, E4) or RNA sequences (e.g., VA). In some embodiments, a host cell disclosed herein comprises a nucleic acid sequence encoding one or more AdV5 helper protein sequences. Certain exemplary AdV5 helper protein sequences are provided below.

AdV5 E1A:
(SEQ ID NO: 40)
MRHIICHGGVITEEMAASLLDQLIEEVLADNLPPPSHFEPPTLHELYDLDVTAPEDPNEEAVS
QIFPDSVMLAVQEGIDLLTFPPAPGSPEPPHLSRQPEQPEQRALGPVSMPNLVPEVIDLTCH
EAGFPPSDDEDEEGEEFVLDYVEHPGHGCRSCHYHRRNTGDPDIMCSLCYMRTCGMFVY
SPVSEPEPEPEPEPEPARPTRRPKMAPAILRRPTSPVSRECNSSTDSCDSGPSNTPPEIHP
WPLCPIKPVAVRVGGRRQAVECIEDLLNEPGQPLDLSCKRPRP
AdV5 E1B 19K:
(SEQ ID NO: 41)
MEAWECLEDFSAVRNLLEQSSNSTSWFWRFLWGSSQAKLVCRIKEDYKWEFEELLKSCGE
LFDSLNLGHQALFQEKVIKTLDFSTPGRAAAAVAFLSFIKDKWSEETHLSGGYLLDFLAMHL
WRAVVRHKNRLLLLSSVRPAIIPTEEQQQQQEEARRRRQEQSPWNPRAGLDPRE
AdV5 E1B 55K:
(SEQ ID NO: 42)
MERRNPSERGVPAGFSGHASVESGCETQESPATVVFRPPGDNTDGGAAAAAGGSQAAAA
GAEPMEPESRPGPSGMNVVQVAELYPELRRILTITEDGQGLKGVKRERGACEATEEARNLA
FSLMTRHRPECITFQQIKDNCANELDLLAQKYSIEQLTTYWLQPGDDFEEAIRVYAKVALRPD
CKYKISKLVNIRNCCYISGNGAEVEIDTEDRVAFRCSMINMWPGVLGMDGVVIMNVRFTGPN
FSGTVFLANTNLILHGVSFYGFNNTCVEAWTDVRVRGCAFYCCWKGVVCRPKSRASIKKCL
FERCTLGILSEGNSRVRHNVASDCGCFMLVKSVAVIKHNMVCGNCEDRASQMLTCSDGNC
HLLKTIHVASHSRKAWPVFEHNILTRCSLHLGNRRGVFLPYQCNLSHTKILLEPESMSKVNLN
GVFDMTMKIWKVLRYDETRTRCRPCECGGKHIRNQPVMLDVTEELRPDHLVLACTRAEFG
SSDEDTD
AdV5 E3 12.5K:
(SEQ ID NO: 43)
MLSGEAEQLRLKHLVHCRRHKCFARDSGEFCYFELPEDHIEGPAHGVRLTAQGELARSLIRE
FTQRPLLVERDRGPCVLTVICNCPNLGLHQDLCCHLCAEYNKYRN
AdV5 E3 CR1-alpha0:
(SEQ ID NO: 44)
MNNSSNSTGYSNSGFSRIGVGVILCLVILFILILTLLCLRLAACCVHICIYCQLFKRWGRHPR
AdV5 E3 gp19K:
(SEQ ID NO: 45)
MIRYIILGLLTLASAHGTTQKVDFKEPACNVTFAAEANECTTLIKCTTEHEKLLIRHKNKIGKYA
VYAIWQPGDTTEYNVTVFQGKSHKTFMYTFPFYEMCDITMYMSKQYKLWPPQNCVENTGT
FCCTAMLITVLALVCTLLYIKYKSRRSFIEEKKMP
AdV5 E3 CR1-beta0:
(SEQ ID NO: 46)
MTNTTNAAAATGLTSTTNTPQVSAFVNNWDNLGMWWFSIALMFVCLIIMWLICCLKRKRAR
PPIYSPIIVLHPNNDGIHRLDGLKHMFFSLTV
AdV5 E3 RID-alpha:
(SEQ ID NO: 47)
MIPRVFILLTLVALFCACSTLAAVSHIEVDCIPAFTVYLLYGFVTLTLICSLITVVIAFIQCIDWVCV
RFAYLRHHPQYRDRTIAELLRIL
AdV5 E3 RID-beta:
(SEQ ID NO: 48)
MKFTVTFLLIICTLSAFCSPTSKPQRHISCRFTRIWNIPSCYNEKSDLSEAWLYAIISVMVFCST
ILALAIYPYLDIGWKRIDAMNHPTFPAPAMLPLQQVVAGGFVPANQPRPTSPTPTEISYFNLT
GGDD
AdV5 E3 14.7K:
(SEQ ID NO: 49)
MTDTLDLEMDGIITEQRLLERRRAAAEQQRMNQELQDMVNLHQCKRGIFCLVKQAKVTYDS
NTTGHRLSYKLPTKRQKLVVMVGEKPITITQHSVETEGCIHSPCQGPEDLCTLIKTLCGLKDL
IPFN
AdV5 E4 ORF6/7:
(SEQ ID NO: 50)
MTTSGVPFGMTLRPTRSRLSRRTPYSRDRLPPFETETRATILEDHPLLPECNTLTMHNAWT
SPSPPVKQPQVGQQPVAQQLDSDMNLSELPGEFINITDERLARQETVWNITPKNMSVTHD
MMLFKASRGERTVYSVCWEGGGRLNTRVL
AdV5 E4 34K:
(SEQ ID NO: 51)
MTTSGVPFGMTLRPTRSRLSRRTPYSRDRLPPFETETRATILEDHPLLPECNTLTMHNVSYV
RGLPCSVGFTLIQEWVVPWDMVLTREELVILRKCMHVCLCCANIDIMTSMMIHGYESWALH
CHCSSPGSLQCIAGGQVLASWFRMVVDGAMFNQRFIWYREVVNYNMPKEVMFMSSVFM
RGRHLIYLRLWYDGHVGSVVPAMSFGYSALHCGILNNIVVLCCSYCADLSEIRVRCCARRTR
RLMLRAVRIIAEETTAMLYSCRTERRRQQFIRALLQHHRPILMHDYDSTPM
AdV5 E4 ORF4:
(SEQ ID NO: 52)
MVLPALPAPPVCDSQNECVGWLGVAYSAVVDVIRAAAHEGVYIEPEARGRLDALREWIYYN
YYTERSKRRDRRRRSVCHARTWFCFRKYDYVRRSIWHDTTTNTISVVSAHSVQ
AdV5 E4 ORF3:
(SEQ ID NO: 53)
MIRCLRLKVEGALEQIFTMAGLNIRDLLRDILRRWRDENYLGMVEGAGMFIEEIHPEGFSLYV
HLDVRAVCLLEAIVQHLTNAIICSLAVEFDHATGGERVHLIDLHFEVLDNLLE
AdV5 E4 ORFB:
(SEQ ID NO: 54)
MFERKMVSFSVVVPELTCLYLHEHDYDVLSFLREALPDFLSSTLHFISPPMQQAYIGATLVSIA
PSMRVIISVGSFVMVPGGEVAALVRADLHDYVQLALRRDLRDRGIFVNVPLLNLIQVCEEPEF
LQS
AdV5 E4 ORF1:
(SEQ ID NO: 55)
MAAAVEALYVVLEREGAILPRQEGFSGVYVFFSPINFVIPPMGAVMLSLRLRVCIPPGYFGRF
LALTDVNQPDVFTESYIMTPDMTEELSVVLFNHGDQFFYGHAGMAVVRLMLIRVVFPVVRQ
ASNV

In some embodiments, the helper sequences include AdV2 helper sequences. An exemplary AdV2 genome is disclosed in the art as NCBI Reference Sequence AC_000007.1 (disclosed as SEQ ID NO:2 of PCT Patent Application Publication No. WO/2022/079429 A1). In some embodiments, a host cell disclosed herein comprises a portion of an AdV2 genome encoding for one or more helper protein sequences (e.g., E1a, E1b, E2a, E4) or RNA sequences (e.g., VA). In some embodiments, a host cell disclosed herein comprises a nucleic acid sequence encoding one or more AdV2 helper protein sequences. Certain exemplary AdV2 helper protein sequences are provided below.

AdV2 E1A:
(SEQ ID NO: 56)
MRHIICHGGVITEEMAASLLDQLIEEVLADNLPPPSHFEPPTLHELYDLDVTAPEDPNEEAVS
QIFPESVMLAVQEGIDLFTFPPAPGSPEPPHLSRQPEQPEQRALGPVSMPNLVPEVIDLTCH
EAGFPPSDDEDEEGEEFVLDYVEHPGHGCRSCHYHRRNTGDPDIMCSLCYMRTCGMFVY
SPVSEPEPEPEPEPEPARPTRRPKLVPAILRRPTSPVSRECNSSTDSCDSGPSNTPPEIHPV
VPLCPIKPVAVRVGGRRQAVECIEDLLNESGQPLDLSCKRPRP
AdV2 E1B 19K:
(SEQ ID NO: 57)
MEAWECLEDFSAVRNLLEQSSNSTSWFWRFLWGSSQAKLVCRIKEDYKWEFEELLKSCGE
LFDSLNLGHQALFQEKVIKTLDFSTPGRAAAAVAFLSFIKDKWSEETHLSGGYLLDFLAMHL
WRAVVRHKNRLLLLSSVRPAIIPTEEQQQEEARRRRRQEQSPWNPRAGLDPRE
AdV2 E1B 55K:
(SEQ ID NO: 58)
MERRNPSERGVPAGFSGHASVESGGETQESPATVVFRPPGNNTDGGATAGGSQAAAAAG
AEPMEPESRPGPSGMNVVQVAELFPELRRILTINEDGQGLKGVKRERGASEATEEARNLTF
SLMTRHRPECVTFQQIKDNCANELDLLAQKYSIEQLTTYWLQPGDDFEEAIRVYAKVALRPD
CKYKISKLVNIRNCCYISGNGAEVEIDTEDRVAFRCSMINMWPGVLGMDGVVIMNVRFTGPN
FSGTVFLANTNLILHGVSFYGFNNTCVEAWTDVRVRGCAFYCCWKGVVCRPKSRASIKKCL
FERCTLGILSEGNSRVRHNVASDCGCFMLVKSVAVIKHNMVCGNCEDRASQMLTCSDGNC
HLLKTIHVASHSRKAWPVFEHNILTRCSLHLGNRRGVFLPYQCNLSHTKILLEPESMSKVNLN
GVFDMTMKIWKVLRYDETRTRCRPCECGGKHIRNQPVMLDVTEELRPDHLVLACTRAEFG
SSDEDTD
AdV2 E3 12.5K:
(SEQ ID NO: 59)
MTSGEAERLRLTHLDHCRRHKCFARGSGEFCYFELPEEHIEGPAHGVRLTTQVELTRSLIRE
FTKRPLLVERERGPCVLTVVCNCPNPGLHQDLCCHLCAEYNKYRN
AdV2 E3 CR1-alphap0:
(SEQ ID NO: 60)
MSNSSNSTSLSNFSGIGVGVILTLVILFILILALLCLRVAACCTHVCTYCQLFKRWGQHPR
AdV2 E3 gp19K:
(SEQ ID NO: 61)
MRYMILGLLALAAVCSAAKKVEFKEPACNVTFKSEANECTTLIKCTTEHEKLIIRHKDKIGKYA
VYAIWQPGDTNDYNVTVFQGENRKTFMYKFPFYEMCDITMYMSKQYKLWPPQKCLENTGT
FCSTALLITALALVCTLLYLKYKSRRSFIDEKKMP
AdV2 E3 CR1-beta0:
(SEQ ID NO: 62)
MTGSTIAPTTDYRNTTATGLTSALNLPQVHAFVNDWASLDMWWFSIALMFVCLIIMWLICCLK
RRRARPPIYRPIIVLNPHNEKIHRLDGLKPCSLLLQYD
AdV2 E3 RID alpha:
(SEQ ID NO: 63)
MIPRVLILLTLVALFCACSTLAAVAHIEVDCIPPFTVYLLYGFVTLILICSLVTVVIAFIQFIDWVCV
RIAYLRHHPQYRDRTIADLLRIL
AdV2 E3 RID beta:
(SEQ ID NO: 64)
MKRSVIFVLLIFCALPVLCSQTSAPPKRHISCRFTQIWNIPSCYNKQSDLSEAWLYAIISVMVF
CSTIFALAIYPYLDIGWNAIDAMNHPTFPVPAVIPLQQVIAPINQPRPPSPTPTEISYFNLTGGD
D
AdV2 E3 14.7K:
(SEQ ID NO: 65)
MTESLDLELDGINTEQRLLERRKAASERERLKQEVEDMVNLHQCKRGIFCVVKQAKLTYEK
TTTGNRLSYKLPTQRQKLVLMVGEKPITVTQHSAETEGCLHFPYQGPEDLCTLIKTMCGIRD
LIPFN
AdV2 E4 ORF6/7:
(SEQ ID NO: 66)
MTTSGVPFGMTLRPTRSRLSRRTPYSRDRLPPFETETRATILEDHPLLPECNTLTMHNAWT
SPSPPVEQPQVGQQPVAQQLDSDMNLSELPGEFINITDERLARQETVWNITPKNMSVTHD
MMLFKASRGERTVYSVCWEGGGRLNTRVL
AdV2 E4 34K:
(SEQ ID NO: 67)
MTTSGVPFGMTLRPTRSRLSRRTPYSRDRLPPFETETRATILEDHPLLPECNTLTMHNVSYV
RGLPCSVGFTLIQEWVVPWDMVLTREELVILRKCMHVCLCCANIDIMTSMMIHGYESWALH
CHCSSPGSLQCIAGGQVLASWFRMVVDGAMFNQRFIWYREVVNYNMPKEVMFMSSVFM
RGRHLIYLRLWYDGHVGSVVPAMSFGYSALHCGILNNIVVLCCSYCADLSEIRVRCCARRTR
RLMLRAVRIIAEETTAMLYSCRTERRRQQFIRALLQHHRPILMHDYDSTPM
AdV2 E4 ORF4:
(SEQ ID NO: 68)
MVLPALPAPPVCDSQNECVGWLGVAYSAVVDVIRAAAHEGVYIEPEARGRLDALREWIYYN
YYTERAKRRDRRRRSVCHARTWFCFRKYDYVRRSIWHDTTTNTISVVSAHSVQ
AdV2 E4 ORF3:
(SEQ ID NO: 69)
MIRCLRLKVEGALEQIFTMAGLNIRDLLRDILIRWRDENYLGMVEGAGMFIEEIHPEGFSLYV
HLDVRAVCLLEAIVQHLTNAIICSLAVEFDHATGGERVHLIDLHFEVLDNLLE
AdV2 E4 ORF2:
(SEQ ID NO: 70)
MFERKMVSFSVVVPELTCLYLHEHDYDVLAFLREALPDFLSSTLHFISPPMQQAYIGATLVSIA
PSMRVIISVGSFVMVPGGEVAALVRADLHDYVQLALRRDLRDRGIFVNVPLLNLIQVCEEPEF
LQS
AdV2 E4 ORF1:
(SEQ ID NO: 71)
MAAAVEALYVVLEREGAILPRQEGFSGVYVFFSPINFVIPPMGAVMLSLRLRVCIPPGYFGRF
LALTDVNQPDVFTESYIMTPDMTEELSVVLFNHGDQFFYGHAGMAVVRLMLIRVVFPVVRQ
ASNV

Additional AAV helper sequences are recognized in the art and include, for example, those described in U.S. Patent Application Publication Nos. 2004/0248288 A1 and 2022/0259572A1, and in PCT Patent Application Publication Nos. WO/1997/017458 A1, WO/2024/143429 A1, and WO/2020/208379 A1, each of which is incorporated herein by reference.

Expression control sequences include efficient RNA processing signals such as splicing and polyadenylation (polyA) signals; appropriate transcription initiation, termination, promoter and enhancer sequences; sequences that stabilize cytoplasmic mRNA; sequences that enhance protein stability; sequences that enhance translation efficiency (e.g., Kozak consensus sequence); and in some embodiments, sequences that enhance secretion of the encoded transgene product. Expression control sequences, including promoters which are native, constitutive, inducible and/or tissue-specific, are known in the art and can be utilized with the compositions and methods disclosed herein.

In some embodiments, the native promoter for the transgene is used. Without wishing to be bound by theory, the native promoter can mimic native expression of the transgene, or provide temporal, developmental, or tissue-specific expression, or expression in response to specific transcriptional stimuli. In some embodiments, the transgene is operably linked to other native expression control elements, such as enhancer elements, polyadenylation sites or Kozak consensus sequences, e.g., to mimic the native expression.

In some embodiments, the transgene is operably linked to a tissue-specific promoter, e.g., a promoter active specifically in one or more liver cell types.

In some embodiments, a vector, e.g., a plasmid, carrying a transgene includes a selectable marker or a reporter gene. Such selectable reporters or marker genes can be used to signal the presence of the vector, e.g., plasmid, in bacterial cells. Other components of the vector, e.g., plasmid, include an origin of replication. Selection of these and other promoters and vector elements are conventional and many such sequences are available (see, e.g., Sambrook et al, Molecular Cloning: A Laboratory Manual, Cold Spring Harbor Press, Cold Spring Harbor, NY, and references cited therein).

In some embodiments, an insect cell may be used in production of the compositions described herein or in the methods of making a dependoparvovirus particle described herein. For example, an insect cell line used can be from Spodoptera frugiperda, such as Sf9, SF21, SF900+, drosophila cell lines, mosquito cell lines, e.g., Aedes albopictus derived cell lines, domestic silkworm cell lines, e.g., Bombyxmori cell lines, Trichoplusia ni cell lines such as High Five cells or Lepidoptera cell lines such as Ascalapha odorata cell lines. In some embodiments, the insect cells are susceptible to baculovirus infection, including High Five, Sf9, Se301, SeIZD2109, SeUCR1, SP900+, Sf21, BTI-TN-5B1-4, MG-1, Tn368, HzAml, BM-N, Ha2302, Hz2E5 and Ao38. Use of insect cells for expression of heterologous proteins is well recognized in the art, as are methods of introducing nucleic acids, such as vectors, e.g., insect-cell compatible vectors, into such cells and methods of maintaining such cells in culture. See, for example, O'Reilly et al., 1994, Baculovirus Expression Vectors, A Laboratory Manual. Oxford Univ. Press; Satnulski et al., 1989, J. Vir.63:3822-8; Kajigaya et al., 1991 PNAS 88:4646-50; Ruffin et al., 1992, J. Vir. 66:6922-30; Kimbauer et al., 1996, Vir.21.9:37-44; Zhao et al., 2000, Vir.272:382-93; and U.S. Pat. No. 6,204,059, the contents of each of which are incorporated herein by reference in their entireties.

In certain embodiments, insect host cell systems, in combination with baculoviral systems (e.g., as described by Luckow et al., 1988, Bio/Technology 6:47) is used. In certain embodiments, the expression system is a Trichoplusia ni, Tn 5B1-4 insect cells/baculoviral system, which can be used for production of high levels of proteins, as described in U.S. Pat. No. 6,660,521, incorporated herein by reference in its entirety.

Expansion, culture, transfection, infection, and storage of insect cells can be carried out in any cell culture media, cell transfection media or storage media known in the art. Nonlimiting examples of media are Hyclone SFX Insect Cell Culture Media, Expression System ESF AF Insect Cell Culture Medium, Basal IPL-41 Insect Cell Culture Media, ThermoFisher Sf90011 media, ThermoFisher Sf900111 media, and ThermoFisher Grace's Insect Media. Insect cell mixtures and/or media can also comprise appropriate formulation additives or elements, including but not limited to salts, acids, bases, buffers, and surfactants (such as Poloxatner 188/Pluronic F-68).

In some embodiments, the methods of the disclosure can be carried out with a mammalian cell type which allows for replication of dependoparvovirus or production of biologic products, and which can be maintained in culture. Host cells include cells derived from mammalian species including but not limited to, human, monkey, mouse, rat, rabbit, and hamster. Host cells can be of any suitable cell type, including but not limited to cell lines, fibroblasts, hepatocytes, tumor cells, and transformed cells. The mammalian cells used can be HEK293, HEK293T, HeLa, CHO, NSO, SP2/0, PER.C6, Vero, RD, BHK, HT 1080, A549, Cos-7, ARPE-19, MRC-5, WEH1, 3T3, 1.0T1/2, MDCK, COS 1, COS 7, BSC 1, BSC 40, BMT 10, W138, Saos, C2C12, HepG2, L cells, primary fibroblast, hepatocyte and myoblast cells derived from mammals, COS cells, C127, 3T3, CHO, HeLa cells, KB cells, BHK, and other mammalian cell lines as described in U.S. Pat. Nos. 6,156,303, 5,387,484, 5,741,683, 5,691,176, 6,428,988 and 5,688,676, 6,541,258, the contents of each of which are incorporated herein by reference.

In some embodiments, the host cell comprises a nucleic acid encoding a variant capsid polypeptide disclosed herein, where the nucleic acid is integrated into the host cell genome. Such host cells include adenovirus rep and cap genes integrated into the genome. Transcription of the integrated rep and cap genes may be dependent upon introduction of certain helper virus sequences (e.g., adenovirus E4, E2a and/or VA RNA) into the cell by transduction or other suitable means. Example plasmid free host cells are described in U.S. Patent Application Publication No. 2022/0025396 A1, and U.S. Pat. No. 5,658,785, incorporated herein by reference.

In some embodiments, the host cells are trans-complementing packaging cell lines that provide functions deleted from a replication-defective helper virus, e.g., HEK293 cells or other Ea trans-complementing cells. In some embodiments, the packaging cell line 293-10-3 is used as described in U.S. Pat. No. 6,281,010, incorporated herein by reference.

In some embodiments, mammalian host cells (e.g. 293T cells) can be in an adherent state (e.g., adhered/attached to a suitable surface of a cell culture flask, vial, tray, well, tube, etc.). In other embodiments, mammalian host cells can be in a suspended state (e.g., suspended in a medium).

### 6.5.2. Viral Particle Production

In embodiments, the nucleic acids of the disclosure are situated as a part of any genetic element (vector) which can be delivered to a host cell, e.g., naked DNA, a plasmid, phage, transposon, cosmid, episome, a protein in a non-viral delivery vehicle (e.g., a lipid-based carrier), virus, etc., which transfer the sequences carried thereon. Such a vector can be delivered into a host cell by any suitable method, including transfection, liposome delivery, electroporation, membrane fusion techniques, viral infection, high velocity DNA-coated pellets, and protoplast fusion. A person of skill in the art possesses the knowledge and skill in nucleic acid manipulation to construct any embodiment of this invention and said skills include genetic engineering, recombinant engineering, and synthetic techniques. See, e.g., Sambrook et al, Molecular Cloning: A Laboratory Manual, Cold Spring Harbor Press, Cold Spring Harbor, NY.

In some embodiments, a vector of the disclosure comprises sequences encoding a dependoparvovirus variant capsid polypeptide as provided for herein or a fragment thereof. In some embodiments, a vector of the disclosure comprises sequences encoding a dependoparvovirus Rep protein or a fragment thereof. Such a Rep coding region encodes at least for AAV Rep78, Rep68, Rep52, and Rep40, or functional homologs thereof. The Rep coding region is not required to include all wild-type genes but may be altered (e.g., by insertion, deletion, or mutation of one or more nucleotides) so long as the rep genes present provide for sufficient replication functions when expressed in the recombinant cell. The Rep coding region may be derived from any AAV serotype. In some embodiments, the Rep coding region is or comprises a rep gene encoding AAV2 Rep proteins, exemplary sequences of which are provided below.

AAV2 Rep78:
(SEQ ID NO: 72)
MPGFYEIVIKVPSDLDGHLPGISDSFVNWVAEKEWELPPDSDMDLNLIEQAPLTVAEKLQRD
FLTEWRRVSKAPEALFFVQFEKGESYFHMHVLVETTGVKSMVLGRFLSQIREKLIQRIYRGIE
PTLPNWFAVTKTRNGAGGGNKVVDECYIPNYLLPKTQPELQWAWTNMEQYLSACLNLTER
KRLVAQHLTHVSQTQEQNKENQNPNSDAPVIRSKTSARYMELVGWLVDKGITSEKQWIQED
QASYISFNAASNSRSQIKAALDNAGKIMSLTKTAPDYLVGQQPVEDISSNRIYKILELNGYDP
QYAASVFLGWATKKFGKRNTIWLFGPATTGKTNIAEAIAHTVPFYGCVNWTNENFPFNDCV
DKMVIWWEEGKMTAKVVESAKAILGGSKVRVDQKCKSSAQIDPTPVIVTSNTNMCAVIDGN
STTFEHQQPLQDRMFKFELTRRLDHDFGKVTKQEVKDFFRWAKDHVVEVEHEFYVKKGGA
KKRPAPSDADISEPKRVRESVAQPSTSDAEASINYADRYQNKCSRHVGMNLMLFPCRQCE
RMNQNSNICFTHGQKDCLECFPVSESQPVSVVKKAYQKLCYIHHIMGKVPDACTACDLVNV
DLDDCIFEQ
AAV2 Rep68:
(SEQ ID NO: 73)
MPGFYEIVIKVPSDLDGHLPGISDSFVNWVAEKEWELPPDSDMDLNLIEQAPLTVAEKLQRD
FLTEWRRVSKAPEALFFVQFEKGESYFHMHVLVETTGVKSMVLGRFLSQIREKLIQRIYRGIE
PTLPNWFAVTKTRNGAGGGNKVVDECYIPNYLLPKTQPELQWAWTNMEQYLSACLNLTER
KRLVAQHLTHVSQTQEQNKENQNPNSDAPVIRSKTSARYMELVGWLVDKGITSEKQWIQED
QASYISFNAASNSRSQIKAALDNAGKIMSLTKTAPDYLVGQQPVEDISSNRIYKILELNGYDP
QYAASVFLGWATKKFGKRNTIWLFGPATTGKTNIAEAIAHTVPFYGCVNWTNENFPFNDCV
DKMVIWWEEGKMTAKVVESAKAILGGSKVRVDQKCKSSAQIDPTPVIVTSNTNMCAVIDGN
STTFEHQQPLQDRMFKFELTRRLDHDFGKVTKQEVKDFFRWAKDHVVEVEHEFYVKKGGA
KKRPAPSDADISEPKRVRESVAQPSTSDAEASINYADRLARGHSL
AAV2 Rep52:
(SEQ ID NO: 74)
MELVGWLVDKGITSEKQWIQEDQASYISFNAASNSRSQIKAALDNAGKIMSLTKTAPDYLVG
QQPVEDISSNRIYKILELNGYDPQYAASVFLGWATKKFGKRNTIWLFGPATTGKTNIAEAIAHT
VPFYGCVNWTNENFPFNDCVDKMVIWWEEGKMTAKVVESAKAILGGSKVRVDQKCKSSA
QIDPTPVIVTSNTNMCAVIDGNSTTFEHQQPLQDRMFKFELTRRLDHDFGKVTKQEVKDFFR
WAKDHWVEVEHEFYVKKGGAKKRPAPSDADISEPKRVRESVAQPSTSDAEASINYADRYQN
KCSRHVGMNLMLFPCRQCERMNQNSNICFTHGQKDCLECFPVSESQPVSVVKKAYQKLC
YIHHIMGKVPDACTACDLVNVDLDDCIFEQ
AAV2 Rep40:
(SEQ ID NO: 75)
MELVGWLVDKGITSEKQWIQEDQASYISFNAASNSRSQIKAALDNAGKIMSLTKTAPDYLVG
QQPVEDISSNRIYKILELNGYDPQYAASVFLGWATKKFGKRNTIWLFGPATTGKTNIAEAIAHT
VPFYGCVNWTNENFPFNDCVDKMVIWWEEGKMTAKVVESAKAILGGSKVRVDQKCKSSA
QIDPTPVIVTSNTNMCAVIDGNSTTFEHQQPLQDRMFKFELTRRLDHDFGKVTKQEVKDFFR
WAKDHVVEVEHEFYVKKGGAKKRPAPSDADISEPKRVRESVAQPSTSDAEASINYADRLAR
GHSL

In some embodiments the Rep coding region encodes for Rep sequences having at least 80%, at least 85%, at least 90%, at least 95%, or at least 99% sequence identity to one or more AAV2 Rep proteins. In some embodiments, the Rep coding region comprises a nucleotide sequence encoding for a Rep78 protein having at least 80%, at least about 85%, at least about 90%, at least about 95%, at least 98%, at least 99%, or 100% sequence identity to AAV2 Rep78. In some embodiments, the Rep coding region comprises a nucleotide sequence encoding for a Rep68 protein having at least 80%, at least about 85%, at least about 90%, at least about 95%, at least 98%, at least 99%, or 100% sequence identity to AAV2 Rep68. In some embodiments, the Rep coding region comprises a nucleotide sequence encoding for a Rep52 protein having at least 80%, at least about 85%, at least about 90%, at least about 95%, at least 98%, at least 99%, or 100% sequence identity to AAV2 Rep52. In some embodiments, the Rep coding region comprises a nucleotide sequence encoding for a Rep40 protein having at least 80%, at least about 85%, at least about 90%, at least about 95%, at least 98%, at least 99%, or 100% sequence identity to AAV2 Rep40.

In some embodiments, the Rep coding sequence encodes for wild type Rep proteins. Alternatively, the Rep coding sequence may encode for one or more mutant Rep proteins having improved properties compared with wild type Rep proteins. Examples of such mutant Rep proteins are described in U.S. Pat. No. 11,060,070, incorporated herein by reference.

Additional exemplary Rep gene and protein sequences are disclosed as SEQ ID NOs: 7 to 15 and 20 to 22 of PCT Patent Application Publication No. WO/2022/079429 A1, incorporated herein by reference.

In some embodiments, such vectors contain both dependoparvovirus cap and rep proteins. In vectors in which both AAV rep and cap are provided, in embodiments, the dependoparvovirus rep and dependoparvovirus cap sequences are both of the same dependoparvovirus species or serotype origin. Alternatively, the present embodiments also provide vectors in which the rep sequences are from a dependoparvovirus species or serotype which differs from that which is providing the cap sequences (e.g., AAV2 rep sequences and AAV9 cap sequences). In some embodiments, the rep and cap sequences are expressed from separate sources (e.g., separate vectors, or a host cell genome and a vector). In some embodiments, the rep sequences are fused in frame to cap sequences of a different dependoparvovirus species or serotype to form a chimeric dependoparvovirus vector.

In some embodiments, the vectors of the invention further contain a payload, e.g., a minigene comprising a selected transgene, e.g., flanked by dependoparvovirus 5′ ITR and dependoparvovirus 3′ ITR. In some embodiments, the ITR is from the same serotype as the variant capsid polypeptide. In some embodiments, the ITR is of a different serotype than the variant capsid polypeptide. In some embodiments, the viral genome comprises two ITR sequence regions, wherein the ITRs are of the same serotype as one another. In some embodiments, the viral genome comprises two ITR sequence regions, wherein the ITRs are of different serotypes. Non-limiting examples include zero, one or both of the ITRs having the same serotype as the capsid. In one embodiment both ITRs of the viral genome of the AAV particle are AAV2 ITRs. Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In one embodiment, the ITRs are 140-142 nucleotides in length. Nonlimiting examples of ITR lengths are 102, 105, 130, 140, 141, 142, 145 nucleotides in length.

The vectors described herein, e.g., a plasmid, are useful for a variety of purposes, but are particularly well suited for use in production of recombinant dependoparvovirus particles comprising dependoparvovirus sequences or a fragment thereof, and in some embodiments, a payload.

In one aspect, the disclosure provides a method of making a dependoparvovirus particle (e.g., a dependoparvovirus B particle, e.g., an AAV9 particle), or a portion thereof. In some embodiments, the method comprises culturing a host cell which contains a nucleic acid sequence encoding a dependoparvovirus variant capsid protein as provided for herein, or fragment thereof; a functional rep gene (e.g., encoding Rep proteins as described herein); a payload, e.g., a minigene comprising dependoparvovirus inverted terminal repeats (ITRs) and a transgene; and sufficient helper functions to promote packaging of the payload, e.g., minigene, into the dependoparvovirus capsid. In embodiments, the components necessary to be cultured in the host cell to package a payload, e.g., minigene, in a dependoparvovirus capsid are provided to the host cell in trans. In some embodiments, any one or more of the required components (e.g., payload (e.g., minigene), rep sequences, cap sequences, and/or helper functions) are provided by a host cell which has been engineered to stably comprise one or more of the required components using methods known to those of skill in the art. In some embodiments, a host cell which has been engineered to stably comprise the required component(s) comprises it under the control of an inducible promoter. In some embodiments, the required components are under the control of a constitutive promoter. Examples of suitable inducible and constitutive promoters are provided herein, and further examples are known to those of skill in the art. In some embodiments, a selected host cell which has been engineered to stably comprise one or more components comprises a component under the control of a constitutive promoter and another component under the control of one or more inducible promoters. For example, a host cell which has been engineered to stably comprise the required components is generated from HEK 293 cells (e.g., which comprise helper functions under the control of a constitutive promoter), which comprises the rep and/or cap proteins under the control of one or more inducible promoters.

In embodiments, the payload (e.g., minigene), rep sequences, cap sequences, and helper functions required for producing a dependoparvovirus particle of the disclosure are delivered to the packaging host cell in the form of any genetic element which transfers the sequences carried thereon (e.g., in a vector or combination of vectors). The genetic element may be delivered by any suitable method, including those described herein. Methods used to construct genetic elements, vectors, and other nucleic acids of the disclosure are known to those with skill and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, J. Virol, 70:520-532 (1993) and U.S. Pat. No. 5,478,745. Unless otherwise specified, the dependoparvovirus ITRs, and other selected dependoparvovirus components described herein, are readily selected from among any dependoparvovirus species and serotypes, e.g., AAV1, AAV2, AAV3, AAV4, AAV5, AAV6, AAV7, AAV8, AAVrh74, or AAV9.

ITRs or other dependoparvovirus components may be readily isolated using techniques available to those of skill in the art from a dependoparvovirus species or serotype. Dependoparvovirus species and serotypes may be isolated or obtained from academic, commercial, or public sources (e.g., the American Type Culture Collection, Manassas, VA). In some embodiments, the dependoparvovirus sequences may be obtained through synthetic or other suitable means by reference to published sequences such as are available in the literature or in databases such as, e.g., GenBank or PubMed.

Methods of expressing proteins (e.g., recombinant or heterologous proteins, e.g., dependoparvovirus polypeptides) in insect cells are well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kirnbauer et al., Vir. 219:37-44 (1996); Zhao et al., Vir. 272:382-93 (2000); Samulski et al., and U.S. Pat. No. 6,204,059. In some embodiments, a nucleic acid construct encoding dependoparvovirus polypeptides (e.g., a dependoparvovirus genome) in insect cells is an insect cell-compatible vector. An “insect cell-compatible vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell compatible. The vector may integrate into the insect cell's genome or remain present extra-chromosomally. The vector may be present permanently or transiently, e.g., as an episomal vector. Vectors may be introduced by any means known in the art. Such means include but are not limited to chemical treatment of the cells, electroporation, or infection. In some embodiments, the vector is a baculovirus, a viral vector, or a plasmid. Methods of dependoparvovirus capsid (e.g., AAV) production in insect cells include, for example, those described in U.S. Patent Application Publication No. 2024/0093231 A1, U.S. Pat. No. 11,306,291, Joshi et al., 2024, Methods Mol Biol, 2829:203-214, and Marwidi et al., 2024, Mol Ther Methods Clin Dev., 32(2)101228, incorporated herein by reference.

In some embodiments, a nucleic acid sequence encoding a dependoparvovirus polypeptide is operably linked to regulatory expression control sequences for expression in a specific cell type, such as Sf9 or HEK cells. Techniques known to one skilled in the art for expressing foreign genes in insect host cells or mammalian host cells can be used with the compositions and methods of the disclosure. Methods for molecular engineering and expression of polypeptides in insect cells are described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. Promoters suitable for transcription of a nucleotide sequence encoding a dependoparvovirus polypeptide include the polyhedron, p10, p35 or IE-1 promoters and further promoters described in the above references are also contemplated.

In some embodiments, providing a cell comprising a nucleic acid described herein comprises acquiring a cell comprising the nucleic acid.

Methods of cultivating host cells, cell-free systems, and other translation systems are known to those of skill in the art. In some embodiments, cultivating a host cell comprises providing the cell with suitable media and incubating the cell and media for a time suitable to achieve viral particle production.

In some embodiments, a method of making a dependoparvovirus particle further comprises a purification step comprising isolating the dependoparvovirus particle from one or more other components (e.g., from a cell or media component).

In some embodiments, production of the dependoparvovirus particle comprises one or more (e.g., all) of: expression of dependoparvovirus polypeptides, assembly of a dependoparvovirus capsid, expression (e.g., duplication) of a dependoparvovirus genome, and packaging of the dependoparvovirus genome into the dependoparvovirus capsid to produce a dependoparvovirus particle. In some embodiments, production of the dependoparvovirus particle further comprises secretion of the dependoparvovirus particle into the media. The dependoparvovirus particle can be isolated from the collected media. In other embodiments, dependoparvovirus particles are isolated from host cells. For instance, adherent host cells can subsequently be collected by scraping and/or pelleting and suspended cells can be collected by pelleting and transferred into a receptacle. Collection steps can be repeated as necessary for full collection of produced cells. If necessary, host cell lysis can be achieved by consecutive freeze-thaw cycles (−80° C. to 37° C.), chemical lysis (such as adding detergent, e.g., triton), mechanical lysis, or by allowing the cell culture to degrade after reaching ˜0% viability. Cellular debris can be removed by centrifugation and/or depth filtration.

In some embodiments, and as described elsewhere herein, the nucleic acid molecule encoding the variant capsid polypeptide is disposed in a dependoparvovirus genome. In some embodiments, and as described elsewhere herein, the nucleic acid molecule encoding the variant capsid polypeptide is packaged into a dependoparvovirus particle along with the dependoparvovirus genome as part of a method of making a dependoparvovirus particle described herein. In other embodiments, the nucleic acid molecule encoding the variant capsid polypeptide is not packaged into a dependoparvovirus particle made by a method described herein.

In some embodiments, a method of making a dependoparvovirus particle described herein produces a dependoparvovirus particle comprising a payload (e.g., a payload described herein) and the variant capsid polypeptide. In some embodiments, the payload comprises a second nucleic acid (e.g., in addition to the dependoparvovirus genome), and production of the dependoparvovirus particle comprises packaging the second nucleic acid into the dependoparvovirus particle. In some embodiments, a cell, cell-free system, or other translation system for use in a method of making a dependoparvovirus particle comprises the second nucleic acid. In some embodiments, the second nucleic acid comprises an exogenous sequence (e.g., exogenous to the dependoparvovirus, the cell, or to a target cell or subject who will be administered the dependoparvovirus particle). In some embodiments, the exogenous sequence encodes an exogenous polypeptide. In some embodiments, the exogenous sequence encodes a therapeutic product.

In some embodiments, virus particles of the disclosure have a similar production efficiency to viral particles with a reference capsid polypeptide, for example, with the wild-type capsid polypeptide (SEQ ID NO:1). In some embodiments, production efficiency of viral particles of the disclosure is (a) at least 0.1-fold, at least 0.2-fold, at least 0.3-fold, at least 0.4-fold, at least 0.5-fold, at least 0.6-fold, at least 0.7-fold, at least 0.8-fold, or at least 0.9-fold and/or (b) up to 1-fold, e.g., as compared to a viral particle with a reference capsid polypeptide, for example, with the wild-type capsid polypeptide (SEQ ID NO:1), or the production efficiency is within any range bounded by a value in (a) and a value in (b). In some embodiments, production efficiency is at least 0.5-fold as compared to a viral particle with the wild-type capsid polypeptide (SEQ ID NO:1).

Production efficiency can be evaluated by producing viral particles having a variant capsid with a genome encoding a unique barcode and a fluorescent reporter gene under the control of a ubiquitous (e.g., CBh) or a neuronal cell-type specific promoter (e.g., human synapsin) via transient triple transfection of adherent HEK293T cells followed by iodixanol gradient purification.

Various methods and systems for dependoparvovirus production in host cells are recognized in the art and are contemplated herein including, for example, those described in U.S. Patent Application Publication Nos. 20220064671 A1, 20220259572 A1, 20220025396 A1 and PCT Patent Application Publication Nos. WO/1999/011764 A2, WO/2023/178220 A1, WO/2020/208379 A1, WO/2023/143063 A1, WO/2023/239627 A2, WO/2021/156609 A1, and WO/2021/113767 A1, each of which is incorporated herein by reference in its entirety.

### 6.6. Applications

The disclosure is directed, in part, to compositions comprising a nucleic acid, polypeptide, or particles described herein. The disclosure is further directed, in part, to methods utilizing a composition, nucleic acid, polypeptide, or particles described herein. As will be apparent based on the disclosure, nucleic acids, polypeptides, particles, and methods disclosed herein have a variety of utilities.

The disclosure is directed, in part, to a vector comprising a nucleic acid described herein, e.g., a nucleic acid encoding a variant capsid polypeptide. Many types of vectors are known to those of skill in the art. In some embodiments, a vector comprises a plasmid. In some embodiments, the vector is an isolated vector, e.g., removed from a cell or other biological components.

The disclosure is directed, in part to a cell, cell-free system, or other translation system, comprising a nucleic acid or vector described herein, e.g., a nucleic acid or vector comprising a nucleic acid molecule encoding a variant capsid polypeptide. In some embodiments, the cell, cell-free system, or other translation system is capable of producing dependoparvovirus particles comprising the variant capsid polypeptides. In some embodiments, the cell, cell-free system, or other translation system comprises a nucleic acid comprising a dependoparvovirus genome or components of a dependoparvovirus genome sufficient to promote production of dependoparvovirus particles comprising the variant capsid polypeptides.

In some embodiments, the cell, cell-free system, or other translation system further comprises one or more non-dependoparvovirus nucleic acid sequences that promote dependoparvovirus particle production and/or secretion. Said sequences are referred to herein as helper sequences. In some embodiments, a helper sequence comprises one or more genes from another virus, e.g., an adenovirus or herpes virus. In some embodiments, the presence of a helper sequence is necessary for production and/or secretion of a dependoparvovirus particle. In some embodiments, a cell, cell-free system, or other translation system comprises a vector, e.g., plasmid, comprising one or more helper sequences.

In some embodiments, a cell, cell-free system, or other translation system comprises a first nucleic acid and a second nucleic acid, wherein the first nucleic acid comprises sequences encoding one or more dependoparvovirus genes (e.g., a Cap gene, a Rep gene, or a complete dependoparvovirus genome) and a helper sequence, and wherein the second nucleic acid comprises a payload. In some embodiments, a cell, cell-free system, or other translation system comprises a first nucleic acid and a second nucleic acid, wherein the first nucleic acid comprises sequences encoding one or more dependoparvovirus genes (e.g., a Cap gene, a Rep gene, or a complete dependoparvovirus genome) and a payload, and wherein the second nucleic acid comprises a helper sequence. In some embodiments, a cell, cell-free system, or other translation system comprises a first nucleic acid and a second nucleic acid, wherein the first nucleic acid comprises a helper sequence and a payload, and wherein the second nucleic acid comprises sequences encoding one or more dependoparvovirus genes (e.g., a Cap gene, a Rep gene, or a complete dependoparvovirus genome). In some embodiments, a cell, cell-free system, or other translation system comprises a first nucleic acid, a second nucleic acid, and a third nucleic acid, wherein the first nucleic acid comprises sequences encoding one or more dependoparvovirus genes (e.g., a Cap gene, a Rep gene, or a complete dependoparvovirus genome), the second nucleic acid comprises a helper sequence, and the third nucleic acid comprises a payload.

In some embodiments, the first nucleic acid, second nucleic acid, and optionally third nucleic acid are situated in separate molecules, e.g., separate vectors or a vector and genomic DNA. In some embodiments, one, two, or all of the first nucleic acid, second nucleic acid, and optionally third nucleic acid are integrated (e.g., stably integrated) into the genome of a cell.

In embodiments, a cell of the disclosure is generated by transfecting a suitable cell with a nucleic acid described herein. In some embodiments, a method of making a dependoparvovirus particle comprising a variant capsid polypeptide as provided for herein or improving a method of making a dependoparvovirus particle comprises providing a cell described herein. In some embodiments, providing a cell comprises transfecting a suitable cell with one or more nucleic acids described herein.

Many types and kinds of cells suitable for use with the nucleic acids and vectors described herein are known in the art. In some embodiments, the cell is a human cell. In some embodiments, the cell is an immortalized cell or a cell from a cell line known in the art.

In some embodiments, the cell is an HEK293 cell. In some embodiments, the cell is an HEK293T cell.

### 6.6.1. Methods of Delivering a Payload

The disclosure is directed, in part, to a method of delivering a payload to a cell, e.g., a cell in a subject or in a sample. In some embodiments, a method of delivering a payload to a cell comprises contacting the cell with a dependoparvovirus particle comprising a variant capsid polypeptide (e.g., described herein) comprising the payload. The disclosure also includes a dependoparvovirus particle comprising a variant capsid polypeptide (e.g., described herein) comprising a payload described herein for use in the methods of delivering a payload described herein. In some embodiments, the dependoparvovirus particle is a dependoparvovirus particle described herein and comprises a payload described herein. In some embodiments, the cell is a CNS cell. In some embodiments, the method is conducted ex vivo. In some embodiments, the cell is a cell in an ex vivo sample that has been obtained from a subject.

In some embodiments, the payload comprises a transgene. In some embodiments, the transgene is a nucleic acid sequence heterologous to the vector sequences flanking the transgene which encodes a polypeptide, RNA (e.g., a miRNA or siRNA) or other product of interest. In embodiments, the nucleic acid of the transgene is operatively linked to a regulatory component in a manner sufficient to promote transgene transcription, translation, and/or expression in a host cell.

In aspects, a transgene is any polypeptide- or RNA-encoding sequence and the transgene selected will depend upon the use envisioned. In some embodiments, a transgene comprises a reporter sequence, which upon expression produces a detectable signal. Such reporter sequences include, without limitation, DNA sequences encoding colorimetric reporters (e.g., β-lactamase, β-galactosidase (LacZ), alkaline phosphatase), cell division reporters (e.g., thymidine kinase), fluorescent or luminescence reporters (e.g., green fluorescent protein (GFP) or luciferase), resistance conveying sequences (e.g., chloramphenicol acetyltransferase (CAT)), or membrane bound proteins including to which high affinity antibodies directed thereto exist or can be produced by conventional means, e.g., comprising an antigen tag, e.g., hemagglutinin or Myc.

In some embodiments, a reporter sequence operably linked with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. In some embodiments, the transgene encodes a product which is useful in biology and medicine, such as RNA, proteins, peptides, enzymes, dominant negative mutants. In some embodiments, the RNA comprises a tRNA, ribosomal RNA, dsRNA, catalytic RNAs, small hairpin RNA, siRNA, trans-splicing RNA, and antisense RNAs. In some embodiments, the RNA inhibits or abolishes expression of a targeted nucleic acid sequence in a treated subject (e.g., a human or animal subject).

In some embodiments, the transgene is used to correct or ameliorate gene deficiencies. In some embodiments, gene deficiencies include deficiencies in which normal genes are expressed at less than normal levels or deficiencies in which the functional gene product is not expressed. In some embodiments, the transgene encodes a therapeutic protein or polypeptide which is expressed in a host cell. In some embodiments, a dependoparvovirus particle comprises or delivers multiple transgenes, e.g., to correct or ameliorate a gene defect caused by a multi-subunit protein. In some embodiments, a different transgene (e.g., each situated/delivered in a different dependoparvovirus particle, or in a single dependoparvovirus particle) is used to encode each subunit of a protein, or to encode different peptides or proteins, e.g., when the size of the DNA encoding the protein subunit is large, e.g., for immunoglobulin, platelet-derived growth factor, or dystrophin protein. In some embodiments, different subunits of a protein are encoded by the same transgene, e.g., a single transgene encoding each of the subunits with the DNA for each subunit separated by an internal ribozyme entry site (IRES). In some embodiments, the DNA is separated by sequences encoding a 2A peptide, which self-cleaves in a post-translational event. See, e.g., Donnelly et al., 1997, J. Gen. Virol. 78(Pt 1):13-21; Furler et al., 2001, Gene Ther. 8(11):864-873; Klump et al., 2001, Gene Ther 8(10):811-817(each of which is incorporated herein by reference in its entirety).

In some embodiments, virus particles comprising a genome are provided, wherein the genome includes a nucleic acid expression construct. The nucleic acid expression construct can include a heterologous transgene and one or more regulatory elements.

In some embodiments, the regulatory elements include a promoter. In some embodiments, the promoter is a ubiquitous or constitutive promoter active in a mammalian cell, for example a human cell, for example, in a human cell type of interest. In some embodiments, the cell type is a CNS cell such as, for example, a neuronal cell, a glial cell, an endothelia cell, and the like. Examples of ubiquitous promoters include, but are not limited, to a CAG promoter (hybrid from a cytomegalovirus early enhancer element, a chicken-beta actin promoter, e.g., the first exon and the first intron of the chicken beta actin gene, and the splice acceptor of the rabbit beta globin gene), chicken-beta actin promoter, CBA promoter, CBh promoter, CB6 promoter, CMV promoter, human EF1-alpha promoter, PGK promoter, ubiquitin C (UBC) promoter and fragments thereof. In some embodiments, the promoter is a tissue-specific promoter, for example, a promoter specific in CNS tissue or cells of the CNS. Examples of CNS-specific promoters include but are not limited to a synapsin (SYN or SYN1) promoter, a neuron-specific enolase (NSE) promoter, a Ca²+/calmodulin-dependent kinase subunit a (CaMKII) promoter, a synapsin I with a minimal CMV sequence (Synl-minCMV) promoter, a glial fibrillary acidic protein (GFAP) promoter, a internexin neuronal intermediate filament protein alpha (INA) promoter, a nestin (NES) promoter, a neurofilament light chain (NfL) promoter, a neurofilament heavy chain (NfH) promoter, a myelin-associated oligodendrocyte basic protein (MOBP) promoter, a myelin basic protein (MBP) promoter, a tyrosine hydroxylase (TH) promoter, a forkhead box A2 (FOXA2) promoter, a aldehyde dehydrogenase 1 family member L1 (ALDH1L1) promoter, a glutamate decarboxylase 2 (GAD2) promoter, a riken gene A930098C07Rik (A93) promoter, a somatostatin (SST) promoter, a platelet derived growth factor receptor alpha (PDGFRA) promoter, a glutamate receptor metabotropic 1 (GRM1) promoter, a C-type natriuretic peptide precursor (NPPC) promoter, a adrenomedullin (ADM) promoter, a type 2 lactosamine alpha-2,3-sialyltransferase (ST3GAL6) promoter, a ras responsive element binding protein 1 (RREB1) promoter, a deiodinase iodothyronine type II (DI02) promoter, an excitatory amino acid transporter 2 (EAAT2) promoter, a nuclear receptor subfamily 2 group F member 2 (NR2F2) promoter, a platelet-derived growth factor (PDGF) promoter, a methyl-CpG binding protein 2 (MeCP2) promoter, and mouse, primate or human homologs of any of the forgoing, and fragments (e.g., active fragments) of any of the foregoing. In embodiments, the CNS-specific promoter is a neuron specific promoter. In embodiments, the CNS-specific promoter is a astrocyte-specific promoter.

In some embodiments, the promoter is a CBh promoter. An exemplary CBh promoter sequence is set forth as SEQ ID NO:24. In some embodiments, the CBh promoter comprises a nucleotide sequence having at least 90%, at least 95%, at least 96%, at least 97%, or at least 98%, at least 99%, or 100% sequence identity to SEQ ID NO:24.

In other embodiments, the promoter is a synapsin promoter, for example a human synapsin promoter (hSYN). An exemplary hSYN promoter sequence is set forth as SEQ ID NO:25. In some embodiments, the hSYN promoter comprises a nucleotide sequence having at least 90%, at least 95%, at least 96%, at least 97%, or at least 98%, at least 99%, or 100% sequence identity to SEQ ID NO:25.

In some embodiments, the nucleic acid expression construct comprises an intron. In embodiments, the intron is disposed between the promoter and the heterologous transgene. In some aspects, the intron is disposed 5′ to the heterologous transgene on the expression construct, for example immediately 5′ to the heterologous transgene or 100 nucleotides or less 5′ to the heterologous transgene. In some aspects, the intron is a chimeric intron derived from human b-globin and Ig heavy chain (also known as b-globin splice donor/immunoglobulin heavy chain splice acceptor intron, or b-globin/IgG chimeric intron; Reed, R., et al. Genes and Development, 1989, incorporated herein by reference in its entirety). In other aspects, the intron is a VH4 intron or a SV40 intron.

As provided herein, in some embodiments, virus particles comprising a payload, wherein the payload includes a nucleic acid that includes a heterologous transgene are provided. In some embodiments, the heterologous transgene encodes an RNA interference agent, for example a siRNA, shRNA or other interfering nucleic acid.

In some embodiments, the payload includes a heterologous transgene that encodes a therapeutic polypeptide. In some aspects, the heterologous transgene is a human gene or fragment thereof. In some aspects, the therapeutic polypeptide is a human protein. In some embodiments, the heterologous transgene of the virus particle encodes a molecule useful in treating a disease, and the virus particle is administered to a patient in need thereof to treat said disease. In some aspects the payload comprises a molecule that is effective in treating chronic CNS disease, such as, for example, an RNA interference nucleotide (e.g., shRNA, siRNA or miRNA that inhibits APOL-1). Examples of diseases (and heterologous transgenes or molecules encoded by said heterologous transgenes) according to the present disclosure include: MPSI (alpha-L-iduronidase (IDUA)); MPS II—Hunter syndrome (iduronate-2-sulfatase (IDS)); Ceroid lipofuscinosis-Batten disease (CLN1, CLN2, CLN10, CLN13, CLN5, CLN11, CLN4, CNL14, CLN3, CLN6, CLN7, CLN8, CLN12); MPS IIIa—Sanfilippo TypeA syndrome (heparin sulfate sulfatase (also called N-sulfoglucosamine sulfohydrolase (SGSH)); MPS IIIB—Sanfilippo Type b syndrome (N-acetyl-alpha-D-glucosaminidase (NAGLU)); MPS VI—Maroteaux-Lamy syndrome (arylsulfatase B); MPS IVA—Morquio syndrome type A (GALNS); MPS IV B—Morquio syndrome type B (GLB1); chronic or neuropathic pain; Osteogenesis Imperfecgta Type I, II, III or IV (COL1AI and/or COL1A2); hereditary angioedema (SERPING1, C1NH); Osteogenesis Imperfecta Type V (IFITM5); Osteogenesis Imperfecta Type VI (SERPINF1); Osteogenesis Imperfecta Type VII (CRTAP); Osteogenesis Imperfecta Type VIII (LEPRE1 and/or P3H1); Osteogenesis Imperfecta Type IX (PPIB); Gaucher disease type I, II and III (Glucocerebrosidase; GBAI); Parkinson's Disease (Glucocerebrosidase; GBAI and/or dopamine decarboxylase); Pompe (acid maltase; GAA; hGAA); Metachromatic leukodystrophy (Aryl sulfatase A); MPS VII—Sly syndrome (beta-glucuronidase); MPS VIII (glucosamine-6-sulfate sulfatase); MPS IX (Hyaluronidase); maple syrup urine disease (BCKDHA, BCKDHB, and/or DBT); Niemann-Pick disease (Sphingomyelinase); Parkinson's disease (anti-alpha synuclein RNAi); Alzheimer's disease (anti-mutant APP RNAi); Niemann-Pick disease without sphingomyelinase deficiency (NPC1 or NPC gene encoding a cholesterol metabolizing enzyme); Tay-Sachs disease (alpha subunit of beta-hexosaminidase); Sandhoff disease (both alpha and beta subunit of beta-hexosaminidase); Fabry Disease (alpha-galactosidase); Fucosidosis (fucosidase (FUCAI)); Alpha-mannosidosis (alpha-mannosidase); Beta-mannosidosis (beta-mannosidase); Wolman disease (cholesterol ester hydrolase); Dravet syndrome (SCN1A, SCN1B, SCN2A, GABRG2); Parkinson's disease (Neurturin); Parkinson's disease (glial derived growth factor (GDGF)); Parkinson's disease (tyrosine hydroxylase); frontotemporal dementia (progranulin); Angleman syndrome (ubiquitin protein ligase 3A (UBE3A), gene editing systems targeting a UBE3A inhibitory RNA (UBE3A-anitsense transcript)); Parkinson's disease (glutamic acid decarboxylase; FGF-2; BDGF); Spinal MuscularAtrophy (SMN, including SMN1 or SMN2); Friedreich's ataxia (Frataxin); Amyotrophic lateral sclerosis (ALS) (SOD1 inhibitor, e.g., anti-SOD1 RNAi); Glycogen Storage Disease Ia (Glucose-6-phosphatase); XLM™ (MTMI); Crigler Najjar (UGTIAI); CPVT (CASQ2); spinocerebellar ataxia (ATXN2; ATXN3 or other ATXN gene; anti-mutant Machado-Joseph disease/SCA3 allele RNAi); Rett syndrome (MECP2 or fragment thereof); Achromatopsia (CNGB3, CNGA3, GNAT2, PDE6C); Choroidermia (CDM); Danon Disease (LAMP2); Cystic Fibrosis (CFTR or fragment thereof); Duchenne Muscular Dystrophy (Mini-/Micro-Dystrophin Gene); SARS-Cov-2 infection (anti-SARS-Cov-2 RNAi, SARS-Cov-2 genome fragments or S protein (including variants)); Limb Girdle Muscular Dystrophy Type 2C—Gamma-sarcoglycanopathy (human-alpha-sarcoglycan); Advanced Heart Failure (SERCA2a); Rheumatoid Arthritis (TNFR:Fc Fusion; anti-TNF antibody or fragment thereof); Leber Congenital Amaurosis (GAA); X-linked adrenoleukodystrophy (ABCD1); Limb Girdle Muscular Dystrophy Type 2C—Gamma-sarcoglycanopathy (gamma-sarcoglycan); Angelman syndrome (UBE3A); Retinitis Pigmentosa (hMERTK); Age-Related Macular Degeneration (sFLTO1); Phelan-McDermid syndrome (SHANK3; 22q13.3 replacement); Becker Muscular Dystrophy and Sporadic Inclusion Body Myositis (huFollistatin344); Parkinson's Disease (GDNF); Metachromatic Leukodystrophy—MLD (cuARSA); Hepatitis C (anti-HCV RNAi); Limb Girdle Muscular Dystrophy Type 2D (hSGCA); Human Immunodeficiency Virus Infections; (PG9DP); Acute Intermittant Porphyria (PBGD); Leber's Hereditary Optical Neuropathy (PIND4v2); Alpha-1 Antitrypsin Deficiency (alphalAT); X-linked Retinoschisis (RS1); Choroideremia (hCHM); Giant Axonal Neuropathy (GAN); Hemophilia B (Factor IX); Homozygous FH (hLDLR); Dysferlinopathies (DYSF); Achromatopsia (CNGA3 or CNGB3); Progressive supranuclear palsy (MAPT; anti-Tau; anti-MAPT RNAi); Omithine Transcarbamylase deficiency (OTC); Hemophilia A (Factor VIII); Age-related macular degeneration (AMD), including wetAMD (anti-VEGF antibody or RNAi); X-Linked Retinitis Pigmentosa (RPGR); Myotonic dystrophy Type 1 (DMPK; anti-DMPK RNAi, including anti-CTG trinucleotide repeat RNAi); Myotonic dystrophy Type 2 (CNBP); Facioscapulohumeral muscular dystrophy (D4Z4 DNA); oculopharynggeal muscular dystrophy (PABPN1; mutated PABPN1 inhibitor (e.g., RNAi)); Mucopolysaccharidosis Type VI (hARSB); Leber Hereditary Optic Neuropathy (ND4); X-Linked myotubular Myopathy (MTMI); Crigler-Najjar Syndrome (UGTIAI); Retinitis Pigmentosa (hPDE6B); Mucopolysaccharidosis Type 3B (hNAGLU); Duchenne Muscular Dystrophy (GALGT2); Alzheimer's Disease (NGF; ApoE4; ApoE2; ApoE3; Anti-ApoE RNAi, MAPT, anti-Tau antibody, anti-amyloid beta antibody (e.g., aducanumab)); multiple system atrophy; Familial Lipoprotein Lipase Deficiency (LPL); Alpha-1 Antitrypsin Deficiency (hAAT); Leber Congenital Amaurosis 2 (hRPE65v2); Batten Disease; Late Infantile Neuronal Lipofuscinosis (CLN2); Huntington's disease (HTT; anti-HTT RNAi); Fragile X syndrome (FMR1); Fragile X-associated tremor/ataxia syndrome (FMR1), Premature ovarian aging (FMR1), Polycystic ovarian syndrome (FMR1), Leber's Hereditary Optical Neuropathy (PIND4v2); Aromatic Amino Acid Decarboxylase Deficiency (hAADC); Retinitis Pigmentosa (hMERKTK); and Retinitis Pigmentosa (RLBPI). In some embodiments, the CNS disease is a tauopathy (e.g., Alzheimers' disease, progressive supranuclear palsy, frontotemporal dementia (Pick disease), corticobasal degeneration, argyrophilic grain disease, globular glial tauopathies, neurofibrillary tangle dementia, chronic traumatic encephalopathy (CTE), or aging-related tau astrogliopathy) and the payload is an anti-Tau antibody or antisense oligonucleotide targeting human MAPT.

In some embodiments, the heterologous transgene encodes a therapeutic polypeptide. In some aspects, the heterologous transgene is a human gene or fragment thereof. In some aspects, the therapeutic polypeptide is a human protein. In some aspects, the heterologous transgene encodes an antibody or fragment thereof (for example an antibody light chain, an antibody heavy chain, a Fab or an scFv). Examples of antibodies or fragments thereof that are encoded by the heterologous transgene include but are not limited to; and an anti-Ab antibody (e.g., solanezumab, GSK933776, and lecanemab), anti-sortilin (e.g., AL-001), anti-Tau (e.g., ABBV-8E12, UCB-0107, and NI-105), anti-SEMA4D (e.g., VX15/2503), anti-alpha synuclein (e.g., prasinezumab, NI-202, and MED-1341), anti-SOD1 (e.g., NI-204), anti-CGRP receptor (e.g., eptinezumab, fremanezumab, or galcanezumab), anti-VEGF (e.g., sevacizumab, ranibizumab, bevacizumab, and brolucizumab), anti-EpoR (e.g., LKA-651), anti-ALKI (e.g., ascrinvacumab), anti-C5 (e.g., tesidolumab, ravulizumab, and eculizumab), anti-CD105 (e.g., carotuximab), anti-CCIQ (e.g., ANX-007), anti-TNFa (e.g., adalimumab, infliximab, and golimumab), anti-RGMa (e.g., elezanumab), anti-TTR (e.g., NI-301 and PRX-004), anti-CTGF (e.g., pamrevlumab), anti-IL6R (e.g., satralizumab, tocilizumab, and sarilumab), anti-IL6 (e.g., siltuximab, clazakizumab, sirukumab, olokizumab, and gerilimzumab), anti-IL4R (e.g., dupilumab), anti-IL17A (e.g., ixekizumab and secukinumab), anti-IL5R (e.g., reslizumab), anti-IL-5 (e.g., benralizumab and mepolizumab), anti-IL13 (e.g., tralokinumab), anti-IL12/IL23 (e.g., ustekinumab), anti-CD 19 (e.g., inebilizumab), anti-IL31RA (e.g., nemolizumab), anti-ITGF7 mAb (e.g., etrolizumab), anti-SOST mAb (e.g., romosozumab), anti-IgE (e.g., omalizumab), anti-TSLP (e.g., nemolizumab), anti-pKal mAb (e.g., Ianadelumab), anti-ITGA4 (e.g., natalizumab), anti-ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab), optionally wherein the heavy chain (Fab and Fc region) and the light chain are separated by a self-cleaving furin (F)/F2A or furin (F)/T2A, IRES site, or flexible linker, for example, ensuring expression of equal amounts of the heavy and the light chain polypeptides.

In some embodiments, the virus particle comprises a heterologous transgene encoding a genome editing system. Examples include a CRISPR genome editing system (e.g., one or more components of a CRISPR genome editing system such as, for example, a guide RNA molecule and/or a RNA-guided nuclease such as a Cas enzyme such as Cas9, Cpf1 and the like), a zinc finger nuclease genome editing system, a TALEN genome editing system or a meganuclease genome editing system. In embodiments, the genome editing system targets a mammalian, e.g., human, genomic target sequence. In embodiments, the virus particle includes a heterologous transgene encoding a targetable transcription regulator. Examples include a CRISPR-based transcription regulator (for example, one or more components of a CRISPR-based transcription regulator, for example, a guide RNA molecule and/or a enzymatically-inactive RNA-guided nuclease/transcription factor (“TF”) fusion protein such as a dCas9-TF fusion, dCpf1-TF fusion and the like), a zinc finger transcription factor fusion protein, a TALEN transcription regulator or a meganuclease transcription regulator.

In some embodiments, components of a therapeutic molecule or system are delivered by more than one unique virus particle (e.g., a population that includes more than one unique virus particles). In other embodiments, the therapeutic molecule or components of a therapeutic molecule or system are delivered by a single unique virus particle (e.g., a population that includes a single unique virus particle).

In embodiments, the transgene encodes any biologically active product or other product, e.g., a product desirable for study. Suitable transgenes may be readily selected by persons of skill in the art, such as those, but not limited to, those described herein.

Other examples of proteins encoded for by the transgene include, but are not limited to, colony stimulating factors (CSF); blood factors, such as β-globin, hemoglobin, tissue plasminogen activator or an analog thereof such as reteplase, Ianoteplase or tenecteplase, and coagulation factors; interleukins; soluble receptors, such as soluble TNF-α receptors, soluble VEGF receptors, soluble interleukin receptors (e.g., soluble IL-1 receptors and soluble type II IL-1 receptors), or ligand-binding fragments of a soluble receptor; growth factors, such as keratinocyte growth factor (KGF), stem cell factor (SCF), or fibroblast growth factor (FGF, such as basic FGF and acidic FGF); enzymes; chemokines; enzyme activators, such as tissue plasminogen activator; angiogenic agents, such as vascular endothelial growth factors, glioma-derived growth factor, angiogenin, or angiogenin-2; anti-angiogenic agents, such as a soluble VEGF receptor; a protein vaccine; neuroactive peptides, such as nerve growth factor (NGF) or oxytocin; thrombolytic agents; tissue factors; macrophage activating factors; tissue inhibitors of metalloproteinases; or IL-1 receptor antagonists.

In embodiments, the disclosure provides a nucleotide sequence which encodes a molecule for the treatment of Alzheimer's disease. In embodiments, the molecule for the treatment of Alzheimer's disease comprises an inhibitory nucleic acid molecule (e.g., an antisense oligonucleotide or inhibitory RNA (e.g., siRNA, miRNA or shRNA molecule). In embodiments, the molecule for the treatment of Alzheimer's disease comprises an inhibitory nucleic acid molecule (e.g., an antisense oligonucleotide or inhibitory RNA (e.g., siRNA, miRNA or shRNA molecule) targeting beta-amyloid, alpha-synuclein, Tau, TREM, e.g., TREM2, or an apolipoprotein (APO) E protein, e.g. APOE1, APOE2, APOE3 orAPOE4.

In embodiments, the molecule for the treatment of Alzheimer's disease comprises a genome editing system (for example a zinc finger nuclease, a meganuclease, a TALEN, or an RNA-guided genome editing system (e.g. a Cas polypeptide and a guide RNA molecule). In embodiments the genome editing system targets a genetic region encoding a beta-amyloid protein or a Tau protein. In embodiments, the genome editing system targets MSA4.

In embodiments, the molecule for the treatment of Alzheimer's disease is an antibody or antigen-binding fragment thereof (e.g., as scFV). In embodiments, the molecule for the treatment of Alzheimer's disease is a human protein or fragment or variant thereof. In embodiments, the molecule for the treatment of Alzheimer's disease is an inhibitor of beta-amyloid aggregation. In embodiments, the molecule for the treatment of Alzheimer's disease is an inhibitor of alpha-synuclein. In embodiments, the molecule for the treatment of Alzheimer's disease is an anti-beta amyloid antibody, e.g., gantenerumab, crenezumab, aducanumab, lecanemab, bapineuzumab, solanezumab, donanemab or trontinemab (which is anti-beta amyloid/anti-transferrin receptor bispecific antibody). In embodiments, the molecule for the treatment of Alzheimer's disease is a Tau inhibitor, e.g., an anti-tau antibody (e.g., semorinemab). In embodiments, the molecule for the treatment of Alzheimer's disease is an anti-TREM antibody or antigen-binding fragment thereof. In embodiments, the molecule for the treatment of Alzheimer's disease is human nerve growth factor or a fragment or variant thereof. In embodiments, the molecule for the treatment of Alzheimer's disease is human brain-derived neurotrophic factor or a fragment or variant thereof. In embodiments, the molecule for the treatment of Alzheimer's disease is human synapsin-caveolin-1 (SynCav1) or a fragment or variant thereof.

Accordingly, in certain aspects, the disclosure provides a virus particle comprising (a) a variant capsid polypeptide described herein, e.g., a capsid polypeptide described in Section 6.2 and (b) an engineered viral genome comprising (i) a nucleotide sequence encoding a molecule for the treatment of Alzheimer's disease, for example as described herein, and (ii) a promoter operably linked to said nucleotide sequence. The viral genome can further comprise one or more (e.g., two, three, four or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described in Section 6.2; (b) an engineered viral genome comprising (i) a nucleotide sequence encoding a molecule for the treatment of Alzheimer's disease, for example as described herein, and (ii) a promoter operably linked to said nucleotide sequence.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Alzheimer's disease, for example gantenerumab or an antigen binding fragment thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Alzheimer's disease, for example crenezumab or an antigen binding fragment thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of myasthenia Gravis disease, for example an anti-IL-6 antibody (e.g., satralizumab) or antigen-binding fragment thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In embodiments, the disclosure provides a nucleotide sequence which encodes a molecule for the treatment of Parkinson's disease. In embodiments, the molecule for the treatment of Parkinson's disease comprises an inhibitory nucleic acid molecule (e.g., an antisense oligonucleotide or inhibitory RNA (e.g., siRNA, miRNA or shRNA molecule). In embodiments, the molecule for the treatment of Parkinson's disease comprises an inhibitory nucleic acid molecule (e.g., an antisense oligonucleotide or inhibitory RNA (e.g., siRNA, miRNA or shRNA molecule) targeting SNCA. An exemplary accession number for human SNCA is set forth in Table 3, together with example antisense oligonucleotide targeting SNCA.

In embodiments, the molecule for the treatment of Parkinson's disease comprises a genome editing system (for example a zinc finger nuclease, a meganuclease, a TALEN, or an RNA-guided genome editing system (e.g. a Cas polypeptide and a guide RNA molecule). In embodiments the genome editing system targets a genetic region encoding an alpha-synuclein protein (e.g., SNCA gene).

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Parkinson's disease, for example an anti-alpha synuclein antibody (e.g., prasinezumab or BIIB054) or antigen-binding fragment thereof, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence (e.g., a human GBA1 gene or fragment or variant thereof, e.g., a variant thereof having at least 90% or at least 95% sequence identity to human GBA1) encoding a molecule for the treatment of Parkinson's disease, for example human beta-glucocerebrosidase or a fragment thereof, and (ii) a promoter operably linked to said nucleotide sequence. An exemplary accession number for human GBA1 is set forth in Table 2. Example human GBA1 variants (e.g., single nucleotide polymorphism containing variants) are disclosed in PCT Patent Application Publication No. WO/2023/004370/A1, incorporated herein by reference in its entirety. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Parkinson's disease, for example an inhibitor of LRRK2, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Parkinson's disease, for example a trophic factor (e.g., glial cell line-derived neurotrophic factor (GDNF) or cerebral dopamine neurotrophic factor (CDNF)) or a fragment thereof, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Parkinson's disease, for example an antisense RNA (e.g., antisense RNA targeting alpha-synuclein, or SNCA, gene) or a fragment thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence (e.g., a human GBA gene, e.g., a human GBA1 gene) encoding a molecule for the treatment of Gaucher's disease, for example a human glucocerebrosidase (GCase, e.g. beta-glucosylceramidase-1) or fragment or variant thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of multiple sclerosis, for example an anti-CD20 antibody (e.g., ocrelizumab, rituximab,ofatumumab, or RG6035/R07121932 (an anti-CD20, anti-transferrin receptor bispecific antibody sometimes known as Brainshuttle (BS) CD20-Multiple Sclerosis)) or antigen-binding fragment thereof, and (ii) a promoter operably linked to said nucleotide sequence. In embodiments the multiple sclerosis is relapsing remitting multiple sclerosis. In embodiments the multiple sclerosis is primary progressing multiple sclerosis. In embodiments the multiple sclerosis is secondary progressive multiple sclerosis. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Huntington's disease, for example an inhibitory nucleic acid directed to mutated huntingtin protein (HTT) (e.g., tominersen, WVE-120101 or WVE-120102), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Phelan McDermid syndrome, for example human SHANK3 or fragment or variant thereof (e.g., a variant thereof having at least 90% or at least 95% sequence identity to human SHANK3), and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Phelan McDermid syndrome, for example a human growth hormone, e.g., human insulin like growth factor 1 (IGF-1), or fragment or variant thereof (e.g., a variant thereof having at least 90% or at least 95% sequence identity to human IGF-1), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of frontotemporal dementia, for example human progranulin or granulin, or fragment or variant thereof, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of frontotemporal dementia, for example an anti-tau antibody (e.g., semorinemab), or fragment or variant thereof, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of frontotemporal dementia, for example an inhibitory nucleic acid which targets SOD-1 (e.g., tofersen), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of amyotrophic lateral sclerosis (ALS), for example an inhibitory nucleic acid which targets SOD-1 (e.g., tofersen), and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of ALS, for example an inhibitory nucleic acid which targets C9orf72 (e.g., B11B078), and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment ofALS, for example an inhibitory nucleic acid which targetsATXN2 (e.g., B1IIB105), and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of ALS, for example an inhibitory nucleic acid which targets FUS, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of multiple system atrophy, for example an anti-alpha synuclein antibody (e.g., prasinezumab), and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of multiple system atrophy, for example an antisense oligonucleotide targeting human SNCA, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of multiple system atrophy, for example human glial cell-derived neurotrophic factor (GDNF) or fragment or variant thereof (e.g., a variant thereof having at least 90% or at least 95% sequence identity to human GDNF), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of progressive supranuclear palsy (PSP), for example an anti-Tau antibody (e.g., semorinemab), and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of progressive supranuclear palsy (PSP), for example an anti-alpha-synuclein antibody (e.g., prasinezumab), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Freidrich's ataxia, for example human frataxin (FRXN) or fragment or variant thereof (e.g., a variant thereof having at least 90% or at least 95% sequence identity to human FRXN), and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a nucleotide sequence which encodes a molecule for the treatment of Angelman syndrome. In embodiments, the molecule for the treatment of Angelman syndrome comprises an inhibitory nucleic acid molecule (e.g., an antisense oligonucleotide or inhibitory RNA (e.g., siRNA, miRNA or shRNA molecule). In embodiments, the molecule for the treatment of Angelman syndrome comprises an inhibitory nucleic acid molecule (e.g., an antisense oligonucleotide or inhibitory RNA (e.g., siRNA, miRNA or shRNA molecule) targeting UBE3A.

In embodiments, the molecule for the treatment of Angelman syndrome comprises a genome editing system (for example a zinc finger nuclease, a meganuclease, a TALEN, or an RNA-guided genome editing system (e.g. a Cas polypeptide and a guide RNA molecule). In embodiments the genome editing system targets UBE3A.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Angelman syndrome, for example an inhibitor of a UBE3A antisense nucleic acid (e.g., rugonersen) or a human UBE3A or fragment or variant thereof (e.g., a variant thereof having at least 90% or at least 95% sequence identity to human UBE3A), and (ii) a promoter operably linked to said nucleotide sequence. An exemplary accession number for human UBE3A is set forth in Table 2. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Fragile X syndrome, for example human fragile X mental retardation protein (FMRP) or fragment or variant thereof (e.g., a nucleotide sequence comprising an FMR1 gene or fragment or variant thereof), and (ii) a promoter operably linked to said nucleotide sequence. An exemplary accession number for human FMRP is set forth in Table 2. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Fragile X syndrome, for example an inhibitor of transcriptional silencing of FMRP, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Fragile X syndrome, for example human diacylglycerol kinase (DGKk) or fragment or variant thereof (e.g., a variant thereof having at least 90% or at least 95% sequence identity to human DGKk), and (ii) a promoter operably linked to said nucleotide sequence. An exemplary accession number for human DGKk is set forth in Table 2. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 4.2 and (b) an engineered viral genome comprising (i) a nucleotide sequence encoding a molecule for the treatment of Rett syndrome, for example a human MECP2 or fragment or variant thereof, e.g., a variant thereof having at least 90% or at least 95% sequence identity to human MECP2, and (ii) a promoter operably linked to said nucleotide sequence. An exemplary accession number for human MECP2 is set forth in Table 2. The viral genome can further comprise one or more (e.g., two, three, four or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Dravet syndrome, for example human sodium channel, voltage gated, type 1-alpha (SCN1A or Nav1.1) or fragment or variant thereof, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Dravet syndrome, for example an inhibitory nucleic acid targeting a mutant SCN1A transcript, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Dravet syndrome, for example an anti-tau antibody (e.g., semorinemab), or fragment or variant thereof, and (ii) a promoter operably linked to said nucleotide sequence. In embodiments, described herein is a virus particle comprising (a) a capsid polypeptide described herein, for example, a capsid polypeptide described in Section 6.2 and (b) an engineered viral genome comprising (i) a nucleotide sequence encoding a molecule for the treatment of Dravet syndrome, for example human Syntaxin-binding protein 1 (STXBP1) or fragment or variant thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

In certain aspects, the disclosure provides a virus particle comprising (a) a capsid polypeptide described herein, for example, a capsid polypeptide described in Section 6.2 and (b) an engineered viral genome comprising (i) a nucleotide sequence encoding a molecule for the treatment of Friedreich's ataxia, for example a human FXN gene or fragment or variant thereof, and (ii) a promoter operably linked to said nucleotide sequence. The viral genome can further comprise one or more (e.g., two, three, four, or all five) of (a) a pair of dependoparvovirus ITRs, (b) an intron, (c) an enhancer or repressor sequence, (d) a stuffer sequence, and (e) a polyA sequence. Preferably, the viral genome comprises ITRs flanking the nucleotide sequence and a polyA sequence operably linked to the nucleotide sequence. Typically, the viral genome lacks rep and cap sequences, which are in trans by the host cell in which the virus particle is produced. In some embodiments, the viral genome is self-complementary.

Further exemplary diseases that can be treated and further exemplary heterologous transgenes that can be delivered via the viral particles of the disclosure are provided in Table 2.

TABLE 2
Exemplary CNS indications and transgenes

| | | UniProt |
|---|---|---|
| | | Accession # |
| Indication | Transgene | (Human) |
| Achromatopsia - color blindness | Cyclic nucleotide-gated cation | Q16281 |
| | channel alpha-3 (CNGA3) | |
| | Cyclic nucleotide-gated cation | Q9NQW8 |
| | channel beta-3 (CNGB3) | |
| | Guanine nucleotide-binding protein | P19087 |
| | G(t) subunit alpha-2 (GNAT2) | |
| | Cone cGMP-specific 3′,5′-cyclic | P51160 |
| | phosphodiesterase subunit alpha | |
| | (PDE6C) | |
| Acute Intermittent Porphyria | Porphobilinogen deaminase | P08397 |
| | (PBGD), HMBS | |
| Adie syndrome - Adie's pupil | MPZ | P25189 |
| Age-Related Macular | Vascular endothelial growth factor | P17948 |
| Degeneration | receptor 1 (FLT1) | |
| | Vascular endothelial growth factor | P15692 |
| | A (VEGFA) | |
| Agenesis of the Corpus Callosum | SLC12A6 | Q9UHW9 |
| (ACCPN) | | |
| Aicardi-Goutieres syndrome | TREX1 | Q9NSU2 |
| Alexander disease | GFAP | P14136 |
| Alpers syndrome | POLG | P54098 |
| Alternating hemiplegia | ATP1A2 | P50993 |
| | ATP1A3 | P13637 |
| Alzheimer's disease | NGF | P01138 |
| | ApoE | P02649 |
| | Presenilin (PSEN1) | A0A024R6A3 |
| | Presenilin-2 (PSEN2) | P49810 |
| | Amyloid-beta precursor protein (APP) | P05067 |
| | ADAM10 | O14672 |
| | MAPT, Tau | P10636 |
| Amyotrophic lateral sclerosis | Superoxide dismutase-1 (SOD1) | P00441 |
| (ALS) - Lou Gehrig's disease | | |
| Amyotrophy, hereditary neuralgic | SEPT9 | Q9UHD8 |
| Angelman syndrome | Ubiquitin-protein ligase E3A (UBE3A) | Q05086 |
| Aromatic L-amino acid | DDC | P20711 |
| decarboxylase deficiency | | |
| (AADCD) | | |
| Ataxia | APTX | Q7Z2E3 |
| | KCNA1 | Q09470 |
| | CACNA1A | O00555 |
| Ataxia Telangiectasia - Louis-Bar | Serine-protein kinase ATM (ATM) | Q13315 |
| syndrome | | |
| Attention deficit hyperactivity | DRD4 | P21917 |
| disorder (ADHD) | CDH2 | P19022 |
| Becker muscular dystrophy | Follistatin (FST) | P19883 |
| | DMD | P11532 |
| Benign essential blepharospasm | DRD5 | P21918 |
| Bradbury-Eggleston syndrome - | COQ2 | Q96H96 |
| pure autonomic failure | | |
| Bulbar palsy (BVVLS1) | SLC52A3 | Q9NQ40 |
| Canavan disease - aminoacylase | ASPA | P45381 |
| 2 deficiency | | |
| Carpal tunnel syndrome | TTR | P02766 |
| Cavernoma - Cavernous angioma- | KRIT1 | O00522 |
| Cavernous malformations | | |
| Cerebellar Hypoplasia (CHEGDD) | OXR1 | Q8N573 |
| Cerebellar ataxia (CAMRQ2) | WDR81 | Q562E7 |
| Cerebral Arteriopathy with SCI | NOTCH3 | Q9UM47 |
| and Leukoencephalopathy (CADASIL) | | |
| Cerebral gigantism - Sotos | NSD1 | Q96L73 |
| syndrome 1 | | |
| Cerebro-oculo-facio-skeletal | ERCC6 | Q03468 |
| syndrome (COFS) | | |
| Ceroid lipofuscinosis - Batten | CLN1 (PPT1) | P50897 |
| disease | CLN2 (TPP1) | O14773 |
| | CLN3 (battenin) | Q13286 |
| | CLN4 | Q9H3Z4 |
| | CLN5 | O75503 |
| | CLN6 | Q9NWW5 |
| | CLN7 (MFSD8) | Q8NHS3 |
| | CLN8 | Q9UBY8 |
| | CLN10 (cathepsin D) | P07339 |
| | CLN11 (progranulin) | P28799 |
| | CLN12 (ATP13A2) | Q9NQ11 |
| | CLN13 (cathepsin F) | Q9UBX1 |
| | CLN14 (KCTD7) | Q96MP8 |
| Charcot-Marie-Tooth disease | PMP22 | Q01453 |
| | MPZ | P25189 |
| | DNM2 | P50570 |
| | MFN2 | O95140 |
| | KIF1B | O60333 |
| | SBF2 | Q86WG5 |
| | PNKP | Q96T60 |
| | GDAP1 | Q8TB36 |
| | LMNA | P02545 |
| | FGD4 | Q96M96 |
| | MTMR2 | Q13614 |
| Chorea | NKX2-1 | P43699 |
| Choreoacanthocytosis | VPS13A | Q96RL7 |
| Choroideremia | Rab escort protein (Rep1), CHM | P24386 |
| Chronic Inflammatory | PMP22 | Q01453 |
| Demyelinating Polyneuropathy (CIDP) | | |
| Cockayne syndrome B (CSB) | ERCC6 | Q03468 |
| Coffin-Lowry syndrome | RPS6KA3 | P51812 |
| Craniosynostosis | MSX2 | P35548 |
| | TWIST1 | Q15672 |
| | SKI | P12755 |
| | SMAD6 | O43541 |
| Creutzfeldt-Jakob disease | PRNP | P04156 |
| | HLA-DQB1 | P01920 |
| Crigler-Najjar Syndrome - | UDP-glucuronosyltransferase | P22309 |
| hyperbilirubinemia | 1A1 (UGT1A1) | |
| Cushing Syndrome | PRKACA | P17612 |
| Dentatorubral atrophy (DRPLA) | ATN1 | P54259 |
| Developmental and Epileptic | ARX | Q96QS3 |
| Encephalopathy | FGF12 | P61328 |
| | PIGP | P57054 |
| | GABRB3 | P28472 |
| | NECAP1 | Q8NC96 |
| Developmental Dyspraxia - speech- | FOXP2 | O15409 |
| language disorder 1 (SPCH1) | | |
| Dravet syndrome | Sodium channel protein type 1 | P35498 |
| | subunit alpha (SCN1A) | |
| | SCN1B | Q07699 |
| | SCN2A | Q99250 |
| | GABA receptor subunit gamma-2 | P18507 |
| | (GABRG2) | |
| Dysautonomia - -Day syndrome | ELP1 | O95163 |
| Dystonias | GCH1 | P30793 |
| | TOR1A | O14656 |
| | SGCE | O43556 |
| | TUBB4A | P04350 |
| Encephalocele | COL18A1 | P39060 |
| Epilepsy disorders | GRIN2A | Q12879 |
| | CSTB | P04080 |
| | STARD7 | Q9NQZ5 |
| | DEPDC5 | O75140 |
| | PCDH19 | Q8TAB3 |
| Essential tremor | DRD3 | P35462 |
| | NOTCH2NLC | P0DPK4 |
| | FUS | P35637 |
| Fabry disease | alpha-galactosidase A (GLA) | P06280 |
| Farber disease - ceramidase | ASAH1 | Q13510 |
| deficiency | | |
| Fahr disease | SLC20A2 | Q08357 |
| Febrile Seizures | GABRG2 | P18507 |
| | ADGRV1 | Q8WXG9 |
| | CPA6 | P11509 |
| | SCN1A | P35498 |
| Fragile X syndrome | FMR1 (FMRP) | Q06787 |
| | Diacylglycerol kinase (DGKk) | Q5KSL6 |
| Friedreich's ataxia | Frataxin (FXN) | Q16595 |
| Frontotemporal dementia | Progranulin (GRN) | P28799 |
| | MAPT (tau) | P10636 |
| | PSEN1 | A0A024R6A3 |
| Fucosidosis | alpha-L-fucosidase (FUCA1) | P04066 |
| Fundus albipunctatus | RLBP1 | P12271 |
| Gaucher disease, types I, II and III | Glucocerebrosidase (GBA1) | P04062 |
| Generalized gangliosidoses | GLB1 | P16278 |
| (GM1, GM2, GM3) | | |
| Gerstmann-Straussler-Scheinker disease | PRNP | P04156 |
| Giant axonal neuropathy | Gigaxonin (GAN) | Q9H2C0 |
| Glycogen storage disease II - | Acid maltase, lysosomal alpha- | P10253 |
| Pompe disease - Acid Maltase | glucosidase (LYAG, GAA) | |
| Deficiency | | |
| Guillain-Barre syndrome | PMP22 | Q01453 |
| Hallervorden-Spatz disease - | PANK2 | Q9BZ23 |
| PKAN - NBIA1 | | |
| Hemiplegia Alterans | ATP1A2 | P50993 |
| | ATP1A3 | P13637 |
| Hereditary Neuropathies | WNK1 | Q9H4A3 |
| | MFN2 | O95140 |
| | HK1 | P19367 |
| | TFG | Q92734 |
| | SPTLC1 | O15269 |
| Heredopathia Atactica | PHYH | O14832 |
| Polyneuritiformis - Refsum | | |
| disease | | |
| Holoprosencephalies | GLI2 | P10070 |
| | TGIF1 | Q15583 |
| | ZIC2 | O95409 |
| | PTCH1 | Q13635 |
| | SHH | Q15465 |
| Huntington's disease | HTT | P42858 |
| Hydrocephalus disorders | CCDC88C | Q9P219 |
| | WDR81 | Q562E7 |
| | TRIM71 | Q2Q1W2 |
| | MPDZ | O75970 |
| Incontinentia Pigmenti | IKBKG | Q9Y6K9 |
| Infantile Hypotonia | NALCN | Q8IZF0 |
| | TBCK | Q8TEA7 |
| | CCDC174 | Q6PII3 |
| | UNC80 | Q8N2C7 |
| Infantile Neuroaxonal Dystrophy | PLA2G6 | O60733 |
| Infantile Phytanic Acid Storage | PEX1 | O43933 |
| Disease (PBD1B) | | |
| Joubert Syndrome | INPP5E | Q9NRR6 |
| Kennedy Disease | Androgen receptor (AR) | P10275 |
| Klippel-Feil Syndrome | GDF6 | Q6KF10 |
| Krabbe disease - GALC deficiency | GALC | P54803 |
| Lambert-Eaton Myasthenic Syndrome | CACNA1A | O00555 |
| | CACNB2 | Q13936 |
| Landau-Kleffner Syndrome | GRIN2A | Q12879 |
| Late infantile neuronal | TPP1 | O14773 |
| lipofuscinosis (CLN2) | | |
| Lesch-Nyhan Syndrome | HPRT1 | P00492 |
| Leber congenital amaurosis - | Retinal guanylyl cyclase 1 (GUCY2D) | Q02846 |
| retinal blindness | Retinoid isomerohydrolase (RPE65) | Q16518 |
| | Centrosomal protein of 290 kDa | O15078 |
| | (CEP290) | |
| | Protein crumbs homolog 1 (CRB1) | P82279 |
| Leber's hereditary optical | NADH-ubiquinone oxidoreductase | P03905 |
| neuropathy | chain 4 (ND4) | |
| Leukodystrophy | ARSA | P15289 |
| Levine-Critchley Syndrome - | VPS13A | Q96RL7 |
| choreoacanthocytosis | | |
| Lewy body dementia | SNCA | P37840 |
| | SNCB | Q16143 |
| Lipoid Proteinosis - Urbach- | ECM1 | Q16610 |
| Wiethe disease | | |
| Lissencephaly | PAFAH1B1 | Q9PTR5 |
| | NDE1 | Q9NXR1 |
| | TUBA1A | Q71U36 |
| | LAMB1 | LAMB1 |
| | KATNB1 | Q9BVA0 |
| | RELN | P78509 |
| Macrocephaly/Megalencephaly | TBC1D7 | Q9P0N9 |
| Menkes Disease | ATP7A | Q04656 |
| Metachromatic Leukodystrophy - MLD | Arylsulfatase A (ARSA) | P15289 |
| Microcephaly diseases | KIF11 | P52732 |
| | MCPH1 | Q8NEM0 |
| | SLC25A19 | Q9HC21 |
| Migraine, familial hemiplegic | CACNA1A | O00555 |
| | ATP1A2 | P50993 |
| | SCN1A | P35498 |
| Mitochondrial DNA depletion | RRM2B | Q7LG56 |
| syndromes | DGUOK | Q16854 |
| | POLG | P54098 |
| | TYMP | P19971 |
| | TK2 | O00142 |
| Morvan disease | WNK1 | Q9H4A3 |
| Mucolipidosis | GNPTAB | Q3T906 |
| | MCOLN1 | Q9GZU1 |
| Mucopolysaccharidosis Type I | alpha-L-iduronidase (IDUA) | P35475 |
| (MPS I) - Hurler syndrome | | |
| MPS II - Hunter syndrome | iduronate-2-sulfatase (IDS) | P22304 |
| MPS IIIa - Sanfilippo Type A | heparan sulfate sulfatase (HSS) or | P51688 |
| syndrome | N-sulfoglucosamine sulfohydrolase | |
| | (SGSH) | |
| MPS IIIB - Sanfilippo Type B | N-acetyl-alpha-D-glucosaminidase | P54802 |
| syndrome | (NAGLU) | |
| MPS VI - Maroteaux-Lamy | arylsulfatase B (ARSB) | P15848 |
| syndrome | | |
| MPS IV A - Morquio syndrome | N-acetylgalactosamine-6-sulfatase | P34059 |
| type A | (GALNS) | |
| MPS IV B - Morquio syndrome | Beta-galactosidase 1 (GLB1) | P16278 |
| type B | | |
| MPS VII - Sly syndrome | beta-glucuronidase | P08236 |
| MPS VIII | glucosamine-6-sulfate sulfatase | P15586 |
| MPS IX | Hyaluronidase-1 (HYAL1) | Q12794 |
| Multiple Sclerosis | PDCD1 | Q15116 |
| Multiple system atrophy | COQ2 | Q96H96 |
| Myasthenic syndrome, congenital | CHAT | P28329 |
| presynaptic | | |
| Myoclonus | NOL3 | O60936 |
| Myoclonic epilepsy (FAME2) | STARD7 | Q9NQZ5 |
| Narcolepsy | HCRT, OX | O43612 |
| | MOG | Q16653 |
| Neuroacanthocytosis - McLeod syndrome | XK | P51811 |
| Neurodevelopmental disorder with | TTC5 | Q8N0Z6 |
| cerebral atrophy and facial | | |
| dysmorphism (NEDCAFD) | | |
| Neurodevelopmental disorder with | NCDN | Q9UBB6 |
| infantile epileptic spasms | | |
| Neurofibromatosis | NF1 | P21359 |
| Neuromyotonia | HINT1 | P49773 |
| Neuronal Ceroid Lipofuscinosis | PPT1 | P50897 |
| | TPP1 | O14773 |
| | CLN5 | O75503 |
| | CLN3 | Q13286 |
| | CLN6 | Q9NWW5 |
| | CLN8 | Q9UBY8 |
| | DNAJC5 | Q9H3Z4 |
| | MFSD8 | Q8NHS3 |
| | CTSD | P07339 |
| Neuropathy, ataxia and retinitis | MTATP6 | P00846 |
| pigmentosa (NARP) | | |
| Neuropathy, hereditary sensory | WNK1 | Q9H4A3 |
| and autonomic, type II | | |
| Neuropathy, hypomyelinating | EGR2 | P11161 |
| congenital 1 | | |
| Niemann-Pick disease | Sphingomyelin phosphodiesterase | P17405 |
| | 1 (SMPD1) | |
| | NPC intracellular cholesterol | O15118 |
| | transporter 1 (NPC1) | |
| Ohtahara Syndrome - Developmental | ARX | Q96QS3 |
| and epileptic encephalopathy 1 | | |
| Omithine Transcarbamylase | OTC | P00480 |
| deficiency | | |
| Orthostatic intolerance | SLC6A2 | P23975 |
| Parkinson's disease | Glucocerebrosidase (GBA1) | P04062 |
| | Dopamine decarboxylase (DDC) | P20711 |
| | Neurturin | Q99748 |
| | Glial derived growth factor (GDGF) | P39905 |
| | Tyrosine hydroxylase (TH), | P07101 |
| | tyrosine 3-monooxygenase | |
| | Glutamic acid decarboxylase (GAD) | Q99259 |
| | Fibroblast growth factor 2 (FGF2) | P09038 |
| | Brain-derived neurotrophic factor | P23560 |
| | (BDNF) | |
| Paroxysmal Choreoathetosis | PNKD | Q8N490 |
| Pelizaeus-Merzbacher Disease | PLP1 | P60201 |
| Pena-Shokeir Type II Syndrome | ERCC6 | Q03468 |
| Periodic Paralyses | SCN4A | P35499 |
| Phelan-McDermid syndrome | SH3 and multiple ankyrin repeat | Q9BYB0 |
| | domains protein 3 (SHANK3) | |
| Phytanic Acid Storage Disease - | PEX1 | O43933 |
| peroxisome biogenesis disorder 1B | | |
| Pick disease | PSEN1 | A0A024R6A3 |
| | MAPT (tau) | P10636 |
| Porencephaly type 1 | COL4A1 | P02462 |
| Primary Lateral Sclerosis, juvenile | ALS2 | Q96Q42 |
| Primary Progressive Aphasia | GRN | P28799 |
| Progressive external | POLG | P54098 |
| ophtalmoplegia | POLG2 | Q9UHN1 |
| | SLC25A4 | P12235 |
| | TWNK | Q96RR1 |
| Progressive bulbar palsy | SLC52A3 | Q9NQ40 |
| Progressive supranuclear palsy | Microtubule-associated protein tau | P10636 |
| | (MAPT), Tau | |
| Pseudo-Torch syndrome | OCLN | Q16625 |
| | STAT2 | P52630 |
| | USP18 | Q9UMW8 |
| Retinitis Pigmentosa 38 - rod- | Tyrosine-protein kinase Mer | Q12866 |
| cone dystrophy | (MERTK) | |
| Retinitis Pigmentosa 40 | PDE6B | P35913 |
| Rett syndrome | Methyl-CpG-binding protein 2 | P51608 |
| | (MECP2) | |
| Sandhoff disease | Beta-hexosaminidase subunit | P06865 |
| | alpha (HEXA) | |
| | Beta-hexosaminidase subunit beta | P07686 |
| | (HEXB) | |
| Schizencephaly | SIX3 | O95343 |
| | EMX2 | Q04743 |
| | SHH | Q15465 |
| Seitelberger Disease | PLA2G6 | O60733 |
| Septo-optic dysplasia - De | HESX1 | Q9UBX0 |
| Morsier syndrome | | |
| Snijders Blok-Fisher syndrome | POU3F3 | P20264 |
| Spastic Paraplegias | SPG11 | Q96JI7 |
| | SPAST | Q9UBP0 |
| | KIF5A | Q12840 |
| | NIPA1 | Q7RTP0 |
| | CYP7B1 | O75881 |
| | ATL1 | Q8WXF7 |
| Spinal Muscular Atrophy - | Survival motor neuron protein | Q16637 |
| Kugelberg-Welander Disease | (SMN), SMN1 | |
| Spinocerebellar ataxia | Ataxin-1 (ATXN1), SCA1 | P54253 |
| | Ataxin-2 (ATXN2), SCA2 | Q99700 |
| | Ataxin-3 (ATXN3), SCA3 | P54252 |
| | ZFHX3 | Q15911 |
| | CACNA1A | O00555 |
| | ATXN7, SCA7 | O15265 |
| | TMEM240 | Q5SV17 |
| Sporadic Inclusion Body Myositis | Follistatin (FST) | P19883 |
| Steele-Richardson-Olszewski | MAPT(Tau) | P10636 |
| syndrome - Parkinson-dementia | | |
| syndrome | | |
| Stiff-Person Syndrome, congenital | GLRA1 | P23415 |
| | GLRB | P48167 |
| Striatonigral degeneration | NUP62 | P37198 |
| | PDE8B | O95263 |
| | MTATP6 | P00846 |
| | VAC14 | Q08AM6 |
| Stroke | Tissue-type plasminogen activator | P00750 |
| | (PA) | |
| | Neurogenic differentiation factor 1 | Q13562 |
| | (NeuroD1) | |
| Sturge-Weber Syndrome | GNAQ | P50148 |
| Subcortical Vascular Encephalopathy - | HTRA1 | Q92743 |
| Cerebral Arteriopathy | | |
| Systemic Lupus Erythematosus | DNASE1L3 | Q13609 |
| | TLR7 | Q9NYK1 |
| Tardive Dyskinesia | CYP2D6 | P10635 |
| Tay-Sachs disease | Beta-hexosaminidase subunit | P06865 |
| | alpha (HEXA) | |
| Tourette Syndrome | HDC | P19113 |
| | SLITRK1 | Q96PX8 |
| Tremor, hereditary type 1 | DRD3 | P35462 |
| Troyer Syndrome | SPART | Q8N0X7 |
| Tuberous Sclerosis | TSC1 | Q92574 |
| | TSC2 | P49815 |
| | IFNG | P01579 |
| Von Hippel-Lindau Disease | VHL | P40337 |
| | CCND1 | P24385 |
| Von Recklinghausen Disease | NF1 | P21359 |
| Werdnig-Hoffman Disease | SMN1 | Q16637 |
| West Syndrome, X-linked | ARX | Q96QS3 |
| Wilson disease | ATP7B | P35670 |
| Wolman's disease - acid lipase | LIPA | P38571 |
| disease | | |
| X-linked adrenoleukodystrophy | ATP-binding cassette sub-family D | P33897 |
| | member 1 (ABCD1) | |
| X-linked Retinoschisis | Retinoschisin (RS1) | O15537 |
| X-Linked Retinitis Pigmentosa | X-linked retinitis pigmentosa | Q92834 |
| | GTPase regulator (RPGR) | |
| X-Linked Spinal and Bulbar | UBA1 | P22314 |
| Muscular Atrophy | | |

In some embodiments, the payload is an antisense oligonucleotide effective in treating a CNS disease, for example by modulating expression of a target gene. Exemplary CNS diseases which may be treated using an antisense oligonucleotide include amyotrophic lateral sclerosis, Huntington's disease, and Alzheimer's disease. Exemplary target genes of such antisense oligonucleotides fortreatment of CNS disease include SOD1, C9orf72, Ataxin 2, huntingtin (HTT), Sortilin-related receptor, Microtubule-Associated Protein Tau (MAPT), and neutral sphingomyelinase (N-SMase). Various antisense oligonucleotides effective in treatment of CNS diseases are described in the art and include, for example, those described in Bennett et al., 2019 Annu Rev Neurosci. 42:385-406, Rinaldi et al., 2018, Nature Reviews Neurology, 14:9-21, and Rook et al., 2022, BioDrugs 36(2):105-119, each incorporated herein by reference.

Non-limiting, example antisense oligonucleotides that can be delivered via the viral particles of the disclosure (along with associated target genes and CNS tissue related diseases that can be treated) and are provided in Table 3. Where such antisense oligonucleotides are indicated by reference to a patent or patent application publication, such patent and patent applications are incorporated herein by reference in their entirety for their disclosed antisense oligonucleotide structures and sequences.

TABLE 3
Exemplary CNS Indications, Target Genes, and Antisense Oligonucleotide Transgenes

| | | Uniprot | |
|---|---|---|---|
| | | Accession | |
| Indication | Target Gene | # (Human) | Exemplary transgene(s) |
| Amyotrophic | Superoxide | P00441 | Antisense oligonucleotides described |
| lateral sclerosis | dismutase 1 | | in U.S. Patent Application Publication |
| | (SOD 1) | | Nos. 2024/0182903 A1, |
| | | | 2022/0073930 A1, 2017/0152517 A1, |
| | | | 2020/0354723 A1, 2022/0315930 A1, |
| | | | and 2023/0193281 A1. |
| | C9orf72 | Q96LT7 | Antisense oligonucleotides described |
| | | | in U.S. Patent Application Publication |
| | | | Nos. 2018/0023077 A1, |
| | | | 2015/0267197 A1, 2020/0385723 A1, |
| | | | 2016/0237432 A1, 2023/0098111 A1, |
| | | | and 20190231808 A1. |
| Huntington's | Huntingtin (HTT) | P42858 | Antisense oligonucleotides described in: |
| disease | | | U.S. Patent Application Publication |
| | | | Nos. 20140303238 A1, |
| | | | 2017/0253877 A1, and |
| | | | 2022/0098585 A1; and |
| | | | PCT Patent Application Publication |
| | | | No. WO/2024/035946 A1. |
| Alzheimer's | Sortilin-related | Q92673 | Antisense oligonucleotides described |
| disease | receptor (SORL1) | | in PCT Patent Application Publication |
| | | | No. WO/2023/275376 A1. |
| | Neutral | O60906 | Antisense oligonucleotides described |
| | sphingomyelinase | | in U.S. Patent Application Publication |
| | (N-SMase), also | | No. 2014/0275210 A1. |
| | known as | | |
| | Sphingomyelin | | |
| | phosphodiesterase | | |
| | 2 (SMPD2) | | |
| Spinocerebellar | Ataxin-2 (ATXN2) | Q99700 | Antisense oligonucleotides described |
| ataxia type 2 | | | in U.S. Patent Application Publication |
| (SCA2) | | | No. 2020/0024600 A1 |
| Parkinson's | SNCA | P37840 | Antisense oligonucleotides described |
| Disease | | | in U.S. Patent Application Publication |
| | | | Nos. 20050137155 A1, 20050186591 |
| | | | A1, 20200362347 A1, 20040219671 |
| | | | A1, 20220119811 A1, 20210180065 |
| | | | A1, and 20200392494 A1 |

The disclosure is further directed, in part, to a method of delivering a payload to a subject, e.g., an animal or human subject. In some embodiments, a method of delivering a payload to a subject comprises administering to the subject a dependoparvovirus particle comprising a variant polypeptide (e.g., described herein) comprising the payload, e.g., in a quantity and for a time sufficient to deliver the payload. In some embodiments, the dependoparvovirus particle is a dependoparvovirus particle described herein and comprises a payload described herein. In some embodiments, the particle delivers the payload to the CNS. In some embodiments, the delivery to the CNS is increased as compared to a particle without the variant capsid polypeptide or as compared to a wild-type capsid polypeptide.

### 6.6.2. Methods of Treatment

The disclosure is directed, in part, to a method of treating a disease or condition in a subject, e.g., an animal or human subject. In some embodiments, a method of treating a disease or condition in a subject comprises administering to the subject a dependoparvovirus particle comprising a variant capsid polypeptide described herein, e.g., as described in Section 6.2, comprising a payload described herein, e.g., as described in Section 6.6.1. In some embodiments, the dependoparvovirus particle, which comprises a variant capsid polypeptide as described in Section 6.2, comprising a payload described herein, e.g., as described in Section 6.6.1, is administered in an amount and/or time effective to treat the disease or condition. In some embodiments, the payload is a therapeutic product. In some embodiments, the payload is a nucleic acid, e.g., encoding an exogenous polypeptide. In some embodiments, the payload is a nucleic acid, e.g., encoding an interfering RNA, e.g., an antisense RNA or a micro RNA (miR). The disclosure is also directed to a dependoparvovirus particle comprising a variant polypeptide described herein, e.g., comprising a payload described herein, for use in the methods of treatment described herein. The disclosure is also directed to the use of a dependoparvovirus particle comprising a variant polypeptide described herein, e.g., comprising a payload described herein for the manufacture of a medicament for the treatment of a disease or condition as described herein.

The dependoparvovirus particles comprising a variant polypeptide described herein or produced by the methods described herein can be used to express one or more therapeutic proteins to treat various diseases or disorders. In some embodiments, the disease or disorder is a cancer, e.g., a cancer such as carcinoma, sarcoma, leukemia, lymphoma; or an autoimmune disease, e.g., multiple sclerosis. Non-limiting examples of carcinomas include esophageal carcinoma; bronchogenic carcinoma; colon carcinoma; colorectal carcinoma; gastric carcinoma; hepatocellular carcinoma; basal cell carcinoma, squamous cell carcinoma (various tissues); bladder carcinoma, including transitional cell carcinoma; lung carcinoma, including small cell carcinoma and non-small cell carcinoma of the lung; adrenocortical carcinoma; sweat gland carcinoma; sebaceous gland carcinoma; thyroid carcinoma; pancreatic carcinoma; breast carcinoma; ovarian carcinoma; prostate carcinoma; adenocarcinoma; papillary carcinoma; papillary adenocarcinoma; cystadenocarcinoma; medullary carcinoma; renal cell carcinoma; uterine carcinoma; testicular carcinoma; osteogenic carcinoma; ductal carcinoma in situ or bile duct carcinoma; choriocarcinoma; seminoma; embryonal carcinoma; Wilm's tumor; cervical carcinoma; epithelial carcinoma; and nasopharyngeal carcinoma. Non-limiting examples of sarcomas include fibrosarcoma, myxosarcoma, liposarcoma, angiosarcoma, endotheliosarcoma, lymphangiosarcoma, chondrosarcoma, chordoma, osteogenic sarcoma, osteosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include ependymoma, pinealoma, hemangioblastoma, acoustic neuroma, oligodendroglioma, glioma, astrocytoma, medulloblastoma, craniopharyngioma, menangioma, melanoma, neuroblastoma, and retinoblastoma. Non-limiting examples of leukemias include chronic myeloproliferative syndromes; T-cell CLL prolymphocytic leukemia, acute myelogenous leukemias; chronic lymphocytic leukemias, including B-cell CLL, hairy cell leukemia; and acute lymphoblastic leukemias. Examples of lymphomas include, but are not limited to, B-cell lymphomas, such as Burkitt's lymphoma; and Hodgkin's lymphoma. In some embodiments, the disease or disorder is a genetic disorder. In some embodiments, the genetic disorder is sickle cell anemia, Glycogen storage diseases (GSD, e.g., GSD types I, II, III, IV, V, VI, VII, VIII, IX, X, XI, XII, XIII, and XIV), cystic fibrosis, lysosomal acid lipase (LAL) deficiency 1, Tay-Sachs disease, Phenylketonuria, Mucopolysaccharidoses, Galactosemia, muscular dystrophy (e.g., Duchenne muscular dystrophy), hemophilia such as hemophilia A (classic hemophilia) or hemophilia B (Christmas Disease), Wilson's disease, Fabry Disease, Gaucher Disease hereditary angioedema (HAE), and alpha 1 antitrypsin deficiency. Examples of other diseases or disorders are provided above in Section 6.6.1.

In some aspects, the disease or condition is a disease of the CNS. Exemplary diseases of the CNS include, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Adult polyglucosan body disease, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Angleman syndrome, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Baló's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X syndrome, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy—Congenital, Myopathy—Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Schwannomatosis, Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia (including SCA3 and SCA2), Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy. Examples of other diseases or disorders are provided above in Section 6.6.1.

In some embodiments, administration of a dependoparvovirus particle comprising a variant polypeptide and comprising a payload (e.g., a transgene as described in Section 6.6.1) to a subject induces expression of the payload (e.g., transgene) in a subject. In some embodiments, the expression is induced in the CNS. In some embodiments, the production is similar in the CNS as compared to a similar particle with the wild-type capsid protein. In some embodiments, the production is increased in the CNS as compared to a similar particle with the wild-type capsid protein. The amount of a payload, e.g., transgene, e.g., heterologous protein, e.g., therapeutic polypeptide, expressed in a subject (e.g., the serum of the subject) can vary. For example, in some embodiments the payload, e.g., protein or RNA product of a transgene, can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload, e.g., protein or RNA product of a transgene, is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values.

In some embodiments, for therapeutic applications, a viral particle comprising a capsid polypeptide as described herein is prepared as a pharmaceutical composition. As used herein the term “pharmaceutical composition” refers to a composition comprising at least one active ingredient (e.g., the viral particle) and optionally, one or more pharmaceutically acceptable carriers or excipients.

Relative amounts of the active ingredient, pharmaceutically acceptable carrier or excipient, and/or any additional ingredients in a pharmaceutical composition in accordance with the present disclosure may vary. Differences in the constitution of a pharmaceutical composition may depend upon the identity, size, and/or condition of the subject being treated, the route by which the composition is to be administered, and/or any other factor. The composition may comprise between 0.0001% and 99% (w/w) of the active ingredient. Byway of example, the composition may comprise between 0.0001% and 100%, e.g., between 0.5 and 50%, between 1-30%, between 5-80%, or at least 80% (w/w) active ingredient. Non limiting examples of carriers and/or excipients include solvents, dispersion media, diluents, or other liquid vehicles, dispersion or suspension aids, surface active agents, isotonic agents, thickening or emulsifying agents, preservatives, or combination thereof.

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.
1. A capsid polypeptide comprising:
   (a) a serine at a position corresponding to A472 of the VP1 capsid polypeptide of SEQ ID NO:1;
   (b) an alanine at a position corresponding to V473 of the VP1 capsid polypeptide of SEQ ID NO:1;
   (c) a phenylalanine at a position corresponding to S483 of the VP1 capsid polypeptide of SEQ ID NO:1;
   (d) a serine at a position corresponding to T492 of the VP1 capsid polypeptide of SEQ ID NO:1; or
   (e) any combination of two, three, or all four of (a), (b), (c), and (d).
2. The capsid polypeptide of embodiment 1, which comprises a serine at a position corresponding to A472 of the VP1 capsid polypeptide of SEQ ID NO:1.
3. The capsid polypeptide of embodiment 1 or 2, which comprises an alanine at a position corresponding to V473 of the VP1 capsid polypeptide of SEQ ID NO:1.
4. The capsid polypeptide of any one of embodiments 1 to 3, which comprises a phenylalanine at a position corresponding to S483 of the VP1 capsid polypeptide of SEQ ID NO:1.
5. The capsid polypeptide of any one of embodiments 1 to 4, which comprises a serine at a position corresponding to T492 of the VP1 capsid polypeptide of SEQ ID NO:1.
6. The capsid polypeptide of embodiment 1, which comprises a serine at a position corresponding to A472 of the VP1 capsid polypeptide of SEQ ID NO:1 and an alanine at a position corresponding to V473 of the VP1 capsid polypeptide of SEQ ID NO:1.
7. The capsid polypeptide of embodiment 1, which comprises a serine at a position corresponding to A472 of the VP1 capsid polypeptide of SEQ ID NO:1 and a phenylalanine at a position corresponding to S483 of the VP1 capsid polypeptide of SEQ ID NO:1.
8. The capsid polypeptide of embodiment 1, which comprises a serine at a position corresponding to A472 of the VP1 capsid polypeptide of SEQ ID NO:1 and a serine at a position corresponding to T492 of the VP1 capsid polypeptide of SEQ ID NO:1.
9. The capsid polypeptide of embodiment 1, which comprises an alanine at a position corresponding to V473 of the VP1 capsid polypeptide of SEQ ID NO:1 and a phenylalanine at a position corresponding to S483 of the VP1 capsid polypeptide of SEQ ID NO:1.
10. The capsid polypeptide of embodiment 1, which comprises an alanine at a position corresponding to V473 of the VP1 capsid polypeptide of SEQ ID NO:1 and a serine at a position corresponding to T492 of the VP1 capsid polypeptide of SEQ ID NO:1.
11. The capsid polypeptide of embodiment 1, which comprises a phenylalanine at a position corresponding to S483 of the VP1 capsid polypeptide of SEQ ID NO:1 and a serine at a position corresponding to T492 of the VP1 capsid polypeptide of SEQ ID NO:1.
12. The capsid polypeptide of embodiment 1, which comprises a serine at a position corresponding to A472 of the VP1 capsid polypeptide of SEQ ID NO:1, an alanine at a position corresponding to V473 of the VP1 capsid polypeptide of SEQ ID NO:1, and a phenylalanine at a position corresponding to S483 of the VP1 capsid polypeptide of SEQ ID NO:1.
13. The capsid polypeptide of embodiment 1, which comprises a serine at a position corresponding to A472 of the VP1 capsid polypeptide of SEQ ID NO:1, an alanine at a position corresponding to V473 of the VP1 capsid polypeptide of SEQ ID NO:1, and a serine at a position corresponding to T492 of the VP1 capsid polypeptide of SEQ ID NO:1.
14. The capsid polypeptide of embodiment 1, which comprises a serine at a position corresponding to A472 of the VP1 capsid polypeptide of SEQ ID NO:1, a phenylalanine at a position corresponding to S483 of the VP1 capsid polypeptide of SEQ ID NO:1, and a serine at a position corresponding to T492 of the VP1 capsid polypeptide of SEQ ID NO:1.
15. The capsid polypeptide of embodiment 1, which comprises an alanine at a position corresponding to V473 of the VP1 capsid polypeptide of SEQ ID NO:1, a phenylalanine at a position corresponding to S483 of the VP1 capsid polypeptide of SEQ ID NO:1, and a serine at a position corresponding to T492 of the VP1 capsid polypeptide of SEQ ID NO:1.
16. The capsid polypeptide of embodiment 1, which comprises a serine at a position corresponding to A472 of the VP1 capsid polypeptide of SEQ ID NO:1, an alanine at a position corresponding to V473 of the VP1 capsid polypeptide of SEQ ID NO:1, a phenylalanine at a position corresponding to S483 of the VP1 capsid polypeptide of SEQ ID NO:1, and a serine at a position corresponding to T492 of the VP1 capsid polypeptide of SEQ ID NO:1.
17. A capsid polypeptide comprising:
   (a) a serine at a position corresponding to A472 of the VP1 capsid polypeptide of SEQ ID NO:1;
   (b) an alanine at a position corresponding to V473 of the VP1 capsid polypeptide of SEQ ID NO:1;
   (c) a phenylalanine at a position corresponding to S483 of the VP1 capsid polypeptide of SEQ ID NO:1; and
   (d) a serine at a position corresponding to T492 of the VP1 capsid polypeptide of SEQ ID NO:1.
18. The capsid polypeptide of any one of embodiments 1 to 17, further comprising:
   (a) a valine at a position corresponding to Q579 of the VP1 capsid polypeptide of SEQ ID NO:1;
   (b) an isoleucine at a position corresponding to Q592 of the VP1 capsid polypeptide of SEQ ID NO:1
   (c) a valine at a position corresponding to T593 of the VP1 capsid polypeptide of SEQ ID NO:1;
   (d) an alanine at a position corresponding to W595 of the VP1 capsid polypeptide of SEQ ID NO:1;
   (e) a leucine at a position corresponding to V596 of the VP1 capsid polypeptide of SEQ ID NO:1;
   (f) a serine at a position corresponding to N598 of the VP1 capsid polypeptide of SEQ ID NO:1;
   (g) an alanine at a position corresponding to I601 of the VP1 capsid polypeptide of SEQ ID NO:1; or
   (h) any combination of two, three, four, five, six, or all seven of (a), (b), (c), (d), (e), (f), and (g).
19. The capsid polypeptide of embodiment 18, which comprises a valine at a position corresponding to Q579 of the VP1 capsid polypeptide of SEQ ID NO:1.
20. The capsid polypeptide of embodiment 18 or 19, which comprises an isoleucine at a position corresponding to Q592 of the VP1 capsid polypeptide of SEQ ID NO:1.
21. The capsid polypeptide of any one of embodiments 18 to 20, which comprises a valine at a position corresponding to T593 of the VP1 capsid polypeptide of SEQ ID NO:1.
22. The capsid polypeptide of any one of embodiments 18 to 21, which comprises an alanine at a position corresponding to W595 of the VP1 capsid polypeptide of SEQ ID NO:1.
23. The capsid polypeptide of any one of embodiments 18 to 22, which comprises a leucine at a position corresponding to V596 of the VP1 capsid polypeptide of SEQ ID NO:1.
24. The capsid polypeptide of any one of embodiments 18 to 23, which comprises a serine at a position corresponding to N598 of the VP1 capsid polypeptide of SEQ ID NO:1.
25. The capsid polypeptide of any one of embodiments 18 to 24, which comprises an alanine at a position corresponding to I601 of the VP1 capsid polypeptide of SEQ ID NO:1.
26. A capsid polypeptide comprising:
   (a) a phenylalanine at a position corresponding to S483 of the VP1 capsid polypeptide of SEQ ID NO:1; and
   (b) a serine at a position corresponding to N598 of the VP1 capsid polypeptide of SEQ ID NO:1.
27. A capsid polypeptide comprising:
   (a) a serine at a position corresponding to A472 of the VP1 capsid polypeptide of SEQ ID NO:1;
   (b) an alanine at a position corresponding to V473 of the VP1 capsid polypeptide of SEQ ID NO:1;
   (c) a phenylalanine at a position corresponding to S483 of the VP1 capsid polypeptide of SEQ ID NO:1;
   (d) a serine at a position corresponding to T492 of the VP1 capsid polypeptide of SEQ ID NO:1;
   (e) a valine at a position corresponding to Q579 of the VP1 capsid polypeptide of SEQ ID NO:1;
   (f) an isoleucine at a position corresponding to Q592 of the VP1 capsid polypeptide of SEQ ID NO:1
   (g) a valine at a position corresponding to T593 of the VP1 capsid polypeptide of SEQ ID NO:1;
   (h) an alanine at a position corresponding to W595 of the VP1 capsid polypeptide of SEQ ID NO:1;
   (i) a leucine at a position corresponding to V596 of the VP1 capsid polypeptide of SEQ ID NO:1;
   (j) a serine at a position corresponding to N598 of the VP1 capsid polypeptide of SEQ ID NO:1; and
   (k) an alanine at a position corresponding to I601 of the VP1 capsid polypeptide of SEQ ID NO:1.
28. The capsid polypeptide of any one of embodiments 1 to 27, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
29. The capsid polypeptide of any one of embodiments 1 to 27, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
30. The capsid polypeptide of any one of embodiments 1 to 27, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
31. The capsid polypeptide of any one of embodiments 1 to 27, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
32. The capsid polypeptide of any one of embodiments 1 to 27, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
33. The capsid polypeptide of any one of embodiments 1 to 27, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
34. The capsid polypeptide of any one of embodiments 1 to 27, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
35. The capsid polypeptide of any one of embodiments 1 to 27, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
36. The capsid polypeptide of any one of embodiments 1 to 27, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
37. The capsid polypeptide of any one of embodiments 1 to 27, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
38. The capsid polypeptide of any one of embodiments 1 to 27, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
39. The capsid polypeptide of any one of embodiments 1 to 27, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
40. The capsid polypeptide of any one of embodiments 1 to 27, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
41. The capsid polypeptide of any one of embodiments 1 to 27, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
42. The capsid polypeptide of any one of embodiments 1 to 27, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
43. The capsid polypeptide of any one of embodiments 1 to 27, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
44. The capsid polypeptide of any one of embodiments 1 to 27, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
45. The capsid polypeptide of any one of embodiments 1 to 27, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
46. The capsid polypeptide of any one of embodiments 1 to 27, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
47. The capsid polypeptide of any one of embodiments 1 to 27, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
48. The capsid polypeptide of any one of embodiments 1 to 27, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
49. The capsid polypeptide of any one of embodiments 1 to 27, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
50. The capsid polypeptide of any one of embodiments 1 to 27, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
51. The capsid polypeptide of any one of embodiments 1 to 27, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
52. The capsid polypeptide of any one of embodiments 1 to 27, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
53. The capsid polypeptide of any one of embodiments 1 to 27, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
54. The capsid polypeptide of any one of embodiments 1 to 27, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
55. The capsid polypeptide of any one of embodiments 1 to 27, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:3 or the VP2 or VP3 portion thereof.
56. The capsid polypeptide of any one of embodiments 1 to 27, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
57. The capsid polypeptide of any one of embodiments 1 to 27, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
58. The capsid polypeptide of any one of embodiments 1 to 27, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
59. The capsid polypeptide of any one of embodiments 1 to 27, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
60. The capsid polypeptide of any one of embodiments 1 to 27, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
61. The capsid polypeptide of any one of embodiments 1 to 27, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
62. The capsid polypeptide of any one of embodiments 1 to 27, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
63. The capsid polypeptide of any one of embodiments 1 to 27, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
64. The capsid polypeptide of any one of embodiments 1 to 27, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
65. The capsid polypeptide of any one of embodiments 1 to 27, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
66. The capsid polypeptide of any one of embodiments 1 to 27, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
67. The capsid polypeptide of any one of embodiments 1 to 27, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
68. The capsid polypeptide of any one of embodiments 1 to 27, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
69. The capsid polypeptide of any one of embodiments 1 to 27, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:5 or the VP2 or VP3 portion thereof.
70. The capsid polypeptide of any one of embodiments 1 to 27, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
71. The capsid polypeptide of any one of embodiments 1 to 27, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
72. The capsid polypeptide of any one of embodiments 1 to 27, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
73. The capsid polypeptide of any one of embodiments 1 to 27, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
74. The capsid polypeptide of any one of embodiments 1 to 27, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
75. The capsid polypeptide of any one of embodiments 1 to 27, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
76. The capsid polypeptide of any one of embodiments 1 to 27, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
77. The capsid polypeptide of any one of embodiments 1 to 27, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
78. The capsid polypeptide of any one of embodiments 1 to 27, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
79. The capsid polypeptide of any one of embodiments 1 to 27, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
80. The capsid polypeptide of any one of embodiments 1 to 27, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
81. The capsid polypeptide of any one of embodiments 1 to 27, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
82. The capsid polypeptide of any one of embodiments 1 to 27, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
83. The capsid polypeptide of any one of embodiments 1 to 27, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:7 or the VP2 or VP3 portion thereof.
84. The capsid polypeptide of any one of embodiments 1 to 27, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
85. The capsid polypeptide of any one of embodiments 1 to 27, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
86. The capsid polypeptide of any one of embodiments 1 to 27, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
87. The capsid polypeptide of any one of embodiments 1 to 27, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
88. The capsid polypeptide of any one of embodiments 1 to 27, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
89. The capsid polypeptide of any one of embodiments 1 to 27, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
90. The capsid polypeptide of any one of embodiments 1 to 27, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
91. The capsid polypeptide of any one of embodiments 1 to 27, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
92. The capsid polypeptide of any one of embodiments 1 to 27, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
93. The capsid polypeptide of any one of embodiments 1 to 27, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
94. The capsid polypeptide of any one of embodiments 1 to 27, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
95. The capsid polypeptide of any one of embodiments 1 to 27, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
96. The capsid polypeptide of any one of embodiments 1 to 27, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
97. The capsid polypeptide of any one of embodiments 1 to 27, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:9 or the VP2 or VP3 portion thereof.
98. The capsid polypeptide of any one of embodiments 1 to 27, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
99. The capsid polypeptide of any one of embodiments 1 to 27, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
100. The capsid polypeptide of any one of embodiments 1 to 27, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
101. The capsid polypeptide of any one of embodiments 1 to 27, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
102. The capsid polypeptide of any one of embodiments 1 to 27, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
103. The capsid polypeptide of any one of embodiments 1 to 27, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
104. The capsid polypeptide of any one of embodiments 1 to 27, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
105. The capsid polypeptide of any one of embodiments 1 to 27, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
106. The capsid polypeptide of any one of embodiments 1 to 27, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
107. The capsid polypeptide of any one of embodiments 1 to 27, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
108. The capsid polypeptide of any one of embodiments 1 to 27, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
109. The capsid polypeptide of any one of embodiments 1 to 27, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
110. The capsid polypeptide of any one of embodiments 1 to 27, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
111. The capsid polypeptide of any one of embodiments 1 to 27, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:11 or the VP2 or VP3 portion thereof.
112. The capsid polypeptide of any one of embodiments 1 to 27, which comprises an amino acid sequence having at least 70% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
113. The capsid polypeptide of any one of embodiments 1 to 27, which comprises an amino acid sequence having at least 75% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
114. The capsid polypeptide of any one of embodiments 1 to 27, which comprises an amino acid sequence having at least 80% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
115. The capsid polypeptide of any one of embodiments 1 to 27, which comprises an amino acid sequence having at least 85% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
116. The capsid polypeptide of any one of embodiments 1 to 27, which comprises an amino acid sequence having at least 90% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
117. The capsid polypeptide of any one of embodiments 1 to 27, which comprises an amino acid sequence having at least 91% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
118. The capsid polypeptide of any one of embodiments 1 to 27, which comprises an amino acid sequence having at least 92% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
119. The capsid polypeptide of any one of embodiments 1 to 27, which comprises an amino acid sequence having at least 93% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
120. The capsid polypeptide of any one of embodiments 1 to 27, which comprises an amino acid sequence having at least 94% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
121. The capsid polypeptide of any one of embodiments 1 to 27, which comprises an amino acid sequence having at least 95% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
122. The capsid polypeptide of any one of embodiments 1 to 27, which comprises an amino acid sequence having at least 96% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
123. The capsid polypeptide of any one of embodiments 1 to 27, which comprises an amino acid sequence having at least 97% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
124. The capsid polypeptide of any one of embodiments 1 to 27, which comprises an amino acid sequence having at least 98% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
125. The capsid polypeptide of any one of embodiments 1 to 27, which comprises an amino acid sequence having at least 99% sequence identity ((a) calculated taking targeting peptide insertions into account or (b) calculated without taking targeting peptide insertions into account) to a VP1 capsid polypeptide of SEQ ID NO:14 or the VP2 or VP3 portion thereof.
126. The capsid polypeptide of any one of embodiments 1 to 125, which comprises an amino acid sequence having at least 75% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
127. The capsid polypeptide of any one of embodiments 1 to 125, which comprises an amino acid sequence having at least 80% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
128. The capsid polypeptide of any one of embodiments 1 to 125, which comprises an amino acid sequence having at least 85% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
129. The capsid polypeptide of any one of embodiments 1 to 125, which comprises an amino acid sequence having at least 90% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
130. The capsid polypeptide of any one of embodiments 1 to 125, which comprises an amino acid sequence having at least 91% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
131. The capsid polypeptide of any one of embodiments 1 to 125, which comprises an amino acid sequence having at least 92% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
132. The capsid polypeptide of any one of embodiments 1 to 125, which comprises an amino acid sequence having at least 93% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
133. The capsid polypeptide of any one of embodiments 1 to 125, which comprises an amino acid sequence having at least 94% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
134. The capsid polypeptide of any one of embodiments 1 to 125, which comprises an amino acid sequence having at least 95% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
135. The capsid polypeptide of any one of embodiments 1 to 125, which comprises an amino acid sequence having at least 96% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
136. The capsid polypeptide of any one of embodiments 1 to 125, which comprises an amino acid sequence having at least 97% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
137. The capsid polypeptide of any one of embodiments 1 to 125, which comprises an amino acid sequence having at least 98% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
138. The capsid polypeptide of any one of embodiments 1 to 125, which comprises an amino acid sequence having at least 99% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
139. The capsid polypeptide of any one of embodiments 1 to 125, which comprises an amino acid sequence having at least 99.5% sequence identity ((a) calculated taking targeting peptide insertions into account or (b) calculated without taking targeting peptide insertions into account) to the VP1 capsid polypeptide of SEQ ID NO:12 or the VP2 or VP3 portion thereof.
140. A capsid polypeptide, which is optionally a capsid polypeptide according to any one of embodiments 1 to 139, which comprises the amino acid sequence of a VP1 capsid polypeptide of SEQ ID NO:12 or the VP2 or VP3 portion thereof.
141. The capsid polypeptide of any one of embodiments 1 to 125, which comprises an amino acid sequence having at least 75% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
142. The capsid polypeptide of any one of embodiments 1 to 125, which comprises an amino acid sequence having at least 80% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
143. The capsid polypeptide of any one of embodiments 1 to 125, which comprises an amino acid sequence having at least 85% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
144. The capsid polypeptide of any one of embodiments 1 to 125, which comprises an amino acid sequence having at least 90% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
145. The capsid polypeptide of any one of embodiments 1 to 125, which comprises an amino acid sequence having at least 91% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
146. The capsid polypeptide of any one of embodiments 1 to 125, which comprises an amino acid sequence having at least 92% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
147. The capsid polypeptide of any one of embodiments 1 to 125, which comprises an amino acid sequence having at least 93% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
148. The capsid polypeptide of any one of embodiments 1 to 125, which comprises an amino acid sequence having at least 94% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
149. The capsid polypeptide of any one of embodiments 1 to 125, which comprises an amino acid sequence having at least 95% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
150. The capsid polypeptide of any one of embodiments 1 to 125, which comprises an amino acid sequence having at least 96% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
151. The capsid polypeptide of any one of embodiments 1 to 125, which comprises an amino acid sequence having at least 97% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
152. The capsid polypeptide of any one of embodiments 1 to 125, which comprises an amino acid sequence having at least 98% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
153. The capsid polypeptide of any one of embodiments 1 to 125, which comprises an amino acid sequence having at least 99% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
154. The capsid polypeptide of any one of embodiments 1 to 125, which comprises an amino acid sequence having at least 99.5% sequence identity ((a) calculated taking targeting peptide insertions into account or (b) calculated without taking targeting peptide insertions into account) to the VP1 capsid polypeptide of SEQ ID NO:26 or the VP2 or VP3 portion thereof.
155. A capsid polypeptide, which is optionally a capsid polypeptide according to any one of embodiments 1 to 154, which comprises the amino acid sequence of a VP1 capsid polypeptide of SEQ ID NO:26 or the VP2 or VP3 portion thereof.
156. A capsid polypeptide which:
   (a) comprises at least 70%, at least 80%, or at least 90% (and preferably at least 70%) of the mutations in the mutation set present in the amino acid sequence of SEQ ID NO:12 as compared to the amino acid sequence of SEQ ID NO:1, and
   (b) comprises an amino acid sequence with an edit distance of 15 or less to the amino acid sequence of SEQ ID NO:12 (or to the VP2 or VP3 portion thereof).
157. The capsid polypeptide of embodiment 156, comprising an amino acid sequence having at least 70% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
158. The capsid polypeptide of embodiment 156, comprising an amino acid sequence having at least 75% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
159. The capsid polypeptide of embodiment 156, comprising an amino acid sequence having at least 80% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
160. The capsid polypeptide of embodiment 156, comprising an amino acid sequence having at least 85% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
161. The capsid polypeptide of embodiment 156, comprising an amino acid sequence having at least 90% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
162. The capsid polypeptide of embodiment 156, comprising an amino acid sequence having at least 91% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
163. The capsid polypeptide of embodiment 156, comprising an amino acid sequence having at least 92% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
164. The capsid polypeptide of embodiment 156, comprising an amino acid sequence having at least 93% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
165. The capsid polypeptide of embodiment 156, comprising an amino acid sequence having at least 94% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
166. The capsid polypeptide of embodiment 156, comprising an amino acid sequence having at least 95% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
167. The capsid polypeptide of embodiment 156, comprising an amino acid sequence having at least 96% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
168. The capsid polypeptide of embodiment 156, comprising an amino acid sequence having at least 97% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
169. The capsid polypeptide of embodiment 156, comprising an amino acid sequence having at least 98% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
170. The capsid polypeptide of embodiment 156, comprising an amino acid sequence having at least 99% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
171. The capsid polypeptide of any one of embodiments 1 to 170, whose sequence has an edit distance of (a) 12 or lower, (b) 11 or lower, or (c) 10 or lower to a VP1 capsid polypeptide of SEQ ID NO:1 or the VP2 or VP3 portion thereof.
172. The capsid polypeptide of any one of embodiments 1 to 171, whose sequence has an edit distance of (a) 12 or lower, (b) 11 or lower, or (c) 10 or lower to:
   (a) a VP1 capsid polypeptide of SEQ ID NO:12;
   (b) a VP2 capsid polypeptide corresponding to the VP2 portion of SEQ ID NO:12; or
   (c) a VP3 capsid polypeptide corresponding to the VP3 portion of SEQ ID NO:12.
173. A capsid polypeptide which:
   (a) comprises at least 70%, at least 80%, or at least 90% (and preferably at least 70%) of the mutations in the mutation set present in the amino acid sequence of SEQ ID NO:26 as compared to the amino acid sequence of SEQ ID NO:1; and
   (b) comprises an amino acid sequence with an edit distance of 15 or less to the amino acid sequence of SEQ ID NO:26 (or to the VP2 or VP3 portion thereof).
174. The capsid polypeptide of embodiment 173, comprising an amino acid sequence having at least 70% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
175. The capsid polypeptide of embodiment 156, comprising an amino acid sequence having at least 75% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
176. The capsid polypeptide of embodiment 156, comprising an amino acid sequence having at least 80% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
177. The capsid polypeptide of embodiment 156, comprising an amino acid sequence having at least 85% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
178. The capsid polypeptide of embodiment 156, comprising an amino acid sequence having at least 90% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
179. The capsid polypeptide of embodiment 156, comprising an amino acid sequence having at least 91% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
180. The capsid polypeptide of embodiment 156, comprising an amino acid sequence having at least 92% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
181. The capsid polypeptide of embodiment 156, comprising an amino acid sequence having at least 93% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
182. The capsid polypeptide of embodiment 156, comprising an amino acid sequence having at least 94% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
183. The capsid polypeptide of embodiment 156, comprising an amino acid sequence having at least 95% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
184. The capsid polypeptide of embodiment 156, comprising an amino acid sequence having at least 96% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
185. The capsid polypeptide of embodiment 156, comprising an amino acid sequence having at least 97% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
186. The capsid polypeptide of embodiment 156, comprising an amino acid sequence having at least 98% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
187. The capsid polypeptide of embodiment 156, comprising an amino acid sequence having at least 99% sequence identity ((a) calculated taking targeting peptide insertions into account or (b) calculated without taking targeting peptide insertions into account) to a VP1 capsid polypeptide of SEQ ID NO:1 or the VP2 or VP3 portion thereof.
188. The capsid polypeptide of any one of embodiments 1 to 187, whose sequence has an edit distance of (a) 12 or lower, (b) 11 or lower, or (c) 10 or lower to a VP1 capsid polypeptide of SEQ ID NO:1 or the VP2 or VP3 portion thereof.
189. The capsid polypeptide of any one of embodiments 1 to 188, whose sequence has an edit distance of (a) 12 or lower, (b) 11 or lower, or (c) 10 or lower to:
   (a) a VP1 capsid polypeptide of SEQ ID NO:26;
   (b) a VP2 capsid polypeptide corresponding to the VP2 portion of SEQ ID NO:26; or
   (c) a VP3 capsid polypeptide corresponding to the VP3 portion of SEQ ID NO:26
190. A nucleic acid molecule encoding a capsid polypeptide of any one of embodiments 1 to 189.
191. The nucleic acid molecule of embodiment 190, wherein the nucleic acid molecule comprises a nucleotide sequence having at least 70% sequence identity to the nucleotide sequence of SEQ ID NO:13 or a fragment thereof (e.g., a VP1-encoding, a VP2-encoding or a VP3-encoding fragment thereof).
192. The nucleic acid molecule of embodiment 190 or 191, wherein the nucleic acid molecule is double-stranded or single-stranded, and wherein the nucleic acid molecule is linear or circular, e.g., wherein the nucleic acid molecule is a plasmid.
193. A virus particle (e.g., adeno-associated virus (“AAV”) particle) comprising a capsid polypeptide of any one of embodiments 1 to 173, or comprising a capsid polypeptide encoded by the nucleic acid molecule of any one of embodiments 157 to 159.
194. The virus particle of embodiment 193, comprising a nucleic acid comprising a payload (e.g., a heterologous transgene) and one or more regulatory elements.
195. The virus particle of embodiment 194, wherein the one or more regulatory elements comprise a promoter.
196. The virus particle of embodiment 195, wherein the promoter is a constitutive promoter.
197. The virus particle of embodiment 196, wherein the promoter is a CBh promoter.
198. The virus particle of embodiment 197, wherein the CBh promoter comprises a nucleotide sequence having at least 90%, at least 95%, at least 96%, at least 97%, or at least 98%, at least 99%, or 100% sequence identity to SEQ ID NO:24.
199. The virus particle of embodiment 195, wherein the promoter is a CNS-specific promoter.
200. The virus particle of embodiment 199, wherein the promoter is a hSYN promoter.
201. The virus particle of embodiment 200, wherein the hSYN promoter comprises a nucleotide sequence having at least 90%, at least 95%, at least 96%, at least 97%, or at least 98%, at least 99%, or 100% sequence identity to SEQ ID NO:25.
202. The virus particle of any one of embodiments 193 to 201, wherein said virus particle exhibits increased brain biodistribution, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the increased brain biodistribution (a) is in an amount described in Section 6.4 e.g., as set forth in any one of embodiments A-1 to A-43 and/or (b) is measured using a method described in Section 6.4.
203. The virus particle of embodiment 202, wherein the increased brain biodistribution is exhibited upon systemic, e.g., intravenous, administration of said virus particle.
204. The virus particle of embodiment 202 or embodiment 203, wherein the virus particle exhibits brain biodistribution that is at least 5-fold the brain biodistribution of a virus particle comprising capsid polypeptides of SEQ ID NO:1.
205. The virus particle of embodiment 202 or embodiment 203, wherein the virus particle exhibits brain biodistribution that is at least 10-fold the brain biodistribution of a virus particle comprising capsid polypeptides of SEQ ID NO:1.
206. The virus particle of embodiment 202 or embodiment 203, wherein the virus particle exhibits brain biodistribution that is at least 20-fold the brain biodistribution of a virus particle comprising capsid polypeptides of SEQ ID NO:1.
207. The virus particle of embodiment 202 or embodiment 203, wherein the virus particle exhibits brain biodistribution that is at least 30-fold the brain biodistribution of a virus particle comprising capsid polypeptides of SEQ ID NO:1.
208. The virus particle of embodiment 202 or embodiment 203, wherein the virus particle exhibits brain biodistribution that is at least 50-fold the brain biodistribution of a virus particle comprising capsid polypeptides of SEQ ID NO:1.
209. The virus particle of embodiment 202 or embodiment 203, wherein the virus particle exhibits brain biodistribution that is at least 100-fold the brain biodistribution of a virus particle comprising capsid polypeptides of SEQ ID NO:1.
210. The virus particle of embodiment 202 or embodiment 203, wherein the virus particle exhibits brain biodistribution that is at least 200-fold the brain biodistribution of a virus particle comprising capsid polypeptides of SEQ ID NO:1.
211. The virus particle of embodiment 202 or embodiment 203, wherein the virus particle exhibits brain biodistribution that is at least 400-fold the brain biodistribution of a virus particle comprising capsid polypeptides of SEQ ID NO:1.
212. The virus particle of any one of embodiments 193 to 201, wherein said virus particle exhibits increased brain transduction, e.g., as measured in a mammal, e.g., in mouse or in NHP, e.g., as described herein, relative to wild-type AAV9 (e.g., a virus particle comprising capsid polypeptides of SEQ ID NO:1 or encoded by SEQ ID NO:2), optionally wherein the increased brain transduction (a) is in an amount described in Section 6.4 e.g., as set forth in any one of embodiments B-1 to B-43 and/or (b) is measured using a method described in Section 6.4.
213. The virus particle of embodiment 212, wherein the increased brain transduction is exhibited upon systemic, e.g., intravenous, administration of said virus particle.
214. The virus particle of embodiment 212 or embodiment 213, wherein the virus particle exhibits brain transduction that is at least 5-fold the brain transduction of a virus particle comprising capsid polypeptides of SEQ ID NO:1.
215. The virus particle of embodiment 212 or embodiment 213, wherein the virus particle exhibits brain transduction that is at least 10-fold the brain transduction of a virus particle comprising capsid polypeptides of SEQ ID NO:1.
216. The virus particle of embodiment 212 or embodiment 213, wherein the virus particle exhibits brain transduction that is at least 20-fold the brain transduction of a virus particle comprising capsid polypeptides of SEQ ID NO:1.
217. The virus particle of embodiment 212 or embodiment 213, wherein the virus particle exhibits brain transduction that is at least 30-fold the brain transduction of a virus particle comprising capsid polypeptides of SEQ ID NO:1.
218. The virus particle of embodiment 212 or embodiment 213, wherein the virus particle exhibits brain transduction that is at least 50-fold the brain transduction of a virus particle comprising capsid polypeptides of SEQ ID NO:1.
219. The virus particle of embodiment 212 or embodiment 213, wherein the virus particle exhibits brain transduction that is at least 100-fold the brain transduction of a virus particle comprising capsid polypeptides of SEQ ID NO:1.
220. The virus particle of embodiment 212 or embodiment 213, wherein the virus particle exhibits brain transduction that is at least 200-fold the brain transduction of a virus particle comprising capsid polypeptides of SEQ ID NO:1.
221. The virus particle of embodiment 212 or embodiment 213, wherein the virus particle exhibits brain transduction that is at least 400-fold the brain transduction of a virus particle comprising capsid polypeptides of SEQ ID NO:1.
222. A method of treating a disease or condition in a subject, comprising administering to the subject in an amount effective to treat the disease or condition:
   (a) a virus particle:
      (i) comprising the capsid polypeptide of any one of embodiments 1 to 173 and a heterologous nucleic acid sequence encoding a therapeutic product suitable for treating the disease or condition;
      (ii) comprising a capsid polypeptide encoded by the nucleic acid molecule of any one of embodiments 190 to 192 and a heterologous nucleic acid sequence encoding a therapeutic product suitable for treating the disease or condition, or
      (iii) the virus particle of any one of embodiments 193 to 221; or
   (b) a composition, e.g., a pharmaceutical composition, comprising the virus particle of (a) and, optionally, a pharmaceutically acceptable carrier.
223. The method of embodiment 222, wherein the disease or condition is a disease or condition of the CNS.
224. The method of embodiment 222 or 223, wherein the disease or condition is selected from Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Angleman syndrome, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors (including, but not limited to those that have metastasized to the brain, for example, metastatic breast cancer), Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Bal6's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia—Multi-Infarct, Dementia—Semantic, Dementia—Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X disease, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy—Congenital, Myopathy—Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy—Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phelan McDermid syndrome, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, and X-Linked Spinal or Bulbar Muscular Atrophy.
225. The method of any one of embodiments 222 to 224, wherein the disease or condition is Parkinson's disease.
226. The method of any one of embodiments 222 to 224, wherein the therapeutic product is a human GBA1 gene or portion thereof (e.g., a portion having at least 95% sequence identity to a human GBA1 gene).
227. The method of any one of embodiments 222 to 224, wherein the disease or condition is Acid Lipase Disease.
228. The method of any one of embodiments 222 to 224, wherein the disease or condition is Acid Maltase Deficiency.
229. The method of any one of embodiments 222 to 224, wherein the disease or condition is Acquired Epileptiform Aphasia.
230. The method of any one of embodiments 222 to 224, wherein the disease or condition is Acute Disseminated Encephalomyelitis.
231. The method of any one of embodiments 222 to 224, wherein the disease or condition is Attention Deficit-Hyperactivity Disorder (ADHD).
232. The method of any one of embodiments 222 to 224, wherein the disease or condition is Adie's Pupil.
233. The method of any one of embodiments 222 to 224, wherein the disease or condition is Adie's Syndrome.
234. The method of any one of embodiments 222 to 224, wherein the disease or condition is Adrenoleukodystrophy.
235. The method of any one of embodiments 222 to 224, wherein the disease or condition is Agenesis of the Corpus Callosum.
236. The method of any one of embodiments 222 to 224, wherein the disease or condition is Agnosia.
237. The method of any one of embodiments 222 to 224, wherein the disease or condition is Aicardi Syndrome.
238. The method of any one of embodiments 222 to 224, wherein the disease or condition is Aicardi-Goutieres Syndrome Disorder.
239. The method of any one of embodiments 222 to 224, wherein the disease or condition is AIDS Neurological Complications.
240. The method of any one of embodiments 222 to 224, wherein the disease or condition is Alexander Disease.
241. The method of any one of embodiments 222 to 224, wherein the disease or condition is Alpers' Disease.
242. The method of any one of embodiments 222 to 224, wherein the disease or condition is Alternating Hemiplegia.
243. The method of any one of embodiments 222 to 224, wherein the disease or condition is Alzheimer's Disease.
244. The method of any one of embodiments 222 to 224, wherein the disease or condition is Amyotrophic Lateral Sclerosis (ALS).
245. The method of any one of embodiments 222 to 224, wherein the disease or condition is Anencephaly.
246. The method of any one of embodiments 222 to 224, wherein the disease or condition is Aneurysm.
247. The method of any one of embodiments 222 to 224, wherein the disease or condition is Angelman Syndrome.
248. The method of any one of embodiments 222 to 224, wherein the disease or condition is Angiomatosis.
249. The method of any one of embodiments 222 to 224, wherein the disease or condition is Angleman syndrome.
250. The method of any one of embodiments 222 to 224, wherein the disease or condition is Anoxia.
251. The method of any one of embodiments 222 to 224, wherein the disease or condition is Antiphospholipid Syndrome.
252. The method of any one of embodiments 222 to 224, wherein the disease or condition is Aphasia.
253. The method of any one of embodiments 222 to 224, wherein the disease or condition is Apraxia.
254. The method of any one of embodiments 222 to 224, wherein the disease or condition is Arachnoid Cysts.
255. The method of any one of embodiments 222 to 224, wherein the disease or condition is Arachnoiditis.
256. The method of any one of embodiments 222 to 224, wherein the disease or condition is Arnold-Chiari Malformation.
257. The method of any one of embodiments 222 to 224, wherein the disease or condition is Arteriovenous Malformation.
258. The method of any one of embodiments 222 to 224, wherein the disease or condition is Asperger Syndrome.
259. The method of any one of embodiments 222 to 224, wherein the disease or condition is Ataxia.
260. The method of any one of embodiments 222 to 224, wherein the disease or condition is Ataxia Telangiectasia.
261. The method of any one of embodiments 222 to 224, wherein the disease or condition is Ataxia and Cerebellar or Spinocerebellar Degeneration.
262. The method of any one of embodiments 222 to 224, wherein the disease or condition is Atrial Fibrillation and Stroke.
263. The method of any one of embodiments 222 to 224, wherein the disease or condition is Attention Deficit-Hyperactivity Disorder.
264. The method of any one of embodiments 222 to 224, wherein the disease or condition is Autism Spectrum Disorder.
265. The method of any one of embodiments 222 to 224, wherein the disease or condition is Autonomic Dysfunction.
266. The method of any one of embodiments 222 to 224, wherein the disease or condition is Back Pain.
267. The method of any one of embodiments 222 to 224, wherein the disease or condition is Barth Syndrome.
268. The method of any one of embodiments 222 to 224, wherein the disease or condition is Batten Disease.
269. The method of any one of embodiments 222 to 224, wherein the disease or condition is Becker's Myotonia.
270. The method of any one of embodiments 222 to 224, wherein the disease or condition is Bechet's Disease.
271. The method of any one of embodiments 222 to 224, wherein the disease or condition is Bell's Palsy.
272. The method of any one of embodiments 222 to 224, wherein the disease or condition is Benign Essential Blepharospasm.
273. The method of any one of embodiments 222 to 224, wherein the disease or condition is Benign Focal Amyotrophy.
274. The method of any one of embodiments 222 to 224, wherein the disease or condition is Benign Intracranial Hypertension.
275. The method of any one of embodiments 222 to 224, wherein the disease or condition is Bernhardt-Roth Syndrome.
276. The method of any one of embodiments 222 to 224, wherein the disease or condition is Binswanger's Disease.
277. The method of any one of embodiments 222 to 224, wherein the disease or condition is Blepharospasm.
278. The method of any one of embodiments 222 to 224, wherein the disease or condition is Bloch-Sulzberger Syndrome.
279. The method of any one of embodiments 222 to 224, wherein the disease or condition is Brachial Plexus Birth Injury.
280. The method of any one of embodiments 222 to 224, wherein the disease or condition is Brachial Plexus Injury.
281. The method of any one of embodiments 222 to 224, wherein the disease or condition is Bradbury-Eggleston Syndrome.
282. The method of any one of embodiments 222 to 224, wherein the disease or condition is a Brain or Spinal Tumor (including, but not limited to a tumor that has metastasized to the brain, for example, metastatic breast cancer).
283. The method of any one of embodiments 222 to 224, wherein the disease or condition is Brain Aneurysm.
284. The method of any one of embodiments 222 to 224, wherein the disease or condition is Brain Injury.
285. The method of any one of embodiments 222 to 224, wherein the disease or condition is Brown-Sequard Syndrome.
286. The method of any one of embodiments 222 to 224, wherein the disease or condition is Bulbar palsy.
287. The method of any one of embodiments 222 to 224, wherein the disease or condition is Bulbospinal Muscular Atrophy.
288. The method of any one of embodiments 222 to 224, wherein the disease or condition is Cerebral Autosomal Dominant Arteriopathy with Sub-cortical Infarcts and Leukoencephalopathy (CADASIL).
289. The method of any one of embodiments 222 to 224, wherein the disease or condition is Canavan Disease.
290. The method of any one of embodiments 222 to 224, wherein the disease or condition is Carpal Tunnel Syndrome.
291. The method of any one of embodiments 222 to 224, wherein the disease or condition is Causalgia.
292. The method of any one of embodiments 222 to 224, wherein the disease or condition is Cavernomas.
293. The method of any one of embodiments 222 to 224, wherein the disease or condition is Cavernous Angioma.
294. The method of any one of embodiments 222 to 224, wherein the disease or condition is Cavernous Malformation.
295. The method of any one of embodiments 222 to 224, wherein the disease or condition is Central Cervical Cord Syndrome.
296. The method of any one of embodiments 222 to 224, wherein the disease or condition is Central Cord Syndrome.
297. The method of any one of embodiments 222 to 224, wherein the disease or condition is Central Pain Syndrome.
298. The method of any one of embodiments 222 to 224, wherein the disease or condition is Central Pontine Myelinolysis.
299. The method of any one of embodiments 222 to 224, wherein the disease or condition is a Cephalic Disorder.
300. The method of any one of embodiments 222 to 224, wherein the disease or condition is Ceramidase Deficiency.
301. The method of any one of embodiments 222 to 224, wherein the disease or condition is Cerebellar Degeneration.
302. The method of any one of embodiments 222 to 224, wherein the disease or condition is Cerebellar Hypoplasia.
303. The method of any one of embodiments 222 to 224, wherein the disease or condition is a Cerebral Aneurysm.
304. The method of any one of embodiments 222 to 224, wherein the disease or condition is Cerebral Arteriosclerosis.
305. The method of any one of embodiments 222 to 224, wherein the disease or condition is Cerebral Atrophy.
306. The method of any one of embodiments 222 to 224, wherein the disease or condition is Cerebral Beriberi.
307. The method of any one of embodiments 222 to 224, wherein the disease or condition is Cerebral Cavernous Malformation.
308. The method of any one of embodiments 222 to 224, wherein the disease or condition is Cerebral Gigantism.
309. The method of any one of embodiments 222 to 224, wherein the disease or condition is Cerebral Hypoxia.
310. The method of any one of embodiments 222 to 224, wherein the disease or condition is Cerebral Palsy.
311. The method of any one of embodiments 222 to 224, wherein the disease or condition is Cerebro-Oculo-Facio-Skeletal Syndrome (COFS).
312. The method of any one of embodiments 222 to 224, wherein the disease or condition is Charcot-Marie-Tooth Disease.
313. The method of any one of embodiments 222 to 224, wherein the disease or condition is Chiari Malformation.
314. The method of any one of embodiments 222 to 224, wherein the disease or condition is Cholesterol Ester Storage Disease.
315. The method of any one of embodiments 222 to 224, wherein the disease or condition is Chorea.
316. The method of any one of embodiments 222 to 224, wherein the disease or condition is Choreoacanthocytosis.
317. The method of any one of embodiments 222 to 224, wherein the disease or condition is Chronic Inflammatory Demyelinating Polyneuropathy (CIDP).
318. The method of any one of embodiments 222 to 224, wherein the disease or condition is Chronic Orthostatic Intolerance.
319. The method of any one of embodiments 222 to 224, wherein the disease or condition is Chronic Pain.
320. The method of any one of embodiments 222 to 224, wherein the disease or condition is Cockayne Syndrome Type II.
321. The method of any one of embodiments 222 to 224, wherein the disease or condition is Coffin Lowry Syndrome.
322. The method of any one of embodiments 222 to 224, wherein the disease or condition is Colpocephaly.
323. The method of any one of embodiments 222 to 224, wherein the disease or condition is Coma.
324. The method of any one of embodiments 222 to 224, wherein the disease or condition is Complex Regional Pain Syndrome.
325. The method of any one of embodiments 222 to 224, wherein the disease or condition is Concentric sclerosis (Bal6's sclerosis).
326. The method of any one of embodiments 222 to 224, wherein the disease or condition is Congenital Facial Diplegia.
327. The method of any one of embodiments 222 to 224, wherein the disease or condition is Congenital Myasthenia.
328. The method of any one of embodiments 222 to 224, wherein the disease or condition is Congenital Myopathy.
329. The method of any one of embodiments 222 to 224, wherein the disease or condition is Congenital Vascular Cavernous Malformations.
330. The method of any one of embodiments 222 to 224, wherein the disease or condition is Corticobasal Degeneration.
331. The method of any one of embodiments 222 to 224, wherein the disease or condition is Cranial Arteritis.
332. The method of any one of embodiments 222 to 224, wherein the disease or condition is Craniosynostosis.
333. The method of any one of embodiments 222 to 224, wherein the disease or condition is Cree encephalitis.
334. The method of any one of embodiments 222 to 224, wherein the disease or condition is Creutzfeldt-Jakob Disease.
335. The method of any one of embodiments 222 to 224, wherein the disease or condition is Chronic progressive external ophtalmoplegia.
336. The method of any one of embodiments 222 to 224, wherein the disease or condition is a Cumulative Trauma Disorder.
337. The method of any one of embodiments 222 to 224, wherein the disease or condition is Cushing's Syndrome.
338. The method of any one of embodiments 222 to 224, wherein the disease or condition is Cytomegalic Inclusion Body Disease.
339. The method of any one of embodiments 222 to 224, wherein the disease or condition is Cytomegalovirus Infection.
340. The method of any one of embodiments 222 to 224, wherein the disease or condition is Dancing Eyes-Dancing Feet Syndrome.
341. The method of any one of embodiments 222 to 224, wherein the disease or condition is Dandy-Walker Syndrome.
342. The method of any one of embodiments 222 to 224, wherein the disease or condition is Dawson Disease.
343. The method of any one of embodiments 222 to 224, wherein the disease or condition is De Morsier's Syndrome.
344. The method of any one of embodiments 222 to 224, wherein the disease or condition is Dejerine-Klumpke Palsy.
345. The method of any one of embodiments 222 to 224, wherein the disease or condition is Dementia.
346. The method of any one of embodiments 222 to 224, wherein the disease or condition is Dementia—Multi-Infarct.
347. The method of any one of embodiments 222 to 224, wherein the disease or condition is Dementia—Semantic.
348. The method of any one of embodiments 222 to 224, wherein the disease or condition is Dementia—Subcortical.
349. The method of any one of embodiments 222 to 224, wherein the disease or condition is Dementia With Lewy Bodies.
350. The method of any one of embodiments 222 to 224, wherein the disease or condition is a Demyelination disease.
351. The method of any one of embodiments 222 to 224, wherein the disease or condition is Dentate Cerebellar Ataxia.
352. The method of any one of embodiments 222 to 224, wherein the disease or condition is Dentatorubral Atrophy.
353. The method of any one of embodiments 222 to 224, wherein the disease or condition is Dermatomyositis.
354. The method of any one of embodiments 222 to 224, wherein the disease or condition is Developmental Dyspraxia.
355. The method of any one of embodiments 222 to 224, wherein the disease or condition is Devic's Syndrome.
356. The method of any one of embodiments 222 to 224, wherein the disease or condition is Diabetic Neuropathy.
357. The method of any one of embodiments 222 to 224, wherein the disease or condition is Diffuse Sclerosis.
358. The method of any one of embodiments 222 to 224, wherein the disease or condition is a Distal hereditary motor neuronopathy.
359. The method of any one of embodiments 222 to 224, wherein the disease or condition is Dravet Syndrome.
360. The method of any one of embodiments 222 to 224, wherein the disease or condition is Dysautonomia.
361. The method of any one of embodiments 222 to 224, wherein the disease or condition is Dysgraphia.
362. The method of any one of embodiments 222 to 224, wherein the disease or condition is Dyslexia.
363. The method of any one of embodiments 222 to 224, wherein the disease or condition is Dysphagia.
364. The method of any one of embodiments 222 to 224, wherein the disease or condition is Dyspraxia.
365. The method of any one of embodiments 222 to 224, wherein the disease or condition is Dyssynergia Cerebellaris Myoclonica.
366. The method of embodiment 747, wherein the disease or condition is Dyssynergia Cerebellaris Progressiva.
367. The method of any one of embodiments 222 to 224, wherein the disease or condition is Dystonias.
368. The method of any one of embodiments 222 to 224, wherein the disease or condition is Early Infantile Epileptic Encephalopathy.
369. The method of any one of embodiments 222 to 224, wherein the disease or condition is Empty Sella Syndrome.
370. The method of any one of embodiments 222 to 224, wherein the disease or condition is Encephalitis.
371. The method of any one of embodiments 222 to 224, wherein the disease or condition is Encephalitis Lethargica.
372. The method of any one of embodiments 222 to 224, wherein the disease or condition is Encephaloceles.
373. The method of any one of embodiments 222 to 224, wherein the disease or condition is Encephalomyelitis.
374. The method of any one of embodiments 222 to 224, wherein the disease or condition is Encephalopathy.
375. The method of any one of embodiments 222 to 224, wherein the disease or condition is Encephalopathy (familial infantile).
376. The method of any one of embodiments 222 to 224, wherein the disease or condition is Encephalotrigeminal Angiomatosis.
377. The method of any one of embodiments 222 to 224, wherein the disease or condition is Epilepsy.
378. The method of any one of embodiments 222 to 224, wherein the disease or condition is Epileptic Hemiplegia.
379. The method of any one of embodiments 222 to 224, wherein the disease or condition is Episodic ataxia.
380. The method of any one of embodiments 222 to 224, wherein the disease or condition is Erb's Palsy.
381. The method of any one of embodiments 222 to 224, wherein the disease or condition is Erb-Duchenne Palsy
382. The method of any one of embodiments 222 to 224, wherein the disease or condition is Dejerine-Klumpke Palsy.
383. The method of any one of embodiments 222 to 224, wherein the disease or condition is Essential Tremor.
384. The method of any one of embodiments 222 to 224, wherein the disease or condition is Extrapontine Myelinolysis.
385. The method of any one of embodiments 222 to 224, wherein the disease or condition is Faber's disease.
386. The method of any one of embodiments 222 to 224, wherein the disease or condition is Fabry Disease.
387. The method of any one of embodiments 222 to 224, wherein the disease or condition is Fahr's Syndrome.
388. The method of any one of embodiments 222 to 224, wherein the disease or condition is Fainting.
389. The method of any one of embodiments 222 to 224, wherein the disease or condition is Familial Dysautonomia.
390. The method of any one of embodiments 222 to 224, wherein the disease or condition is Familial Hemangioma.
391. The method of any one of embodiments 222 to 224, wherein the disease or condition is Familial Idiopathic Basal Ganglia Calcification.
392. The method of any one of embodiments 222 to 224, wherein the disease or condition is Familial Periodic Paralyses.
393. The method of any one of embodiments 222 to 224, wherein the disease or condition is Familial Spastic Paralysis.
394. The method of any one of embodiments 222 to 224, wherein the disease or condition is Farber's Disease.
395. The method of any one of embodiments 222 to 224, wherein the disease or condition is Febrile Seizures.
396. The method of any one of embodiments 222 to 224, wherein the disease or condition is Fibromuscular Dysplasia.
397. The method of any one of embodiments 222 to 224, wherein the disease or condition is Fisher Syndrome.
398. The method of any one of embodiments 222 to 224, wherein the disease or condition is Floppy Infant Syndrome.
399. The method of any one of embodiments 222 to 224, wherein the disease or condition is Foot Drop.
400. The method of any one of embodiments 222 to 224, wherein the disease or condition is Fragile X disease.
401. The method of any one of embodiments 222 to 224, wherein the disease or condition is Friedreich's Ataxia.
402. The method of embodiment 748, wherein the disease or condition is Frontotemporal Dementia.
403. The method of any one of embodiments 222 to 224, wherein the disease or condition is Gaucher Disease.
404. The method of any one of embodiments 222 to 224, wherein the disease or condition is Generalized Gangliosidoses (GM1, GM2).
405. The method of any one of embodiments 222 to 224, wherein the disease or condition is Gerstmann's Syndrome.
406. The method of any one of embodiments 222 to 224, wherein the disease or condition is Gerstmann-Straussler-Scheinker Disease.
407. The method of any one of embodiments 222 to 224, wherein the disease or condition is Giant Axonal Neuropathy.
408. The method of any one of embodiments 222 to 224, wherein the disease or condition is Giant Cell Arteritis.
409. The method of any one of embodiments 222 to 224, wherein the disease or condition is Giant Cell Inclusion Disease.
410. The method of any one of embodiments 222 to 224, wherein the disease or condition is Globoid Cell Leukodystrophy.
411. The method of any one of embodiments 222 to 224, wherein the disease or condition is Glossopharyngeal Neuralgia.
412. The method of any one of embodiments 222 to 224, wherein the disease or condition is Glycogen Storage Disease.
413. The method of any one of embodiments 222 to 224, wherein the disease or condition is Guillain-Barre Syndrome.
414. The method of any one of embodiments 222 to 224, wherein the disease or condition is Hallervorden-Spatz Disease.
415. The method of any one of embodiments 222 to 224, wherein the disease or condition is a Head Injury.
416. The method of any one of embodiments 222 to 224, wherein the disease or condition is Headache.
417. The method of any one of embodiments 222 to 224, wherein the disease or condition is Hemicrania Continua.
418. The method of any one of embodiments 222 to 224, wherein the disease or condition is Hemifacial Spasm.
419. The method of any one of embodiments 222 to 224, wherein the disease or condition is Hemiplegia Alterans.
420. The method of any one of embodiments 222 to 224, wherein the disease or condition is a Hereditary Neuropathy.
421. The method of any one of embodiments 222 to 224, wherein the disease or condition is Hereditary Spastic Paraplegia.
422. The method of any one of embodiments 222 to 224, wherein the disease or condition is Heredopathia Atactica Polyneuritiformis.
423. The method of any one of embodiments 222 to 224, wherein the disease or condition is Herpes Zoster.
424. The method of any one of embodiments 222 to 224, wherein the disease or condition is Herpes Zoster Oticus.
425. The method of any one of embodiments 222 to 224, wherein the disease or condition is Hirayama Syndrome.
426. The method of any one of embodiments 222 to 224, wherein the disease or condition is Holmes-Adie syndrome.
427. The method of any one of embodiments 222 to 224, wherein the disease or condition is Holoprosencephaly.
428. The method of any one of embodiments 222 to 224, wherein the disease or condition is HTLV-1 Associated Myelopathy.
429. The method of any one of embodiments 222 to 224, wherein the disease or condition is Hughes Syndrome.
430. The method of any one of embodiments 222 to 224, wherein the disease or condition is Huntington's Disease.
431. The method of any one of embodiments 222 to 224, wherein the disease or condition is Hurler syndrome.
432. The method of any one of embodiments 222 to 224, wherein the disease or condition is Hydranencephaly.
433. The method of any one of embodiments 222 to 224, wherein the disease or condition is Hydrocephalus.
434. The method of any one of embodiments 222 to 224, wherein the disease or condition is Hydrocephalus—Normal Pressure.
435. The method of any one of embodiments 222 to 224, wherein the disease or condition is Hydromyelia.
436. The method of any one of embodiments 222 to 224, wherein the disease or condition is Hypercortisolism.
437. The method of any one of embodiments 222 to 224, wherein the disease or condition is Hypersomnia.
438. The method of any one of embodiments 222 to 224, wherein the disease or condition is Hypertonia.
439. The method of any one of embodiments 222 to 224, wherein the disease or condition is Hypotonia.
440. The method of any one of embodiments 222 to 224, wherein the disease or condition is Hypoxia.
441. The method of any one of embodiments 222 to 224, wherein the disease or condition is Immune-Mediated Encephalomyelitis.
442. The method of any one of embodiments 222 to 224, wherein the disease or condition is Inclusion Body Myositis.
443. The method of any one of embodiments 222 to 224, wherein the disease or condition is Incontinentia Pigmenti.
444. The method of any one of embodiments 222 to 224, wherein the disease or condition is Infantile Hypotonia.
445. The method of any one of embodiments 222 to 224, wherein the disease or condition is Infantile Neuroaxonal Dystrophy.
446. The method of any one of embodiments 222 to 224, wherein the disease or condition is Infantile Phytanic Acid Storage Disease.
447. The method of any one of embodiments 222 to 224, wherein the disease or condition is Infantile Refsum Disease.
448. The method of any one of embodiments 222 to 224, wherein the disease or condition is Infantile Spasms.
449. The method of any one of embodiments 222 to 224, wherein the disease or condition is an Inflammatory Myopathy.
450. The method of any one of embodiments 222 to 224, wherein the disease or condition is Iniencephaly.
451. The method of any one of embodiments 222 to 224, wherein the disease or condition is Intestinal Lipodystrophy.
452. The method of any one of embodiments 222 to 224, wherein the disease or condition is Intracranial Cysts.
453. The method of any one of embodiments 222 to 224, wherein the disease or condition is Intracranial Hypertension.
454. The method of embodiment 747, wherein the disease or condition is Isaacs' Syndrome.
455. The method of any one of embodiments 222 to 224, wherein the disease or condition is Joubert Syndrome.
456. The method of any one of embodiments 222 to 224, wherein the disease or condition is Kearns-Sayre Syndrome.
457. The method of any one of embodiments 222 to 224, wherein the disease or condition is Kennedy's Disease.
458. The method of any one of embodiments 222 to 224, wherein the disease or condition is Kinsbourne syndrome.
459. The method of any one of embodiments 222 to 224, wherein the disease or condition is Kleine-Levin Syndrome.
460. The method of any one of embodiments 222 to 224, wherein the disease or condition is Klippel-Feil Syndrome.
461. The method of any one of embodiments 222 to 224, wherein the disease or condition is Klippel-Trenaunay Syndrome (KTS).
462. The method of any one of embodiments 222 to 224, wherein the disease or condition is Kluver-Bucy Syndrome.
463. The method of any one of embodiments 222 to 224, wherein the disease or condition is Korsakoffs Amnesic Syndrome.
464. The method of any one of embodiments 222 to 224, wherein the disease or condition is Krabbe Disease.
465. The method of any one of embodiments 222 to 224, wherein the disease or condition is Kugelberg-Welander Disease.
466. The method of any one of embodiments 222 to 224, wherein the disease or condition is Kuru.
467. The method of any one of embodiments 222 to 224, wherein the disease or condition is Lambert-Eaton Myasthenic Syndrome.
468. The method of any one of embodiments 222 to 224, wherein the disease or condition is Landau-Kleffner Syndrome.
469. The method of any one of embodiments 222 to 224, wherein the disease or condition is Lateral Femoral Cutaneous Nerve Entrapment.
470. The method of any one of embodiments 222 to 224, wherein the disease or condition is Lateral Medullary Syndrome.
471. The method of any one of embodiments 222 to 224, wherein the disease or condition is a Learning Disability.
472. The method of any one of embodiments 222 to 224, wherein the disease or condition is Leigh's Disease.
473. The method of any one of embodiments 222 to 224, wherein the disease or condition is Lennox-Gastaut Syndrome.
474. The method of any one of embodiments 222 to 224, wherein the disease or condition is Lesch-Nyhan Syndrome.
475. The method of any one of embodiments 222 to 224, wherein the disease or condition is Leukodystrophy.
476. The method of any one of embodiments 222 to 224, wherein the disease or condition is Levine-Critchley Syndrome.
477. The method of any one of embodiments 222 to 224, wherein the disease or condition is Lewy Body Dementia.
478. The method of any one of embodiments 222 to 224, wherein the disease or condition is Lichtheim's disease.
479. The method of any one of embodiments 222 to 224, wherein the disease or condition is a Lipid Storage Disease.
480. The method of any one of embodiments 222 to 224, wherein the disease or condition is Lipoid Proteinosis.
481. The method of any one of embodiments 222 to 224, wherein the disease or condition is Lissencephaly.
482. The method of any one of embodiments 222 to 224, wherein the disease or condition is Locked-In Syndrome.
483. The method of any one of embodiments 222 to 224, wherein the disease or condition is Lou Gehrig's Disease.
484. The method of any one of embodiments 222 to 224, wherein the disease or condition is Lupus—Neurological Sequelae.
485. The method of any one of embodiments 222 to 224, wherein the disease or condition is Lyme Disease—Neurological Complications.
486. The method of any one of embodiments 222 to 224, wherein the disease or condition is Lysosomal storage disorders.
487. The method of any one of embodiments 222 to 224, wherein the disease or condition is Machado-Joseph Disease.
488. The method of any one of embodiments 222 to 224, wherein the disease or condition is Macrencephaly.
489. The method of any one of embodiments 222 to 224, wherein the disease or condition is Megalencephaly.
490. The method of any one of embodiments 222 to 224, wherein the disease or condition is Melkersson-Rosenthal Syndrome.
491. The method of any one of embodiments 222 to 224, wherein the disease or condition is Meningitis.
492. The method of any one of embodiments 222 to 224, wherein the disease or condition is Meningitis and Encephalitis.
493. The method of any one of embodiments 222 to 224, wherein the disease or condition is Menkes Disease.
494. The method of any one of embodiments 222 to 224, wherein the disease or condition is Meralgia Paresthetica.
495. The method of any one of embodiments 222 to 224, wherein the disease or condition is Metachromatic Leukodystrophy.
496. The method of any one of embodiments 222 to 224, wherein the disease or condition is Microcephaly.
497. The method of any one of embodiments 222 to 224, wherein the disease or condition is Migraine.
498. The method of any one of embodiments 222 to 224, wherein the disease or condition is Miller Fisher Syndrome.
499. The method of any one of embodiments 222 to 224, wherein the disease or condition is Mini Stroke.
500. The method of any one of embodiments 222 to 224, wherein the disease or condition is Mitochondrial Myopathy.
501. The method of any one of embodiments 222 to 224, wherein the disease or condition is a Mitochondrial DNA depletion syndrome.
502. The method of any one of embodiments 222 to 224, wherein the disease or condition is Moebius Syndrome.
503. The method of any one of embodiments 222 to 224, wherein the disease or condition is Monomelic Amyotrophy.
504. The method of any one of embodiments 222 to 224, wherein the disease or condition is Morvan Syndrome.
505. The method of any one of embodiments 222 to 224, wherein the disease or condition is a Motor Neuron Disease.
506. The method of any one of embodiments 222 to 224, wherein the disease or condition is Moyamoya Disease.
507. The method of any one of embodiments 222 to 224, wherein the disease or condition is Mucolipidoses.
508. The method of any one of embodiments 222 to 224, wherein the disease or condition is Mucopolysaccharidoses.
509. The method of any one of embodiments 222 to 224, wherein the disease or condition is Multi-Infarct Dementia.
510. The method of any one of embodiments 222 to 224, wherein the disease or condition is Multifocal Motor Neuropathy.
511. The method of any one of embodiments 222 to 224, wherein the disease or condition is Multiple Sclerosis.
512. The method of any one of embodiments 222 to 224, wherein the disease or condition is Multiple System Atrophy.
513. The method of any one of embodiments 222 to 224, wherein the disease or condition is Multiple System Atrophy with Orthostatic Hypotension.
514. The method of any one of embodiments 222 to 224, wherein the disease or condition is Muscular Dystrophy.
515. The method of any one of embodiments 222 to 224, wherein the disease or condition is Myasthenia—Congenital.
516. The method of any one of embodiments 222 to 224, wherein the disease or condition is Myasthenia Gravis.
517. The method of any one of embodiments 222 to 224, wherein the disease or condition is Myelinoclastic Diffuse Sclerosis.
518. The method of any one of embodiments 222 to 224, wherein the disease or condition is Myelitis.
519. The method of any one of embodiments 222 to 224, wherein the disease or condition is Myoclonic Encephalopathy of Infants.
520. The method of any one of embodiments 222 to 224, wherein the disease or condition is Myoclonus.
521. The method of any one of embodiments 222 to 224, wherein the disease or condition is Myoclonus epilepsy.
522. The method of any one of embodiments 222 to 224, wherein the disease or condition is Myopathy.
523. The method of any one of embodiments 222 to 224, wherein the disease or condition is Myopathy—Congenital.
524. The method of any one of embodiments 222 to 224, wherein the disease or condition is Myopathy—Thyrotoxic.
525. The method of any one of embodiments 222 to 224, wherein the disease or condition is Myotonia.
526. The method of any one of embodiments 222 to 224, wherein the disease or condition is Myotonia Congenita.
527. The method of any one of embodiments 222 to 224, wherein the disease or condition is Narcolepsy.
528. The method of any one of embodiments 222 to 224, wherein the disease or condition is NARP (neuropathy, ataxia and retinitis pigmentosa).
529. The method of any one of embodiments 222 to 224, wherein the disease or condition is Neuroacanthocytosis.
530. The method of any one of embodiments 222 to 224, wherein the disease or condition is Neurodegeneration with Brain Iron Accumulation.
531. The method of any one of embodiments 222 to 224, wherein the disease or condition is a Neurodegenerative disease.
532. The method of any one of embodiments 222 to 224, wherein the disease or condition is Neurofibromatosis.
533. The method of any one of embodiments 222 to 224, wherein the disease or condition is Neuroleptic Malignant Syndrome.
534. The method of any one of embodiments 222 to 224, wherein the disease or condition is Neurological Complications of AIDS.
535. The method of any one of embodiments 222 to 224, wherein the disease or condition is Neurological Complications of Lyme Disease.
536. The method of any one of embodiments 222 to 224, wherein the disease or condition is Neurological Consequences of Cytomegalovirus Infection.
537. The method of any one of embodiments 222 to 224, wherein the disease or condition is Neurological Manifestations of Pompe Disease.
538. The method of any one of embodiments 222 to 224, wherein the disease or condition is Neurological Sequelae Of Lupus.
539. The method of any one of embodiments 222 to 224, wherein the disease or condition is Neuromyelitis Optica.
540. The method of any one of embodiments 222 to 224, wherein the disease or condition is Neuromyotonia.
541. The method of any one of embodiments 222 to 224, wherein the disease or condition is Neuronal Ceroid Lipofuscinosis.
542. The method of any one of embodiments 222 to 224, wherein the disease or condition is Neuronal Migration Disorders.
543. The method of any one of embodiments 222 to 224, wherein the disease or condition is Neuropathic pain.
544. The method of any one of embodiments 222 to 224, wherein the disease or condition is Neuropathy—Hereditary.
545. The method of any one of embodiments 222 to 224, wherein the disease or condition is Neuropathy.
546. The method of any one of embodiments 222 to 224, wherein the disease or condition is Neurosarcoidosis.
547. The method of any one of embodiments 222 to 224, wherein the disease or condition is Neurosyphilis.
548. The method of any one of embodiments 222 to 224, wherein the disease or condition is Neurotoxicity.
549. The method of any one of embodiments 222 to 224, wherein the disease or condition is Nevus Cavernosus.
550. The method of any one of embodiments 222 to 224, wherein the disease or condition is Niemann-Pick Disease.
551. The method of any one of embodiments 222 to 224, wherein the disease or condition is O'Sullivan-McLeod Syndrome.
552. The method of any one of embodiments 222 to 224, wherein the disease or condition is Occipital Neuralgia.
553. The method of any one of embodiments 222 to 224, wherein the disease or condition is Ohtahara Syndrome.
554. The method of any one of embodiments 222 to 224, wherein the disease or condition is Olivopontocerebellar Atrophy.
555. The method of any one of embodiments 222 to 224, wherein the disease or condition is Opsoclonus Myoclonus.
556. The method of any one of embodiments 222 to 224, wherein the disease or condition is Orthostatic Hypotension.
557. The method of any one of embodiments 222 to 224, wherein the disease or condition is Overuse Syndrome.
558. The method of any one of embodiments 222 to 224, wherein the disease or condition is Pain—Chronic.
559. The method of any one of embodiments 222 to 224, wherein the disease or condition is Pantothenate Kinase-Associated Neurodegeneration.
560. The method of any one of embodiments 222 to 224, wherein the disease or condition is Paraneoplastic Syndromes.
561. The method of any one of embodiments 222 to 224, wherein the disease or condition is Paresthesia.
562. The method of any one of embodiments 222 to 224, wherein the disease or condition is Parkinson's Disease.
563. The method of any one of embodiments 222 to 224, wherein the disease or condition is Paroxysmal Choreoathetosis.
564. The method of any one of embodiments 222 to 224, wherein the disease or condition is Paroxysmal Hemicrania.
565. The method of any one of embodiments 222 to 224, wherein the disease or condition is Parry-Romberg.
566. The method of any one of embodiments 222 to 224, wherein the disease or condition is Pelizaeus-Merzbacher Disease.
567. The method of any one of embodiments 222 to 224, wherein the disease or condition is Pena Shokeir II Syndrome.
568. The method of any one of embodiments 222 to 224, wherein the disease or condition is Perineural Cysts.
569. The method of any one of embodiments 222 to 224, wherein the disease or condition is Peroneal muscular atrophy.
570. The method of any one of embodiments 222 to 224, wherein the disease or condition is Periodic Paralyses.
571. The method of any one of embodiments 222 to 224, wherein the disease or condition is Peripheral Neuropathy.
572. The method of any one of embodiments 222 to 224, wherein the disease or condition is Periventricular Leukomalacia.
573. The method of any one of embodiments 222 to 224, wherein the disease or condition is Persistent Vegetative State.
574. The method of any one of embodiments 222 to 224, wherein the disease or condition is a Pervasive Developmental Disorder.
575. The method of any one of embodiments 222 to 224, wherein the disease or condition is Phelan McDermid syndrome.
576. The method of any one of embodiments 222 to 224, wherein the disease or condition is Phytanic Acid Storage Disease.
577. The method of any one of embodiments 222 to 224, wherein the disease or condition is Pick's Disease.
578. The method of any one of embodiments 222 to 224, wherein the disease or condition is Pinched Nerve.
579. The method of any one of embodiments 222 to 224, wherein the disease or condition is Piriformis Syndrome.
580. The method of any one of embodiments 222 to 224, wherein the disease or condition is a Pituitary Tumor.
581. The method of any one of embodiments 222 to 224, wherein the disease or condition is Polymyositis.
582. The method of any one of embodiments 222 to 224, wherein the disease or condition is Pompe Disease.
583. The method of any one of embodiments 222 to 224, wherein the disease or condition is Porencephaly.
584. The method of any one of embodiments 222 to 224, wherein the disease or condition is Post-Polio Syndrome.
585. The method of any one of embodiments 222 to 224, wherein the disease or condition is Postherpetic Neuralgia.
586. The method of any one of embodiments 222 to 224, wherein the disease or condition is Postinfectious Encephalomyelitis.
587. The method of any one of embodiments 222 to 224, wherein the disease or condition is Postural Hypotension.
588. The method of any one of embodiments 222 to 224, wherein the disease or condition is Postural Orthostatic Tachycardia Syndrome.
589. The method of any one of embodiments 222 to 224, wherein the disease or condition is Postural Tachycardia Syndrome.
590. The method of any one of embodiments 222 to 224, wherein the disease or condition is Primary Dentatum Atrophy.
591. The method of any one of embodiments 222 to 224, wherein the disease or condition is Primary Lateral Sclerosis.
592. The method of any one of embodiments 222 to 224, wherein the disease or condition is Primary Progressive Aphasia.
593. The method of any one of embodiments 222 to 224, wherein the disease or condition is a Prion Disease.
594. The method of any one of embodiments 222 to 224, wherein the disease or condition is Progressive bulbar palsy.
595. The method of any one of embodiments 222 to 224, wherein the disease or condition is Progressive Hemifacial Atrophy.
596. The method of any one of embodiments 222 to 224, wherein the disease or condition is Progressive Locomotor Ataxia.
597. The method of any one of embodiments 222 to 224, wherein the disease or condition is Progressive Multifocal Leukoencephalopathy.
598. The method of any one of embodiments 222 to 224, wherein the disease or condition is Progressive Muscular Atrophy.
599. The method of any one of embodiments 222 to 224, wherein the disease or condition is Progressive Sclerosing Poliodystrophy.
600. The method of any one of embodiments 222 to 224, wherein the disease or condition is Progressive Supranuclear Palsy.
601. The method of any one of embodiments 222 to 224, wherein the disease or condition is Prosopagnosia.
602. The method of any one of embodiments 222 to 224, wherein the disease or condition is Pseudobulbar palsy.
603. The method of any one of embodiments 222 to 224, wherein the disease or condition is Pseudo-Torch syndrome.
604. The method of any one of embodiments 222 to 224, wherein the disease or condition is Pseudotoxoplasmosis syndrome.
605. The method of any one of embodiments 222 to 224, wherein the disease or condition is Pseudotumor Cerebri.
606. The method of any one of embodiments 222 to 224, wherein the disease or condition is Psychogenic Movement.
607. The method of any one of embodiments 222 to 224, wherein the disease or condition is Ramsay Hunt Syndrome I.
608. The method of any one of embodiments 222 to 224, wherein the disease or condition is Ramsay Hunt Syndrome II.
609. The method of any one of embodiments 222 to 224, wherein the disease or condition is Rasmussen's Encephalitis.
610. The method of any one of embodiments 222 to 224, wherein the disease or condition is Reflex Sympathetic Dystrophy Syndrome.
611. The method of any one of embodiments 222 to 224, wherein the disease or condition is Refsum Disease.
612. The method of any one of embodiments 222 to 224, wherein the disease or condition is Refsum Disease—Infantile.
613. The method of any one of embodiments 222 to 224, wherein the disease or condition is a Repetitive Motion Disorder.
614. The method of any one of embodiments 222 to 224, wherein the disease or condition is a Repetitive Stress Injury.
615. The method of any one of embodiments 222 to 224, wherein the disease or condition is Restless Legs Syndrome.
616. The method of any one of embodiments 222 to 224, wherein the disease or condition is Retrovirus-Associated Myelopathy.
617. The method of any one of embodiments 222 to 224, wherein the disease or condition is Rett Syndrome.
618. The method of any one of embodiments 222 to 224, wherein the disease or condition is Reye's Syndrome.
619. The method of any one of embodiments 222 to 224, wherein the disease or condition is Rheumatic Encephalitis.
620. The method of any one of embodiments 222 to 224, wherein the disease or condition is Riley-Day Syndrome.
621. The method of any one of embodiments 222 to 224, wherein the disease or condition is Sacral Nerve Root Cysts.
622. The method of any one of embodiments 222 to 224, wherein the disease or condition is Saint Vitus Dance.
623. The method of any one of embodiments 222 to 224, wherein the disease or condition is Salivary Gland Disease.
624. The method of any one of embodiments 222 to 224, wherein the disease or condition is Sandhoff Disease.
625. The method of any one of embodiments 222 to 224, wherein the disease or condition is Schilder's Disease.
626. The method of any one of embodiments 222 to 224, wherein the disease or condition is Schizencephaly.
627. The method of any one of embodiments 222 to 224, wherein the disease or condition is Seitelberger Disease.
628. The method of any one of embodiments 222 to 224, wherein the disease or condition is Seizure Disorder.
629. The method of any one of embodiments 222 to 224, wherein the disease or condition is Semantic Dementia.
630. The method of any one of embodiments 222 to 224, wherein the disease or condition is Septo-Optic Dysplasia.
631. The method of any one of embodiments 222 to 224, wherein the disease or condition is Severe Myoclonic Epilepsy of Infancy (SMEI).
632. The method of any one of embodiments 222 to 224, wherein the disease or condition is Shaken Baby Syndrome.
633. The method of any one of embodiments 222 to 224, wherein the disease or condition is Shingles.
634. The method of any one of embodiments 222 to 224, wherein the disease or condition is Shy-Drager Syndrome.
635. The method of any one of embodiments 222 to 224, wherein the disease or condition is Sjogren's Syndrome.
636. The method of any one of embodiments 222 to 224, wherein the disease or condition is Sleep Apnea.
637. The method of any one of embodiments 222 to 224, wherein the disease or condition is Sleeping Sickness.
638. The method of any one of embodiments 222 to 224, wherein the disease or condition is Sotos Syndrome.
639. The method of any one of embodiments 222 to 224, wherein the disease or condition is Spasticity.
640. The method of any one of embodiments 222 to 224, wherein the disease or condition is Spina Bifida.
641. The method of any one of embodiments 222 to 224, wherein the disease or condition is Spinal Cord Infarction.
642. The method of any one of embodiments 222 to 224, wherein the disease or condition is Spinal Cord Injury.
643. The method of any one of embodiments 222 to 224, wherein the disease or condition is a Spinal Cord Tumor.
644. The method of any one of embodiments 222 to 224, wherein the disease or condition is Spinal Muscular Atrophy.
645. The method of any one of embodiments 222 to 224, wherein the disease or condition is Spinocerebellar Ataxia.
646. The method of any one of embodiments 222 to 224, wherein the disease or condition is Spinocerebellar Atrophy.
647. The method of any one of embodiments 222 to 224, wherein the disease or condition is Spinocerebellar Degeneration.
648. The method of any one of embodiments 222 to 224, wherein the disease or condition is Sporadic ataxia.
649. The method of any one of embodiments 222 to 224, wherein the disease or condition is Steele-Richardson-Olszewski Syndrome.
650. The method of any one of embodiments 222 to 224, wherein the disease or condition is Stiff-Person Syndrome.
651. The method of any one of embodiments 222 to 224, wherein the disease or condition is Striatonigral Degeneration.
652. The method of any one of embodiments 222 to 224, wherein the disease or condition is Stroke.
653. The method of any one of embodiments 222 to 224, wherein the disease or condition is Sturge-Weber Syndrome.
654. The method of any one of embodiments 222 to 224, wherein the disease or condition is Subacute Sclerosing Panencephalitis.
655. The method of any one of embodiments 222 to 224, wherein the disease or condition is Subcortical Arteriosclerotic Encephalopathy.
656. The method of any one of embodiments 222 to 224, wherein the disease or condition is Short-lasting, Unilateral, Neuralgiform (SUNCT) Headache.
657. The method of any one of embodiments 222 to 224, wherein the disease or condition is a Swallowing Disorder.
658. The method of any one of embodiments 222 to 224, wherein the disease or condition is Sydenham Chorea.
659. The method of any one of embodiments 222 to 224, wherein the disease or condition is Syncope.
660. The method of any one of embodiments 222 to 224, wherein the disease or condition is Syphilitic Spinal Sclerosis.
661. The method of any one of embodiments 222 to 224, wherein the disease or condition is Syringohydromyelia.
662. The method of any one of embodiments 222 to 224, wherein the disease or condition is Syringomyelia.
663. The method of any one of embodiments 222 to 224, wherein the disease or condition is Systemic Lupus Erythematosus.
664. The method of any one of embodiments 222 to 224, wherein the disease or condition is Tabes Dorsalis.
665. The method of any one of embodiments 222 to 224, wherein the disease or condition is Tardive Dyskinesia.
666. The method of any one of embodiments 222 to 224, wherein the disease or condition is Tarlov Cysts.
667. The method of any one of embodiments 222 to 224, wherein the disease or condition is Tay-Sachs Disease.
668. The method of any one of embodiments 222 to 224, wherein the disease or condition is Temporal Arteritis.
669. The method of any one of embodiments 222 to 224, wherein the disease or condition is Tethered Spinal Cord Syndrome.
670. The method of any one of embodiments 222 to 224, wherein the disease or condition is Thomsen's Myotonia.
671. The method of any one of embodiments 222 to 224, wherein the disease or condition is Thoracic Outlet Syndrome.
672. The method of any one of embodiments 222 to 224, wherein the disease or condition is Thyrotoxic Myopathy.
673. The method of any one of embodiments 222 to 224, wherein the disease or condition is Tic Douloureux.
674. The method of any one of embodiments 222 to 224, wherein the disease or condition is Todd's Paralysis.
675. The method of any one of embodiments 222 to 224, wherein the disease or condition is Tourette Syndrome.
676. The method of any one of embodiments 222 to 224, wherein the disease or condition is Transient Ischemic Attack.
677. The method of any one of embodiments 222 to 224, wherein the disease or condition is Transmissible Spongiform Encephalopathies.
678. The method of any one of embodiments 222 to 224, wherein the disease or condition is Transverse Myelitis.
679. The method of any one of embodiments 222 to 224, wherein the disease or condition is Traumatic Brain Injury.
680. The method of any one of embodiments 222 to 224, wherein the disease or condition is Tremor.
681. The method of any one of embodiments 222 to 224, wherein the disease or condition is Trigeminal Neuralgia.
682. The method of any one of embodiments 222 to 224, wherein the disease or condition is Tropical Spastic Paraparesis.
683. The method of any one of embodiments 222 to 224, wherein the disease or condition is Troyer Syndrome.
684. The method of any one of embodiments 222 to 224, wherein the disease or condition is Tuberous Sclerosis.
685. The method of any one of embodiments 222 to 224, wherein the disease or condition is Vascular Erectile Tumor.
686. The method of any one of embodiments 222 to 224, wherein the disease or condition is a Vasculitis Syndrome of the Central or Peripheral Nervous System.
687. The method of any one of embodiments 222 to 224, wherein the disease or condition is Vitamin B12 deficiency.
688. The method of any one of embodiments 222 to 224, wherein the disease or condition is Von Economo's Disease.
689. The method of any one of embodiments 222 to 224, wherein the disease or condition is Von Hippel-Lindau Disease (VHL).
690. The method of any one of embodiments 222 to 224, wherein the disease or condition is Von Recklinghausen's Disease.
691. The method of any one of embodiments 222 to 224, wherein the disease or condition is Wallenberg's Syndrome.
692. The method of any one of embodiments 222 to 224, wherein the disease or condition is Werdnig-Hoffman Disease.
693. The method of any one of embodiments 222 to 224, wherein the disease or condition is Wernicke-Korsakoff Syndrome.
694. The method of any one of embodiments 222 to 224, wherein the disease or condition is West Syndrome.
695. The method of any one of embodiments 222 to 224, wherein the disease or condition is Whiplash.
696. The method of any one of embodiments 222 to 224, wherein the disease or condition is Whipple's Disease.
697. The method of any one of embodiments 222 to 224, wherein the disease or condition is Williams Syndrome.
698. The method of any one of embodiments 222 to 224, wherein the disease or condition is Wilson Disease.
699. The method of any one of embodiments 222 to 224, wherein the disease or condition is Wolman's Disease.
700. The method of any one of embodiments 222 to 224, wherein the disease or condition is X-Linked Spinal or Bulbar Muscular Atrophy.
701. The method of any one of embodiments 222 to 224, wherein the disease or condition is Achromatopsia (color blindness) and/or wherein the heterologous nucleic acid sequence encodes Cyclic nucleotide-gated cation channel alpha-3 (CNGA3) (e.g., a polypeptide represented by UniProt Accession number Q16281 or a polypeptide comprising an amino acid sequence having at least 95% sequence identity thereto).
702. The method of any one of embodiments 222 to 224, wherein the disease or condition is Achromatopsia (color blindness) and/or wherein the heterologous nucleic acid sequence encodes Cyclic nucleotide-gated cation channel beta-3 (CNGB3) (e.g., a polypeptide represented by UniProt Accession number Q9NQW8 or a polypeptide comprising an amino acid sequence having at least 95% sequence identity thereto).
703. The method of any one of embodiments 222 to 224, wherein the disease or condition is Achromatopsia (color blindness) and/or wherein the heterologous nucleic acid sequence encodes Guanine nucleotide-binding protein G(t) subunit alpha-2 (GNAT2) (e.g., a polypeptide represented by UniProt Accession number P19087 or a polypeptide comprising an amino acid sequence having at least 95% sequence identity thereto).
704. The method of any one of embodiments 222 to 224, wherein the disease or condition is Achromatopsia (color blindness) and/or wherein the heterologous nucleic acid sequence encodes Cone cGMP-specific 3′,5′-cyclic phosphodiesterase subunit alpha (PDE6C) (e.g., a polypeptide represented by UniProt Accession number P51160 or a polypeptide comprising an amino acid sequence having at least 95% sequence identity thereto).
705. The method of any one of embodiments 222 to 224, wherein the disease or condition is Acute Intermittent Porphyria and/or wherein the heterologous nucleic acid sequence encodes Porphobilinogen deaminase (PBGD), HMBS (e.g., a polypeptide represented by UniProt Accession number P08397 or a polypeptide comprising an amino acid sequence having at least 95% sequence identity thereto).
706. The method of any one of embodiments 222 to 224, wherein the disease or condition is Adie syndrome (Adie's pupil) and/or wherein the heterologous nucleic acid sequence encodes MPZ (e.g., a polypeptide represented by UniProt Accession number P25189 or a polypeptide comprising an amino acid sequence having at least 95% sequence identity thereto).
707. The method of any one of embodiments 222 to 224, wherein the disease or condition is Age-Related Macular Degeneration and/or wherein the heterologous nucleic acid sequence encodes Vascular endothelial growth factor receptor 1 (FLT1) (e.g., a polypeptide represented by UniProt Accession number P17948 or a polypeptide comprising an amino acid sequence having at least 95% sequence identity thereto).
708. The method of any one of embodiments 222 to 224, wherein the disease or condition is Age-Related Macular Degeneration and/or wherein the heterologous nucleic acid sequence encodes Vascular endothelial growth factor A (VEGFA) (e.g., a polypeptide represented by UniProt Accession number P15692 or a polypeptide comprising an amino acid sequence having at least 95% sequence identity thereto).
709. The method of any one of embodiments 222 to 224, wherein the disease or condition is Agenesis of the Corpus Callosum (ACCPN) and/or wherein the heterologous nucleic acid sequence encodes SLC12A6 (e.g., a polypeptide represented by UniProt Accession number Q9UHW9 or a polypeptide comprising an amino acid sequence having at least 95% sequence identity thereto).
710. The method of any one of embodiments 222 to 224, wherein the disease or condition is Aicardi-Goutieres syndrome and/or wherein the heterologous nucleic acid sequence encodes TREX1 (e.g., a polypeptide represented by UniProt Accession number Q9NSU2 or a polypeptide comprising an amino acid sequence having at least 95% sequence identity thereto).
711. The method of any one of embodiments 222 to 224, wherein the disease or condition is Alexander disease and/or wherein the heterologous nucleic acid sequence encodes GFAP (e.g., a polypeptide represented by UniProt Accession number P14136 or a polypeptide comprising an amino acid sequence having at least 95% sequence identity thereto).
712. The method of any one of embodiments 222 to 224, wherein the disease or condition is Alpers syndrome and/or wherein the heterologous nucleic acid sequence encodes POLG (e.g., a polypeptide represented by UniProt Accession number P54098 or a polypeptide comprising an amino acid sequence having at least 95% sequence identity thereto).
713. The method of any one of embodiments 222 to 224, wherein the disease or condition is Alternating hemiplegia and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
714. The method of any one of embodiments 222 to 224, wherein the disease or condition is Alternating hemiplegia and/or wherein the heterologous nucleic acid sequence encodes ATP1A3 (e.g., a polypeptide represented by UniProt Accession number P13637 or a polypeptide comprising an amino acid sequence having at least 95% sequence identity thereto).
715. The method of any one of embodiments 222 to 224, wherein the disease or condition is Alzheimer's disease and/or wherein the heterologous nucleic acid sequence encodes NGF (e.g., a polypeptide represented by UniProt Accession number P01138 or a polypeptide comprising an amino acid sequence having at least 95% sequence identity thereto).
716. The method of any one of embodiments 222 to 224, wherein the disease or condition is Alzheimer's disease and/or wherein the heterologous nucleic acid sequence encodes ApoE (e.g., a polypeptide represented by UniProt Accession number P02649 or a polypeptide comprising an amino acid sequence having at least 95% sequence identity thereto).
717. The method of any one of embodiments 222 to 224, wherein the disease or condition is Alzheimer's disease and/or wherein the heterologous nucleic acid sequence encodes Presenilin (PSEN1) (e.g., a polypeptide represented by UniProt Accession number A0A024R6A3 or a polypeptide comprising an amino acid sequence having at least 95% sequence identity thereto).
718. The method of any one of embodiments 222 to 224, wherein the disease or condition is Alzheimer's disease and/or wherein the heterologous nucleic acid sequence encodes Presenilin-2 (PSEN2) (e.g., a polypeptide represented by UniProt Accession number P49810 or a polypeptide comprising an amino acid sequence having at least 95% sequence identity thereto).
719. The method of any one of embodiments 222 to 224, wherein the disease or condition is Alzheimer's disease and/or wherein the heterologous nucleic acid sequence encodes Amyloid-beta precursor protein (APP) (e.g., a polypeptide represented by UniProt Accession number P05067 or a polypeptide comprising an amino acid sequence having at least 95% sequence identity thereto).
720. The method of any one of embodiments 222 to 224, wherein the disease or condition is Alzheimer's disease and/or wherein the heterologous nucleic acid sequence encodes ADAM10 (e.g., a polypeptide represented by UniProt Accession number 014672 or a polypeptide comprising an amino acid sequence having at least 95% sequence identity thereto).
721. The method of any one of embodiments 222 to 224, wherein the disease or condition is Alzheimer's disease and/or wherein the heterologous nucleic acid sequence encodes MAPT, Tau (e.g., a polypeptide represented by UniProt Accession number P10636 or a polypeptide comprising an amino acid sequence having at least 95% sequence identity thereto).
722. The method of any one of embodiments 222 to 224, wherein the disease or condition is Alzheimer's disease and/or wherein the heterologous nucleic acid sequence encodes anti-amyloid-p antibody (e.g., bapineuzumab, solanezumab, aducanumab, lecanemab, gantenerumab, donanemab, or trontinemab, or an antigen binding portion thereof).
723. The method of any one of embodiments 222 to 224, wherein the disease or condition is Alzheimer's disease and/or wherein the heterologous nucleic acid sequence encodes anti-Tau antibody (e.g., Bepranemab, Zagotenemab, or Semorinemab, or an antigen binding portion thereof).
724. The method of any one of embodiments 222 to 224, wherein the disease or condition is Amyotrophic lateral sclerosis (ALS) (Lou Gehrig's disease) and/or wherein the heterologous nucleic acid sequence encodes Superoxide dismutase-1 (SOD1) (e.g., a polypeptide represented by UniProt Accession number P00441 or a polypeptide comprising an amino acid sequence having at least 95% sequence identity thereto).
725. The method of any one of embodiments 222 to 224, wherein the disease or condition is Amyotrophy, hereditary neuralgic and/or wherein the heterologous nucleic acid sequence encodes 45544 (e.g., a polypeptide represented by UniProt Accession number Q9UHD8 or a polypeptide comprising an amino acid sequence having at least 95% sequence identity thereto).
726. The method of any one of embodiments 222 to 224, wherein the disease or condition is Angleman syndrome and/or wherein the heterologous nucleic acid sequence encodes Ubiquitin-protein ligase E3A (UBE3A) (e.g., a polypeptide represented by UniProt Accession number Q05086 or a polypeptide comprising an amino acid sequence having at least 95% sequence identity thereto).
727. The method of any one of embodiments 222 to 224, wherein the disease or condition is Aromatic L-amino acid decarboxylase deficiency (AADCD) and/or wherein the heterologous nucleic acid sequence encodes DDC (e.g., a polypeptide represented by UniProt Accession number P20711 or a polypeptide comprising an amino acid sequence having at least 95% sequence identity thereto).
728. The method of any one of embodiments 222 to 224, wherein the disease or condition is Ataxia and/or wherein the heterologous nucleic acid sequence encodes APTX (e.g., a polypeptide represented by UniProt Accession number Q7Z2E3 or a polypeptide comprising an amino acid sequence having at least 95% sequence identity thereto).
729. The method of any one of embodiments 222 to 224, wherein the disease or condition is Ataxia and/or wherein the heterologous nucleic acid sequence encodes KCNA1 (e.g., a polypeptide represented by UniProt Accession number Q09470 or a polypeptide comprising an amino acid sequence having at least 95% sequence identity thereto).
730. The method of any one of embodiments 222 to 224, wherein the disease or condition is Ataxia and/or wherein the heterologous nucleic acid sequence encodes CACNA1A (e.g., a polypeptide represented by UniProt Accession number 000555 or a polypeptide comprising an amino acid sequence having at least 95% sequence identity thereto).
731. The method of any one of embodiments 222 to 224, wherein the disease or condition is Ataxia Telangiectasia (Louis-Bar syndrome) and/or wherein the heterologous nucleic acid sequence encodes Serine-protein kinase ATM (ATM) (e.g., a polypeptide represented by UniProt Accession number Q13315 or a polypeptide comprising an amino acid sequence having at least 95% sequence identity thereto).
732. The method of any one of embodiments 222 to 224, wherein the disease or condition is Attention deficit hyperactivity disorder (ADHD) and/or wherein the heterologous nucleic acid sequence encodes DRD4 (e.g., a polypeptide represented by UniProt Accession number P21917 or a polypeptide comprising an amino acid sequence having at least 95% sequence identity thereto).
733. The method of any one of embodiments 222 to 224, wherein the disease or condition is Attention deficit hyperactivity disorder (ADHD) and/or wherein the heterologous nucleic acid sequence encodes CDH2 (e.g., a polypeptide represented by UniProt Accession number P19022 or a polypeptide comprising an amino acid sequence having at least 95% sequence identity thereto).
734. The method of any one of embodiments 222 to 224, wherein the disease or condition is Becker muscular dystrophy and/or wherein the heterologous nucleic acid sequence encodes Follistatin (FST) (e.g., a polypeptide represented by UniProt Accession number P19883 or a polypeptide comprising an amino acid sequence having at least 95% sequence identity thereto).
735. The method of any one of embodiments 222 to 224, wherein the disease or condition is Becker muscular dystrophy and/or wherein the heterologous nucleic acid sequence encodes DMD (e.g., a polypeptide represented by UniProt Accession number P11532 or a polypeptide comprising an amino acid sequence having at least 95% sequence identity thereto).
736. The method of any one of embodiments 222 to 224, wherein the disease or condition is Benign essential blepharospasm and/or wherein the heterologous nucleic acid sequence encodes DRD5 (e.g., a polypeptide represented by UniProt Accession number P21918 or a polypeptide comprising an amino acid sequence having at least 95% sequence identity thereto).
737. The method of any one of embodiments 222 to 224, wherein the disease or condition is Bradbury-Eggleston syndrome (pure autonomic failure) and/or wherein the heterologous nucleic acid sequence encodes COQ2 (e.g., a polypeptide represented by UniProt Accession number Q96H96 or a polypeptide comprising an amino acid sequence having at least 95% sequence identity thereto).
738. The method of any one of embodiments 222 to 224, wherein the disease or condition is Bulbar palsy (BVVLS1) and/or wherein the heterologous nucleic acid sequence encodes SLC52A3 (e.g., a polypeptide represented by UniProt Accession number Q9NQ40 or a polypeptide comprising an amino acid sequence having at least 95% sequence identity thereto).
739. The method of any one of embodiments 222 to 224, wherein the disease or condition is Canavan disease (aminoacylase 2 deficiency) and/or wherein the heterologous nucleic acid sequence encodes ASPA (e.g., a polypeptide represented by UniProt Accession number P45381 or a polypeptide comprising an amino acid sequence having at least 95% sequence identity thereto).
740. The method of any one of embodiments 222 to 224, wherein the disease or condition is Carpal tunnel syndrome and/or wherein the heterologous nucleic acid sequence encodes TTR (e.g., a polypeptide represented by UniProt Accession number P02766 or a polypeptide comprising an amino acid sequence having at least 95% sequence identity thereto).
741. The method of any one of embodiments 222 to 224, wherein the disease or condition is Cavernoma (Cavernous angioma, Cavernous malformations) and/or wherein the heterologous nucleic acid sequence encodes KRIT1 (e.g., a polypeptide represented by UniProt Accession number 000522 or a polypeptide comprising an amino acid sequence having at least 95% sequence identity thereto).
742. The method of any one of embodiments 222 to 224, wherein the disease or condition is Cerebellar Hypoplasia (CHEGDD) and/or wherein the heterologous nucleic acid sequence encodes OXR1 (e.g., a polypeptide represented by UniProt Accession number Q8N573 or a polypeptide comprising an amino acid sequence having at least 95% sequence identity thereto).
743. The method of any one of embodiments 222 to 224, wherein the disease or condition is Cerebellar ataxia (CAMRQ2) and/or wherein the heterologous nucleic acid sequence encodes WDR81 (e.g., a polypeptide represented by UniProt Accession number Q562E7 or a polypeptide comprising an amino acid sequence having at least 95% sequence identity thereto).
744. The method of any one of embodiments 222 to 224, wherein the disease or condition is Cerebral Arteriopathy with SCI and Leukoencephalopathy (CADASIL) and/or wherein the heterologous nucleic acid sequence encodes NOTCH3 (e.g., a polypeptide represented by UniProt Accession number Q9UM47 or a polypeptide comprising an amino acid sequence having at least 95% sequence identity thereto).
745. The method of any one of embodiments 222 to 224, wherein the disease or condition is Cerebral gigantism (Sotos syndrome 1) and/or wherein the heterologous nucleic acid sequence encodes NSD1 (e.g., a polypeptide represented by UniProt Accession number Q96L73 or a polypeptide comprising an amino acid sequence having at least 95% sequence identity thereto).
746. The method of any one of embodiments 222 to 224, wherein the disease or condition is Cerebro-oculo-facio-skeletal syndrome (COFS) and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
747. The method of any one of embodiments 222 to 224, wherein the disease or condition is Ceroid lipofuscinosis (Batten disease) and/or wherein the heterologous nucleic acid sequence encodes CLN1 (PPT1) (e.g., a polypeptide represented by UniProt Accession number P50897 or a polypeptide comprising an amino acid sequence having at least 95% sequence identity thereto).
748. The method of any one of embodiments 222 to 224, wherein the disease or condition is Ceroid lipofuscinosis (Batten disease) and/or wherein the heterologous nucleic acid sequence encodes CLN2 (TPP1) (e.g., a polypeptide represented by UniProt Accession number 014773 or a polypeptide comprising an amino acid sequence having at least 95% sequence identity thereto).
749. The method of any one of embodiments 222 to 224, wherein the disease or condition is Ceroid lipofuscinosis (Batten disease) and/or wherein the heterologous nucleic acid sequence encodes CLN3 (battenin) (e.g., a polypeptide represented by UniProt Accession number Q13286 or a polypeptide comprising an amino acid sequence having at least 95% sequence identity thereto).
750. The method of any one of embodiments 222 to 224, wherein the disease or condition is Ceroid lipofuscinosis (Batten disease) and/or wherein the heterologous nucleic acid sequence encodes CLN4 (e.g., a polypeptide represented by UniProt Accession number Q9H3Z4 or a polypeptide comprising an amino acid sequence having at least 95% sequence identity thereto).
751. The method of any one of embodiments 222 to 224, wherein the disease or condition is Ceroid lipofuscinosis (Batten disease) and/or wherein the heterologous nucleic acid sequence encodes CLN5 (e.g., a polypeptide represented by UniProt Accession number 075503 or a polypeptide comprising an amino acid sequence having at least 95% sequence identity thereto).
752. The method of any one of embodiments 222 to 224, wherein the disease or condition is Ceroid lipofuscinosis (Batten disease) and/or wherein the heterologous nucleic acid sequence encodes CLN6 (e.g., a polypeptide represented by UniProt Accession number Q9NWW5 or a polypeptide comprising an amino acid sequence having at least 95% sequence identity thereto).
753. The method of any one of embodiments 222 to 224, wherein the disease or condition is Ceroid lipofuscinosis (Batten disease) and/or wherein the heterologous nucleic acid sequence encodes CLN7 (MFSD8) (e.g., a polypeptide represented by UniProt Accession number Q8NHS3 or a polypeptide comprising an amino acid sequence having at least 95% sequence identity thereto).
754. The method of any one of embodiments 222 to 224, wherein the disease or condition is Ceroid lipofuscinosis (Batten disease) and/or wherein the heterologous nucleic acid sequence encodes CLN8 (e.g., a polypeptide represented by UniProt Accession number Q9UBY8 or a polypeptide comprising an amino acid sequence having at least 95% sequence identity thereto).
755. The method of any one of embodiments 222 to 224, wherein the disease or condition is Ceroid lipofuscinosis (Batten disease) and/or wherein the heterologous nucleic acid sequence encodes CLN10 (cathepsin D) (e.g., a polypeptide represented by UniProt Accession number P07339 or a polypeptide comprising an amino acid sequence having at least 95% sequence identity thereto).
756. The method of any one of embodiments 222 to 224, wherein the disease or condition is Ceroid lipofuscinosis (Batten disease) and/or wherein the heterologous nucleic acid sequence encodes CLN11 (progranulin) (e.g., a polypeptide represented by UniProt Accession number P28799 or a polypeptide comprising an amino acid sequence having at least 95% sequence identity thereto).
757. The method of any one of embodiments 222 to 224, wherein the disease or condition is Ceroid lipofuscinosis (Batten disease) and/or wherein the heterologous nucleic acid sequence encodes CLN12 (ATP13A2) (e.g., a polypeptide represented by UniProt Accession number Q9NQ11 or a polypeptide comprising an amino acid sequence having at least 95% sequence identity thereto).
758. The method of any one of embodiments 222 to 224, wherein the disease or condition is Ceroid lipofuscinosis (Batten disease) and/or wherein the heterologous nucleic acid sequence encodes CLN13 (cathepsin F) (e.g., a polypeptide represented by UniProt Accession number Q9UBX1 or a polypeptide comprising an amino acid sequence having at least 95% sequence identity thereto).
759. The method of any one of embodiments 222 to 224, wherein the disease or condition is Ceroid lipofuscinosis (Batten disease) and/or wherein the heterologous nucleic acid sequence encodes CLN14 (KCTD7) (e.g., a polypeptide represented by UniProt Accession number Q96MP8 or a polypeptide comprising an amino acid sequence having at least 95% sequence identity thereto).
760. The method of any one of embodiments 222 to 224, wherein the disease or condition is Charcot-Marie-Tooth disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
761. The method of any one of embodiments 222 to 224, wherein the disease or condition is Charcot-Marie-Tooth disease and/or wherein the heterologous nucleic acid sequence encodes MPZ (e.g., a polypeptide represented by UniProt Accession number P25189 or a polypeptide comprising an amino acid sequence having at least 95% sequence identity thereto).
762. The method of any one of embodiments 222 to 224, wherein the disease or condition is Charcot-Marie-Tooth disease and/or wherein the heterologous nucleic acid sequence encodes DNM2 (e.g., a polypeptide represented by UniProt Accession number P50570 or a polypeptide comprising an amino acid sequence having at least 95% sequence identity thereto).
763. The method of any one of embodiments 222 to 224, wherein the disease or condition is Charcot-Marie-Tooth disease and/or wherein the heterologous nucleic acid sequence encodes MFN2 (e.g., a polypeptide represented by UniProt Accession number 095140 or a polypeptide comprising an amino acid sequence having at least 95% sequence identity thereto).
764. The method of any one of embodiments 222 to 224, wherein the disease or condition is Charcot-Marie-Tooth disease and/or wherein the heterologous nucleic acid sequence encodes KIF1B (e.g., a polypeptide represented by UniProt Accession number 060333 or a polypeptide comprising an amino acid sequence having at least 95% sequence identity thereto).
765. The method of any one of embodiments 222 to 224, wherein the disease or condition is Charcot-Marie-Tooth disease and/or wherein the heterologous nucleic acid sequence encodes SBF2 (e.g., a polypeptide represented by UniProt Accession number Q86WG5 or a polypeptide comprising an amino acid sequence having at least 95% sequence identity thereto).
766. The method of any one of embodiments 222 to 224, wherein the disease or condition is Charcot-Marie-Tooth disease and/or wherein the heterologous nucleic acid sequence encodes PNKP (e.g., a polypeptide represented by UniProt Accession number Q96T60 or a polypeptide comprising an amino acid sequence having at least 95% sequence identity thereto).
767. The method of any one of embodiments 222 to 224, wherein the disease or condition is Charcot-Marie-Tooth disease and/or wherein the heterologous nucleic acid sequence encodes GDAP1 (e.g., a polypeptide represented by UniProt Accession number Q8TB36 or a polypeptide comprising an amino acid sequence having at least 95% sequence identity thereto).
768. The method of any one of embodiments 222 to 224, wherein the disease or condition is Charcot-Marie-Tooth disease and/or wherein the heterologous nucleic acid sequence encodes LMNA (e.g., a polypeptide represented by UniProt Accession number P02545 or a polypeptide comprising an amino acid sequence having at least 95% sequence identity thereto).
769. The method of any one of embodiments 222 to 224, wherein the disease or condition is Charcot-Marie-Tooth disease and/or wherein the heterologous nucleic acid sequence encodes FGD4 (e.g., a polypeptide represented by UniProt Accession number Q96M96 or a polypeptide comprising an amino acid sequence having at least 95% sequence identity thereto).
770. The method of any one of embodiments 222 to 224, wherein the disease or condition is Charcot-Marie-Tooth disease and/or wherein the heterologous nucleic acid sequence encodes MTMR2 (e.g., a polypeptide represented by UniProt Accession number Q13614 or a polypeptide comprising an amino acid sequence having at least 95% sequence identity thereto).
771. The method of any one of embodiments 222 to 224, wherein the disease or condition is Chorea and/or wherein the heterologous nucleic acid sequence encodes NKX2-1 (e.g., a polypeptide represented by UniProt Accession number P43699 or a polypeptide comprising an amino acid sequence having at least 95% sequence identity thereto).
772. The method of any one of embodiments 222 to 224, wherein the disease or condition is Choreoacanthocytosis and/or wherein the heterologous nucleic acid sequence encodes VPS13A (e.g., a polypeptide represented by UniProt Accession number Q96RL7 or a polypeptide comprising an amino acid sequence having at least 95% sequence identity thereto).
773. The method of any one of embodiments 222 to 224, wherein the disease or condition is Choroideremia and/or wherein the heterologous nucleic acid sequence encodes Rab escort protein (Rep1), CHM (e.g., a polypeptide represented by UniProt Accession number P24386 or a polypeptide comprising an amino acid sequence having at least 95% sequence identity thereto).
774. The method of any one of embodiments 222 to 224, wherein the disease or condition is Cockayne syndrome B (CSB) and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
775. The method of any one of embodiments 222 to 224, wherein the disease or condition is Coffin-Lowry syndrome and/or wherein the heterologous nucleic acid sequence encodes RPS6KA3 (e.g., a polypeptide represented by UniProt Accession number P51812 or a polypeptide comprising an amino acid sequence having at least 95% sequence identity thereto).
776. The method of any one of embodiments 222 to 224, wherein the disease or condition is Craniosynostosis and/or wherein the heterologous nucleic acid sequence encodes MSX2 (e.g., a polypeptide represented by UniProt Accession number P35548 or a polypeptide comprising an amino acid sequence having at least 95% sequence identity thereto).
777. The method of any one of embodiments 222 to 224, wherein the disease or condition is Craniosynostosis and/or wherein the heterologous nucleic acid sequence encodes TWIST1 (e.g., a polypeptide represented by UniProt Accession number Q15672 or a polypeptide comprising an amino acid sequence having at least 95% sequence identity thereto).
778. The method of any one of embodiments 222 to 224, wherein the disease or condition is Craniosynostosis and/or wherein the heterologous nucleic acid sequence encodes SKI (e.g., a polypeptide represented by UniProt Accession number P12755 or a polypeptide comprising an amino acid sequence having at least 95% sequence identity thereto).
779. The method of any one of embodiments 222 to 224, wherein the disease or condition is Craniosynostosis and/or wherein the heterologous nucleic acid sequence encodes SMAD6 (e.g., a polypeptide represented by UniProt Accession number 043541 or a polypeptide comprising an amino acid sequence having at least 95% sequence identity thereto).
780. The method of any one of embodiments 222 to 224, wherein the disease or condition is Creutzfeldt-Jakob disease and/or wherein the heterologous nucleic acid sequence encodes PRNP (e.g., a polypeptide represented by UniProt Accession number P04156 or a polypeptide comprising an amino acid sequence having at least 95% sequence identity thereto).
781. The method of any one of embodiments 222 to 224, wherein the disease or condition is Creutzfeldt-Jakob disease and/or wherein the heterologous nucleic acid sequence encodes HLA-DQB1 (e.g., a polypeptide represented by UniProt Accession number P01920 or a polypeptide comprising an amino acid sequence having at least 95% sequence identity thereto).
782. The method of any one of embodiments 222 to 224, wherein the disease or condition is Crigler-Najjar Syndrome (hyperbilirubinemia) and/or wherein the heterologous nucleic acid sequence encodes UDP-glucuronosyltransferase 1A1 (UGT1A1) (e.g., a polypeptide represented by UniProt Accession number P22309 or a polypeptide comprising an amino acid sequence having at least 95% sequence identity thereto).
783. The method of any one of embodiments 222 to 224, wherein the disease or condition is Cushing Syndrome and/or wherein the heterologous nucleic acid sequence encodes PRKACA (e.g., a polypeptide represented by UniProt Accession number P17612 or a polypeptide comprising an amino acid sequence having at least 95% sequence identity thereto).
784. The method of any one of embodiments 222 to 224, wherein the disease or condition is Dentatorubral atrophy (DRPLA) and/or wherein the heterologous nucleic acid sequence encodes ATN1 (e.g., a polypeptide represented by UniProt Accession number P54259 or a polypeptide comprising an amino acid sequence having at least 95% sequence identity thereto).
785. The method of any one of embodiments 222 to 224, wherein the disease or condition is Developmental and Epileptic Encephalopathy and/or wherein the heterologous nucleic acid sequence encodes ARX (e.g., a polypeptide represented by UniProt Accession number Q96QS3 or a polypeptide comprising an amino acid sequence having at least 95% sequence identity thereto).
786. The method of any one of embodiments 222 to 224, wherein the disease or condition is Developmental and Epileptic Encephalopathy and/or wherein the heterologous nucleic acid sequence encodes FGF12 (e.g., a polypeptide represented by UniProt Accession number P61328 or a polypeptide comprising an amino acid sequence having at least 95% sequence identity thereto).
787. The method of any one of embodiments 222 to 224, wherein the disease or condition is Developmental and Epileptic Encephalopathy and/or wherein the heterologous nucleic acid sequence encodes PIGP (e.g., a polypeptide represented by UniProt Accession number P57054 or a polypeptide comprising an amino acid sequence having at least 95% sequence identity thereto).
788. The method of any one of embodiments 222 to 224, wherein the disease or condition is Developmental and Epileptic Encephalopathy and/or wherein the heterologous nucleic acid sequence encodes GABRB3 (e.g., a polypeptide represented by UniProt Accession number P28472 or a polypeptide comprising an amino acid sequence having at least 95% sequence identity thereto).
789. The method of any one of embodiments 222 to 224, wherein the disease or condition is Developmental and Epileptic Encephalopathy and/or wherein the heterologous nucleic acid sequence encodes NECAP1 (e.g., a polypeptide represented by UniProt Accession number Q8NC96 or a polypeptide comprising an amino acid sequence having at least 95% sequence identity thereto).
790. The method of any one of embodiments 222 to 224, wherein the disease or condition is Developmental Dyspraxia (speech-language disorder 1 (SPCH1)) and/or wherein the heterologous nucleic acid sequence encodes FOXP2 (e.g., a polypeptide represented by UniProt Accession number 015409 or a polypeptide comprising an amino acid sequence having at least 95% sequence identity thereto).
791. The method of any one of embodiments 222 to 224, wherein the disease or condition is Dravet syndrome and/or wherein the heterologous nucleic acid sequence encodes Sodium channel protein type 1 subunit alpha (SCN1A) (e.g., a polypeptide represented by UniProt Accession number P35498 or a polypeptide comprising an amino acid sequence having at least 95% sequence identity thereto).
792. The method of any one of embodiments 222 to 224, wherein the disease or condition is Dravet syndrome and/or wherein the heterologous nucleic acid sequence encodes SCN1B (e.g., a polypeptide represented by UniProt Accession number Q07699 or a polypeptide comprising an amino acid sequence having at least 95% sequence identity thereto).
793. The method of any one of embodiments 222 to 224, wherein the disease or condition is Dravet syndrome and/or wherein the heterologous nucleic acid sequence encodes SCN2A (e.g., a polypeptide represented by UniProt Accession number Q99250 or a polypeptide comprising an amino acid sequence having at least 95% sequence identity thereto).
794. The method of any one of embodiments 222 to 224, wherein the disease or condition is Dravet syndrome and/or wherein the heterologous nucleic acid sequence encodes GABA receptor subunit gamma-2 (GABRG2) (e.g., a polypeptide represented by UniProt Accession number P18507 or a polypeptide comprising an amino acid sequence having at least 95% sequence identity thereto).
795. The method of any one of embodiments 222 to 224, wherein the disease or condition is Dysautonomia (Day syndrome) and/or wherein the heterologous nucleic acid sequence encodes ELP1 (e.g., a polypeptide represented by UniProt Accession number 095163 or a polypeptide comprising an amino acid sequence having at least 95% sequence identity thereto).
796. The method of any one of embodiments 222 to 224, wherein the disease or condition is Dystonia and/or wherein the heterologous nucleic acid sequence encodes GCH1 (e.g., a polypeptide represented by UniProt Accession number P30793 or a polypeptide comprising an amino acid sequence having at least 95% sequence identity thereto).
797. The method of any one of embodiments 222 to 224, wherein the disease or condition is Dystonia and/or wherein the heterologous nucleic acid sequence encodes TOR1A (e.g., a polypeptide represented by UniProt Accession number 014656 or a polypeptide comprising an amino acid sequence having at least 95% sequence identity thereto).
798. The method of any one of embodiments 222 to 224, wherein the disease or condition is Dystonia and/or wherein the heterologous nucleic acid sequence encodes SGCE (e.g., a polypeptide represented by UniProt Accession number 043556 or a polypeptide comprising an amino acid sequence having at least 95% sequence identity thereto).
799. The method of any one of embodiments 222 to 224, wherein the disease or condition is Dystonia and/or wherein the heterologous nucleic acid sequence encodes TUBB4A (e.g., a polypeptide represented by UniProt Accession number P04350 or a polypeptide comprising an amino acid sequence having at least 95% sequence identity thereto).
800. The method of any one of embodiments 222 to 224, wherein the disease or condition is Encephalocele and/or wherein the heterologous nucleic acid sequence encodes COL18A1 (e.g., a polypeptide represented by UniProt Accession number P39060 or a polypeptide comprising an amino acid sequence having at least 95% sequence identity thereto).
801. The method of any one of embodiments 222 to 224, wherein the disease or condition is Epilepsy disorder and/or wherein the heterologous nucleic acid sequence encodes GRIN2A (e.g., a polypeptide represented by UniProt Accession number Q12879 or a polypeptide comprising an amino acid sequence having at least 95% sequence identity thereto).
802. The method of any one of embodiments 222 to 224, wherein the disease or condition is Epilepsy disorder and/or wherein the heterologous nucleic acid sequence encodes CSTB (e.g., a polypeptide represented by UniProt Accession number P04080 or a polypeptide comprising an amino acid sequence having at least 95% sequence identity thereto).
803. The method of any one of embodiments 222 to 224, wherein the disease or condition is Epilepsy disorder and/or wherein the heterologous nucleic acid sequence encodes STARD7 (e.g., a polypeptide represented by UniProt Accession number Q9NQZ5 or a polypeptide comprising an amino acid sequence having at least 95% sequence identity thereto).
804. The method of any one of embodiments 222 to 224, wherein the disease or condition is Epilepsy disorder and/or wherein the heterologous nucleic acid sequence encodes DEPDC5 (e.g., a polypeptide represented by UniProt Accession number 075140 or a polypeptide comprising an amino acid sequence having at least 95% sequence identity thereto).
805. The method of any one of embodiments 222 to 224, wherein the disease or condition is Epilepsy disorder and/or wherein the heterologous nucleic acid sequence encodes PCDH19 (e.g., a polypeptide represented by UniProt Accession number Q8TAB3 or a polypeptide comprising an amino acid sequence having at least 95% sequence identity thereto).
806. The method of any one of embodiments 222 to 224, wherein the disease or condition is Essential tremor and/or wherein the heterologous nucleic acid sequence encodes DRD3 (e.g., a polypeptide represented by UniProt Accession number P35462 or a polypeptide comprising an amino acid sequence having at least 95% sequence identity thereto).
807. The method of any one of embodiments 222 to 224, wherein the disease or condition is Essential tremor and/or wherein the heterologous nucleic acid sequence encodes NOTCH2NLC (e.g., a polypeptide represented by UniProt Accession number PODPK4 or a polypeptide comprising an amino acid sequence having at least 95% sequence identity thereto).
808. The method of any one of embodiments 222 to 224, wherein the disease or condition is Essential tremor and/or wherein the heterologous nucleic acid sequence encodes FUS (e.g., a polypeptide represented by UniProt Accession number P35637 or a polypeptide comprising an amino acid sequence having at least 95% sequence identity thereto).
809. The method of any one of embodiments 222 to 224, wherein the disease or condition is Fabry disease and/or wherein the heterologous nucleic acid sequence encodes alpha-galactosidase A (GLA) (e.g., a polypeptide represented by UniProt Accession number P06280 or a polypeptide comprising an amino acid sequence having at least 95% sequence identity thereto).
810. The method of any one of embodiments 222 to 224, wherein the disease or condition is Farber disease (ceramidase deficiency) and/or wherein the heterologous nucleic acid sequence encodes ASAH1 (e.g., a polypeptide represented by UniProt Accession number Q13510 or a polypeptide comprising an amino acid sequence having at least 95% sequence identity thereto).
811. The method of any one of embodiments 222 to 224, wherein the disease or condition is Fahr disease and/or wherein the heterologous nucleic acid sequence encodes SLC20A2 (e.g., a polypeptide represented by UniProt Accession number Q08357 or a polypeptide comprising an amino acid sequence having at least 95% sequence identity thereto).
812. The method of any one of embodiments 222 to 224, wherein the disease or condition is Febrile Seizure and/or wherein the heterologous nucleic acid sequence encodes GABRG2 (e.g., a polypeptide represented by UniProt Accession number P18507 or a polypeptide comprising an amino acid sequence having at least 95% sequence identity thereto).
813. The method of any one of embodiments 222 to 224, wherein the disease or condition is Febrile Seizure and/or wherein the heterologous nucleic acid sequence encodes ADGRV1 (e.g., a polypeptide represented by UniProt Accession number Q8WXG9 or a polypeptide comprising an amino acid sequence having at least 95% sequence identity thereto).
814. The method of any one of embodiments 222 to 224, wherein the disease or condition is Febrile Seizure and/or wherein the heterologous nucleic acid sequence encodes CPA6 (e.g., a polypeptide represented by UniProt Accession number P11509 or a polypeptide comprising an amino acid sequence having at least 95% sequence identity thereto).
815. The method of any one of embodiments 222 to 224, wherein the disease or condition is Febrile Seizure and/or wherein the heterologous nucleic acid sequence encodes SCN1A (e.g., a polypeptide represented by UniProt Accession number P35498 or a polypeptide comprising an amino acid sequence having at least 95% sequence identity thereto).
816. The method of any one of embodiments 222 to 224, wherein the disease or condition is Friedreich's ataxia and/or wherein the heterologous nucleic acid sequence encodes Frataxin (FXN) (e.g., a polypeptide represented by UniProt Accession number Q16595 or a polypeptide comprising an amino acid sequence having at least 95% sequence identity thereto).
817. The method of any one of embodiments 222 to 224, wherein the disease or condition is Frontotemporal dementia and/or wherein the heterologous nucleic acid sequence encodes Progranulin (GRN) (e.g., a polypeptide represented by UniProt Accession number P28799 or a polypeptide comprising an amino acid sequence having at least 95% sequence identity thereto).
818. The method of any one of embodiments 222 to 224, wherein the disease or condition is Frontotemporal dementia and/or wherein the heterologous nucleic acid sequence encodes MAPT (tau) (e.g., a polypeptide represented by UniProt Accession number P10636 or a polypeptide comprising an amino acid sequence having at least 95% sequence identity thereto).
819. The method of any one of embodiments 222 to 224, wherein the disease or condition is Frontotemporal dementia and/or wherein the heterologous nucleic acid sequence encodes PSEN1 (e.g., a polypeptide represented by UniProt Accession number A0A024R6A3 or a polypeptide comprising an amino acid sequence having at least 95% sequence identity thereto).
820. The method of any one of embodiments 222 to 224, wherein the disease or condition is Fucosidosis and/or wherein the heterologous nucleic acid sequence encodes alpha-L-fucosidase (FUCA1) (e.g., a polypeptide represented by UniProt Accession number P04066 or a polypeptide comprising an amino acid sequence having at least 95% sequence identity thereto).
821. The method of any one of embodiments 222 to 224, wherein the disease or condition is Fundus albipunctatus and/or wherein the heterologous nucleic acid sequence encodes RLBP1 (e.g., a polypeptide represented by UniProt Accession number P12271 or a polypeptide comprising an amino acid sequence having at least 95% sequence identity thereto).
822. The method of any one of embodiments 222 to 224, wherein the disease or condition is Gaucher disease (e.g., types I, II or Ill) and/or wherein the heterologous nucleic acid sequence encodes Glucocerebrosidase (GBA1) (e.g., a polypeptide represented by UniProt Accession number P04062 or a polypeptide comprising an amino acid sequence having at least 95% sequence identity thereto).
823. The method of any one of embodiments 222 to 224, wherein the disease or condition is Generalized gangliosidose (e.g., GM1, GM2 or GM3) and/or wherein the heterologous nucleic acid sequence encodes GLB1 (e.g., a polypeptide represented by UniProt Accession number P16278 or a polypeptide comprising an amino acid sequence having at least 95% sequence identity thereto).
824. The method of any one of embodiments 222 to 224, wherein the disease or condition is Gerstmann-Straussler-Scheinker disease and/or wherein the heterologous nucleic acid sequence encodes PRNP (e.g., a polypeptide represented by UniProt Accession number P04156 or a polypeptide comprising an amino acid sequence having at least 95% sequence identity thereto).
825. The method of any one of embodiments 222 to 224, wherein the disease or condition is Giant axonal neuropathy and/or wherein the heterologous nucleic acid sequence encodes Gigaxonin (GAN) (e.g., a polypeptide represented by UniProt Accession number Q9H2C0 or a polypeptide comprising an amino acid sequence having at least 95% sequence identity thereto).
826. The method of any one of embodiments 222 to 224, wherein the disease or condition is Glycogen storage disease II (Pompe disease or Acid Maltase Deficiency) and/or wherein the heterologous nucleic acid sequence encodes Acid maltase, lysosomal alpha-glucosidase (LYAG, GAA) (e.g., a polypeptide represented by UniProt Accession number P10253 or a polypeptide comprising an amino acid sequence having at least 95% sequence identity thereto).
827. The method of any one of embodiments 222 to 224, wherein the disease or condition is Guillain-Barre syndrome and/or wherein the heterologous nucleic acid sequence encodes PMP22 (e.g., a polypeptide represented by UniProt Accession number Q01453 or a polypeptide comprising an amino acid sequence having at least 95% sequence identity thereto).
828. The method of any one of embodiments 222 to 224, wherein the disease or condition is Chronic Inflammatory Demyelinating Polyneuropathy (CIDP) and/or wherein the heterologous nucleic acid sequence encodes PMP22 (e.g., a polypeptide represented by UniProt Accession number Q01453 or a polypeptide comprising an amino acid sequence having at least 95% sequence identity thereto).
829. The method of any one of embodiments 222 to 224, wherein the disease or condition is Hallervorden-Spatz disease (PKAN or NBIA1) and/or wherein the heterologous nucleic acid sequence encodes PANK2 (e.g., a polypeptide represented by UniProt Accession number Q9BZ23 or a polypeptide comprising an amino acid sequence having at least 95% sequence identity thereto).
830. The method of any one of embodiments 222 to 224, wherein the disease or condition is Hemiplegia Alterans and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
831. The method of any one of embodiments 222 to 224, wherein the disease or condition is Hemiplegia Alterans and/or wherein the heterologous nucleic acid sequence encodes ATP1A3 (e.g., a polypeptide represented by UniProt Accession number P13637 or a polypeptide comprising an amino acid sequence having at least 95% sequence identity thereto).
832. The method of any one of embodiments 222 to 224, wherein the disease or condition is Hereditary Neuropathy and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
833. The method of any one of embodiments 222 to 224, wherein the disease or condition is Hereditary Neuropathy and/or wherein the heterologous nucleic acid sequence encodes MFN2 (e.g., a polypeptide represented by UniProt Accession number 095140 or a polypeptide comprising an amino acid sequence having at least 95% sequence identity thereto).
834. The method of any one of embodiments 222 to 224, wherein the disease or condition is Hereditary Neuropathy and/or wherein the heterologous nucleic acid sequence encodes HK1 (e.g., a polypeptide represented by UniProt Accession number P19367 or a polypeptide comprising an amino acid sequence having at least 95% sequence identity thereto).
835. The method of any one of embodiments 222 to 224, wherein the disease or condition is Hereditary Neuropathy and/or wherein the heterologous nucleic acid sequence encodes TFG (e.g., a polypeptide represented by UniProt Accession number Q92734 or a polypeptide comprising an amino acid sequence having at least 95% sequence identity thereto).
836. The method of any one of embodiments 222 to 224, wherein the disease or condition is Hereditary Neuropathy and/or wherein the heterologous nucleic acid sequence encodes SPTLC1 (e.g., a polypeptide represented by UniProt Accession number 015269 or a polypeptide comprising an amino acid sequence having at least 95% sequence identity thereto).
837. The method of any one of embodiments 222 to 224, wherein the disease or condition is Heredopathia Atactica Polyneuritiformis (Refsum disease) and/or wherein the heterologous nucleic acid sequence encodes PHYH (e.g., a polypeptide represented by UniProt Accession number 014832 or a polypeptide comprising an amino acid sequence having at least 95% sequence identity thereto).
838. The method of any one of embodiments 222 to 224, wherein the disease or condition is Holoprosencephaly and/or wherein the heterologous nucleic acid sequence encodes GI12 (e.g., a polypeptide represented by UniProt Accession number P10070 or a polypeptide comprising an amino acid sequence having at least 95% sequence identity thereto).
839. The method of any one of embodiments 222 to 224, wherein the disease or condition is Holoprosencephaly and/or wherein the heterologous nucleic acid sequence encodes TGIF1 (e.g., a polypeptide represented by UniProt Accession number Q15583 or a polypeptide comprising an amino acid sequence having at least 95% sequence identity thereto).
840. The method of any one of embodiments 222 to 224, wherein the disease or condition is Holoprosencephaly and/or wherein the heterologous nucleic acid sequence encodes ZIC2 (e.g., a polypeptide represented by UniProt Accession number 095409 or a polypeptide comprising an amino acid sequence having at least 95% sequence identity thereto).
841. The method of any one of embodiments 222 to 224, wherein the disease or condition is Holoprosencephaly and/or wherein the heterologous nucleic acid sequence encodes PTCH1 (e.g., a polypeptide represented by UniProt Accession number Q13635 or a polypeptide comprising an amino acid sequence having at least 95% sequence identity thereto).
842. The method of any one of embodiments 222 to 224, wherein the disease or condition is Holoprosencephaly and/or wherein the heterologous nucleic acid sequence encodes SHH (e.g., a polypeptide represented by UniProt Accession number Q15465 or a polypeptide comprising an amino acid sequence having at least 95% sequence identity thereto).
843. The method of any one of embodiments 222 to 224, wherein the disease or condition is Huntington's disease and/or wherein the heterologous nucleic acid sequence encodes HTT (e.g., a polypeptide represented by UniProt Accession number P42858 or a polypeptide comprising an amino acid sequence having at least 95% sequence identity thereto).
844. The method of any one of embodiments 222 to 224, wherein the disease or condition is Hydrocephalus disorder and/or wherein the heterologous nucleic acid sequence encodes CCDC88C (e.g., a polypeptide represented by UniProt Accession number Q9P219 or a polypeptide comprising an amino acid sequence having at least 95% sequence identity thereto).
845. The method of any one of embodiments 222 to 224, wherein the disease or condition is Hydrocephalus disorder and/or wherein the heterologous nucleic acid sequence encodes WDR81 (e.g., a polypeptide represented by UniProt Accession number Q562E7 or a polypeptide comprising an amino acid sequence having at least 95% sequence identity thereto).
846. The method of any one of embodiments 222 to 224, wherein the disease or condition is Hydrocephalus disorder and/or wherein the heterologous nucleic acid sequence encodes TRIM71 (e.g., a polypeptide represented by UniProt Accession number Q2Q1W2 or a polypeptide comprising an amino acid sequence having at least 95% sequence identity thereto).
847. The method of any one of embodiments 222 to 224, wherein the disease or condition is Hydrocephalus disorder and/or wherein the heterologous nucleic acid sequence encodes MPDZ (e.g., a polypeptide represented by UniProt Accession number 075970 or a polypeptide comprising an amino acid sequence having at least 95% sequence identity thereto).
848. The method of any one of embodiments 222 to 224, wherein the disease or condition is Incontinentia Pigmenti and/or wherein the heterologous nucleic acid sequence encodes IKBKG (e.g., a polypeptide represented by UniProt Accession number Q9Y6K9 or a polypeptide comprising an amino acid sequence having at least 95% sequence identity thereto).
849. The method of any one of embodiments 222 to 224, wherein the disease or condition is Infantile Hypotonia and/or wherein the heterologous nucleic acid sequence encodes NALCN (e.g., a polypeptide represented by UniProt Accession number Q81ZFO or a polypeptide comprising an amino acid sequence having at least 95% sequence identity thereto).
850. The method of any one of embodiments 222 to 224, wherein the disease or condition is Infantile Hypotonia and/or wherein the heterologous nucleic acid sequence encodes TBCK (e.g., a polypeptide represented by UniProt Accession number Q8TEA7 or a polypeptide comprising an amino acid sequence having at least 95% sequence identity thereto).
851. The method of any one of embodiments 222 to 224, wherein the disease or condition is Infantile Hypotonia and/or wherein the heterologous nucleic acid sequence encodes CCDC174 (e.g., a polypeptide represented by UniProt Accession number Q6P113 or a polypeptide comprising an amino acid sequence having at least 95% sequence identity thereto).
852. The method of any one of embodiments 222 to 224, wherein the disease or condition is Infantile Hypotonia and/or wherein the heterologous nucleic acid sequence encodes UNC80 (e.g., a polypeptide represented by UniProt Accession number Q8N2C7 or a polypeptide comprising an amino acid sequence having at least 95% sequence identity thereto).
853. The method of any one of embodiments 222 to 224, wherein the disease or condition is Infantile Neuroaxonal Dystrophy and/or wherein the heterologous nucleic acid sequence encodes PLA2G6 (e.g., a polypeptide represented by UniProt Accession number 060733 or a polypeptide comprising an amino acid sequence having at least 95% sequence identity thereto).
854. The method of any one of embodiments 222 to 224, wherein the disease or condition is Infantile Phytanic Acid Storage Disease (PBD1B) and/or wherein the heterologous nucleic acid sequence encodes PEX1 (e.g., a polypeptide represented by UniProt Accession number 043933 or a polypeptide comprising an amino acid sequence having at least 95% sequence identity thereto).
855. The method of any one of embodiments 222 to 224, wherein the disease or condition is Joubert Syndrome and/or wherein the heterologous nucleic acid sequence encodes INPP5E (e.g., a polypeptide represented by UniProt Accession number Q9NRR6 or a polypeptide comprising an amino acid sequence having at least 95% sequence identity thereto).
856. The method of any one of embodiments 222 to 224, wherein the disease or condition is Kennedy Disease and/or wherein the heterologous nucleic acid sequence encodes Androgen receptor (AR) (e.g., a polypeptide represented by UniProt Accession number P10275 or a polypeptide comprising an amino acid sequence having at least 95% sequence identity thereto).
857. The method of any one of embodiments 222 to 224, wherein the disease or condition is Klippel-Feil Syndrome and/or wherein the heterologous nucleic acid sequence encodes GDF6 (e.g., a polypeptide represented by UniProt Accession number Q6KF10 or a polypeptide comprising an amino acid sequence having at least 95% sequence identity thereto).
858. The method of any one of embodiments 222 to 224, wherein the disease or condition is Krabbe disease (GALC deficiency) and/or wherein the heterologous nucleic acid sequence encodes GALC (e.g., a polypeptide represented by UniProt Accession number P54803 or a polypeptide comprising an amino acid sequence having at least 95% sequence identity thereto).
859. The method of any one of embodiments 222 to 224, wherein the disease or condition is Lambert-Eaton Myasthenic Syndrome and/or wherein the heterologous nucleic acid sequence encodes CACNA1A (e.g., a polypeptide represented by UniProt Accession number 000555 or a polypeptide comprising an amino acid sequence having at least 95% sequence identity thereto).
860. The method of any one of embodiments 222 to 224, wherein the disease or condition is Lambert-Eaton Myasthenic Syndrome and/or wherein the heterologous nucleic acid sequence encodes CACNB2 (e.g., a polypeptide represented by UniProt Accession number Q13936 or a polypeptide comprising an amino acid sequence having at least 95% sequence identity thereto).
861. The method of any one of embodiments 222 to 224, wherein the disease or condition is Landau-Kleffner Syndrome and/or wherein the heterologous nucleic acid sequence encodes GRIN2A (e.g., a polypeptide represented by UniProt Accession number Q12879 or a polypeptide comprising an amino acid sequence having at least 95% sequence identity thereto).
862. The method of any one of embodiments 222 to 224, wherein the disease or condition is Late infantile neuronal lipofuscinosis (CLN2) and/or wherein the heterologous nucleic acid sequence encodes TPP1 (e.g., a polypeptide represented by UniProt Accession number 014773 or a polypeptide comprising an amino acid sequence having at least 95% sequence identity thereto).
863. The method of any one of embodiments 222 to 224, wherein the disease or condition is Lesch-Nyhan Syndrome and/or wherein the heterologous nucleic acid sequence encodes HPRT1 (e.g., a polypeptide represented by UniProt Accession number P00492 or a polypeptide comprising an amino acid sequence having at least 95% sequence identity thereto).
864. The method of any one of embodiments 222 to 224, wherein the disease or condition is Leber congenital amaurosis (retinal blindness) and/or wherein the heterologous nucleic acid sequence encodes Retinal guanylyl cyclase 1 (GUCY2D) (e.g., a polypeptide represented by UniProt Accession number Q02846 or a polypeptide comprising an amino acid sequence having at least 95% sequence identity thereto).
865. The method of any one of embodiments 222 to 224, wherein the disease or condition is Leber congenital amaurosis (retinal blindness) and/or wherein the heterologous nucleic acid sequence encodes Retinoid isomerohydrolase (RPE65) (e.g., a polypeptide represented by UniProt Accession number Q16518 or a polypeptide comprising an amino acid sequence having at least 95% sequence identity thereto).
866. The method of any one of embodiments 222 to 224, wherein the disease or condition is Leber congenital amaurosis (retinal blindness) and/or wherein the heterologous nucleic acid sequence encodes Centrosomal protein of 290 kDa (CEP290) (e.g., a polypeptide represented by UniProt Accession number 015078 or a polypeptide comprising an amino acid sequence having at least 95% sequence identity thereto).
867. The method of any one of embodiments 222 to 224, wherein the disease or condition is Leber congenital amaurosis (retinal blindness) and/or wherein the heterologous nucleic acid sequence encodes Protein crumbs homolog 1 (CRB1) (e.g., a polypeptide represented by UniProt Accession number P82279 or a polypeptide comprising an amino acid sequence having at least 95% sequence identity thereto).
868. The method of any one of embodiments 222 to 224, wherein the disease or condition is Leber's hereditary optical neuropathy and/or wherein the heterologous nucleic acid sequence encodes NADH-ubiquinone oxidoreductase chain 4 (ND4) (e.g., a polypeptide represented by UniProt Accession number P03905 or a polypeptide comprising an amino acid sequence having at least 95% sequence identity thereto).
869. The method of any one of embodiments 222 to 224, wherein the disease or condition is Leukodystrophy and/or wherein the heterologous nucleic acid sequence encodes ARSA (e.g., a polypeptide represented by UniProt Accession number P15289 or a polypeptide comprising an amino acid sequence having at least 95% sequence identity thereto).
870. The method of any one of embodiments 222 to 224, wherein the disease or condition is Levine-Critchley Syndrome (choreoacanthocytosis) and/or wherein the heterologous nucleic acid sequence encodes VPS13A (e.g., a polypeptide represented by UniProt Accession number Q96RL7 or a polypeptide comprising an amino acid sequence having at least 95% sequence identity thereto).
871. The method of any one of embodiments 222 to 224, wherein the disease or condition is Lewy body dementia and/or wherein the heterologous nucleic acid sequence encodes SNCA (e.g., a polypeptide represented by UniProt Accession number P37840 or a polypeptide comprising an amino acid sequence having at least 95% sequence identity thereto).
872. The method of any one of embodiments 222 to 224, wherein the disease or condition is Lewy body dementia and/or wherein the heterologous nucleic acid sequence encodes SNCB (e.g., a polypeptide represented by UniProt Accession number Q16143 or a polypeptide comprising an amino acid sequence having at least 95% sequence identity thereto).
873. The method of any one of embodiments 222 to 224, wherein the disease or condition is Lipoid Proteinosis (Urbach-Wiethe disease) and/or wherein the heterologous nucleic acid sequence encodes ECM1 (e.g., a polypeptide represented by UniProt Accession number Q16610 or a polypeptide comprising an amino acid sequence having at least 95% sequence identity thereto).
874. The method of any one of embodiments 222 to 224, wherein the disease or condition is Lissencephaly and/or wherein the heterologous nucleic acid sequence encodes PAFAH1B1 (e.g., a polypeptide represented by UniProt Accession number Q9PTR5 or a polypeptide comprising an amino acid sequence having at least 95% sequence identity thereto).
875. The method of any one of embodiments 222 to 224, wherein the disease or condition is Lissencephaly and/or wherein the heterologous nucleic acid sequence encodes NDE1 (e.g., a polypeptide represented by UniProt Accession number Q9NXR1 or a polypeptide comprising an amino acid sequence having at least 95% sequence identity thereto).
876. The method of any one of embodiments 222 to 224, wherein the disease or condition is Lissencephaly and/or wherein the heterologous nucleic acid sequence encodes TUBA1A (e.g., a polypeptide represented by UniProt Accession number Q71U36 or a polypeptide comprising an amino acid sequence having at least 95% sequence identity thereto).
877. The method of any one of embodiments 222 to 224, wherein the disease or condition is Lissencephaly and/or wherein the heterologous nucleic acid sequence encodes LAMB1 (e.g., a polypeptide represented by UniProt Accession number LAMB1 or a polypeptide comprising an amino acid sequence having at least 95% sequence identity thereto).
878. The method of any one of embodiments 222 to 224, wherein the disease or condition is Lissencephaly and/or wherein the heterologous nucleic acid sequence encodes KATNB1 (e.g., a polypeptide represented by UniProt Accession number Q9BVAO or a polypeptide comprising an amino acid sequence having at least 95% sequence identity thereto).
879. The method of any one of embodiments 222 to 224, wherein the disease or condition is Lissencephaly and/or wherein the heterologous nucleic acid sequence encodes RELN (e.g., a polypeptide represented by UniProt Accession number P78509 or a polypeptide comprising an amino acid sequence having at least 95% sequence identity thereto).
880. The method of any one of embodiments 222 to 224, wherein the disease or condition is Macrocephaly/Megalencephaly and/or wherein the heterologous nucleic acid sequence encodes TBC1 D7 (e.g., a polypeptide represented by UniProt Accession number Q9PON9 or a polypeptide comprising an amino acid sequence having at least 95% sequence identity thereto).
881. The method of any one of embodiments 222 to 224, wherein the disease or condition is Menkes Disease and/or wherein the heterologous nucleic acid sequence encodes ATP7A (e.g., a polypeptide represented by UniProt Accession number Q04656 or a polypeptide comprising an amino acid sequence having at least 95% sequence identity thereto).
882. The method of any one of embodiments 222 to 224, wherein the disease or condition is Metachromatic Leukodystrophy (MLD) and/or wherein the heterologous nucleic acid sequence encodes Arylsulfatase A (ARSA) (e.g., a polypeptide represented by UniProt Accession number P15289 or a polypeptide comprising an amino acid sequence having at least 95% sequence identity thereto).
883. The method of any one of embodiments 222 to 224, wherein the disease or condition is Microcephaly and/or wherein the heterologous nucleic acid sequence encodes KIF11 (e.g., a polypeptide represented by UniProt Accession number P52732 or a polypeptide comprising an amino acid sequence having at least 95% sequence identity thereto).
884. The method of any one of embodiments 222 to 224, wherein the disease or condition is Microcephaly and/or wherein the heterologous nucleic acid sequence encodes MCPH1 (e.g., a polypeptide represented by UniProt Accession number Q8NEMO or a polypeptide comprising an amino acid sequence having at least 95% sequence identity thereto).
885. The method of any one of embodiments 222 to 224, wherein the disease or condition is Microcephaly and/or wherein the heterologous nucleic acid sequence encodes SLC25A19 (e.g., a polypeptide represented by UniProt Accession number Q9HC21 or a polypeptide comprising an amino acid sequence having at least 95% sequence identity thereto).
886. The method of any one of embodiments 222 to 224, wherein the disease or condition is Migraine, familial hemiplegic and/or wherein the heterologous nucleic acid sequence encodes CACNA1A (e.g., a polypeptide represented by UniProt Accession number 000555 or a polypeptide comprising an amino acid sequence having at least 95% sequence identity thereto).
887. The method of any one of embodiments 222 to 224, wherein the disease or condition is Migraine, familial hemiplegic and/or wherein the heterologous nucleic acid sequence encodes ATP1A2 (e.g., a polypeptide represented by UniProt Accession number P50993 or a polypeptide comprising an amino acid sequence having at least 95% sequence identity thereto).
888. The method of any one of embodiments 222 to 224, wherein the disease or condition is Migraine, familial hemiplegic and/or wherein the heterologous nucleic acid sequence encodes SCN1A (e.g., a polypeptide represented by UniProt Accession number P35498 or a polypeptide comprising an amino acid sequence having at least 95% sequence identity thereto).
889. The method of any one of embodiments 222 to 224, wherein the disease or condition is Mitochondrial DNA depletion syndrome and/or wherein the heterologous nucleic acid sequence encodes RRM2B (e.g., a polypeptide represented by UniProt Accession number Q7LG56 or a polypeptide comprising an amino acid sequence having at least 95% sequence identity thereto).
890. The method of any one of embodiments 222 to 224, wherein the disease or condition is Mitochondrial DNA depletion syndrome and/or wherein the heterologous nucleic acid sequence encodes DGUOK (e.g., a polypeptide represented by UniProt Accession number Q16854 or a polypeptide comprising an amino acid sequence having at least 95% sequence identity thereto).
891. The method of any one of embodiments 222 to 224, wherein the disease or condition is Mitochondrial DNA depletion syndrome and/or wherein the heterologous nucleic acid sequence encodes POLG (e.g., a polypeptide represented by UniProt Accession number P54098 or a polypeptide comprising an amino acid sequence having at least 95% sequence identity thereto).
892. The method of any one of embodiments 222 to 224, wherein the disease or condition is Mitochondrial DNA depletion syndrome and/or wherein the heterologous nucleic acid sequence encodes TYMP (e.g., a polypeptide represented by UniProt Accession number P19971 or a polypeptide comprising an amino acid sequence having at least 95% sequence identity thereto).
893. The method of any one of embodiments 222 to 224, wherein the disease or condition is Mitochondrial DNA depletion syndrome and/or wherein the heterologous nucleic acid sequence encodes TK2 (e.g., a polypeptide represented by UniProt Accession number 000142 or a polypeptide comprising an amino acid sequence having at least 95% sequence identity thereto).
894. The method of any one of embodiments 222 to 224, wherein the disease or condition is Morvan disease and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
895. The method of any one of embodiments 222 to 224, wherein the disease or condition is Mucolipidosis and/or wherein the heterologous nucleic acid sequence encodes GNPTAB (e.g., a polypeptide represented by UniProt Accession number Q3T906 or a polypeptide comprising an amino acid sequence having at least 95% sequence identity thereto).
896. The method of any one of embodiments 222 to 224, wherein the disease or condition is Mucolipidosis and/or wherein the heterologous nucleic acid sequence encodes MCOLN1 (e.g., a polypeptide represented by UniProt Accession number Q9GZU1 or a polypeptide comprising an amino acid sequence having at least 95% sequence identity thereto).
897. The method of any one of embodiments 222 to 224, wherein the disease or condition is Mucopolysaccharidosis Type I (MPS I) (Hurler syndrome) and/or wherein the heterologous nucleic acid sequence encodes alpha-L-iduronidase (IDUA) (e.g., a polypeptide represented by UniProt Accession number P35475 or a polypeptide comprising an amino acid sequence having at least 95% sequence identity thereto).
898. The method of any one of embodiments 222 to 224, wherein the disease or condition is MPS II (Hunter syndrome) and/or wherein the heterologous nucleic acid sequence encodes iduronate-2-sulfatase (IDS) (e.g., a polypeptide represented by UniProt Accession number P22304 or a polypeptide comprising an amino acid sequence having at least 95% sequence identity thereto).
899. The method of any one of embodiments 222 to 224, wherein the disease or condition is MPS IIIa (Sanfilippo Type A syndrome) and/or wherein the heterologous nucleic acid sequence encodes heparan sulfate sulfatase (HSS) or N-sulfoglucosamine sulfohydrolase (SGSH) (e.g., a polypeptide represented by UniProt Accession number P51688 or a polypeptide comprising an amino acid sequence having at least 95% sequence identity thereto).
900. The method of any one of embodiments 222 to 224, wherein the disease or condition is MPS IIIB (Sanfilippo Type B syndrome) and/or wherein the heterologous nucleic acid sequence encodes N-acetyl-alpha-D-glucosaminidase (NAGLU) (e.g., a polypeptide represented by UniProt Accession number P54802 or a polypeptide comprising an amino acid sequence having at least 95% sequence identity thereto).
901. The method of any one of embodiments 222 to 224, wherein the disease or condition is MPS VI (Maroteaux-Lamy syndrome) and/or wherein the heterologous nucleic acid sequence encodes arylsulfatase B (ARSB) (e.g., a polypeptide represented by UniProt Accession number P15848 or a polypeptide comprising an amino acid sequence having at least 95% sequence identity thereto).
902. The method of any one of embodiments 222 to 224, wherein the disease or condition is MPS IV A (Morquio syndrome type A) and/or wherein the heterologous nucleic acid sequence encodes N-acetylgalactosamine-6-sulfatase (GALNS) (e.g., a polypeptide represented by UniProt Accession number P34059 or a polypeptide comprising an amino acid sequence having at least 95% sequence identity thereto).
903. The method of any one of embodiments 222 to 224, wherein the disease or condition is MPS IV B (Morquio syndrome type B) and/or wherein the heterologous nucleic acid sequence encodes Beta-galactosidase 1 (GLB1) (e.g., a polypeptide represented by UniProt Accession number P16278 or a polypeptide comprising an amino acid sequence having at least 95% sequence identity thereto).
904. The method of any one of embodiments 222 to 224, wherein the disease or condition is MPS VII (Sly syndrome) and/or wherein the heterologous nucleic acid sequence encodes beta-glucuronidase (e.g., a polypeptide represented by UniProt Accession number P08236 or a polypeptide comprising an amino acid sequence having at least 95% sequence identity thereto).
905. The method of any one of embodiments 222 to 224, wherein the disease or condition is MPS VIII and/or wherein the heterologous nucleic acid sequence encodes glucosamine-6-sulfate sulfatase (e.g., a polypeptide represented by UniProt Accession number P15586 or a polypeptide comprising an amino acid sequence having at least 95% sequence identity thereto).
906. The method of any one of embodiments 222 to 224, wherein the disease or condition is MPS IX and/or wherein the heterologous nucleic acid sequence encodes Hyaluronidase-1 (HYAL1) (e.g., a polypeptide represented by UniProt Accession number Q12794 or a polypeptide comprising an amino acid sequence having at least 95% sequence identity thereto).
907. The method of any one of embodiments 222 to 224, wherein the disease or condition is Multiple Sclerosis and/or wherein the heterologous nucleic acid sequence encodes PDCD1 (e.g., a polypeptide represented by UniProt Accession number Q15116 or a polypeptide comprising an amino acid sequence having at least 95% sequence identity thereto).
908. The method of any one of embodiments 222 to 224, wherein the disease or condition is Multiple Sclerosis and/or wherein the heterologous nucleic acid sequence encodes an anti-CD20 antibody (e.g., rituximab, ocrelizumab, or ofatumumab, ublituximab, or an antigen binding portion thereof).
909. The method of any one of embodiments 222 to 224, wherein the disease or condition is Multiple system atrophy and/or wherein the heterologous nucleic acid sequence encodes COQ2 (e.g., a polypeptide represented by UniProt Accession number Q96H96 or a polypeptide comprising an amino acid sequence having at least 95% sequence identity thereto).
910. The method of any one of embodiments 222 to 224, wherein the disease or condition is Myasthenic syndrome, congenital presynaptic and/or wherein the heterologous nucleic acid sequence encodes CHAT (e.g., a polypeptide represented by UniProt Accession number P28329 or a polypeptide comprising an amino acid sequence having at least 95% sequence identity thereto).
911. The method of any one of embodiments 222 to 224, wherein the disease or condition is Myoclonus and/or wherein the heterologous nucleic acid sequence encodes NOL3 (e.g., a polypeptide represented by UniProt Accession number 060936 or a polypeptide comprising an amino acid sequence having at least 95% sequence identity thereto).
912. The method of any one of embodiments 222 to 224, wherein the disease or condition is Myoclonic epilepsy (FAME2) and/or wherein the heterologous nucleic acid sequence encodes STARD7 (e.g., a polypeptide represented by UniProt Accession number Q9NQZ5 or a polypeptide comprising an amino acid sequence having at least 95% sequence identity thereto).
913. The method of any one of embodiments 222 to 224, wherein the disease or condition is Narcolepsy and/or wherein the heterologous nucleic acid sequence encodes HCRT, OX (e.g., a polypeptide represented by UniProt Accession number 043612 or a polypeptide comprising an amino acid sequence having at least 95% sequence identity thereto).
914. The method of any one of embodiments 222 to 224, wherein the disease or condition is Narcolepsy and/or wherein the heterologous nucleic acid sequence encodes MOG (e.g., a polypeptide represented by UniProt Accession number Q16653 or a polypeptide comprising an amino acid sequence having at least 95% sequence identity thereto).
915. The method of any one of embodiments 222 to 224, wherein the disease or condition is Neuroacanthocytosis (McLeod syndrome) and/or wherein the heterologous nucleic acid sequence encodes XK (e.g., a polypeptide represented by UniProt Accession number P51811 or a polypeptide comprising an amino acid sequence having at least 95% sequence identity thereto).
916. The method of any one of embodiments 222 to 224, wherein the disease or condition is Neurodevelopmental disorder with cerebral atrophy and facial dysmorphism (NEDCAFD) and/or wherein the heterologous nucleic acid sequence encodes TTC5 (e.g., a polypeptide represented by UniProt Accession number Q8NOZ6 or a polypeptide comprising an amino acid sequence having at least 95% sequence identity thereto).
917. The method of any one of embodiments 222 to 224, wherein the disease or condition is Neurodevelopmental disorder with infantile epileptic spasms and/or wherein the heterologous nucleic acid sequence encodes NCDN (e.g., a polypeptide represented by UniProt Accession number Q9UBB6 or a polypeptide comprising an amino acid sequence having at least 95% sequence identity thereto).
918. The method of any one of embodiments 222 to 224, wherein the disease or condition is Neurofibromatosis and/or wherein the heterologous nucleic acid sequence encodes NF1 (e.g., a polypeptide represented by UniProt Accession number P21359 or a polypeptide comprising an amino acid sequence having at least 95% sequence identity thereto).
919. The method of any one of embodiments 222 to 224, wherein the disease or condition is Neuromyotonia and/or wherein the heterologous nucleic acid sequence encodes HINT1 (e.g., a polypeptide represented by UniProt Accession number P49773 or a polypeptide comprising an amino acid sequence having at least 95% sequence identity thereto).
920. The method of any one of embodiments 222 to 224, wherein the disease or condition is Neuronal Ceroid Lipofuscinosis and/or wherein the heterologous nucleic acid sequence encodes PPT1 (e.g., a polypeptide represented by UniProt Accession number P50897 or a polypeptide comprising an amino acid sequence having at least 95% sequence identity thereto).
921. The method of any one of embodiments 222 to 224, wherein the disease or condition is Neuronal Ceroid Lipofuscinosis and/or wherein the heterologous nucleic acid sequence encodes TPP1 (e.g., a polypeptide represented by UniProt Accession number 014773 or a polypeptide comprising an amino acid sequence having at least 95% sequence identity thereto).
922. The method of any one of embodiments 222 to 224, wherein the disease or condition is Neuronal Ceroid Lipofuscinosis and/or wherein the heterologous nucleic acid sequence encodes CLN5 (e.g., a polypeptide represented by UniProt Accession number 075503 or a polypeptide comprising an amino acid sequence having at least 95% sequence identity thereto).
923. The method of any one of embodiments 222 to 224, wherein the disease or condition is Neuronal Ceroid Lipofuscinosis and/or wherein the heterologous nucleic acid sequence encodes CLN3 (e.g., a polypeptide represented by UniProt Accession number Q13286 or a polypeptide comprising an amino acid sequence having at least 95% sequence identity thereto).
924. The method of any one of embodiments 222 to 224, wherein the disease or condition is Neuronal Ceroid Lipofuscinosis and/or wherein the heterologous nucleic acid sequence encodes CLN6 (e.g., a polypeptide represented by UniProt Accession number Q9NWW5 or a polypeptide comprising an amino acid sequence having at least 95% sequence identity thereto).
925. The method of any one of embodiments 222 to 224, wherein the disease or condition is Neuronal Ceroid Lipofuscinosis and/or wherein the heterologous nucleic acid sequence encodes CLN8 (e.g., a polypeptide represented by UniProt Accession number Q9UBY8 or a polypeptide comprising an amino acid sequence having at least 95% sequence identity thereto).
926. The method of any one of embodiments 222 to 224, wherein the disease or condition is Neuronal Ceroid Lipofuscinosis and/or wherein the heterologous nucleic acid sequence encodes DNAJC5 (e.g., a polypeptide represented by UniProt Accession number Q9H3Z4 or a polypeptide comprising an amino acid sequence having at least 95% sequence identity thereto).
927. The method of any one of embodiments 222 to 224, wherein the disease or condition is Neuronal Ceroid Lipofuscinosis and/or wherein the heterologous nucleic acid sequence encodes MFSD8 (e.g., a polypeptide represented by UniProt Accession number Q8NHS3 or a polypeptide comprising an amino acid sequence having at least 95% sequence identity thereto).
928. The method of any one of embodiments 222 to 224, wherein the disease or condition is Neuronal Ceroid Lipofuscinosis and/or wherein the heterologous nucleic acid sequence encodes CTSD (e.g., a polypeptide represented by UniProt Accession number P07339 or a polypeptide comprising an amino acid sequence having at least 95% sequence identity thereto).
929. The method of any one of embodiments 222 to 224, wherein the disease or condition is Neuropathy, ataxia and retinitis pigmentosa (NARP) and/or wherein the heterologous nucleic acid sequence encodes MTATP6 (e.g., a polypeptide represented by UniProt Accession number P00846 or a polypeptide comprising an amino acid sequence having at least 95% sequence identity thereto).
930. The method of any one of embodiments 222 to 224, wherein the disease or condition is Neuropathy, hereditary sensory and autonomic, type II and/or wherein the heterologous nucleic acid sequence encodes WNK1 (e.g., a polypeptide represented by UniProt Accession number Q9H4A3 or a polypeptide comprising an amino acid sequence having at least 95% sequence identity thereto).
931. The method of any one of embodiments 222 to 224, wherein the disease or condition is Neuropathy, hypomyelinating congenital 1 and/or wherein the heterologous nucleic acid sequence encodes EGR2 (e.g., a polypeptide represented by UniProt Accession number P11161 or a polypeptide comprising an amino acid sequence having at least 95% sequence identity thereto).
932. The method of any one of embodiments 222 to 224, wherein the disease or condition is Niemann-Pick disease and/or wherein the heterologous nucleic acid sequence encodes Sphingomyelin phosphodiesterase 1 (SMPD1) (e.g., a polypeptide represented by UniProt Accession number P17405 or a polypeptide comprising an amino acid sequence having at least 95% sequence identity thereto).
933. The method of any one of embodiments 222 to 224, wherein the disease or condition is Niemann-Pick disease and/or wherein the heterologous nucleic acid sequence encodes NPC intracellular cholesterol transporter 1 (NPC1) (e.g., a polypeptide represented by UniProt Accession number 015118 or a polypeptide comprising an amino acid sequence having at least 95% sequence identity thereto).
934. The method of any one of embodiments 222 to 224, wherein the disease or condition is Ohtahara Syndrome (Developmental and epileptic encephalopathy 1) and/or wherein the heterologous nucleic acid sequence encodes ARX (e.g., a polypeptide represented by UniProt Accession number Q96QS3 or a polypeptide comprising an amino acid sequence having at least 95% sequence identity thereto).
935. The method of any one of embodiments 222 to 224, wherein the disease or condition is Omithine Transcarbamylase deficiency and/or wherein the heterologous nucleic acid sequence encodes OTC (e.g., a polypeptide represented by UniProt Accession number P00480 or a polypeptide comprising an amino acid sequence having at least 95% sequence identity thereto).
936. The method of any one of embodiments 222 to 224, wherein the disease or condition is Orthostatic intolerance and/or wherein the heterologous nucleic acid sequence encodes SLC6A2 (e.g., a polypeptide represented by UniProt Accession number P23975 or a polypeptide comprising an amino acid sequence having at least 95% sequence identity thereto).
937. The method of any one of embodiments 222 to 224, wherein the disease or condition is Parkinson's disease and/or wherein the heterologous nucleic acid sequence encodes Glucocerebrosidase (GBA1) (e.g., a polypeptide represented by UniProt Accession number P04062 or a polypeptide comprising an amino acid sequence having at least 95% sequence identity thereto).
938. The method of any one of embodiments 222 to 224, wherein the disease or condition is Parkinson's disease and/or wherein the heterologous nucleic acid sequence encodes Dopamine decarboxylase (DDC) (e.g., a polypeptide represented by UniProt Accession number P20711 or a polypeptide comprising an amino acid sequence having at least 95% sequence identity thereto).
939. The method of any one of embodiments 222 to 224, wherein the disease or condition is Parkinson's disease and/or wherein the heterologous nucleic acid sequence encodes Neurturin (e.g., a polypeptide represented by UniProt Accession number Q99748 or a polypeptide comprising an amino acid sequence having at least 95% sequence identity thereto).
940. The method of any one of embodiments 222 to 224, wherein the disease or condition is Parkinson's disease and/or wherein the heterologous nucleic acid sequence encodes Glial derived growth factor (GDGF) (e.g., a polypeptide represented by UniProt Accession number P39905 or a polypeptide comprising an amino acid sequence having at least 95% sequence identity thereto).
941. The method of any one of embodiments 222 to 224, wherein the disease or condition is Parkinson's disease and/or wherein the heterologous nucleic acid sequence encodes Tyrosine hydroxylase (TH), tyrosine 3-monooxygenase (e.g., a polypeptide represented by UniProt Accession number P07101 or a polypeptide comprising an amino acid sequence having at least 95% sequence identity thereto).
942. The method of any one of embodiments 222 to 224, wherein the disease or condition is Parkinson's disease and/or wherein the heterologous nucleic acid sequence encodes Glutamic acid decarboxylase (GAD) (e.g., a polypeptide represented by UniProt Accession number Q99259 or a polypeptide comprising an amino acid sequence having at least 95% sequence identity thereto).
943. The method of any one of embodiments 222 to 224, wherein the disease or condition is Parkinson's disease and/or wherein the heterologous nucleic acid sequence encodes Fibroblast growth factor 2 (FGF2) (e.g., a polypeptide represented by UniProt Accession number P09038 or a polypeptide comprising an amino acid sequence having at least 95% sequence identity thereto).
944. The method of any one of embodiments 222 to 224, wherein the disease or condition is Parkinson's disease and/or wherein the heterologous nucleic acid sequence encodes Brain-derived neurotrophic factor (BDNF) (e.g., a polypeptide represented by UniProt Accession number P23560 or a polypeptide comprising an amino acid sequence having at least 95% sequence identity thereto).
945. The method of any one of embodiments 222 to 224, wherein the disease or condition is Paroxysmal Choreoathetosis and/or wherein the heterologous nucleic acid sequence encodes PNKD (e.g., a polypeptide represented by UniProt Accession number Q8N490 or a polypeptide comprising an amino acid sequence having at least 95% sequence identity thereto).
946. The method of any one of embodiments 222 to 224, wherein the disease or condition is Pelizaeus-Merzbacher Disease and/or wherein the heterologous nucleic acid sequence encodes PLP1 (e.g., a polypeptide represented by UniProt Accession number P60201 or a polypeptide comprising an amino acid sequence having at least 95% sequence identity thereto).
947. The method of any one of embodiments 222 to 224, wherein the disease or condition is Pena-Shokeir Type II Syndrome and/or wherein the heterologous nucleic acid sequence encodes ERCC6 (e.g., a polypeptide represented by UniProt Accession number Q03468 or a polypeptide comprising an amino acid sequence having at least 95% sequence identity thereto).
948. The method of any one of embodiments 222 to 224, wherein the disease or condition is Periodic Paralyses and/or wherein the heterologous nucleic acid sequence encodes SCN4A (e.g., a polypeptide represented by UniProt Accession number P35499 or a polypeptide comprising an amino acid sequence having at least 95% sequence identity thereto).
949. The method of any one of embodiments 222 to 224, wherein the disease or condition is Phelan-McDermid syndrome and/or wherein the heterologous nucleic acid sequence encodes SH3 and multiple ankyrin repeat domains protein 3 (SHANK3) (e.g., a polypeptide represented by UniProt Accession number Q9BYBO or a polypeptide comprising an amino acid sequence having at least 95% sequence identity thereto).
950. The method of any one of embodiments 222 to 224, wherein the disease or condition is Phytanic Acid Storage Disease (peroxisome biogenesis disorder 1B) and/or wherein the heterologous nucleic acid sequence encodes PEX1 (e.g., a polypeptide represented by UniProt Accession number O43933 or a polypeptide comprising an amino acid sequence having at least 95% sequence identity thereto).
951. The method of any one of embodiments 222 to 224, wherein the disease or condition is Pick disease and/or wherein the heterologous nucleic acid sequence encodes PSEN1 (e.g., a polypeptide represented by UniProt Accession number A0A024R6A3 or a polypeptide comprising an amino acid sequence having at least 95% sequence identity thereto).
952. The method of any one of embodiments 222 to 224, wherein the disease or condition is Pick disease and/or wherein the heterologous nucleic acid sequence encodes MAPT (tau) (e.g., a polypeptide represented by UniProt Accession number P10636 or a polypeptide comprising an amino acid sequence having at least 95% sequence identity thereto).
953. The method of any one of embodiments 222 to 224, wherein the disease or condition is Porencephaly type 1 and/or wherein the heterologous nucleic acid sequence encodes COL4A1 (e.g., a polypeptide represented by UniProt Accession number P02462 or a polypeptide comprising an amino acid sequence having at least 95% sequence identity thereto).
954. The method of any one of embodiments 222 to 224, wherein the disease or condition is Primary Lateral Sclerosis, juvenile and/or wherein the heterologous nucleic acid sequence encodes ALS2 (e.g., a polypeptide represented by UniProt Accession number Q96Q42 or a polypeptide comprising an amino acid sequence having at least 95% sequence identity thereto).
955. The method of any one of embodiments 222 to 224, wherein the disease or condition is Primary Progressive Aphasia and/or wherein the heterologous nucleic acid sequence encodes GRN (e.g., a polypeptide represented by UniProt Accession number P28799 or a polypeptide comprising an amino acid sequence having at least 95% sequence identity thereto).
956. The method of any one of embodiments 222 to 224, wherein the disease or condition is Progressive external ophtalmoplegia and/or wherein the heterologous nucleic acid sequence encodes POLG (e.g., a polypeptide represented by UniProt Accession number P54098 or a polypeptide comprising an amino acid sequence having at least 95% sequence identity thereto).
957. The method of any one of embodiments 222 to 224, wherein the disease or condition is Progressive external ophtalmoplegia and/or wherein the heterologous nucleic acid sequence encodes POLG2 (e.g., a polypeptide represented by UniProt Accession number Q9UHN1 or a polypeptide comprising an amino acid sequence having at least 95% sequence identity thereto).
958. The method of any one of embodiments 222 to 224, wherein the disease or condition is Progressive external ophtalmoplegia and/or wherein the heterologous nucleic acid sequence encodes SLC25A4 (e.g., a polypeptide represented by UniProt Accession number P12235 or a polypeptide comprising an amino acid sequence having at least 95% sequence identity thereto).
959. The method of any one of embodiments 222 to 224, wherein the disease or condition is Progressive external ophtalmoplegia and/or wherein the heterologous nucleic acid sequence encodes TWNK (e.g., a polypeptide represented by UniProt Accession number Q96RR1 or a polypeptide comprising an amino acid sequence having at least 95% sequence identity thereto).
960. The method of any one of embodiments 222 to 224, wherein the disease or condition is Progressive bulbar palsy and/or wherein the heterologous nucleic acid sequence encodes SLC52A3 (e.g., a polypeptide represented by UniProt Accession number Q9NQ40 or a polypeptide comprising an amino acid sequence having at least 95% sequence identity thereto).
961. The method of any one of embodiments 222 to 224, wherein the disease or condition is Progressive supranuclear palsy and/or wherein the heterologous nucleic acid sequence encodes Microtubule-associated protein tau (MAPT), Tau (e.g., a polypeptide represented by UniProt Accession number P10636 or a polypeptide comprising an amino acid sequence having at least 95% sequence identity thereto).
962. The method of any one of embodiments 222 to 224, wherein the disease or condition is Pseudo-Torch syndrome and/or wherein the heterologous nucleic acid sequence encodes OCLN (e.g., a polypeptide represented by UniProt Accession number Q16625 or a polypeptide comprising an amino acid sequence having at least 95% sequence identity thereto).
963. The method of any one of embodiments 222 to 224, wherein the disease or condition is Pseudo-Torch syndrome and/or wherein the heterologous nucleic acid sequence encodes STAT2 (e.g., a polypeptide represented by UniProt Accession number P52630 or a polypeptide comprising an amino acid sequence having at least 95% sequence identity thereto).
964. The method of any one of embodiments 222 to 224, wherein the disease or condition is Pseudo-Torch syndrome and/or wherein the heterologous nucleic acid sequence encodes USP18 (e.g., a polypeptide represented by UniProt Accession number Q9UMW8 or a polypeptide comprising an amino acid sequence having at least 95% sequence identity thereto).
965. The method of any one of embodiments 222 to 224, wherein the disease or condition is Refsum Disease and/or wherein the heterologous nucleic acid sequence encodes PHYH (e.g., a polypeptide represented by UniProt Accession number 014832 or a polypeptide comprising an amino acid sequence having at least 95% sequence identity thereto).
966. The method of any one of embodiments 222 to 224, wherein the disease or condition is Retinitis Pigmentosa 38 (rod-cone dystrophy) and/or wherein the heterologous nucleic acid sequence encodes Tyrosine-protein kinase Mer (MERTK) (e.g., a polypeptide represented by UniProt Accession number Q12866 or a polypeptide comprising an amino acid sequence having at least 95% sequence identity thereto).
967. The method of any one of embodiments 222 to 224, wherein the disease or condition is Retinitis Pigmentosa 40 and/or wherein the heterologous nucleic acid sequence encodes PDE6B (e.g., a polypeptide represented by UniProt Accession number P35913 or a polypeptide comprising an amino acid sequence having at least 95% sequence identity thereto).
968. The method of any one of embodiments 222 to 224, wherein the disease or condition is Rett syndrome and/or wherein the heterologous nucleic acid sequence encodes Methyl-CpG-binding protein 2 (MECP2) (e.g., a polypeptide represented by UniProt Accession number P51608 or a polypeptide comprising an amino acid sequence having at least 95% sequence identity thereto).
969. The method of any one of embodiments 222 to 224, wherein the disease or condition is Sandhoff disease and/or wherein the heterologous nucleic acid sequence encodes Beta-hexosaminidase subunit alpha (HEXA) (e.g., a polypeptide represented by UniProt Accession number P06865 or a polypeptide comprising an amino acid sequence having at least 95% sequence identity thereto).
970. The method of any one of embodiments 222 to 224, wherein the disease or condition is Sandhoff disease and/or wherein the heterologous nucleic acid sequence encodes Beta-hexosaminidase subunit beta (HEXB) (e.g., a polypeptide represented by UniProt Accession number P07686 or a polypeptide comprising an amino acid sequence having at least 95% sequence identity thereto).
971. The method of any one of embodiments 222 to 224, wherein the disease or condition is Schizencephaly and/or wherein the heterologous nucleic acid sequence encodes SIX3 (e.g., a polypeptide represented by UniProt Accession number 095343 or a polypeptide comprising an amino acid sequence having at least 95% sequence identity thereto).
972. The method of any one of embodiments 222 to 224, wherein the disease or condition is Schizencephaly and/or wherein the heterologous nucleic acid sequence encodes EMX2 (e.g., a polypeptide represented by UniProt Accession number Q04743 or a polypeptide comprising an amino acid sequence having at least 95% sequence identity thereto).
973. The method of any one of embodiments 222 to 224, wherein the disease or condition is Schizencephaly and/or wherein the heterologous nucleic acid sequence encodes SHH (e.g., a polypeptide represented by UniProt Accession number Q15465 or a polypeptide comprising an amino acid sequence having at least 95% sequence identity thereto).
974. The method of any one of embodiments 222 to 224, wherein the disease or condition is Seitelberger Disease and/or wherein the heterologous nucleic acid sequence encodes PLA2G6 (e.g., a polypeptide represented by UniProt Accession number 060733 or a polypeptide comprising an amino acid sequence having at least 95% sequence identity thereto).
975. The method of any one of embodiments 222 to 224, wherein the disease or condition is Septo-optic dysplasia (De Morsier syndrome) and/or wherein the heterologous nucleic acid sequence encodes HESX1 (e.g., a polypeptide represented by UniProt Accession number Q9UBX0 or a polypeptide comprising an amino acid sequence having at least 95% sequence identity thereto).
976. The method of any one of embodiments 222 to 224, wherein the disease or condition is Snijders Blok-Fisher syndrome and/or wherein the heterologous nucleic acid sequence encodes POU3F3 (e.g., a polypeptide represented by UniProt Accession number P20264 or a polypeptide comprising an amino acid sequence having at least 95% sequence identity thereto).
977. The method of any one of embodiments 222 to 224, wherein the disease or condition is Spastic Paraplegias and/or wherein the heterologous nucleic acid sequence encodes SPG11 (e.g., a polypeptide represented by UniProt Accession number Q96J17 or a polypeptide comprising an amino acid sequence having at least 95% sequence identity thereto).
978. The method of any one of embodiments 222 to 224, wherein the disease or condition is Spastic Paraplegias and/or wherein the heterologous nucleic acid sequence encodes SPAST (e.g., a polypeptide represented by UniProt Accession number Q9UBP0 or a polypeptide comprising an amino acid sequence having at least 95% sequence identity thereto).
979. The method of any one of embodiments 222 to 224, wherein the disease or condition is Spastic Paraplegias and/or wherein the heterologous nucleic acid sequence encodes KIFSA (e.g., a polypeptide represented by UniProt Accession number Q12840 or a polypeptide comprising an amino acid sequence having at least 95% sequence identity thereto).
980. The method of any one of embodiments 222 to 224, wherein the disease or condition is Spastic Paraplegias and/or wherein the heterologous nucleic acid sequence encodes NIPA1 (e.g., a polypeptide represented by UniProt Accession number Q7RTP0 or a polypeptide comprising an amino acid sequence having at least 95% sequence identity thereto).
981. The method of any one of embodiments 222 to 224, wherein the disease or condition is Spastic Paraplegias and/or wherein the heterologous nucleic acid sequence encodes CYP7B1 (e.g., a polypeptide represented by UniProt Accession number 075881 or a polypeptide comprising an amino acid sequence having at least 95% sequence identity thereto).
982. The method of any one of embodiments 222 to 224, wherein the disease or condition is Spastic Paraplegias and/or wherein the heterologous nucleic acid sequence encodes ATL1 (e.g., a polypeptide represented by UniProt Accession number Q8WXF7 or a polypeptide comprising an amino acid sequence having at least 95% sequence identity thereto).
983. The method of any one of embodiments 222 to 224, wherein the disease or condition is Spinal Muscular Atrophy (Kugelberg-Welander Disease) and/or wherein the heterologous nucleic acid sequence encodes Survival motor neuron protein (SMN), SMN1 (e.g., a polypeptide represented by UniProt Accession number Q16637 or a polypeptide comprising an amino acid sequence having at least 95% sequence identity thereto).
984. The method of any one of embodiments 222 to 224, wherein the disease or condition is Spinocerebellar ataxia and/or wherein the heterologous nucleic acid sequence encodes Ataxin-1 (ATXN1), SCA1 (e.g., a polypeptide represented by UniProt Accession number P54253 or a polypeptide comprising an amino acid sequence having at least 95% sequence identity thereto).
985. The method of any one of embodiments 222 to 224, wherein the disease or condition is Spinocerebellar ataxia and/or wherein the heterologous nucleic acid sequence encodes Ataxin-2 (ATXN2), SCA2 (e.g., a polypeptide represented by UniProt Accession number Q99700 or a polypeptide comprising an amino acid sequence having at least 95% sequence identity thereto).
986. The method of any one of embodiments 222 to 224, wherein the disease or condition is Spinocerebellar ataxia and/or wherein the heterologous nucleic acid sequence encodes Ataxin-3 (ATXN3), SCA3 (e.g., a polypeptide represented by UniProt Accession number P54252 or a polypeptide comprising an amino acid sequence having at least 95% sequence identity thereto).
987. The method of any one of embodiments 222 to 224, wherein the disease or condition is Spinocerebellar ataxia and/or wherein the heterologous nucleic acid sequence encodes ZFHX3 (e.g., a polypeptide represented by UniProt Accession number Q15911 or a polypeptide comprising an amino acid sequence having at least 95% sequence identity thereto).
988. The method of any one of embodiments 222 to 224, wherein the disease or condition is Spinocerebellar ataxia and/or wherein the heterologous nucleic acid sequence encodes CACNA1A (e.g., a polypeptide represented by UniProt Accession number 000555 or a polypeptide comprising an amino acid sequence having at least 95% sequence identity thereto).
989. The method of any one of embodiments 222 to 224, wherein the disease or condition is Spinocerebellar ataxia and/or wherein the heterologous nucleic acid sequence encodes ATXN7, SCA7 (e.g., a polypeptide represented by UniProt Accession number 015265 or a polypeptide comprising an amino acid sequence having at least 95% sequence identity thereto).
990. The method of any one of embodiments 222 to 224, wherein the disease or condition is Spinocerebellar ataxia and/or wherein the heterologous nucleic acid sequence encodes TMEM240 (e.g., a polypeptide represented by UniProt Accession number Q5SV17 or a polypeptide comprising an amino acid sequence having at least 95% sequence identity thereto).
991. The method of any one of embodiments 222 to 224, wherein the disease or condition is Sporadic Inclusion Body Myositis and/or wherein the heterologous nucleic acid sequence encodes Follistatin (FST) (e.g., a polypeptide represented by UniProt Accession number P19883 or a polypeptide comprising an amino acid sequence having at least 95% sequence identity thereto).
992. The method of any one of embodiments 222 to 224, wherein the disease or condition is Steele-Richardson-Olszewski syndrome (Parkinson-dementia syndrome) and/or wherein the heterologous nucleic acid sequence encodes MAPT(Tau) (e.g., a polypeptide represented by UniProt Accession number P10636 or a polypeptide comprising an amino acid sequence having at least 95% sequence identity thereto).
993. The method of any one of embodiments 222 to 224, wherein the disease or condition is Stiff-Person Syndrome, congenital and/or wherein the heterologous nucleic acid sequence encodes GLRA1 (e.g., a polypeptide represented by UniProt Accession number P23415 or a polypeptide comprising an amino acid sequence having at least 95% sequence identity thereto).
994. The method of any one of embodiments 222 to 224, wherein the disease or condition is Stiff-Person Syndrome, congenital and/or wherein the heterologous nucleic acid sequence encodes GLRB (e.g., a polypeptide represented by UniProt Accession number P48167 or a polypeptide comprising an amino acid sequence having at least 95% sequence identity thereto).
995. The method of any one of embodiments 222 to 224, wherein the disease or condition is Striatonigral degeneration and/or wherein the heterologous nucleic acid sequence encodes NUP62 (e.g., a polypeptide represented by UniProt Accession number P37198 or a polypeptide comprising an amino acid sequence having at least 95% sequence identity thereto).
996. The method of any one of embodiments 222 to 224, wherein the disease or condition is Striatonigral degeneration and/or wherein the heterologous nucleic acid sequence encodes PDE8B (e.g., a polypeptide represented by UniProt Accession number 095263 or a polypeptide comprising an amino acid sequence having at least 95% sequence identity thereto).
997. The method of any one of embodiments 222 to 224, wherein the disease or condition is Striatonigral degeneration and/or wherein the heterologous nucleic acid sequence encodes MTATP6 (e.g., a polypeptide represented by UniProt Accession number P00846 or a polypeptide comprising an amino acid sequence having at least 95% sequence identity thereto).
998. The method of any one of embodiments 222 to 224, wherein the disease or condition is Striatonigral degeneration and/or wherein the heterologous nucleic acid sequence encodes VAC14 (e.g., a polypeptide represented by UniProt Accession number Q08AM6 or a polypeptide comprising an amino acid sequence having at least 95% sequence identity thereto).
999. The method of any one of embodiments 222 to 224, wherein the disease or condition is Sturge-Weber Syndrome and/or wherein the heterologous nucleic acid sequence encodes GNAQ (e.g., a polypeptide represented by UniProt Accession number P50148 or a polypeptide comprising an amino acid sequence having at least 95% sequence identity thereto).
1000. The method of any one of embodiments 222 to 224, wherein the disease or condition is Stroke and/or wherein the heterologous nucleic acid sequence encodes tPA (e.g., a polypeptide represented by UniProt Accession number P00750 or a polypeptide comprising an amino acid sequence having at least 95% sequence identity thereto).
1001. The method of any one of embodiments 222 to 224, wherein the disease or condition is Stroke and/or wherein the heterologous nucleic acid sequence encodes NeuroD1 (e.g., a polypeptide represented by UniProt Accession number Q13562 or a polypeptide comprising an amino acid sequence having at least 95% sequence identity thereto).
1002. The method of any one of embodiments 222 to 224, wherein the disease or condition is Subcortical Vascular Encephalopathy (Cerebral Arteriopathy) and/or wherein the heterologous nucleic acid sequence encodes HTRA1 (e.g., a polypeptide represented by UniProt Accession number Q92743 or a polypeptide comprising an amino acid sequence having at least 95% sequence identity thereto).
1003. The method of any one of embodiments 222 to 224, wherein the disease or condition is Systemic Lupus Erythematosus and/or wherein the heterologous nucleic acid sequence encodes DNASE1L3 (e.g., a polypeptide represented by UniProt Accession number Q13609 or a polypeptide comprising an amino acid sequence having at least 95% sequence identity thereto).
1004. The method of any one of embodiments 222 to 224, wherein the disease or condition is Systemic Lupus Erythematosus and/or wherein the heterologous nucleic acid sequence encodes TLR7 (e.g., a polypeptide represented by UniProt Accession number Q9NYK1 or a polypeptide comprising an amino acid sequence having at least 95% sequence identity thereto).
1005. The method of any one of embodiments 222 to 224, wherein the disease or condition is Tardive Dyskinesia and/or wherein the heterologous nucleic acid sequence encodes CYP2D6 (e.g., a polypeptide represented by UniProt Accession number P10635 or a polypeptide comprising an amino acid sequence having at least 95% sequence identity thereto).
1006. The method of any one of embodiments 222 to 224, wherein the disease or condition is Tay-Sachs disease and/or wherein the heterologous nucleic acid sequence encodes Beta-hexosaminidase subunit alpha (HEXA) (e.g., a polypeptide represented by UniProt Accession number P06865 or a polypeptide comprising an amino acid sequence having at least 95% sequence identity thereto).
1007. The method of any one of embodiments 222 to 224, wherein the disease or condition is Tourette Syndrome and/or wherein the heterologous nucleic acid sequence encodes HDC (e.g., a polypeptide represented by UniProt Accession number P19113 or a polypeptide comprising an amino acid sequence having at least 95% sequence identity thereto).
1008. The method of any one of embodiments 222 to 224, wherein the disease or condition is Tourette Syndrome and/or wherein the heterologous nucleic acid sequence encodes SLITRK1 (e.g., a polypeptide represented by UniProt Accession number Q96PX8 or a polypeptide comprising an amino acid sequence having at least 95% sequence identity thereto).
1009. The method of any one of embodiments 222 to 224, wherein the disease or condition is Tremor, hereditary type 1 and/or wherein the heterologous nucleic acid sequence encodes DRD3 (e.g., a polypeptide represented by UniProt Accession number P35462 or a polypeptide comprising an amino acid sequence having at least 95% sequence identity thereto).
1010. The method of any one of embodiments 222 to 224, wherein the disease or condition is Troyer Syndrome and/or wherein the heterologous nucleic acid sequence encodes SPART (e.g., a polypeptide represented by UniProt Accession number Q8NOX7 or a polypeptide comprising an amino acid sequence having at least 95% sequence identity thereto).
1011. The method of any one of embodiments 222 to 224, wherein the disease or condition is Tuberous Sclerosis and/or wherein the heterologous nucleic acid sequence encodes TSC1 (e.g., a polypeptide represented by UniProt Accession number Q92574 or a polypeptide comprising an amino acid sequence having at least 95% sequence identity thereto).
1012. The method of any one of embodiments 222 to 224, wherein the disease or condition is Tuberous Sclerosis and/or wherein the heterologous nucleic acid sequence encodes TSC2 (e.g., a polypeptide represented by UniProt Accession number P49815 or a polypeptide comprising an amino acid sequence having at least 95% sequence identity thereto).
1013. The method of any one of embodiments 222 to 224, wherein the disease or condition is Tuberous Sclerosis and/or wherein the heterologous nucleic acid sequence encodes IFNG (e.g., a polypeptide represented by UniProt Accession number P01579 or a polypeptide comprising an amino acid sequence having at least 95% sequence identity thereto).
1014. The method of any one of embodiments 222 to 224, wherein the disease or condition is Von Hippel-Lindau Disease and/or wherein the heterologous nucleic acid sequence encodes VHL (e.g., a polypeptide represented by UniProt Accession number P40337 or a polypeptide comprising an amino acid sequence having at least 95% sequence identity thereto).
1015. The method of any one of embodiments 222 to 224, wherein the disease or condition is Von Hippel-Lindau Disease and/or wherein the heterologous nucleic acid sequence encodes CCND1 (e.g., a polypeptide represented by UniProt Accession number P24385 or a polypeptide comprising an amino acid sequence having at least 95% sequence identity thereto).
1016. The method of any one of embodiments 222 to 224, wherein the disease or condition is Von Recklinghausen Disease and/or wherein the heterologous nucleic acid sequence encodes NF1 (e.g., a polypeptide represented by UniProt Accession number P21359 or a polypeptide comprising an amino acid sequence having at least 95% sequence identity thereto).
1017. The method of any one of embodiments 222 to 224, wherein the disease or condition is Werdnig-Hoffman Disease and/or wherein the heterologous nucleic acid sequence encodes SMN1 (e.g., a polypeptide represented by UniProt Accession number Q16637 or a polypeptide comprising an amino acid sequence having at least 95% sequence identity thereto).
1018. The method of any one of embodiments 222 to 224, wherein the disease or condition is West Syndrome, X-linked and/or wherein the heterologous nucleic acid sequence encodes ARX (e.g., a polypeptide represented by UniProt Accession number Q96QS3 or a polypeptide comprising an amino acid sequence having at least 95% sequence identity thereto).
1019. The method of any one of embodiments 222 to 224, wherein the disease or condition is Wilson disease and/or wherein the heterologous nucleic acid sequence encodes ATP7B (e.g., a polypeptide represented by UniProt Accession number P35670 or a polypeptide comprising an amino acid sequence having at least 95% sequence identity thereto).
1020. The method of any one of embodiments 222 to 224, wherein the disease or condition is Wolman's disease (acid lipase disease) and/or wherein the heterologous nucleic acid sequence encodes LIPA (e.g., a polypeptide represented by UniProt Accession number P38571 or a polypeptide comprising an amino acid sequence having at least 95% sequence identity thereto).
1021. The method of any one of embodiments 222 to 224, wherein the disease or condition is X-linked adrenoleukodystrophy and/or wherein the heterologous nucleic acid sequence encodes ATP-binding cassette sub-family D member 1 (ABCD1) (e.g., a polypeptide represented by UniProt Accession number P33897 or a polypeptide comprising an amino acid sequence having at least 95% sequence identity thereto).
1022. The method of any one of embodiments 222 to 224, wherein the disease or condition is X-linked Retinoschisis and/or wherein the heterologous nucleic acid sequence encodes Retinoschisin (RS1) (e.g., a polypeptide represented by UniProt Accession number 015537 or a polypeptide comprising an amino acid sequence having at least 95% sequence identity thereto).
1023. The method of any one of embodiments 222 to 224, wherein the disease or condition is X-Linked Retinitis Pigmentosa and/or wherein the heterologous nucleic acid sequence encodes X-linked retinitis pigmentosa GTPase regulator (RPGR) (e.g., a polypeptide represented by UniProt Accession number Q92834 or a polypeptide comprising an amino acid sequence having at least 95% sequence identity thereto).
1024. The method of any one of embodiments 222 to 224, wherein the disease or condition is X-Linked Spinal and Bulbar Muscular Atrophy and/or wherein the heterologous nucleic acid sequence encodes UBA1 (e.g., a polypeptide represented by UniProt Accession number P22314 or a polypeptide comprising an amino acid sequence having at least 95% sequence identity thereto).
1025. The method of any one of embodiments 222 to 224, wherein the disease or condition is Advanced heart failure and/or wherein the heterologous nucleic acid sequence encodes SERCA2a, ATP2A2 (e.g., a polypeptide represented by UniProt Accession number P16615 or a polypeptide comprising an amino acid sequence having at least 95% sequence identity thereto).
1026. The method of any one of embodiments 222 to 224, wherein the disease or condition is Amyotrophic lateral sclerosis (ALS) (Lou Gehrig's disease) and/or wherein the heterologous nucleic acid sequence encodes Superoxide dismutase-1 (SOD1) (e.g., a polypeptide represented by UniProt Accession number P00441 or a polypeptide comprising an amino acid sequence having at least 95% sequence identity thereto).
1027. The method of any one of embodiments 222 to 224, wherein the disease or condition is Andersen-Tawil Syndrome and/or wherein the heterologous nucleic acid sequence encodes KCNJ2 (e.g., a polypeptide represented by UniProt Accession number P63252 or a polypeptide comprising an amino acid sequence having at least 95% sequence identity thereto).
1028. The method of any one of embodiments 222 to 224, wherein the disease or condition is Barth syndrome and/or wherein the heterologous nucleic acid sequence encodes TAFAZZIN (e.g., a polypeptide represented by UniProt Accession number Q16635 or a polypeptide comprising an amino acid sequence having at least 95% sequence identity thereto).
1029. The method of any one of embodiments 222 to 224, wherein the disease or condition is Becker Muscular Dystrophy (BMD) and/or wherein the heterologous nucleic acid sequence encodes DMD (e.g., a polypeptide represented by UniProt Accession number P11532 or a polypeptide comprising an amino acid sequence having at least 95% sequence identity thereto).
1030. The method of any one of embodiments 222 to 224, wherein the disease or condition is Becker Myotonia Congenita and/or wherein the heterologous nucleic acid sequence encodes CLCN1 (e.g., a polypeptide represented by UniProt Accession number P35523 or a polypeptide comprising an amino acid sequence having at least 95% sequence identity thereto).
1031. The method of any one of embodiments 222 to 224, wherein the disease or condition is Bethlem Myopathy and/or wherein the heterologous nucleic acid sequence encodes COL6A3 (e.g., a polypeptide represented by UniProt Accession number P12111 or a polypeptide comprising an amino acid sequence having at least 95% sequence identity thereto).
1032. The method of any one of embodiments 222 to 224, wherein the disease or condition is Bethlem Myopathy and/or wherein the heterologous nucleic acid sequence encodes COL6A2 (e.g., a polypeptide represented by UniProt Accession number P12110 or a polypeptide comprising an amino acid sequence having at least 95% sequence identity thereto).
1033. The method of any one of embodiments 222 to 224, wherein the disease or condition is Bethlem Myopathy and/or wherein the heterologous nucleic acid sequence encodes COL6A1 (e.g., a polypeptide represented by UniProt Accession number P12109 or a polypeptide comprising an amino acid sequence having at least 95% sequence identity thereto).
1034. The method of any one of embodiments 222 to 224, wherein the disease or condition is Bulbospinal Muscular Atrophy and/or wherein the heterologous nucleic acid sequence encodes AR (e.g., a polypeptide represented by UniProt Accession number P10275 or a polypeptide comprising an amino acid sequence having at least 95% sequence identity thereto).
1035. The method of any one of embodiments 222 to 224, wherein the disease or condition is Carnitine Deficiency, systemic primary and/or wherein the heterologous nucleic acid sequence encodes SLC22A5 (e.g., a polypeptide represented by UniProt Accession number 076082 or a polypeptide comprising an amino acid sequence having at least 95% sequence identity thereto).
1036. The method of any one of embodiments 222 to 224, wherein the disease or condition is Carnitine Palmityl Transferase Deficiency, type 1 and/or wherein the heterologous nucleic acid sequence encodes CPT1A (e.g., a polypeptide represented by UniProt Accession number P50416 or a polypeptide comprising an amino acid sequence having at least 95% sequence identity thereto).
1037. The method of any one of embodiments 222 to 224, wherein the disease or condition is Carnitine Palmityl Transferase Deficiency, type 2 and/or wherein the heterologous nucleic acid sequence encodes CPT2 (e.g., a polypeptide represented by UniProt Accession number P23786 or a polypeptide comprising an amino acid sequence having at least 95% sequence identity thereto).
1038. The method of any one of embodiments 222 to 224, wherein the disease or condition is Catecholaminergic polymorphic ventricular tachycardia 2 (CPVT2) and/or wherein the heterologous nucleic acid sequence encodes Calsequestrin-2 (CASQ2) (e.g., a polypeptide represented by UniProt Accession number 014958 or a polypeptide comprising an amino acid sequence having at least 95% sequence identity thereto).
1039. The method of any one of embodiments 222 to 224, wherein the disease or condition is Central Core Disease (congenital myopathy, type 1A) and/or wherein the heterologous nucleic acid sequence encodes RYR1 (e.g., a polypeptide represented by UniProt Accession number P21817 or a polypeptide comprising an amino acid sequence having at least 95% sequence identity thereto).
1040. The method of any one of embodiments 222 to 224, wherein the disease or condition is Centronuclear Myopathy type 1 and/or wherein the heterologous nucleic acid sequence encodes MTMR14 (e.g., a polypeptide represented by UniProt Accession number Q8NCE2 or a polypeptide comprising an amino acid sequence having at least 95% sequence identity thereto).
1041. The method of any one of embodiments 222 to 224, wherein the disease or condition is Centronuclear Myopathy type 2 and/or wherein the heterologous nucleic acid sequence encodes DNM2 (e.g., a polypeptide represented by UniProt Accession number 014717 or a polypeptide comprising an amino acid sequence having at least 95% sequence identity thereto).
1042. The method of any one of embodiments 222 to 224, wherein the disease or condition is Charcot-Marie-Tooth disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
1043. The method of any one of embodiments 222 to 224, wherein the disease or condition is Charcot-Marie-Tooth disease and/or wherein the heterologous nucleic acid sequence encodes MPZ (e.g., a polypeptide represented by UniProt Accession number P25189 or a polypeptide comprising an amino acid sequence having at least 95% sequence identity thereto).
1044. The method of any one of embodiments 222 to 224, wherein the disease or condition is Charcot-Marie-Tooth disease and/or wherein the heterologous nucleic acid sequence encodes DNM2 (e.g., a polypeptide represented by UniProt Accession number P50570 or a polypeptide comprising an amino acid sequence having at least 95% sequence identity thereto).
1045. The method of any one of embodiments 222 to 224, wherein the disease or condition is Charcot-Marie-Tooth disease and/or wherein the heterologous nucleic acid sequence encodes MFN2 (e.g., a polypeptide represented by UniProt Accession number 095140 or a polypeptide comprising an amino acid sequence having at least 95% sequence identity thereto).
1046. The method of any one of embodiments 222 to 224, wherein the disease or condition is Charcot-Marie-Tooth disease and/or wherein the heterologous nucleic acid sequence encodes KIF1B (e.g., a polypeptide represented by UniProt Accession number 060333 or a polypeptide comprising an amino acid sequence having at least 95% sequence identity thereto).
1047. The method of any one of embodiments 222 to 224, wherein the disease or condition is Charcot-Marie-Tooth disease and/or wherein the heterologous nucleic acid sequence encodes SBF2 (e.g., a polypeptide represented by UniProt Accession number Q86WG5 or a polypeptide comprising an amino acid sequence having at least 95% sequence identity thereto).
1048. The method of any one of embodiments 222 to 224, wherein the disease or condition is Charcot-Marie-Tooth disease and/or wherein the heterologous nucleic acid sequence encodes PNKP (e.g., a polypeptide represented by UniProt Accession number Q96T60 or a polypeptide comprising an amino acid sequence having at least 95% sequence identity thereto).
1049. The method of any one of embodiments 222 to 224, wherein the disease or condition is Charcot-Marie-Tooth disease and/or wherein the heterologous nucleic acid sequence encodes GDAP1 (e.g., a polypeptide represented by UniProt Accession number Q8TB36 or a polypeptide comprising an amino acid sequence having at least 95% sequence identity thereto).
1050. The method of any one of embodiments 222 to 224, wherein the disease or condition is Charcot-Marie-Tooth disease and/or wherein the heterologous nucleic acid sequence encodes LMNA (e.g., a polypeptide represented by UniProt Accession number P02545 or a polypeptide comprising an amino acid sequence having at least 95% sequence identity thereto).
1051. The method of any one of embodiments 222 to 224, wherein the disease or condition is Charcot-Marie-Tooth disease and/or wherein the heterologous nucleic acid sequence encodes FGD4 (e.g., a polypeptide represented by UniProt Accession number Q96M96 or a polypeptide comprising an amino acid sequence having at least 95% sequence identity thereto).
1052. The method of any one of embodiments 222 to 224, wherein the disease or condition is Charcot-Marie-Tooth disease and/or wherein the heterologous nucleic acid sequence encodes MTMR2 (e.g., a polypeptide represented by UniProt Accession number Q13614 or a polypeptide comprising an amino acid sequence having at least 95% sequence identity thereto).
1053. The method of any one of embodiments 222 to 224, wherein the disease or condition is Congenital Muscular Dystrophy (Ullrich disease) and/or wherein the heterologous nucleic acid sequence encodes COL6A3 (e.g., a polypeptide represented by UniProt Accession number P12111 or a polypeptide comprising an amino acid sequence having at least 95% sequence identity thereto).
1054. The method of any one of embodiments 222 to 224, wherein the disease or condition is Congenital Muscular Dystrophy (Ullrich disease) and/or wherein the heterologous nucleic acid sequence encodes COL6A2 (e.g., a polypeptide represented by UniProt Accession number P12110 or a polypeptide comprising an amino acid sequence having at least 95% sequence identity thereto).
1055. The method of any one of embodiments 222 to 224, wherein the disease or condition is Congenital Muscular Dystrophy (Ullrich disease) and/or wherein the heterologous nucleic acid sequence encodes COL6A1 (e.g., a polypeptide represented by UniProt Accession number P12109 or a polypeptide comprising an amino acid sequence having at least 95% sequence identity thereto).
1056. The method of any one of embodiments 222 to 224, wherein the disease or condition is Congenital Myasthenic Syndrome and/or wherein the heterologous nucleic acid sequence encodes COLQ (e.g., a polypeptide represented by UniProt Accession number Q9Y215 or a polypeptide comprising an amino acid sequence having at least 95% sequence identity thereto).
1057. The method of any one of embodiments 222 to 224, wherein the disease or condition is Congenital Myasthenic Syndrome and/or wherein the heterologous nucleic acid sequence encodes AGRN (e.g., a polypeptide represented by UniProt Accession number 000468 or a polypeptide comprising an amino acid sequence having at least 95% sequence identity thereto).
1058. The method of any one of embodiments 222 to 224, wherein the disease or condition is Congenital Myasthenic Syndrome and/or wherein the heterologous nucleic acid sequence encodes RAPSN (e.g., a polypeptide represented by UniProt Accession number Q13702 or a polypeptide comprising an amino acid sequence having at least 95% sequence identity thereto).
1059. The method of any one of embodiments 222 to 224, wherein the disease or condition is Congenital Myasthenic Syndrome and/or wherein the heterologous nucleic acid sequence encodes GFPT1 (e.g., a polypeptide represented by UniProt Accession number Q06210 or a polypeptide comprising an amino acid sequence having at least 95% sequence identity thereto).
1060. The method of any one of embodiments 222 to 224, wherein the disease or condition is Congenital Myasthenic Syndrome and/or wherein the heterologous nucleic acid sequence encodes SCN4A (e.g., a polypeptide represented by UniProt Accession number P35499 or a polypeptide comprising an amino acid sequence having at least 95% sequence identity thereto).
1061. The method of any one of embodiments 222 to 224, wherein the disease or condition is Congenital Myasthenic Syndrome and/or wherein the heterologous nucleic acid sequence encodes ALG2 (e.g., a polypeptide represented by UniProt Accession number Q9H553 or a polypeptide comprising an amino acid sequence having at least 95% sequence identity thereto).
1062. The method of any one of embodiments 222 to 224, wherein the disease or condition is Congenital Myasthenic Syndrome and/or wherein the heterologous nucleic acid sequence encodes ALG14 (e.g., a polypeptide represented by UniProt Accession number Q96F25 or a polypeptide comprising an amino acid sequence having at least 95% sequence identity thereto).
1063. The method of any one of embodiments 222 to 224, wherein the disease or condition is Congenital Myasthenic Syndrome and/or wherein the heterologous nucleic acid sequence encodes DPAGT1 (e.g., a polypeptide represented by UniProt Accession number Q9H3H5 or a polypeptide comprising an amino acid sequence having at least 95% sequence identity thereto).
1064. The method of any one of embodiments 222 to 224, wherein the disease or condition is Congenital Myasthenic Syndrome and/or wherein the heterologous nucleic acid sequence encodes CHRNE (e.g., a polypeptide represented by UniProt Accession number Q04844 or a polypeptide comprising an amino acid sequence having at least 95% sequence identity thereto).
1065. The method of any one of embodiments 222 to 224, wherein the disease or condition is Congenital Myasthenic Syndrome and/or wherein the heterologous nucleic acid sequence encodes CHRNA1 (e.g., a polypeptide represented by UniProt Accession number P02708 or a polypeptide comprising an amino acid sequence having at least 95% sequence identity thereto).
1066. The method of any one of embodiments 222 to 224, wherein the disease or condition is Congenital Myasthenic Syndrome and/or wherein the heterologous nucleic acid sequence encodes DOK7 (e.g., a polypeptide represented by UniProt Accession number Q18PE1 or a polypeptide comprising an amino acid sequence having at least 95% sequence identity thereto).
1067. The method of any one of embodiments 222 to 224, wherein the disease or condition is Congenital Myasthenic Syndrome and/or wherein the heterologous nucleic acid sequence encodes CHAT (e.g., a polypeptide represented by UniProt Accession number P28329 or a polypeptide comprising an amino acid sequence having at least 95% sequence identity thereto).
1068. The method of any one of embodiments 222 to 224, wherein the disease or condition is Congenital Myopathy and/or wherein the heterologous nucleic acid sequence encodes ACTA1 (e.g., a polypeptide represented by UniProt Accession number P68133 or a polypeptide comprising an amino acid sequence having at least 95% sequence identity thereto).
1069. The method of any one of embodiments 222 to 224, wherein the disease or condition is Congenital Myopathy and/or wherein the heterologous nucleic acid sequence encodes STAC3 (e.g., a polypeptide represented by UniProt Accession number Q96MF2 or a polypeptide comprising an amino acid sequence having at least 95% sequence identity thereto).
1070. The method of any one of embodiments 222 to 224, wherein the disease or condition is Congenital Myopathy and/or wherein the heterologous nucleic acid sequence encodes TPM3 (e.g., a polypeptide represented by UniProt Accession number P06753 or a polypeptide comprising an amino acid sequence having at least 95% sequence identity thereto).
1071. The method of any one of embodiments 222 to 224, wherein the disease or condition is Congenital Myotonic Dystrophy and/or wherein the heterologous nucleic acid sequence encodes DMPK (e.g., a polypeptide represented by UniProt Accession number Q09013 or a polypeptide comprising an amino acid sequence having at least 95% sequence identity thereto).
1072. The method of any one of embodiments 222 to 224, wherein the disease or condition is Cori Disease (Debrancher Enzyme Deficiency or Forbes Disease) and/or wherein the heterologous nucleic acid sequence encodes AGL (e.g., a polypeptide represented by UniProt Accession number P35573 or a polypeptide comprising an amino acid sequence having at least 95% sequence identity thereto).
1073. The method of any one of embodiments 222 to 224, wherein the disease or condition is Danon disease and/or wherein the heterologous nucleic acid sequence encodes LAMP2 (e.g., a polypeptide represented by UniProt Accession number P13473 or a polypeptide comprising an amino acid sequence having at least 95% sequence identity thereto).
1074. The method of any one of embodiments 222 to 224, wherein the disease or condition is Dejerine-Sottas Disease and/or wherein the heterologous nucleic acid sequence encodes MPZ (e.g., a polypeptide represented by UniProt Accession number P25189 or a polypeptide comprising an amino acid sequence having at least 95% sequence identity thereto).
1075. The method of any one of embodiments 222 to 224, wherein the disease or condition is Dejerine-Sottas Disease and/or wherein the heterologous nucleic acid sequence encodes EGR2 (e.g., a polypeptide represented by UniProt Accession number P11161 or a polypeptide comprising an amino acid sequence having at least 95% sequence identity thereto).
1076. The method of any one of embodiments 222 to 224, wherein the disease or condition is Dejerine-Sottas Disease and/or wherein the heterologous nucleic acid sequence encodes PMP22 (e.g., a polypeptide represented by UniProt Accession number Q01453 or a polypeptide comprising an amino acid sequence having at least 95% sequence identity thereto).
1077. The method of any one of embodiments 222 to 224, wherein the disease or condition is Dejerine-Sottas Disease and/or wherein the heterologous nucleic acid sequence encodes PRX (e.g., a polypeptide represented by UniProt Accession number Q9BXMO or a polypeptide comprising an amino acid sequence having at least 95% sequence identity thereto).
1078. The method of any one of embodiments 222 to 224, wherein the disease or condition is Distal Muscular Dystrophy, Welander and/or wherein the heterologous nucleic acid sequence encodes TIA1 (e.g., a polypeptide represented by UniProt Accession number P31483 or a polypeptide comprising an amino acid sequence having at least 95% sequence identity thereto).
1079. The method of any one of embodiments 222 to 224, wherein the disease or condition is Distal Muscular Dystrophy, Miyoshi and/or wherein the heterologous nucleic acid sequence encodes DYSF (e.g., a polypeptide represented by UniProt Accession number 075923 or a polypeptide comprising an amino acid sequence having at least 95% sequence identity thereto).
1080. The method of any one of embodiments 222 to 224, wherein the disease or condition is Distal myopathy with anterior tibial onset and/or wherein the heterologous nucleic acid sequence encodes DYSF (e.g., a polypeptide represented by UniProt Accession number 075923 or a polypeptide comprising an amino acid sequence having at least 95% sequence identity thereto).
1081. The method of any one of embodiments 222 to 224, wherein the disease or condition is Duchenne Muscular Dystrophy and/or wherein the heterologous nucleic acid sequence encodes Dystrophin (DMD) (e.g., a polypeptide represented by UniProt Accession number P11532 or a polypeptide comprising an amino acid sequence having at least 95% sequence identity thereto).
1082. The method of any one of embodiments 222 to 224, wherein the disease or condition is Duchenne Muscular Dystrophy and/or wherein the heterologous nucleic acid sequence encodes GALGT2, B4GALNT2 (e.g., a polypeptide represented by UniProt Accession number Q8NHY0 or a polypeptide comprising an amino acid sequence having at least 95% sequence identity thereto).
1083. The method of any one of embodiments 222 to 224, wherein the disease or condition is Dysferlinopathy and/or wherein the heterologous nucleic acid sequence encodes Dysferlin (DYSF) (e.g., a polypeptide represented by UniProt Accession number 075923 or a polypeptide comprising an amino acid sequence having at least 95% sequence identity thereto).
1084. The method of any one of embodiments 222 to 224, wherein the disease or condition is Emery-Dreifuss Muscular Dystrophy and/or wherein the heterologous nucleic acid sequence encodes EMD (e.g., a polypeptide represented by UniProt Accession number P50402 or a polypeptide comprising an amino acid sequence having at least 95% sequence identity thereto).
1085. The method of any one of embodiments 222 to 224, wherein the disease or condition is Emery-Dreifuss Muscular Dystrophy and/or wherein the heterologous nucleic acid sequence encodes SYNE1 (e.g., a polypeptide represented by UniProt Accession number Q8NF91 or a polypeptide comprising an amino acid sequence having at least 95% sequence identity thereto).
1086. The method of any one of embodiments 222 to 224, wherein the disease or condition is Emery-Dreifuss Muscular Dystrophy and/or wherein the heterologous nucleic acid sequence encodes SYNE2 (e.g., a polypeptide represented by UniProt Accession number Q8WXHO or a polypeptide comprising an amino acid sequence having at least 95% sequence identity thereto).
1087. The method of any one of embodiments 222 to 224, wherein the disease or condition is Emery-Dreifuss Muscular Dystrophy and/or wherein the heterologous nucleic acid sequence encodes TMEM43 (e.g., a polypeptide represented by UniProt Accession number Q9BTV4 or a polypeptide comprising an amino acid sequence having at least 95% sequence identity thereto).
1088. The method of any one of embodiments 222 to 224, wherein the disease or condition is Emery-Dreifuss Muscular Dystrophy and/or wherein the heterologous nucleic acid sequence encodes LMNA (e.g., a polypeptide represented by UniProt Accession number P02545 or a polypeptide comprising an amino acid sequence having at least 95% sequence identity thereto).
1089. The method of any one of embodiments 222 to 224, wherein the disease or condition is Eulenberg Disease (Paramyotonia Congenita) and/or wherein the heterologous nucleic acid sequence encodes SCN4A (e.g., a polypeptide represented by UniProt Accession number P35499 or a polypeptide comprising an amino acid sequence having at least 95% sequence identity thereto).
1090. The method of any one of embodiments 222 to 224, wherein the disease or condition is Facioscapulohumeral Muscular Dystrophy and/or wherein the heterologous nucleic acid sequence encodes SMCHD1 (e.g., a polypeptide represented by UniProt Accession number A6NHR9 or a polypeptide comprising an amino acid sequence having at least 95% sequence identity thereto).
1091. The method of any one of embodiments 222 to 224, wherein the disease or condition is Facioscapulohumeral Muscular Dystrophy and/or wherein the heterologous nucleic acid sequence encodes LRIF1 (e.g., a polypeptide represented by UniProt Accession number Q5T3J3 or a polypeptide comprising an amino acid sequence having at least 95% sequence identity thereto).
1092. The method of any one of embodiments 222 to 224, wherein the disease or condition is Friedreich's ataxia and/or wherein the heterologous nucleic acid sequence encodes Frataxin (FXN) (e.g., a polypeptide represented by UniProt Accession number Q16595 or a polypeptide comprising an amino acid sequence having at least 95% sequence identity thereto).
1093. The method of any one of embodiments 222 to 224, wherein the disease or condition is Fukuyama Congenital Muscular Dystrophy and/or wherein the heterologous nucleic acid sequence encodes FKTN (e.g., a polypeptide represented by UniProt Accession number 075072 or a polypeptide comprising an amino acid sequence having at least 95% sequence identity thereto).
1094. The method of any one of embodiments 222 to 224, wherein the disease or condition is Glycogen storage disease II (Pompe disease or Acid Maltase Deficiency) and/or wherein the heterologous nucleic acid sequence encodes GAA (e.g., a polypeptide represented by UniProt Accession number P10253 or a polypeptide comprising an amino acid sequence having at least 95% sequence identity thereto).
1095. The method of any one of embodiments 222 to 224, wherein the disease or condition is Glycogenosis Type 10 (Glycogen storage disease X) and/or wherein the heterologous nucleic acid sequence encodes PGAM2 (e.g., a polypeptide represented by UniProt Accession number P15259 or a polypeptide comprising an amino acid sequence having at least 95% sequence identity thereto).
1096. The method of any one of embodiments 222 to 224, wherein the disease or condition is Glycogenosis Type 11 (Glycogen storage disease XI) and/or wherein the heterologous nucleic acid sequence encodes LDHA (e.g., a polypeptide represented by UniProt Accession number P00338 or a polypeptide comprising an amino acid sequence having at least 95% sequence identity thereto).
1097. The method of any one of embodiments 222 to 224, wherein the disease or condition is Glycogenosis Type 2 (Glycogen storage disease II or Pompe Disease or Acid maltase deficiency) and/or wherein the heterologous nucleic acid sequence encodes GAA (e.g., a polypeptide represented by UniProt Accession number P10253 or a polypeptide comprising an amino acid sequence having at least 95% sequence identity thereto).
1098. The method of any one of embodiments 222 to 224, wherein the disease or condition is Glycogenosis Type 3 (Glycogen storage disease Ill) and/or wherein the heterologous nucleic acid sequence encodes AGL (e.g., a polypeptide represented by UniProt Accession number P35573 or a polypeptide comprising an amino acid sequence having at least 95% sequence identity thereto).
1099. The method of any one of embodiments 222 to 224, wherein the disease or condition is Glycogenosis Type 5 (Glycogen storage disease V or McArdle Disease or Myophosphorylase Deficiency) and/or wherein the heterologous nucleic acid sequence encodes PYGM (e.g., a polypeptide represented by UniProt Accession number P11217 or a polypeptide comprising an amino acid sequence having at least 95% sequence identity thereto).
1100. The method of any one of embodiments 222 to 224, wherein the disease or condition is Glycogenosis Type 7 (Glycogen storage disease VII or Phosphofructokinase Deficiency or Tarui Disease) and/or wherein the heterologous nucleic acid sequence encodes PFKM (e.g., a polypeptide represented by UniProt Accession number P08237 or a polypeptide comprising an amino acid sequence having at least 95% sequence identity thereto).
1101. The method of any one of embodiments 222 to 224, wherein the disease or condition is Glycogenosis Type 9 (Glycogen storage disease IX) and/or wherein the heterologous nucleic acid sequence encodes PHKB (e.g., a polypeptide represented by UniProt Accession number Q93100 or a polypeptide comprising an amino acid sequence having at least 95% sequence identity thereto).
1102. The method of any one of embodiments 222 to 224, wherein the disease or condition is Glycogenosis Type 9 (Glycogen storage disease IX) and/or wherein the heterologous nucleic acid sequence encodes PHKA2 (e.g., a polypeptide represented by UniProt Accession number P46019 or a polypeptide comprising an amino acid sequence having at least 95% sequence identity thereto).
1103. The method of any one of embodiments 222 to 224, wherein the disease or condition is Hereditary Inclusion-Body Myositis and/or wherein the heterologous nucleic acid sequence encodes GNE (e.g., a polypeptide represented by UniProt Accession number Q9Y223 or a polypeptide comprising an amino acid sequence having at least 95% sequence identity thereto).
1104. The method of any one of embodiments 222 to 224, wherein the disease or condition is Integrin-Deficient Congenital Muscular Dystrophy and/or wherein the heterologous nucleic acid sequence encodes ITGA7 (e.g., a polypeptide represented by UniProt Accession number Q13683 or a polypeptide comprising an amino acid sequence having at least 95% sequence identity thereto).
1105. The method of any one of embodiments 222 to 224, wherein the disease or condition is Kennedy Disease (Spinal-Bulbar Muscular Atrophy) and/or wherein the heterologous nucleic acid sequence encodes Androgen receptor (AR) (e.g., a polypeptide represented by UniProt Accession number P10275 or a polypeptide comprising an amino acid sequence having at least 95% sequence identity thereto).
1106. The method of any one of embodiments 222 to 224, wherein the disease or condition is Kugelberg-Welander Disease and/or wherein the heterologous nucleic acid sequence encodes SMN1 (e.g., a polypeptide represented by UniProt Accession number Q16637 or a polypeptide comprising an amino acid sequence having at least 95% sequence identity thereto).
1107. The method of any one of embodiments 222 to 224, wherein the disease or condition is Lactate dehydrogenase A deficiency and/or wherein the heterologous nucleic acid sequence encodes LDHA (e.g., a polypeptide represented by UniProt Accession number P00338 or a polypeptide comprising an amino acid sequence having at least 95% sequence identity thereto).
1108. The method of any one of embodiments 222 to 224, wherein the disease or condition is Lactate Dehydrogenase B Deficiency and/or wherein the heterologous nucleic acid sequence encodes LDHB (e.g., a polypeptide represented by UniProt Accession number P07195 or a polypeptide comprising an amino acid sequence having at least 95% sequence identity thereto).
1109. The method of any one of embodiments 222 to 224, wherein the disease or condition is Lambert-Eaton Myasthenic Syndrome and/or wherein the heterologous nucleic acid sequence encodes CACNB2 (e.g., a polypeptide represented by UniProt Accession number Q08289 or a polypeptide comprising an amino acid sequence having at least 95% sequence identity thereto).
1110. The method of any one of embodiments 222 to 224, wherein the disease or condition is Laing Distal Myopathy and/or wherein the heterologous nucleic acid sequence encodes MYH7 (e.g., a polypeptide represented by UniProt Accession number A7E2Y1 or a polypeptide comprising an amino acid sequence having at least 95% sequence identity thereto).
1111. The method of any one of embodiments 222 to 224, wherein the disease or condition is Limb Girdle Muscular Dystrophy Type 2C (LGMD-2C) and/or wherein the heterologous nucleic acid sequence encodes Gamma-sarcoglycan (e.g., a polypeptide represented by UniProt Accession number Q13326 or a polypeptide comprising an amino acid sequence having at least 95% sequence identity thereto).
1112. The method of any one of embodiments 222 to 224, wherein the disease or condition is Limb Girdle Muscular Dystrophy Type 2D (LGMD-2D) and/or wherein the heterologous nucleic acid sequence encodes Alpha-sarcoglycan (e.g., a polypeptide represented by UniProt Accession number Q16586 or a polypeptide comprising an amino acid sequence having at least 95% sequence identity thereto).
1113. The method of any one of embodiments 222 to 224, wherein the disease or condition is Limb Girdle Muscular Dystrophy Type2E (LGMD-2E) and/or wherein the heterologous nucleic acid sequence encodes Beta-sarcoglycan (e.g., a polypeptide represented by UniProt Accession number Q16585 or a polypeptide comprising an amino acid sequence having at least 95% sequence identity thereto).
1114. The method of any one of embodiments 222 to 224, wherein the disease or condition is Limb Girdle Muscular Dystrophy Type 2F (LGMD-2F) and/or wherein the heterologous nucleic acid sequence encodes Delta-sarcoglycan (e.g., a polypeptide represented by UniProt Accession number Q92629 or a polypeptide comprising an amino acid sequence having at least 95% sequence identity thereto).
1115. The method of any one of embodiments 222 to 224, wherein the disease or condition is Merosin-Deficient Congenital Muscular Dystrophy and/or wherein the heterologous nucleic acid sequence encodes LAMA2 (e.g., a polypeptide represented by UniProt Accession number P24043 or a polypeptide comprising an amino acid sequence having at least 95% sequence identity thereto).
1116. The method of any one of embodiments 222 to 224, wherein the disease or condition is Muscle-Eye-Brain Disease and/or wherein the heterologous nucleic acid sequence encodes POMGNT1 (e.g., a polypeptide represented by UniProt Accession number Q8WZA1 or a polypeptide comprising an amino acid sequence having at least 95% sequence identity thereto).
1117. The method of any one of embodiments 222 to 224, wherein the disease or condition is Mitochondrial Myopathy and/or wherein the heterologous nucleic acid sequence encodes CHCHD10 (e.g., a polypeptide represented by UniProt Accession number Q8WYQ3 or a polypeptide comprising an amino acid sequence having at least 95% sequence identity thereto).
1118. The method of any one of embodiments 222 to 224, wherein the disease or condition is Miyoshi myopathy and/or wherein the heterologous nucleic acid sequence encodes DYSF (e.g., a polypeptide represented by UniProt Accession number 075923 or a polypeptide comprising an amino acid sequence having at least 95% sequence identity thereto).
1119. The method of any one of embodiments 222 to 224, wherein the disease or condition is Myoadenylate Deaminase Deficiency and/or wherein the heterologous nucleic acid sequence encodes AMPD1 (e.g., a polypeptide represented by UniProt Accession number P23109 or a polypeptide comprising an amino acid sequence having at least 95% sequence identity thereto).
1120. The method of any one of embodiments 222 to 224, wherein the disease or condition is Myofibrillar Myopathy 1 and/or wherein the heterologous nucleic acid sequence encodes DES (e.g., a polypeptide represented by UniProt Accession number P17661 or a polypeptide comprising an amino acid sequence having at least 95% sequence identity thereto).
1121. The method of any one of embodiments 222 to 224, wherein the disease or condition is Myofibrillar Myopathy 2 and/or wherein the heterologous nucleic acid sequence encodes CRYAB (e.g., a polypeptide represented by UniProt Accession number P02511 or a polypeptide comprising an amino acid sequence having at least 95% sequence identity thereto).
1122. The method of any one of embodiments 222 to 224, wherein the disease or condition is Myofibrillar Myopathy 3 and/or wherein the heterologous nucleic acid sequence encodes MYOT (e.g., a polypeptide represented by UniProt Accession number Q9UBF9 or a polypeptide comprising an amino acid sequence having at least 95% sequence identity thereto).
1123. The method of any one of embodiments 222 to 224, wherein the disease or condition is Myofibrillar Myopathy 4 (ZASP related myopathy) and/or wherein the heterologous nucleic acid sequence encodes LDB3, ZASP (e.g., a polypeptide represented by UniProt Accession number 075112 or a polypeptide comprising an amino acid sequence having at least 95% sequence identity thereto).
1124. The method of any one of embodiments 222 to 224, wherein the disease or condition is Myofibrillar Myopathy 5 and/or wherein the heterologous nucleic acid sequence encodes FLNC (e.g., a polypeptide represented by UniProt Accession number Q14315 or a polypeptide comprising an amino acid sequence having at least 95% sequence identity thereto).
1125. The method of any one of embodiments 222 to 224, wherein the disease or condition is Myofibrillar Myopathy 6 and/or wherein the heterologous nucleic acid sequence encodes BAG3 (e.g., a polypeptide represented by UniProt Accession number 095817 or a polypeptide comprising an amino acid sequence having at least 95% sequence identity thereto).
1126. The method of any one of embodiments 222 to 224, wherein the disease or condition is Myofibrillar Myopathy 7 and/or wherein the heterologous nucleic acid sequence encodes KY (e.g., a polypeptide represented by UniProt Accession number Q8NBH2 or a polypeptide comprising an amino acid sequence having at least 95% sequence identity thereto).
1127. The method of any one of embodiments 222 to 224, wherein the disease or condition is Myofibrillar Myopathy 8 and/or wherein the heterologous nucleic acid sequence encodes PYROXD1 (e.g., a polypeptide represented by UniProt Accession number Q8WU10 or a polypeptide comprising an amino acid sequence having at least 95% sequence identity thereto).
1128. The method of any one of embodiments 222 to 224, wherein the disease or condition is Myofibrillar Myopathy 9 and/or wherein the heterologous nucleic acid sequence encodes TTN (e.g., a polypeptide represented by UniProt Accession number Q8WZ42 or a polypeptide comprising an amino acid sequence having at least 95% sequence identity thereto).
1129. The method of any one of embodiments 222 to 224, wherein the disease or condition is Myofibrillar Myopathy 10 and/or wherein the heterologous nucleic acid sequence encodes SVIL (e.g., a polypeptide represented by UniProt Accession number 095425 or a polypeptide comprising an amino acid sequence having at least 95% sequence identity thereto).
1130. The method of any one of embodiments 222 to 224, wherein the disease or condition is Myofibrillar Myopathy 11 and/or wherein the heterologous nucleic acid sequence encodes UNC45B (e.g., a polypeptide represented by UniProt Accession number Q8IWX7 or a polypeptide comprising an amino acid sequence having at least 95% sequence identity thereto).
1131. The method of any one of embodiments 222 to 224, wherein the disease or condition is Myofibrillar Myopathy 12 and/or wherein the heterologous nucleic acid sequence encodes MYL2 (e.g., a polypeptide represented by UniProt Accession number Q99972 or a polypeptide comprising an amino acid sequence having at least 95% sequence identity thereto).
1132. The method of any one of embodiments 222 to 224, wherein the disease or condition is Myotonic dystrophy Type 1 (Steinert Disease) and/or wherein the heterologous nucleic acid sequence encodes Myotonin-protein kinase (DMPK) (e.g., a polypeptide represented by UniProt Accession number Q09013 or a polypeptide comprising an amino acid sequence having at least 95% sequence identity thereto).
1133. The method of any one of embodiments 222 to 224, wherein the disease or condition is Myotonic dystrophy Type 2 and/or wherein the heterologous nucleic acid sequence encodes CNBP (e.g., a polypeptide represented by UniProt Accession number P62633 or a polypeptide comprising an amino acid sequence having at least 95% sequence identity thereto).
1134. The method of any one of embodiments 222 to 224, wherein the disease or condition is Myotubular Myopathy and/or wherein the heterologous nucleic acid sequence encodes MTM1 (e.g., a polypeptide represented by UniProt Accession number Q13496 or a polypeptide comprising an amino acid sequence having at least 95% sequence identity thereto).
1135. The method of any one of embodiments 222 to 224, wherein the disease or condition is Nemaline Myopathy 1 and/or wherein the heterologous nucleic acid sequence encodes TPM3 (e.g., a polypeptide represented by UniProt Accession number P06753 or a polypeptide comprising an amino acid sequence having at least 95% sequence identity thereto).
1136. The method of any one of embodiments 222 to 224, wherein the disease or condition is Nemaline Myopathy 2 and/or wherein the heterologous nucleic acid sequence encodes NEB (e.g., a polypeptide represented by UniProt Accession number P20929 or a polypeptide comprising an amino acid sequence having at least 95% sequence identity thereto).
1137. The method of any one of embodiments 222 to 224, wherein the disease or condition is Nemaline Myopathy 5A, 5B, 5C and/or wherein the heterologous nucleic acid sequence encodes TNNT1 (e.g., a polypeptide represented by UniProt Accession number P13805 or a polypeptide comprising an amino acid sequence having at least 95% sequence identity thereto).
1138. The method of any one of embodiments 222 to 224, wherein the disease or condition is Nemaline Myopathy 3 and/or wherein the heterologous nucleic acid sequence encodes ACTA1 (e.g., a polypeptide represented by UniProt Accession number P68133 or a polypeptide comprising an amino acid sequence having at least 95% sequence identity thereto).
1139. The method of any one of embodiments 222 to 224, wherein the disease or condition is Nemaline Myopathy 6 and/or wherein the heterologous nucleic acid sequence encodes KBTBD13 (e.g., a polypeptide represented by UniProt Accession number C9JR72 or a polypeptide comprising an amino acid sequence having at least 95% sequence identity thereto).
1140. The method of any one of embodiments 222 to 224, wherein the disease or condition is Nemaline Myopathy 4 and/or wherein the heterologous nucleic acid sequence encodes TPM2 (e.g., a polypeptide represented by UniProt Accession number P07951 or a polypeptide comprising an amino acid sequence having at least 95% sequence identity thereto).
1141. The method of any one of embodiments 222 to 224, wherein the disease or condition is Nemaline Myopathy 7 and/or wherein the heterologous nucleic acid sequence encodes CFL2 (e.g., a polypeptide represented by UniProt Accession number Q9Y281 or a polypeptide comprising an amino acid sequence having at least 95% sequence identity thereto).
1142. The method of any one of embodiments 222 to 224, wherein the disease or condition is Nemaline Myopathy 8 and/or wherein the heterologous nucleic acid sequence encodes KLHL40 (e.g., a polypeptide represented by UniProt Accession number Q2TBAO or a polypeptide comprising an amino acid sequence having at least 95% sequence identity thereto).
1143. The method of any one of embodiments 222 to 224, wherein the disease or condition is Nemaline Myopathy 9 and/or wherein the heterologous nucleic acid sequence encodes KLHL41 (e.g., a polypeptide represented by UniProt Accession number 060662 or a polypeptide comprising an amino acid sequence having at least 95% sequence identity thereto).
1144. The method of any one of embodiments 222 to 224, wherein the disease or condition is Nemaline Myopathy 10 and/or wherein the heterologous nucleic acid sequence encodes LMOD3 (e.g., a polypeptide represented by UniProt Accession number Q0VAK6 or a polypeptide comprising an amino acid sequence having at least 95% sequence identity thereto).
1145. The method of any one of embodiments 222 to 224, wherein the disease or condition is Nonaka Distal Myopathy and/or wherein the heterologous nucleic acid sequence encodes GNE (e.g., a polypeptide represented by UniProt Accession number Q9Y223 or a polypeptide comprising an amino acid sequence having at least 95% sequence identity thereto).
1146. The method of any one of embodiments 222 to 224, wherein the disease or condition is Oculopharynggeal muscular dystrophy and/or wherein the heterologous nucleic acid sequence encodes PABPN1 (e.g., a polypeptide represented by UniProt Accession number Q86U42 or a polypeptide comprising an amino acid sequence having at least 95% sequence identity thereto).
1147. The method of any one of embodiments 222 to 224, wherein the disease or condition is Omithine Transcarbamylase deficiency and/or wherein the heterologous nucleic acid sequence encodes OTC (e.g., a polypeptide represented by UniProt Accession number P00480 or a polypeptide comprising an amino acid sequence having at least 95% sequence identity thereto).
1148. The method of any one of embodiments 222 to 224, wherein the disease or condition is Paramyotonia Congenita and/or wherein the heterologous nucleic acid sequence encodes SCN4A (e.g., a polypeptide represented by UniProt Accession number P35499 or a polypeptide comprising an amino acid sequence having at least 95% sequence identity thereto).
1149. The method of any one of embodiments 222 to 224, wherein the disease or condition is Periodic Paralysis, hypokalemic and/or wherein the heterologous nucleic acid sequence encodes CACNA1S (e.g., a polypeptide represented by UniProt Accession number Q13698 or a polypeptide comprising an amino acid sequence having at least 95% sequence identity thereto).
1150. The method of any one of embodiments 222 to 224, wherein the disease or condition is Periodic Paralysis, hyperkalemic and/or wherein the heterologous nucleic acid sequence encodes SCN4A (e.g., a polypeptide represented by UniProt Accession number P35499 or a polypeptide comprising an amino acid sequence having at least 95% sequence identity thereto).
1151. The method of any one of embodiments 222 to 224, wherein the disease or condition is Phosphoglycerate Kinase Deficiency and/or wherein the heterologous nucleic acid sequence encodes PGK1 (e.g., a polypeptide represented by UniProt Accession number P00558 or a polypeptide comprising an amino acid sequence having at least 95% sequence identity thereto).
1152. The method of any one of embodiments 222 to 224, wherein the disease or condition is Polymyositis and/or wherein the heterologous nucleic acid sequence encodes PMSCL2 (e.g., a polypeptide represented by UniProt Accession number Q01780 or a polypeptide comprising an amino acid sequence having at least 95% sequence identity thereto).
1153. The method of any one of embodiments 222 to 224, wherein the disease or condition is Polymyositis and/or wherein the heterologous nucleic acid sequence encodes PMSCL1 (e.g., a polypeptide represented by UniProt Accession number Q06265 or a polypeptide comprising an amino acid sequence having at least 95% sequence identity thereto).
1154. The method of any one of embodiments 222 to 224, wherein the disease or condition is Progressive External Ophthalmoplegia and/or wherein the heterologous nucleic acid sequence encodes POLG (e.g., a polypeptide represented by UniProt Accession number P54098 or a polypeptide comprising an amino acid sequence having at least 95% sequence identity thereto).
1155. The method of any one of embodiments 222 to 224, wherein the disease or condition is Progressive External Ophthalmoplegia and/or wherein the heterologous nucleic acid sequence encodes POLG2 (e.g., a polypeptide represented by UniProt Accession number Q9UHN1 or a polypeptide comprising an amino acid sequence having at least 95% sequence identity thereto).
1156. The method of any one of embodiments 222 to 224, wherein the disease or condition is Progressive External Ophthalmoplegia and/or wherein the heterologous nucleic acid sequence encodes SLC25A4 (e.g., a polypeptide represented by UniProt Accession number P12235 or a polypeptide comprising an amino acid sequence having at least 95% sequence identity thereto).
1157. The method of any one of embodiments 222 to 224, wherein the disease or condition is Progressive External Ophthalmoplegia and/or wherein the heterologous nucleic acid sequence encodes TWNK (e.g., a polypeptide represented by UniProt Accession number Q96RR1 or a polypeptide comprising an amino acid sequence having at least 95% sequence identity thereto).
1158. The method of any one of embodiments 222 to 224, wherein the disease or condition is Spinal Muscular Atrophy type 3—Kugelberg-Welander Disease and/or wherein the heterologous nucleic acid sequence encodes Survival motor neuron protein (SMN), SMN1 (e.g., a polypeptide represented by UniProt Accession number Q16637 or a polypeptide comprising an amino acid sequence having at least 95% sequence identity thereto).
1159. The method of any one of embodiments 222 to 224, wherein the disease or condition is Thomsen Disease (Myotonia Congenita (autosomal dominant)) and/or wherein the heterologous nucleic acid sequence encodes CLCN1 (e.g., a polypeptide represented by UniProt Accession number P35523 or a polypeptide comprising an amino acid sequence having at least 95% sequence identity thereto).
1160. The method of any one of embodiments 222 to 224, wherein the disease or condition is Walker-Warburg Syndrome and/or wherein the heterologous nucleic acid sequence encodes POMT1 (e.g., a polypeptide represented by UniProt Accession number Q9Y6A1 or a polypeptide comprising an amino acid sequence having at least 95% sequence identity thereto).
1161. The method of any one of embodiments 222 to 224, wherein the disease or condition is X-linked myotubular myopathy and/or wherein the heterologous nucleic acid sequence encodes Myotubularin (MTM1) (e.g., a polypeptide represented by UniProt Accession number Q13496 or a polypeptide comprising an amino acid sequence having at least 95% sequence identity thereto).
1162. The method of any one of embodiments 222 to 224, wherein the disease or condition is Werdnig-Hoffmann Disease—Spinal Muscular Atrophy type 1 and/or wherein the heterologous nucleic acid sequence encodes SMN1 (e.g., a polypeptide represented by UniProt Accession number Q16637 or a polypeptide comprising an amino acid sequence having at least 95% sequence identity thereto).
1163. The method of any one of embodiments 222 to 224, wherein the disease or condition is Amyotrophic lateral sclerosis and/or wherein the heterologous nucleic acid sequence encodes an antisense oligonucleotide targeting Superoxide dismutase 1 (SOD 1) (e.g., targeting an RNA encoding a polypeptide represented by UniProt Accession number P00441 or a polypeptide comprising an amino acid sequence having at least 95% sequence identity thereto).
1164. The method of any one of embodiments 222 to 224, wherein the disease or condition is Amyotrophic lateral sclerosis and/or wherein the heterologous nucleic acid sequence encodes an antisense oligonucleotide targeting C9orf72 (e.g., targeting an RNA encoding a polypeptide represented by UniProt Accession number Q96LT7 or a polypeptide comprising an amino acid sequence having at least 95% sequence identity thereto).
1165. The method of any one of embodiments 222 to 224, wherein the disease or condition is Huntington's disease and/or wherein the heterologous nucleic acid sequence encodes an antisense oligonucleotide targeting Huntingtin (HTT) (e.g., targeting an RNA encoding a polypeptide represented by UniProt Accession number P42858 or a polypeptide comprising an amino acid sequence having at least 95% sequence identity thereto, for example tominersen, WVE-120101, or WVE-120102).
1166. The method of any one of embodiments 222 to 224, wherein the disease or condition is Alzheimer's disease and/or wherein the heterologous nucleic acid sequence encodes an antisense oligonucleotide targeting Sortilin-related receptor (SORL1) (e.g., targeting an RNA encoding a polypeptide represented by UniProt Accession number Q92673 or a polypeptide comprising an amino acid sequence having at least 95% sequence identity thereto).
1167. The method of any one of embodiments 222 to 224, wherein the disease or condition is Alzheimer's disease and/or wherein the heterologous nucleic acid sequence encodes an antisense oligonucleotide targeting Neutral sphingomyelinase (N-SMase; SMPD2) (e.g., targeting an RNA encoding a polypeptide represented by UniProt Accession number 060906 or a polypeptide comprising an amino acid sequence having at least 95% sequence identity thereto).
1168. The method of any one of embodiments 222 to 224, wherein the disease or condition is Spinocerebellar ataxia type 2 and/or wherein the heterologous nucleic acid sequence encodes an antisense oligonucleotide targeting Ataxin-2 (ATXN2) (e.g., targeting an RNA encoding a polypeptide represented by UniProt Accession number Q99700 or a polypeptide comprising an amino acid sequence having at least 95% sequence identity thereto).
1169. The method of any one of embodiments 222 to 224, wherein the disease or condition is Parkinson's Disease and/or wherein the heterologous nucleic acid sequence encodes an antisense oligonucleotide targeting human SNCA (e.g., targeting an RNA encoding a polypeptide represented by UniProt Accession number P37840 or a polypeptide comprising an amino acid sequence having at least 95% sequence identity thereto).
1170. The method of any one of embodiments 222 to 224, wherein the disease or condition is a disease or condition set forth in Table 2.
1171. The method of any one of embodiments 222 to 1170, wherein the subject is a mammal, e.g., a human.
1172. A cell, cell-free system, or other translation system, comprising the capsid polypeptide of any one of embodiments 1 to 173, the nucleic acid molecule of any one of embodiments 190 to 192, or virus particle of any one of embodiments 193 to 221.
1173. A method of making a virus (e.g., a dependoparvovirus particle such as an adeno-associated virus (AAV) particle), comprising:
   (a) providing a cell, cell-free system, or other translation system, comprising the nucleic acid of any one of embodiments 190 to 192; and
   (b) cultivating the cell, cell-free system, or other translation system, under conditions suitable for the production of the virus particle,
   (c) thereby making the virus particle.
1174. The method of embodiment 1173, wherein the cell, cell-free system, or other translation system comprises a second nucleic acid molecule and at least a portion of said second nucleic acid molecule is packaged in the dependoparvovirus particle.
1175. The method of embodiment 1174, wherein the second nucleic acid comprises a payload, e.g., a heterologous nucleic acid sequence encoding a therapeutic product, e.g., as described herein.
1176. The method of embodiment 1175, wherein the therapeutic product is a therapeutic gene product forth in Table 2.
1177. The method of embodiment 1175, wherein the therapeutic product is a human GBA1 gene or portion thereof.
1178. The method of embodiment 1175, wherein the therapeutic product is an antisense oligonucleotide as set forth in Table 3.
1179. The method of embodiment 1175, wherein the therapeutic product is an antisense oligonucleotide complementary to a portion of human alpha-synuclein (SNCA) RNA.
1180. The method of any one of embodiments 1173 to 1179, wherein the nucleic acid molecule of any one of embodiments 190 to 192 mediates the production of a virus particle which does not include said nucleic acid of any one of embodiments 157 to 159 or fragment thereof.
1181. The method of any one of embodiments 1173 to 1180, wherein the nucleic acid molecule of any one of embodiments 190 to 192 mediates the production of a virus particle at a level similar, or at least 10% greater than the production level mediated by a nucleic acid comprising SEQ ID NO:2 in an otherwise similar production system.
1182. A composition, e.g., a pharmaceutical composition, comprising a virus particle of any one of embodiments 193 to 221 or a virus particle produced by the method of any one of embodiments 1173 to 1181, and a pharmaceutically acceptable carrier or excipient.
1183. The capsid polypeptide of any one of embodiments 1 to 173, the nucleic acid molecule of any one of embodiments 190 to 192, the virus particle of any one of embodiments 193 to 221, or the composition of embodiment 1182, for use in treating a disease or condition in a subject, optionally wherein (a) the subject is as defined in embodiment 1170 and/or (b) the disease or condition is as defined in embodiment 223 or 224.
1184. The capsid polypeptide of any one of embodiments 1 to 173, the nucleic acid molecule of any one of embodiments 190 to 192, the virus particle of any one of embodiments 193 to 221, or the composition of embodiment 1182, for use in the manufacture of a medicament for use in treating a disease or condition in a subject, optionally wherein (a) the subject is as defined in embodiment 1170 and/or (b) the disease or condition is as defined in any one of embodiments 223 to 1170.

### 8. EXAMPLES

The invention is further illustrated by the following examples. The examples are provided for illustrative purposes only and are not to be construed as limiting the scope or content of the invention in any way.

Unless otherwise noted, biodistribution, transduction and/or production are presented as fold-improvement over the rates exhibited by a virus particle comprising capsid polypeptides of SEQ ID NO:1. In some cases, rates are presented in log₂ notation.

### 8.1. Example 1: High Throughput AAV9 Library Evaluation in the CNS

### 8.1.1. Materials & Methods

### 8.1.1.1. Library Creation

Using machine learning algorithms trained on data from hundreds of thousands of capsid variants across multiple serotypes, including AAV particle production efficiency, and transduction and biodistribution across multiple tissues, a library of 1E5 capsid variants of wild-type AAV9 was designed with the goals of producing a capsid that would package into AAV particles, transduce central nervous system tissues after intravenous injection with high efficiency, and detarget the liver and other tissue types. The designed capsid polypeptides were cloned into plasmids to create a library of plasmids encoding the capsid variants. A library ofAAV variant genomes encoding each variant's capsid and a unique capsid variant barcode identifier was cloned into two ITR plasmid backbones as described previously (Ogden et al. Science, Nov. 19 2019, 366, (6469):1139-1143; doi: 10.1126/science.aaw2900, which is hereby incorporated by reference in its entirety), one with expression of the capsid gene under the control of a human synapsin 1 promoter (hSYN) and the other with expression of the capsid gene under the control of a CBh promoter. While hSYN drives expression predominantly in neurons, CBh is a ubiquitous promoter that drives expression in CNS and other tissues. Each plasmid backbone contained a unique genomic identifier (“backbone tag”) enabling analysis of biodistribution and transduction efficiencies via each of the two promoters. Each capsid polypeptide variant was included in combination with between 1-500 unique genomic identifiers (“barcodes”) to enable measurement of biological replicates for each virus comprising a unique capsid polypeptide. Each unique barcode was used in combination with a random genomic identifier (“ID tag”) to aid in quantification and to remove polypeptide sequences that contain unintended mutations. A library of AAV capsid variants, each comprising a genome encoding that variant's capsid polypeptides, was produced via transient triple transfection of adherent HEK293T. Transfections were completed at a 1:1:1/150 ratio of Helper, Rep, and Cap-inside-ITR plasmid. which has been optimized with these plasmids to limit cross packaging. Cells were harvested, lysed, and purified by a sequence of steps: (1) clarification via depth and sterilizing filtration, (2) ultrafiltration and diafiltration via tangential flow filtration (TFF), (3) iodixanol gradient purification, and (4) ultrafiltration and diafiltration via TFF. The produced virus was tested for suitable sterility and endotoxin, and titer performed by ddPCR.

### 8.1.1.2. In Vitro Evaluation of Library Packing Efficiency

Data was prepared as described below. To measure each variant's packaging efficiency (or “production”), barcodes from vector genomes in the plasmid library and produced AAV library were prepared for Illumina sequencing using two rounds of PCR. Production efficiency for each of the produced AAV particle variants was normalized for its abundance in the input plasmid library, and was expressed by comparing barcode sequencing levels for each variant in the produced AAV particle vector pool to the barcode sequence levels for each variant in the input plasmid library used to create the AAV vector pool. The measurements of variant frequency in the AAV vector library also enable downstream normalization of biodistribution and transduction measurements by variant frequency in the input vector library.

### 8.1.1.3. In Vivo Evaluation of Library Packing Efficiency

All NHP experiments were conducted in accordance with institutional policies and NIH guidelines. Two Cynomolgus Macaque primates (one female weighing 2.1 kg and one male weighing 2.6 kg) seronegative for anti-AAV9 neutralizing antibodies (serum NAb titers <1:4 based on in vitro NAb assay) were selected for the study. Animals were treated with Triamcinolone (3 mg/kg IM) on Day −8 and Day 1. Prior to test article administration, samples of blood were collected. The animals received an intravenous injection of a mixture of the promoter vector libraries (7.53e13 vg/kg total combined dose for male and 8.24e13 vg/kg for female). During the in-life period the animals were monitored according to the animal facility's SOPs. Serum samples were collected at 1 hour, 12 hours, 1 day, 1 days, 4 days, and weekly after the injections. Due to increased liver enzymes, the two animals received additional Triamcinolone on day 6 (3 mg/kg), and liver enzyme levels reduced to baseline levels over the course of the following 3 weeks. The animals were sacrificed 4 weeks after the injections and tissues were collected for biodistribution and transduction analyses. The tissues collected are shown in Table 2A. All samples were collected into RNAlater® (Sigma-Aldrich) and incubated overnight at 4° C., after which the RNAlater® was drained and samples were frozen at −80° C.

TABLE 2A
List of Tissues Collected and Number of Samples Used
for Biodistribution and Transduction Analysis

| | | # samples for | # samples used for |
|---|---|---|---|
| | | transduction | biodistribution |
| Organ | Tissue | analysis | analysis |
| brain | basal ganglia | 20 | 20 |
| | brainstem | 16 | 12 |
| | cerebellum | 17 | 17 |
| | forebrain | 17 | 17 |
| | hippocampus | 8 | 8 |
| | midbrain | 8 | 8 |
| | parietal cortex | 10 | 10 |
| | substantia nigra | 3 | 3 |
| | temporal cortex | 16 | 16 |
| | thalamus | 10 | 10 |
| | aggregated | 125 | 121 |
| cardiac muscle | | 20 | 18 |
| dorsal root ganglion | | 22 | 22 |
| kidney | | 6 | 6 |
| liver | | 16 | 16 |
| spinal cord | | 29 | 24 |
| skeletal muscle | gastrocnemius | 14 | 10 |
| | quadricep | 13 | 10 |
| | aggregated | 27 | 20 |

| | | |
|---|---|---|
| spleen | 12 | 10 |

Brain slices were dissected to isolate regions including, but not limited to, temporal cortex, motor cortex, hippocampus, basal ganglia, midbrain, brainstem, and cerebellum. For all biodistribution and transduction analyses, total DNA and RNA was extracted from tissue samples with Trizol/chloroform followed by the use of Qiagen DNeasy and RNAeasy kits respectively (DNeasy 96 Blood & Tissue Kit and RNeasy 96 Kit). Reverse transcription was done with Protoscript II Reverse Transcriptase (NEB) utilizing primers that were specific to the vector transgene and included unique molecular identifiers (UMls). Control reactions lacking the reverse transcriptase enzyme (-RT control) were also prepared. Quantification of biodistribution (based on viral DNA quantification) and transduction (based on viral transcript RNA quantification) was done with Luna Universal Probe qPCR Master Mix (NEB) using primers and probes specific to the transgene construct. Finally, samples were prepared for next-generation sequencing by amplifying the transgene barcode regions with primers compatible with Illumina NGS platform and sequenced on a NextSeq2000 (Illumina).

After sequencing, the barcode tags were extracted from reads with the expected amplicon structure, and the abundance (number of reads, number of UMIs, or number of unique combinations of barcode and ID tags) of each barcode was recorded. Analyses were restricted to the set of barcodes that were present in the input plasmid sample, as measured by a separate sequencing assay that targeted the variant regions of the input plasmid sample.

To aggregate packaging replicates, the read counts from replicate virus production samples were summed. To aggregate biodistribution samples, read counts from samples from the same tissue or organ were summed. To aggregate transduction samples, the number of transduction events (measured by the number of unique barcode and ID tag combinations detected) from samples from the same tissue or organ were summed.

Virus packaging was calculated by normalizing aggregated production replicates to input plasmid abundance. Biodistribution and transduction of tissue were calculated by normalizing aggregated biodistribution or transduction samples to input virus abundance. The output was reported as fold change relative to the WT AAV9 (amino acid sequence—SEQ ID NO:1; nucleotide sequence—SEQ ID NO:2) or “VAR-1” as disclosed in WO2023060264A1 (amino acid sequence—SEQ ID NO:14; nucleotide sequence—SEQ ID NO:15), as indicated.

### 8.1.2. Results

Property measurements for V1 (amino acid sequence—SEQ ID NO:12; nucleotide sequence—SEQ ID NO:13), averaged across all tissue pieces for the indicated tissue type and across both NHPs are reported relative to WT AAV9 in Tables 3A and 3B and relative to “VAR-1” as disclosed in WO2023060264A1 in Tables 4A-4B. V1 showed a more than 20-fold increase in transduction and biodistribution across multiple brain regions relative to wild-type AAV9, including over 300-fold increase in certain brain regions (e.g., basal ganglia, forebrain, and hippocampus). V1 also showed a more than 5-fold increase in transduction and biodistribution across multiple brain regions relative to “VAR-1” as disclosed in WO2023060264A1. In addition, measurements of biodistribution and transduction for V1 were well-correlated across the two primates.

TABLE 3A
Fold change in biodistribution of V1 virus particles, relative
to the biodistribution of virus particles comprising WT AAV9
(amino acid sequence - SEQ ID NO: 1; nucleotide sequence - SEQ
ID NO: 2) for the indicated tissues, with the standard deviation
shown in parentheses following each value. “CBh” indicates
measurements from virus particles comprising genomes with the
cap gene under the control of the CBh promoter. “hSyn”
indicates virus particles comprising genomes with the cap gene
under the control of the hSyn promoter. “Aggregated”
in relation to a tissue is an average across all measured areas
in the specified organ. “Aggregated” in relation to a
promoter is an average across the CBh and hSyn promoters.

| | | | Fold Change from | |
|---|---|---|---|---|
| Organ | Tissue | Promoter | WT AAV9 | |
| brain | aggregated | aggregated | 35.3 | (1.06) |
| brain | aggregated | hsyn | 37.12 | (1.05) |
| brain | basal ganglia | aggregated | 20.56 | (1.1) |
| brain | basal ganglia | hsyn | 21.95 | (1.12) |
| brain | brainstem | aggregated | 34.52 | (1.08) |
| brain | brainstem | hsyn | 35.36 | (1.11) |
| brain | cerebellum | aggregated | 57.89 | (1.11) |
| brain | cerebellum | hsyn | 60.88 | (1.1) |
| brain | forebrain | aggregated | 30.8 | (1.1) |
| brain | forebrain | hsyn | 33.84 | (1.12) |
| brain | hippocampus | aggregated | 24.01 | (1.18) |
| brain | hippocampus | hsyn | 23.76 | (1.15) |
| brain | midbrain | aggregated | 44.18 | (1.12) |
| brain | midbrain | hsyn | 46.14 | (1.15) |
| brain | parietal cortex | aggregated | 51.14 | (1.09) |
| brain | parietal cortex | hsyn | 50.15 | (1.12) |
| brain | substantia nigra | aggregated | 82.94 | (1.43) |
| brain | substantia nigra | hsyn | 81.2 | (1.35) |
| brain | temporal cortex | aggregated | 32.56 | (1.17) |
| brain | temporal cortex | hsyn | 37.06 | (1.16) |
| brain | thalamus | aggregated | 39.6 | (1.09) |
| brain | thalamus | hsyn | 40.45 | (1.13) |
| dorsal root | N/A | aggregated | 2.45 | (1.15) |
| ganglion | | | | |
| dorsal root | N/A | hsyn | 2.43 | (1.23) |
| ganglion | | | | |
| kidney | N/A | aggregated | 4.74 | (1.16) |
| kidney | N/A | hsyn | 4.8 | (1.16) |
| liver | N/A | aggregated | 0.03 | (1.31) |
| liver | N/A | hsyn | 0.03 | (1.36) |
| spinal cord | N/A | aggregated | 23.16 | (1.26) |
| spinal cord | N/A | hsyn | 21.11 | (1.25) |
| spleen | N/A | aggregated | 0.52 | (1.3) |
| spleen | N/A | hsyn | 0.56 | (1.2) |

TABLE 3B
Fold change in transduction of V1 virus particles, relative to
the transduction of virus particles comprising WT AAV9 (amino
acid sequence - SEQ ID NO: 1; nucleotide sequence - SEQ ID NO:
2) for the indicated tissues, with the standard deviation shown
in parentheses following each value. “hSyn” indicates
virus particles comprising genomes with the cap gene under the
control of the hSyn promoter. “Aggregated” in relation
to a tissue is an average across all measured areas in the specified
organ. “Aggregated” in relation to a promoter is an
average across the CBh and hSyn promoters.

| | | | Fold Change from | |
|---|---|---|---|---|
| Organ | Tissue | Promoter | WT AAV9 | |
| brain | aggregated | aggregated | 227.85 | (1.07) |
| brain | aggregated | hsyn | 231.61 | (1.08) |
| brain | basal ganglia | aggregated | 326.58 | (1.13) |
| brain | basal ganglia | hsyn | 310.36 | (1.11) |
| brain | brainstem | aggregated | 168.8 | (1.14) |
| brain | brainstem | hsyn | 183.45 | (1.13) |
| brain | cerebellum | aggregated | 180.1 | (1.19) |
| brain | cerebellum | hsyn | 149.41 | (1.23) |
| brain | forebrain | aggregated | 336.93 | (1.1) |
| brain | forebrain | hsyn | 322.92 | (1.14) |
| brain | hippocampus | aggregated | 468.25 | (1.13) |
| brain | hippocampus | hsyn | 537.33 | (1.21) |
| brain | midbrain | aggregated | 177.19 | (1.23) |
| brain | midbrain | hsyn | 179.33 | (1.2) |
| brain | parietal cortex | aggregated | 321.02 | (1.13) |
| brain | parietal cortex | hsyn | 350.12 | (1.14) |
| brain | substantia nigra | aggregated | 241.01 | (1.14) |
| brain | substantia nigra | hsyn | 217.79 | (1.17) |
| brain | temporal cortex | aggregated | 348.2 | (1.2) |
| brain | temporal cortex | hsyn | 387.19 | (1.19) |
| brain | thalamus | aggregated | 167.52 | (1.09) |
| brain | thalamus | hsyn | 168.69 | (1.09) |
| liver | N/A | aggregated | 0.03 | (1.53) |
| liver | N/A | hsyn | 0 | (1.41) |
| spinal cord | N/A | aggregated | 150.23 | (1.13) |
| spinal cord | N/A | hsyn | 178.46 | (1.15) |

TABLE 4A
Fold change in biodistribution of V1 virus particles, relative
to the biodistribution of virus particles comprising “VAR-
1” as disclosed in WO2023060264A1 (amino acid sequence -
SEQ ID NO: 14; nucleotide sequence - SEQ ID NO: 15) for the
indicated tissues, with the standard deviation shown in parentheses
following each value. “CBh” indicates measurements from
virus particles comprising genomes with the cap gene under the
control of the CBh promoter. “hSyn” indicates virus
particles comprising genomes with the cap gene under the control
of the hSyn promoter. “Aggregated” in relation to a
tissue is an average across all measured areas in the specified
organ. “Aggregated” in relation to a promoter is an
average across the CBh and hSyn promoters.

| Organ | Tissue | Promoter | Fold Change from “VAR-1” | |
|---|---|---|---|---|
| brain | aggregated | cbh | 4.94 | (1.08) |
| brain | aggregated | aggregated | 5.03 | (1.04) |
| brain | aggregated | hsyn | 5.03 | (1.04) |
| brain | basal ganglia | cbh | 4.01 | (1.2) |
| brain | basal ganglia | aggregated | 4.32 | (1.1) |
| brain | basal ganglia | hsyn | 4.41 | (1.12) |
| brain | brainstem | cbh | 6.33 | (1.14) |
| brain | brainstem | aggregated | 6 | (1.08) |
| brain | brainstem | hsyn | 5.85 | (1.1) |
| brain | cerebellum | cbh | 4.85 | (1.14) |
| brain | cerebellum | aggregated | 4.85 | (1.09) |
| brain | cerebellum | hsyn | 4.82 | (1.07) |
| brain | forebrain | cbh | 3.88 | (1.33) |
| brain | forebrain | aggregated | 4.61 | (1.1) |
| brain | forebrain | hsyn | 4.84 | (1.11) |
| brain | hippocampus | cbh | 5.92 | (1.43) |
| brain | hippocampus | aggregated | 5.27 | (1.16) |
| brain | hippocampus | hsyn | 5.01 | (1.16) |
| brain | midbrain | cbh | 5.12 | (1.28) |
| brain | midbrain | aggregated | 5.03 | (1.09) |
| brain | midbrain | hsyn | 4.97 | (1.1) |
| brain | parietal cortex | cbh | 7.47 | (1.14) |
| brain | parietal cortex | aggregated | 6.25 | (1.08) |
| brain | parietal cortex | hsyn | 5.8 | (1.12) |
| brain | substantia nigra | cbh | 10.04 | (1.79) |
| brain | substantia nigra | aggregated | 8.76 | (1.41) |
| brain | substantia nigra | hsyn | 8.27 | (1.41) |
| brain | temporal cortex | cbh | 3.32 | (1.28) |
| brain | temporal cortex | aggregated | 4.53 | (1.14) |
| brain | temporal cortex | hsyn | 4.92 | (1.15) |
| brain | thalamus | cbh | 5.23 | (1.11) |
| brain | thalamus | aggregated | 4.77 | (1.12) |
| brain | thalamus | hsyn | 4.59 | (1.15) |
| dorsal root | N/A | cbh | 2.75 | (1.29) |
| ganglion | | | | |
| dorsal root | N/A | aggregated | 2.74 | (1.16) |
| ganglion | | | | |
| dorsal root | N/A | hsyn | 2.74 | (1.2) |
| ganglion | | | | |
| kidney | N/A | aggregated | 3 | (1.41) |
| kidney | N/A | hsyn | 2.82 | (1.15) |
| kidney | N/A | cbh | 2.74 | (1.11) |
| liver | N/A | aggregated | 0.09 | (1.3) |
| liver | N/A | hsyn | 0.09 | (1.3) |
| liver | N/A | cbh | 0.09 | (1.4) |
| spinal cord | N/A | cbh | 8.1 | (1.2) |
| spinal cord | N/A | aggregated | 6.02 | (1.1) |
| spinal cord | N/A | hsyn | 5.26 | (1.16) |
| spleen | N/A | cbh | 0.79 | (1.83) |
| spleen | N/A | aggregated | 0.86 | (1.24) |
| spleen | N/A | hsyn | 0.88 | (1.17) |

TABLE 4B
Fold change in transduction of V1 virus particles, relative
to the transduction of virus particles comprising “VAR-1”
as disclosed in WO2023060264A1 (amino acid sequence - SEQ ID
NO: 14; nucleotide sequence - SEQ ID NO: 15) for the indicated
tissues, with the standard deviation shown in parentheses following
each value. “CBh” indicates measurements from virus
particles comprising genomes with the cap gene under the control
of the CBh promoter. “hSyn” indicates virus particles
comprising genomes with the cap gene under the control of the
hSyn promoter. “Aggregated” in relation to a tissue
is an average across all measured areas in the specified organ.
“Aggregated” in relation to a promoter is an average
across the CBh and hSyn promoters.

| Organ | Tissue | Promoter | Fold Change from “VAR-1” | |
|---|---|---|---|---|
| brain | aggregated | cbh | 8.95 | (1.07) |
| brain | aggregated | aggregated | 8.58 | (1.05) |
| brain | aggregated | hsyn | 8.41 | (1.05) |
| brain | basal ganglia | cbh | 9.46 | (1.19) |
| brain | basal ganglia | aggregated | 8.78 | (1.12) |
| brain | basal ganglia | hsyn | 8.51 | (1.12) |
| brain | brainstem | cbh | 7.75 | (1.17) |
| brain | brainstem | aggregated | 7.95 | (1.07) |
| brain | brainstem | hsyn | 7.94 | (1.07) |
| brain | cerebellum | cbh | 6.23 | (1.22) |
| brain | cerebellum | aggregated | 6.07 | (1.17) |
| brain | cerebellum | hsyn | 6.12 | (1.17) |
| brain | forebrain | cbh | 13.2 | (1.14) |
| brain | forebrain | aggregated | 11.07 | (1.09) |
| brain | forebrain | hsyn | 10.25 | (1.12) |
| brain | hippocampus | cbh | 9 | (1.32) |
| brain | hippocampus | aggregated | 12.73 | (1.08) |
| brain | hippocampus | hsyn | 13.97 | (1.15) |
| brain | midbrain | cbh | 8.56 | (1.4) |
| brain | midbrain | aggregated | 8.22 | (1.19) |
| brain | midbrain | hsyn | 8.06 | (1.19) |
| brain | parietal cortex | cbh | 9.08 | (1.22) |
| brain | parietal cortex | aggregated | 10.24 | (1.09) |
| brain | parietal cortex | hsyn | 10.57 | (1.09) |
| brain | substantia nigra | cbh | 15.13 | (27.17) |
| brain | substantia nigra | aggregated | 8.84 | (1.14) |
| brain | substantia nigra | hsyn | 7.09 | (1.25) |
| brain | temporal cortex | cbh | 9.38 | (1.46) |
| brain | temporal cortex | aggregated | 11.51 | (1.19) |
| brain | temporal cortex | hsyn | 12.17 | (1.18) |
| brain | thalamus | cbh | 9.19 | (1.16) |
| brain | thalamus | aggregated | 7.27 | (1.08) |
| brain | thalamus | hsyn | 6.63 | (1.08) |
| liver | N/A | cbh | 0.09 | (2.26) |
| liver | N/A | aggregated | 0.06 | (1.69) |
| liver | N/A | hsyn | 0.03 | (1.83) |
| spinal | N/A | cbh | 5.68 | (1.31) |
| cord | | | | |
| spinal | N/A | aggregated | 7.93 | (1.13) |
| cord | | | | |
| spinal | N/A | hsyn | 8.44 | (1.15) |

cord

### 8.2. Example 2: Evaluation of AAV Capsid Variants V1-V8

### 8.2.1. Materials and Methods

Measurements were taken and analyzed for AAV virus particles comprising one of capsid variants V2-V8 from the Library described in Example 1, averaged across all tissue pieces for the indicated tissue type and across both NHPs, and reported relative to WT AAV9 in Tables 5A and 5B, and relative to “VAR-1” as disclosed in WO2023060264A1 in Tables 6A and 6B. Sequences for V2-V8 are:
V2—SEQ ID NO:26 (amino acid); SEQ ID NO:27 (nucleotide)
V3—SEQ ID NO:28 (amino acid); SEQ ID NO:29 (nucleotide)
V4—SEQ ID NO:30 (amino acid); SEQ ID NO:31 (nucleotide)
V5—SEQ ID NO:32 (amino acid); SEQ ID NO:33 (nucleotide)
V6—SEQ ID NO:34 (amino acid); SEQ ID NO:35 (nucleotide)
V7—SEQ ID NO:36 (amino acid); SEQ ID NO:37 (nucleotide)
V8—SEQ ID NO:38 (amino acid); SEQ ID NO:39 (nucleotide)

Biodistribution and transduction of tissue were calculated by normalizing aggregated biodistribution or transduction samples to input virus abundance. The output was reported as fold change relative to the WT AAV9 (amino acid sequence—SEQ ID NO:1; nucleotide sequence—SEQ ID NO:2) or “VAR-1” as disclosed in WO2023060264A1 (amino acid sequence—SEQ ID NO:14; nucleotide sequence—SEQ ID NO:15), as indicated. Also evaluated was a reference capsid variant having the amino acid sequence of SEQ ID NO:3636 of WO/2021/230987 A1 (and encoded by the nucleotide sequence of SEQ ID NO:3623 of WO/2021/230987A1).

### 8.2.2. Results

Among these capsid variants, virus particles comprising either V1 or V2 (which is identical to VAR-1 of WO2023060264A1 in amino acid sequence except for the presence of an S483F mutation) showed the highest increase in biodistribution and transduction across multiple brain regions relative to wild-type AAV9, including over 200-fold increase in certain brain regions (e.g., basal ganglia and hippocampus). Virus particles comprising V2 also showed a more than 5-fold increase in transduction and biodistribution across multiple brain regions relative to “VAR-1” as disclosed in WO2023060264A1.

Virus particles comprising the reference capsid variant having the amino acid sequence of SEQ ID NO: 3636 of WO/2021/230987 Ai showed brain transduction (aggregated across tissues, aggregated results from CBh and hSyn promoters) of approximately 18-fold higher than wtAAV9. By comparison, virus particles comprising either V1 or V2 showed brain transduction (aggregated across tissues, aggregated results from CBh and hSyn promoters) of approximately 227-fold and 150-fold, respectively, higher than wtAAV9, demonstrating significantly enhanced brain transduction relative to the reference capsid variant.

Taken together, these findings suggest that capsid polypeptides such as V1 and V2 as described herein are suitable for gene therapies where targeting the brain is important, for example, as described herein. Without being bound by theory, such gene therapies are preferred over existing alternatives, such as those using wild-type AAV9, due to their enhanced specificity for the brain regions of interest and substantial increase in transduction efficiency.

TABLE 5A
Fold change in biodistribution of V1-V8 virus particles, relative
to the biodistribution of virus particles comprising WT AAV9 (amino
acid sequence - SEQ ID NO: 1; nucleotide sequence - SEQ ID NO: 2)
for the indicated tissues, with the standard deviation shown in parentheses
following each value. “hSyn” indicates virus particles comprising
genomes with the cap gene under the control of the hSyn promoter.
“Aggregated” in relation to a tissue is an average across
all measured areas in the specified organ. “Aggregated” in
relation to a promoter is an average across the CBh and hSyn promoters.

| | | | Promoter | | | |
|---|---|---|---|---|---|---|
| Organ | Tissue | Variant | hSyn | | Aggregated | |
| brain | aggregated | V1 | 37.12 | (1.05) | 35.3 | (1.06) |
| | | V2 | 44.04 | (1.12) | 41.91 | (1.09) |
| | | V3 | 6.95 | (1.11) | 6.89 | (1.08) |
| | | V4 | 9.72 | (1.04) | 9.11 | (1.04) |
| | | V5 | 4.72 | (1.16) | 5.34 | (1.13) |
| | | V6 | 7.67 | (1.08) | 6.4 | (1.07) |
| | | V7 | 7.07 | (1.17) | 6.12 | (1.13) |
| | | V8 | 6.37 | (1.05) | 5.76 | (1.05) |
| brain | basal | V1 | 21.95 | (1.12) | 20.56 | (1.1) |
| | ganglia | V2 | 23.11 | (1.28) | 22.71 | (1.15) |
| | | V3 | 5.84 | (1.25) | 4.27 | (1.24) |
| | | V4 | 6.44 | (1.06) | 6.07 | (1.05) |
| | | V5 | 3.12 | (1.43) | 3.02 | (1.32) |
| | | V6 | 4.17 | (1.1) | 3.22 | (1.09) |
| | | V7 | 3.15 | (1.68) | 3.01 | (1.47) |
| | | V8 | 3.98 | (1.07) | 3.73 | (1.07) |
| brain | brainstem | V1 | 35.36 | (1.11) | 34.52 | (1.08) |
| | | V2 | 28.6 | (1.51) | 31.3 | (1.31) |
| | | V3 | 5.13 | (1.33) | 4.45 | (1.3) |
| | | V4 | 7.59 | (1.12) | 7.28 | (1.1) |
| | | V5 | 2.5 | (1.58) | 2.72 | (1.41) |
| | | V6 | 5.26 | (1.24) | 5.03 | (1.28) |
| | | V7 | 8.39 | (1.53) | 5.87 | (1.44) |
| | | V8 | 4.72 | (1.13) | 4.31 | (1.1) |
| brain | cerebellum | V1 | 60.88 | (1.1) | 57.89 | (1.11) |
| | | V2 | 74.64 | (1.18) | 62.85 | (1.16) |
| | | V3 | 13.09 | (1.22) | 14.09 | (1.15) |
| | | V4 | 17.91 | (1.12) | 16.6 | (1.11) |
| | | V5 | 13.07 | (1.34) | 11.72 | (1.24) |
| | | V6 | 14.1 | (1.17) | 10.08 | (1.14) |
| | | V7 | 9.92 | (1.39) | 9.7 | (1.27) |
| | | V8 | 11.96 | (1.13) | 10.35 | (1.11) |
| brain | forebrain | V1 | 33.84 | (1.12) | 30.8 | (1.1) |
| | | V2 | 25.05 | (1.33) | 30.62 | (1.17) |
| | | V3 | 6.4 | (1.25) | 6.18 | (1.19) |
| | | V4 | 9.5 | (1.08) | 8.8 | (1.07) |
| | | V5 | 4.53 | (1.68) | 4.97 | (1.26) |
| | | V6 | 9.14 | (1.28) | 6.97 | (1.2) |
| | | V7 | 9.66 | (1.35) | 7.06 | (1.34) |
| | | V8 | 5.76 | (1.12) | 5.32 | (1.1) |
| brain | hippocampus | V1 | 23.76 | (1.15) | 24.01 | (1.18) |
| | | V2 | 41.83 | (1.48) | 32.98 | (1.28) |
| | | V3 | 5.41 | (1.38) | 4.93 | (1.29) |
| | | V4 | 6.65 | (1.02) | 6.32 | (1.04) |
| | | V5 | 3.54 | (1.99) | 2.77 | (1.85) |
| | | V6 | 4.03 | (1.23) | 3.19 | (1.17) |
| | | V7 | 7.76 | (2.41) | 6.32 | (1.27) |
| | | V8 | 4.49 | (1.08) | 3.92 | (1.08) |
| brain | midbrain | V1 | 46.14 | (1.15) | 44.18 | (1.12) |
| | | V2 | 79.28 | (1.34) | 71.47 | (1.15) |
| | | V3 | 6.89 | (1.56) | 8.73 | (1.87) |
| | | V4 | 12.08 | (1.11) | 11.49 | (1.1) |
| | | V5 | 4.62 | (2.98) | 5.23 | (2.79) |
| | | V6 | 7.57 | (1.19) | 8.96 | (1.24) |
| | | V7 | 12.82 | (6.52) | 7.58 | (9.58) |
| | | V8 | 8.75 | (1.17) | 7.54 | (1.17) |
| brain | parietal | V1 | 50.15 | (1.12) | 51.14 | (1.09) |
| | cortex | V2 | 69.61 | (1.35) | 62.5 | (1.23) |
| | | V3 | 7.36 | (1.4) | 6.32 | (1.28) |
| | | V4 | 10.97 | (1.1) | 10.23 | (1.1) |
| | | V5 | 7.33 | (1.51) | 5.58 | (1.43) |
| | | V6 | 10.58 | (1.14) | 7.93 | (1.13) |
| | | V7 | 12.97 | (1.35) | 11.22 | (1.3) |
| | | V8 | 6.27 | (1.12) | 6.15 | (1.08) |
| brain | substantia | V1 | 81.2 | (1.35) | 82.94 | (1.43) |
| | nigra | V2 | 51.58 | (3.88) | 78.79 | (1.4) |
| | | V3 | 4.8 | (16.96) | 6.33 | (3) |
| | | V4 | 10.41 | (1.04) | 9.34 | (1.1) |
| | | V5 | 0 | | 11.62 | (23.91) |
| | | V6 | 17.5 | (1.43) | 15.13 | (1.28) |
| | | V7 | 0 | | 0 | |
| | | V8 | 9.04 | (1.54) | 8.48 | (1.36) |
| brain | temporal | V1 | 37.06 | (1.16) | 32.56 | (1.17) |
| | cortex | V2 | 44.07 | (1.24) | 49.36 | (1.16) |
| | | V3 | 6.72 | (1.36) | 8.81 | (1.25) |
| | | V4 | 9.04 | (1.08) | 8.66 | (1.07) |
| | | V5 | 3.67 | (1.8) | 5.93 | (1.64) |
| | | V6 | 6.55 | (1.26) | 7.2 | (1.23) |
| | | V7 | 3.5 | (4.18) | 3.19 | (1.99) |
| | | V8 | 6.9 | (1.1) | 6.41 | (1.11) |
| brain | thalamus | V1 | 40.45 | (1.13) | 39.6 | (1.09) |
| | | V2 | 80.44 | (1.42) | 57.6 | (1.25) |
| | | V3 | 7.54 | (1.23) | 7.61 | (1.21) |
| | | V4 | 11.81 | (1.07) | 10.9 | (1.06) |
| | | V5 | 2.52 | (4.63) | 7.39 | (1.4) |
| | | V6 | 8.1 | (1.14) | 6.95 | (1.08) |
| | | V7 | 1.87 | (4.83) | 7.22 | (2.83) |
| | | V8 | 7.79 | (1.11) | 6.65 | (1.09) |
| dorsal | N/A | V1 | 2.43 | (1.23) | 2.45 | (1.15) |
| root | | V2 | 4.95 | (1.78) | 2.18 | (1.67) |
| ganglia | | V3 | 0.93 | (1.38) | 1.27 | (1.36) |
| | | V4 | 0.74 | (1.13) | 0.82 | (1.12) |
| | | V5 | 0.81 | (1.98) | 0.63 | (1.63) |
| | | V6 | 0.55 | (1.41) | 0.49 | (1.35) |
| | | V7 | 2.07 | (1.88) | 1.64 | (1.61) |
| | | V8 | 0.66 | (1.17) | 0.71 | (1.1) |
| kidney | N/A | V1 | 4.8 | (1.16) | 4.74 | (1.16) |
| | | V2 | 5.69 | (2.61) | 6.86 | (1.41) |
| | | V3 | 0.59 | (1.72) | 1.37 | (1.43) |
| | | V4 | 2.25 | (1.09) | 2.27 | (1.07) |
| | | V5 | 0.9 | (3.01) | 1.64 | (2.06) |
| | | V6 | 1.44 | (1.24) | 1.25 | (1.19) |
| | | V7 | 2.97 | (1.55) | 2.7 | (1.46) |
| | | V8 | 1.56 | (1.11) | 1.53 | (1.1) |
| liver | N/A | V1 | 0.03 | (1.36) | 0.03 | (1.31) |
| | | V2 | 0.04 | (1.55) | 0.06 | (1.33) |
| | | V3 | 0.1 | (1.1) | 0.09 | (1.1) |
| | | V4 | 0.14 | (1.02) | 0.15 | (1.02) |
| | | V5 | 0.08 | (1.18) | 0.08 | (1.16) |
| | | V6 | 0.11 | (1.08) | 0.14 | (1.07) |
| | | V7 | 0.17 | (1.24) | 0.17 | (1.17) |
| | | V8 | 0.17 | (1.04) | 0.18 | (1.03) |
| spinal | N/A | V1 | 21.11 | (1.25) | 23.16 | (1.26) |
| cord | | V2 | 22.84 | (1.39) | 16.73 | (1.3) |
| | | V3 | 2.86 | (1.43) | 2.34 | (1.33) |
| | | V4 | 4.55 | (1.2) | 4.4 | (1.19) |
| | | V5 | 3.31 | (2.5) | 2.44 | (2.12) |
| | | V6 | 6.77 | (1.27) | 5.31 | (1.34) |
| | | V7 | 1.61 | (2.74) | 1.27 | (2.05) |
| | | V8 | 3.88 | (1.24) | 3.64 | (1.18) |
| spleen | N/A | V1 | 0.56 | (1.2) | 0.52 | (1.3) |
| | | V2 | 0.78 | (2.06) | 0.54 | (1.39) |
| | | V3 | 0.73 | (1.35) | 0.7 | (1.25) |
| | | V4 | 0.6 | (1.09) | 0.57 | (1.09) |
| | | V5 | 0.68 | (1.26) | 0.62 | (1.15) |
| | | V6 | 0.53 | (1.06) | 0.5 | (1.06) |
| | | V7 | 1.01 | (1.34) | 0.74 | (1.19) |
| | | V8 | 0.62 | (1.11) | 0.57 | (1.09) |

TABLE 5B
Fold change in transduction of V1-V8 virus particles, relative to
the transduction of virus particles comprising WT AAV9 (amino acid
sequence - SEQ ID NO: 1; nucleotide sequence - SEQ ID NO: 2) for
the indicated tissues, with the standard deviation shown in parentheses
following each value. “hSyn” indicates virus particles comprising
genomes with the cap gene under the control of the hSyn promoter.
“Aggregated” in relation to a tissue is an average across
all measured areas in the specified organ. “Aggregated” in
relation to a promoter is an average across the CBh and hSyn promoters.

| | | | Promoter | | | |
|---|---|---|---|---|---|---|
| Organ | Tissue | Variant | hSyn | | Aggregated | |
| brain | aggregated | V1 | 231.61 | (1.08) | 227.85 | (1.07) |
| | | V2 | 179.92 | (1.14) | 150.55 | (1.09) |
| | | V3 | 54.13 | (1.09) | 49.54 | (1.09) |
| | | V4 | 50.79 | (1.04) | 48.26 | (1.05) |
| | | V5 | 49.99 | (1.12) | 46.92 | (1.11) |
| | | V6 | 38.21 | (1.08) | 36.9 | (1.07) |
| | | V7 | 43.27 | (1.17) | 31.12 | (1.15) |
| | | V8 | 32.6 | (1.05) | 30.09 | (1.05) |
| brain | basal | V1 | 310.36 | (1.11) | 326.58 | (1.13) |
| | ganglia | V2 | 269.25 | (1.51) | 234.68 | (1.2) |
| | | V3 | 44.61 | (1.92) | 50.52 | (1.27) |
| | | V4 | 74.63 | (1.14) | 72.37 | (1.12) |
| | | V5 | 43.64 | (2.6) | 51.16 | (1.44) |
| | | V6 | 54.6 | (1.21) | 53.8 | (1.24) |
| | | V7 | 44.33 | (4.14) | 32.78 | (5.71) |
| | | V8 | 41.08 | (1.09) | 39.33 | (1.11) |
| brain | brainstem | V1 | 183.45 | (1.13) | 168.8 | (1.14) |
| | | V2 | 152.23 | (1.35) | 98.43 | (1.23) |
| | | V3 | 41.27 | (1.17) | 32.99 | (1.22) |
| | | V4 | 37.7 | (1.12) | 35.16 | (1.15) |
| | | V5 | 57.68 | (1.3) | 58.25 | (1.31) |
| | | V6 | 28.06 | (1.28) | 24.2 | (1.22) |
| | | V7 | 34.18 | (3.66) | 24.26 | (1.61) |
| | | V8 | 23.17 | (1.08) | 21.14 | (1.08) |
| brain | cerebellum | V1 | 149.41 | (1.23) | 180.1 | (1.19) |
| | | V2 | 115.22 | (6.22) | 204.86 | (1.21) |
| | | V3 | 45.81 | (1.94) | 38.15 | (1.37) |
| | | V4 | 38.32 | (1.1) | 46.45 | (1.09) |
| | | V5 | 32.01 | (5.73) | 20.21 | (5.25) |
| | | V6 | 23.36 | (1.41) | 33.09 | (1.16) |
| | | V7 | 42.69 | (4.04) | 33.66 | (4.71) |
| | | V8 | 17.13 | (1.13) | 22.14 | (1.14) |
| brain | forebrain | V1 | 322.92 | (1.14) | 336.93 | (1.1) |
| | | V2 | 112.06 | (18.53) | 109.39 | (2.81) |
| | | V3 | 74.26 | (1.38) | 103.9 | (1.25) |
| | | V4 | 54.03 | (1.12) | 54.64 | (1.09) |
| | | V5 | 31.14 | (11.29) | 32.76 | (10) |
| | | V6 | 42.42 | (1.5) | 46.65 | (1.25) |
| | | V7 | 55.36 | (3.12) | 43.65 | (2.05) |
| | | V8 | 52.02 | (1.13) | 50.16 | (1.09) |
| brain | hippocampus | V1 | 537.33 | (1.21) | 468.25 | (1.13) |
| | | V2 | 93.23 | (62.78) | 234.03 | (2.29) |
| | | V3 | 55.61 | (13.78) | 49.39 | (3.73) |
| | | V4 | 86.63 | (1.14) | 89.15 | (1.13) |
| | | V5 | 25.91 | (39.5) | 32.7 | (12.01) |
| | | V6 | 83.19 | (1.23) | 55.89 | (1.28) |
| | | V7 | 0 | | 0 | |
| | | V8 | 48.14 | (1.27) | 41.09 | (1.22) |
| brain | midbrain | V1 | 179.33 | (1.2) | 177.19 | (1.23) |
| | | V2 | 138.29 | (3.77) | 106.07 | (1.98) |
| | | V3 | 45.82 | (1.36) | 45.79 | (1.28) |
| | | V4 | 46 | (1.09) | 43.26 | (1.06) |
| | | V5 | 32.02 | (1.52) | 28.3 | (1.42) |
| | | V6 | 28.04 | (1.21) | 25.33 | (1.19) |
| | | V7 | 45.54 | (3.3) | 26.94 | (3.79) |
| | | V8 | 25.61 | (1.09) | 25.04 | (1.12) |
| brain | parietal | V1 | 350.12 | (1.14) | 321.02 | (1.13) |
| | cortex | V2 | 216 | (2.14) | 169.44 | (1.38) |
| | | V3 | 69.78 | (1.39) | 60.79 | (1.38) |
| | | V4 | 65.57 | (1.06) | 60.3 | (1.05) |
| | | V5 | 82.52 | (1.38) | 75.76 | (1.3) |
| | | V6 | 63.51 | (1.15) | 62.04 | (1.12) |
| | | V7 | 80.02 | (1.43) | 55.22 | (1.48) |
| | | V8 | 43.94 | (1.15) | 39.02 | (1.11) |
| brain | substantia | V1 | 217.79 | (1.17) | 241.01 | (1.14) |
| | nigra | V2 | 167.95 | (83.64) | 70.27 | (57.57) |
| | | V3 | 33.39 | (32.47) | 22.25 | (33.41) |
| | | V4 | 63.84 | (1.35) | 52.73 | (1.42) |
| | | V5 | 140 | (5.48) | 88.37 | (2.58) |
| | | V6 | 13.62 | (25.65) | 8.39 | (21.68) |
| | | V7 | 82.96 | (46.66) | 49.07 | (37.7) |
| | | V8 | 31.54 | (1.41) | 27.55 | (1.24) |
| brain | temporal | V1 | 387.19 | (1.19) | 348.2 | (1.2) |
| | cortex | V2 | 253.51 | (1.65) | 212.12 | (1.45) |
| | | V3 | 60.48 | (2.21) | 47.01 | (1.59) |
| | | V4 | 55.14 | (1.13) | 53.55 | (1.11) |
| | | V5 | 84.53 | (1.38) | 71.14 | (1.28) |
| | | V6 | 57.58 | (1.26) | 53.19 | (1.27) |
| | | V7 | 50.09 | (14.85) | 29.63 | (12.11) |
| | | V8 | 42.84 | (1.12) | 37.94 | (1.12) |
| brain | thalamus | V1 | 168.69 | (1.09) | 167.52 | (1.09) |
| | | V2 | 196.44 | (1.26) | 141.35 | (1.11) |
| | | V3 | 60.92 | (1.17) | 49.95 | (1.16) |
| | | V4 | 45.63 | (1.04) | 40.15 | (1.06) |
| | | V5 | 45.85 | (1.18) | 42.72 | (1.19) |
| | | V6 | 31.87 | (1.14) | 31.01 | (1.09) |
| | | V7 | 34.93 | (1.38) | 27.55 | (1.82) |
| | | V8 | 31.35 | (1.07) | 26.98 | (1.06) |
| liver | N/A | V1 | 0 | (1.41) | 0.03 | (1.53) |
| | | V2 | 0 | | 0.12 | (2.01) |
| | | V3 | 0.06 | (1.76) | 0.13 | (1.29) |
| | | V4 | 0.23 | (1.08) | 0.28 | (1.07) |
| | | V5 | 0.14 | (1.59) | 0.21 | (1.4) |
| | | V6 | 0.27 | (1.23) | 0.29 | (1.11) |
| | | V7 | 0.29 | (1.42) | 0.37 | (1.24) |
| | | V8 | 0.28 | (1.1) | 0.36 | (1.09) |
| spinal | N/A | V1 | 178.46 | (1.15) | 150.23 | (1.13) |
| cord | | V2 | 82.57 | (4.14) | 95 | (1.31) |
| | | V3 | 16.42 | (1.89) | 10.94 | (2.7) |
| | | V4 | 35.31 | (1.07) | 29.76 | (1.08) |
| | | V5 | 34.42 | (1.49) | 32.58 | (1.32) |
| | | V6 | 21.77 | (1.95) | 18.56 | (1.43) |
| | | V7 | 10.2 | (28.74) | 18.09 | (2.76) |
| | | V8 | 21.32 | (1.11) | 17.56 | (1.12) |

TABLE 6A
Fold change in biodistribution of V1-V8 virus particles, relative to the biodistribution
of virus particles comprising “VAR-1” as disclosed in WO2023060264A1
(amino acid sequence - SEQ ID NO: 14; nucleotide sequence - SEQ ID NO: 15)
for the indicated tissues, with the standard deviation shown in parentheses
following each value. “CBh” indicates measurements from virus particles
comprising genomes with the cap gene under the control of the CBh promoter.
“hSyn” indicates virus particles comprising genomes with the cap gene
under the control of the hSyn promoter. “Aggregated” in relation to
a tissue is an average across all measured areas in the specified organ. “Aggregated”
in relation to a promoter is an average across the CBh and hSyn promoters.

| | | | Promoter | | | | | |
|---|---|---|---|---|---|---|---|---|
| Organ | Tissue | Variant | Aggregated | | Cbh | | hSyn | |
| brain | aggregated | V1 | 5.03 | (1.04) | 4.94 | (1.08) | 5.03 | (1.04) |
| | | V2 | 5.97 | (1.08) | 6.47 | (1.09) | 5.96 | (1.11) |
| | | V3 | 0.98 | (1.08) | 1.08 | (1.19) | 0.94 | (1.09) |
| | | V4 | 1.3 | (1.02) | 1.25 | (1.03) | 1.32 | (1.02) |
| | | V5 | 0.76 | (1.12) | 1.03 | (1.26) | 0.64 | (1.16) |
| | | V6 | 0.91 | (1.06) | 0.7 | (1.12) | 1.04 | (1.07) |
| | | V7 | 0.87 | (1.13) | 0.76 | (1.26) | 0.96 | (1.16) |
| | | V8 | 0.82 | (1.03) | 0.74 | (1.05) | 0.86 | (1.03) |
| brain | basal ganglia | V1 | 4.32 | (1.1) | 4.01 | (1.2) | 4.41 | (1.12) |
| | | V2 | 4.77 | (1.15) | 5.25 | (1.21) | 4.64 | (1.24) |
| | | V3 | 0.9 | (1.22) | 0.27 | (1.84) | 1.17 | (1.26) |
| | | V4 | 1.28 | (1.04) | 1.24 | (1.09) | 1.29 | (1.06) |
| | | V5 | 0.63 | (1.29) | 0.67 | (1.67) | 0.63 | (1.35) |
| | | V6 | 0.68 | (1.09) | 0.4 | (1.31) | 0.84 | (1.12) |
| | | V7 | 0.63 | (1.47) | 0.66 | (2.77) | 0.63 | (1.56) |
| | | V8 | 0.78 | (1.07) | 0.76 | (1.11) | 0.8 | (1.06) |
| brain | brainstem | V1 | 6 | (1.08) | 6.33 | (1.14) | 5.85 | (1.1) |
| | | V2 | 5.44 | (1.28) | 6.48 | (1.33) | 4.73 | (1.42) |
| | | V3 | 0.77 | (1.29) | 0.6 | (2.42) | 0.85 | (1.33) |
| | | V4 | 1.26 | (1.05) | 1.29 | (1.18) | 1.26 | (1.05) |
| | | V5 | 0.47 | (1.5) | 0.6 | (1.75) | 0.41 | (1.81) |
| | | V6 | 0.87 | (1.22) | 0.91 | (1.37) | 0.87 | (1.2) |
| | | V7 | 1.02 | (1.39) | 0.43 | (2.65) | 1.39 | (1.62) |
| | | V8 | 0.75 | (1.08) | 0.69 | (1.17) | 0.78 | (1.08) |
| brain | cerebellum | V1 | 4.85 | (1.09) | 4.85 | (1.14) | 4.82 | (1.07) |
| | | V2 | 5.27 | (1.14) | 5.22 | (1.2) | 5.91 | (1.14) |
| | | V3 | 1.18 | (1.14) | 1.54 | (1.41) | 1.04 | (1.16) |
| | | V4 | 1.39 | (1.03) | 1.33 | (1.09) | 1.42 | (1.03) |
| | | V5 | 0.98 | (1.19) | 0.9 | (1.44) | 1.04 | (1.27) |
| | | V6 | 0.85 | (1.1) | 0.35 | (1.23) | 1.12 | (1.12) |
| | | V7 | 0.81 | (1.3) | 0.9 | (1.76) | 0.79 | (1.45) |
| | | V8 | 0.87 | (1.05) | 0.7 | (1.11) | 0.95 | (1.06) |
| brain | forebrain | V1 | 4.61 | (1.1) | 3.88 | (1.33) | 4.84 | (1.11) |
| | | V2 | 4.58 | (1.15) | 5.76 | (1.25) | 3.58 | (1.3) |
| | | V3 | 0.92 | (1.2) | 0.96 | (1.43) | 0.92 | (1.28) |
| | | V4 | 1.32 | (1.04) | 1.22 | (1.1) | 1.36 | (1.05) |
| | | V5 | 0.74 | (1.29) | 0.95 | (1.64) | 0.65 | (1.62) |
| | | V6 | 1.04 | (1.17) | 0.58 | (1.28) | 1.31 | (1.21) |
| | | V7 | 1.06 | (1.34) | 0.55 | (1.84) | 1.38 | (1.39) |
| | | V8 | 0.8 | (1.08) | 0.74 | (1.13) | 0.82 | (1.09) |
| brain | hippocampus | V1 | 5.27 | (1.16) | 5.92 | (1.43) | 5.01 | (1.16) |
| | | V2 | 7.24 | (1.3) | 6.4 | (1.56) | 8.82 | (1.45) |
| | | V3 | 1.08 | (1.28) | 0.95 | (1.58) | 1.14 | (1.34) |
| | | V4 | 1.39 | (1.05) | 1.35 | (1.1) | 1.4 | (1.05) |
| | | V5 | 0.61 | (2.06) | 0.35 | (2.76) | 0.75 | (1.93) |
| | | V6 | 0.7 | (1.15) | 0.45 | (2.24) | 0.85 | (1.26) |
| | | V7 | 1.39 | (1.3) | 1.02 | (2.21) | 1.64 | (1.76) |
| | | V8 | 0.86 | (1.07) | 0.68 | (1.18) | 0.95 | (1.08) |
| brain | midbrain | V1 | 5.03 | (1.09) | 5.12 | (1.28) | 4.97 | (1.1) |
| | | V2 | 8.13 | (1.13) | 8.56 | (1.35) | 8.53 | (1.37) |
| | | V3 | 0.99 | (2.02) | 1.61 | (5.12) | 0.74 | (1.72) |
| | | V4 | 1.31 | (1.07) | 1.33 | (1.18) | 1.3 | (1.09) |
| | | V5 | 0.6 | (2.59) | 0.82 | (4.7) | 0.5 | (3.11) |
| | | V6 | 1.02 | (1.22) | 1.46 | (1.69) | 0.81 | (1.18) |
| | | V7 | 0.86 | (4.49) | 0 | | 1.38 | (5.12) |
| | | V8 | 0.86 | (1.13) | 0.68 | (1.13) | 0.94 | (1.14) |
| brain | parietal cortex | V1 | 6.25 | (1.08) | 7.47 | (1.14) | 5.8 | (1.12) |
| | | V2 | 7.64 | (1.25) | 7.99 | (1.27) | 8.05 | (1.32) |
| | | V3 | 0.77 | (1.31) | 0.59 | (2.48) | 0.85 | (1.4) |
| | | V4 | 1.25 | (1.07) | 1.21 | (1.12) | 1.27 | (1.08) |
| | | V5 | 0.68 | (1.47) | 0.36 | (4.13) | 0.85 | (1.61) |
| | | V6 | 0.97 | (1.11) | 0.51 | (1.37) | 1.22 | (1.12) |
| | | V7 | 1.37 | (1.3) | 1.21 | (2.17) | 1.5 | (1.4) |
| | | V8 | 0.75 | (1.08) | 0.82 | (1.15) | 0.73 | (1.12) |
| brain | substantia nigra | V1 | 8.76 | (1.41) | 10.04 | (1.79) | 8.27 | (1.41) |
| | | V2 | 8.33 | (1.35) | 11.32 | (1.19) | 5.25 | (4.41) |
| | | V3 | 0.67 | (2.24) | 1.08 | (8.27) | 0.49 | (5.47) |
| | | V4 | 0.99 | (1.06) | 0.82 | (1.55) | 1.06 | (1.04) |
| | | V5 | 1.23 | (8.35) | 3.63 | (15.07) | 0 | |
| | | V6 | 1.6 | (1.24) | 1.3 | (1.16) | 1.78 | (1.44) |
| | | V7 | 0 | | 0 | | 0 | |
| | | V8 | 0.9 | (1.32) | 0.85 | (1.14) | 0.92 | (1.45) |
| brain | temporal cortex | V1 | 4.53 | (1.14) | 3.32 | (1.28) | 4.92 | (1.15) |
| | | V2 | 6.86 | (1.17) | 8.23 | (1.18) | 5.85 | (1.24) |
| | | V3 | 1.23 | (1.29) | 2.01 | (1.54) | 0.89 | (1.38) |
| | | V4 | 1.2 | (1.05) | 1.22 | (1.1) | 1.2 | (1.07) |
| | | V5 | 0.82 | (1.65) | 1.52 | (3.41) | 0.49 | (1.61) |
| | | V6 | 1 | (1.21) | 1.28 | (1.23) | 0.87 | (1.27) |
| | | V7 | 0.44 | (1.94) | 0.42 | (6.27) | 0.46 | (2.29) |
| | | V8 | 0.89 | (1.09) | 0.85 | (1.19) | 0.92 | (1.09) |
| brain | thalamus | V1 | 4.77 | (1.12) | 5.23 | (1.11) | 4.59 | (1.15) |
| | | V2 | 6.94 | (1.22) | 5.74 | (1.36) | 9.13 | (1.33) |
| | | V3 | 0.92 | (1.23) | 1.08 | (2.08) | 0.85 | (1.22) |
| | | V4 | 1.31 | (1.06) | 1.26 | (1.11) | 1.34 | (1.07) |
| | | V5 | 0.89 | (1.44) | 2.19 | (1.56) | 0.29 | (2.46) |
| | | V6 | 0.84 | (1.07) | 0.71 | (1.32) | 0.92 | (1.17) |
| | | V7 | 0.87 | (2.17) | 2.09 | (5.13) | 0.21 | (2.73) |
| | | V8 | 0.8 | (1.08) | 0.62 | (1.18) | 0.88 | (1.09) |
| dorsal root | N/A | V1 | 2.74 | (1.16) | 2.75 | (1.29) | 2.74 | (1.2) |
| ganglia | | V2 | 2.44 | (1.76) | 0.21 | (0.21) | 5.58 | (1.8) |
| | | V3 | 1.42 | (1.35) | 2.15 | (1.86) | 1.05 | (1.42) |
| | | V4 | 0.91 | (1.11) | 1.07 | (1.16) | 0.84 | (1.11) |
| | | V5 | 0.7 | (1.7) | 0.35 | (3.46) | 0.91 | (1.91) |
| | | V6 | 0.55 | (1.38) | 0.43 | (1.6) | 0.62 | (1.36) |
| | | V7 | 1.83 | (1.55) | 1.11 | (5.39) | 2.34 | (1.74) |
| | | V8 | 0.8 | (1.08) | 0.91 | (1.16) | 0.74 | (1.16) |
| kidney | N/A | V1 | 3 | (1.41) | 2.74 | (1.11) | 2.82 | (1.15) |
| | | V2 | 4.08 | (1.54) | 5.03 | (2.2) | 3.25 | (2.38) |
| | | V3 | 0.81 | (1.38) | 1.91 | (2.1) | 0.34 | (1.73) |
| | | V4 | 1.35 | (1.05) | 1.51 | (1.08) | 1.29 | (1.07) |
| | | V5 | 0.98 | (1.75) | 1.91 | (2.03) | 0.51 | (3.12) |
| | | V6 | 0.74 | (1.18) | 0.62 | (2.1) | 0.82 | (1.23) |
| | | V7 | 1.61 | (1.48) | 1.51 | (4.24) | 1.7 | (1.59) |
| | | V8 | 0.91 | (1.07) | 0.97 | (1.18) | 0.89 | (1.06) |
| liver | N/A | V1 | 0.09 | (1.3) | 0.09 | (1.4) | 0.09 | (1.3) |
| | | V2 | 0.19 | (1.35) | 0.21 | (1.58) | 0.13 | (1.61) |
| | | V3 | 0.28 | (1.09) | 0.17 | (1.29) | 0.35 | (1.1) |
| | | V4 | 0.49 | (1.02) | 0.46 | (1.03) | 0.51 | (1.03) |
| | | V5 | 0.26 | (1.16) | 0.24 | (1.4) | 0.28 | (1.15) |
| | | V6 | 0.46 | (1.06) | 0.52 | (1.09) | 0.41 | (1.09) |
| | | V7 | 0.56 | (1.18) | 0.47 | (1.25) | 0.62 | (1.3) |
| | | V8 | 0.58 | (1.03) | 0.55 | (1.05) | 0.59 | (1.05) |
| spinal cord | N/A | V1 | 6.02 | (1.1) | 8.1 | (1.2) | 5.26 | (1.16) |
| | | V2 | 4.35 | (1.26) | 3.54 | (1.3) | 5.69 | (1.29) |
| | | V3 | 0.61 | (1.35) | 0.38 | (2.74) | 0.71 | (1.39) |
| | | V4 | 1.14 | (1.08) | 1.17 | (1.09) | 1.14 | (1.09) |
| | | V5 | 0.64 | (1.93) | 0.28 | (3.61) | 0.82 | (2.15) |
| | | V6 | 1.38 | (1.27) | 0.85 | (1.81) | 1.69 | (1.24) |
| | | V7 | 0.33 | (1.96) | 0.22 | (4.32) | 0.4 | (2.51) |
| | | V8 | 0.95 | (1.12) | 0.92 | (1.21) | 0.97 | (1.14) |
| spleen | N/A | V1 | 0.86 | (1.24) | 0.79 | (1.83) | 0.88 | (1.17) |
| | | V2 | 0.91 | (1.34) | 0.7 | (1.41) | 1.24 | (1.84) |
| | | V3 | 1.17 | (1.23) | 1.22 | (1.39) | 1.15 | (1.29) |
| | | V4 | 0.94 | (1.05) | 0.96 | (1.07) | 0.94 | (1.06) |
| | | V5 | 1.03 | (1.11) | 0.95 | (1.28) | 1.08 | (1.18) |
| | | V6 | 0.83 | (1.08) | 0.84 | (1.19) | 0.84 | (1.04) |
| | | V7 | 1.23 | (1.18) | 0.67 | (1.43) | 1.59 | (1.25) |
| | | V8 | 0.95 | (1.05) | 0.89 | (1.09) | 0.98 | (1.05) |

TABLE 6B
Fold change in transduction of V1-V8 virus particles, relative to the transduction
of virus particles comprising “VAR-1” as disclosed in WO2023060264A1
(amino acid sequence - SEQ ID NO: 14; nucleotide sequence - SEQ ID NO:
15) for the indicated tissues, with the standard deviation shown in parentheses
following each value. “CBh” indicates measurements from virus
particles comprising genomes with the cap gene under the control of the
CBh promoter. “hSyn” indicates virus particles comprising genomes
with the cap gene under the control of the hSyn promoter. “Aggregated”
in relation to a tissue is an average across all measured areas in the
specified organ. “Aggregated” in relation to a promoter is an
average across the CBh and hSyn promoters.

| | | | Promoter | | | | | |
|---|---|---|---|---|---|---|---|---|
| Organ | Tissue | Variant | Aggregated | | Cbh | | hSyn | |
| brain | aggregated | V1 | 8.58 | (1.05) | 8.95 | (1.07) | 8.41 | (1.05) |
| | | V2 | 5.67 | (1.08) | 5.3 | (1.14) | 6.53 | (1.14) |
| | | V3 | 1.87 | (1.08) | 1.65 | (1.18) | 1.96 | (1.08) |
| | | V4 | 1.82 | (1.02) | 1.76 | (1.04) | 1.84 | (1.02) |
| | | V5 | 1.77 | (1.11) | 1.71 | (1.23) | 1.81 | (1.11) |
| | | V6 | 1.39 | (1.05) | 1.42 | (1.1) | 1.39 | (1.07) |
| | | V7 | 1.17 | (1.15) | 0.55 | (1.5) | 1.57 | (1.15) |
| | | V8 | 1.13 | (1.03) | 1.04 | (1.05) | 1.18 | (1.03) |
| brain | basal ganglia | V1 | 8.78 | (1.12) | 9.46 | (1.19) | 8.51 | (1.12) |
| | | V2 | 6.31 | (1.2) | 5.42 | (1.33) | 7.38 | (1.37) |
| | | V3 | 1.36 | (1.25) | 1.61 | (1.62) | 1.22 | (1.44) |
| | | V4 | 1.95 | (1.07) | 1.75 | (1.05) | 2.05 | (1.08) |
| | | V5 | 1.38 | (1.36) | 1.65 | (2.19) | 1.2 | (2.04) |
| | | V6 | 1.45 | (1.19) | 1.36 | (1.33) | 1.5 | (1.17) |
| | | V7 | 0.88 | (2.44) | 0.41 | (6.17) | 1.22 | (2.76) |
| | | V8 | 1.06 | (1.07) | 0.93 | (1.19) | 1.13 | (1.05) |
| brain | brainstem | V1 | 7.95 | (1.07) | 7.75 | (1.17) | 7.94 | (1.07) |
| | | V2 | 4.64 | (1.18) | 3.49 | (1.78) | 6.59 | (1.35) |
| | | V3 | 1.55 | (1.17) | 0.96 | (3.16) | 1.79 | (1.14) |
| | | V4 | 1.66 | (1.06) | 1.74 | (1.09) | 1.63 | (1.07) |
| | | V5 | 2.74 | (1.2) | 3.46 | (1.6) | 2.5 | (1.2) |
| | | V6 | 1.14 | (1.19) | 1.05 | (1.31) | 1.21 | (1.26) |
| | | V7 | 1.14 | (1.81) | 0.58 | (7.55) | 1.48 | (2.21) |
| | | V8 | 1 | (1.07) | 1.01 | (1.14) | 1 | (1.08) |
| brain | cerebellum | V1 | 6.07 | (1.17) | 6.23 | (1.22) | 6.12 | (1.17) |
| | | V2 | 6.9 | (1.26) | 6.56 | (1.37) | 4.72 | (3.74) |
| | | V3 | 1.29 | (1.3) | 0.56 | (3.53) | 1.88 | (1.5) |
| | | V4 | 1.57 | (1.06) | 1.55 | (1.12) | 1.57 | (1.08) |
| | | V5 | 0.68 | (2.53) | 0 | | 1.31 | (2.97) |
| | | V6 | 1.12 | (1.2) | 1.19 | (1.38) | 0.96 | (1.49) |
| | | V7 | 1.13 | (2.6) | 0.5 | (6.48) | 1.75 | (2.61) |
| | | V8 | 0.75 | (1.12) | 0.77 | (1.2) | 0.7 | (1.11) |
| brain | forebrain | V1 | 11.07 | (1.09) | 13.2 | (1.14) | 10.25 | (1.12) |
| | | V2 | 3.59 | (1.94) | 3.82 | (3.13) | 3.56 | (6.59) |
| | | V3 | 3.41 | (1.3) | 5.8 | (1.4) | 2.36 | (1.32) |
| | | V4 | 1.8 | (1.07) | 1.99 | (1.13) | 1.71 | (1.1) |
| | | V5 | 1.08 | (3.73) | 1.26 | (9.93) | 0.99 | (4.16) |
| | | V6 | 1.53 | (1.21) | 1.9 | (1.32) | 1.35 | (1.47) |
| | | V7 | 1.43 | (2.02) | 0.95 | (8.47) | 1.76 | (2.64) |
| | | V8 | 1.65 | (1.07) | 1.66 | (1.14) | 1.65 | (1.12) |
| brain | hippocampus | V1 | 12.73 | (1.08) | 9 | (1.32) | 13.97 | (1.15) |
| | | V2 | 6.36 | (2.97) | 10.13 | (9.57) | 2.42 | (12.38) |
| | | V3 | 1.34 | (2.93) | 1.12 | (9.14) | 1.45 | (4.98) |
| | | V4 | 2.42 | (1.07) | 2.85 | (1.13) | 2.25 | (1.09) |
| | | V5 | 0.89 | (4.12) | 1.34 | (9.28) | 0.67 | (7.26) |
| | | V6 | 1.52 | (1.25) | 0.37 | (4.98) | 2.16 | (1.23) |
| | | V7 | 0 | | 0 | | 0 | |
| | | V8 | 1.12 | (1.19) | 0.84 | (1.53) | 1.25 | (1.17) |
| brain | midbrain | V1 | 8.22 | (1.19) | 8.56 | (1.4) | 8.06 | (1.19) |
| | | V2 | 4.92 | (1.47) | 4.13 | (1.76) | 6.21 | (2.29) |
| | | V3 | 2.12 | (1.26) | 2.28 | (1.77) | 2.06 | (1.34) |
| | | V4 | 2.01 | (1.06) | 1.87 | (1.21) | 2.07 | (1.04) |
| | | V5 | 1.31 | (1.38) | 1.09 | (8.62) | 1.44 | (1.59) |
| | | V6 | 1.17 | (1.2) | 1.05 | (1.4) | 1.26 | (1.22) |
| | | V7 | 1.25 | (2.85) | 0 | | 2.05 | (2.54) |
| | | V8 | 1.16 | (1.07) | 1.19 | (1.23) | 1.15 | (1.07) |
| brain | parietal cortex | V1 | 10.24 | (1.09) | 9.08 | (1.22) | 10.57 | (1.09) |
| | | V2 | 5.41 | (1.33) | 4.95 | (1.59) | 6.52 | (1.46) |
| | | V3 | 1.94 | (1.43) | 1.56 | (2.25) | 2.11 | (1.38) |
| | | V4 | 1.92 | (1.04) | 1.8 | (1.1) | 1.98 | (1.05) |
| | | V5 | 2.42 | (1.31) | 2.34 | (1.5) | 2.49 | (1.36) |
| | | V6 | 1.98 | (1.16) | 2.17 | (1.41) | 1.92 | (1.15) |
| | | V7 | 1.76 | (1.38) | 0.7 | (7.32) | 2.42 | (1.66) |
| | | V8 | 1.25 | (1.06) | 1.08 | (1.16) | 1.33 | (1.1) |
| brain | substantia nigra | V1 | 8.84 | (1.14) | 15.13 | (27.17) | 7.09 | (1.25) |
| | | V2 | 2.58 | (11.7) | 0 | | 5.46 | (16.94) |
| | | V3 | 0.82 | (6.97) | 0 | | 1.09 | (6.95) |
| | | V4 | 1.93 | (1.31) | 1.5 | (2.33) | 2.08 | (1.2) |
| | | V5 | 3.24 | (2.41) | 0 | | 4.56 | (3.91) |
| | | V6 | 0.31 | (4.59) | 0 | | 0.44 | (5.36) |
| | | V7 | 1.8 | (9.91) | 0 | | 2.7 | (11.32) |
| | | V8 | 1.01 | (1.17) | 1.01 | (1.06) | 1.03 | (1.2) |
| brain | temporal cortex | V1 | 11.51 | (1.19) | 9.38 | (1.46) | 12.17 | (1.18) |
| | | V2 | 7.01 | (1.47) | 6.78 | (4.04) | 7.97 | (1.97) |
| | | V3 | 1.55 | (1.62) | 0.75 | (7.72) | 1.9 | (2.08) |
| | | V4 | 1.77 | (1.07) | 1.87 | (1.08) | 1.73 | (1.08) |
| | | V5 | 2.35 | (1.3) | 1.79 | (6.2) | 2.66 | (1.45) |
| | | V6 | 1.76 | (1.25) | 1.72 | (1.4) | 1.81 | (1.25) |
| | | V7 | 0.98 | (4.16) | 0 | | 1.57 | (5.32) |
| | | V8 | 1.25 | (1.13) | 1.06 | (1.29) | 1.35 | (1.15) |
| brain | thalamus | V1 | 7.27 | (1.08) | 9.19 | (1.16) | 6.63 | (1.08) |
| | | V2 | 6.13 | (1.08) | 5.68 | (1.22) | 7.72 | (1.21) |
| | | V3 | 2.17 | (1.12) | 1.57 | (1.69) | 2.4 | (1.15) |
| | | V4 | 1.74 | (1.03) | 1.6 | (1.09) | 1.79 | (1.02) |
| | | V5 | 1.85 | (1.22) | 2.09 | (1.82) | 1.8 | (1.2) |
| | | V6 | 1.34 | (1.09) | 1.65 | (1.16) | 1.25 | (1.14) |
| | | V7 | 1.19 | (1.3) | 0.94 | (2.2) | 1.37 | (1.45) |
| | | V8 | 1.17 | (1.04) | 1.04 | (1.05) | 1.23 | (1.05) |
| liver | N/A | V1 | 0.06 | (1.69) | 0.09 | (2.26) | 0.03 | (1.83) |
| | | V2 | 0.23 | (2.34) | 0.28 | (2.09) | 0 | |
| | | V3 | 0.24 | (1.26) | 0.36 | (1.52) | 0.14 | (1.8) |
| | | V4 | 0.53 | (1.05) | 0.52 | (1.06) | 0.53 | (1.09) |
| | | V5 | 0.39 | (1.39) | 0.43 | (1.42) | 0.32 | (1.55) |
| | | V6 | 0.54 | (1.13) | 0.42 | (1.15) | 0.63 | (1.21) |
| | | V7 | 0.7 | (1.23) | 0.65 | (1.71) | 0.68 | (1.36) |
| | | V8 | 0.68 | (1.05) | 0.69 | (1.08) | 0.64 | (1.06) |
| spinal | N/A | V1 | 7.93 | (1.13) | 5.68 | (1.31) | 8.44 | (1.15) |
| cord | | V2 | 5.01 | (1.38) | 7.32 | (1.52) | 3.91 | (2.08) |

| V3 | 0.58 | (1.79) | 0 | | 0.78 | (2.08) |
|---|---|---|---|---|---|---|
| V4 | 1.57 | (1.07) | 1.28 | (1.19) | 1.67 | (1.06) |
| V5 | 1.72 | (1.34) | 2.07 | (2.65) | 1.63 | (1.5) |
| V6 | 0.98 | (1.4) | 0.95 | (1.6) | 1.03 | (1.48) |
| V7 | 0.95 | (2.27) | 2.08 | (4.28) | 0.48 | (6.31) |
| V8 | 0.93 | (1.13) | 0.73 | (1.45) | 1.01 | (1.11) |

### 8.3. Example 3: In Vivo Evaluation of Medium-Throughput Library in Non-Human Primate

### 8.3.1. Materials and Methods

### 8.3.1.1. Variant Selection

A library of variants was created utilizing multiple sets of strategies of design and selection. The main objectives were to develop capsids capable of packaging into AAV particles efficiently, transducing central nervous system tissues effectively after intravenous administration, and detargeting the liver and other tissue types. Some of the variants were selected from internal data sets acquired from other non-human primate (NHP) experiments. The study also included variants that contain stop codons in VP1 and VP2 as transduction negative controls (expected to produce virus but not transduce cells) and containing VP3 stop codons as production negative controls (not expected to produce virus). The study also included variants having wildtype AAV9 capsid polypeptides as control.

### 8.3.1.2. Library Creation

The virus particles for the study were produced individually via separate transient triple transfection of adherent HEK293T cells followed by pooling and co-purification by iodixanol gradient. Each variant capsid was included in a virus particle that included a genome bearing identifying unique barcode sets of 8 as well as diverse random sequence IDs for quantification. Each genome further contains a sequence encoding a fluorescent reporter gene under the control of a ubiquitous Cbh promoter. A number of variants in the study were produced multiple times in separate rounds with uniquely barcoded genomes, providing a measure of biological replicates within the study.

The representation of each individual variant within the virus pool is measured via NGS via the unique barcode pools that are associated with each variant. Variants identified with low initial productivity yields were produced again individually in a separate production round and combined with virus from the previous productions to balance the representation of every variant to be within 10-fold range in the final test article. Final test article included each variant at an amount of 9e¹⁰-3e¹¹ vg/kg in the IV test article, as measured by ddPCR for final titer and NGS analysis for variant representation. Production efficiencies for individual variants were calculated by NGS relative to production efficiency of wild-type AAV9, with reads for each variant and for the reference summed over all rounds of production in which that variant was produced. These rates are provided in Table 10.

### 8.3.1.3. In-Vivo Evaluation of Library in NHP

All NHP experiments were conducted in accordance with institutional policies and NIH guidelines. Two 3-4 year old male cynomolgus macaques (Macaca fascicularis) weighing 2.6 and 3.6 kg were selected for the study. Both animals were seronegative for anti-AAV9 neutralizing antibodies (NAb) were selected for the study (seronegativity status being serum NAb titers <1:10 based on in vitro NAb assay). Prior to test article administrations, samples of blood were collected. The animals were dosed by intravenous injection (doses: 1.40e13vg/kg. and 1.52e13vg/kg). During the in-life period the animals were monitored for signs of inflammation and were treated with weekly IM injections of steroids (methylprednisolone, 40 mg) according to the animal facility's SOPs and recommendations from the veterinarian. Serum samples were collected 4 hours, 2 days, 5 days, and weekly after the injections. The animals were sacrificed 4 weeks after the injections and tissues were collected for biodistribution and transduction analyses. The tissues collected are shown in Table 7. Peripheral tissue samples were collected into RNAlater® (Sigma-Aldrich) and incubated overnight at 4° C., after which the RNAlater® was drained and samples were frozen at −80° C. The entire brain was sliced into 4 mm coronal slabs using a chilled species-specific brain matrix. Each slab was bisected into left and right hemispheres along the sagittal midline. The slabs from the right hemisphere were immersed in RNAlater, drained and frozen. The left hemisphere slabs were sub-dissected for sub regions of the brain, placed into cryo tubes and immediately frozen on dry-ice.

TABLE 7
List of non-brain and brain tissues collected

| Non-brain tissues | Brain tissues |
|---|---|
| Dorsal root ganglion: cervical | Basal ganglia |
| Dorsal root ganglion: thoracic | Brainstem |
| Dorsal root ganglion: lumbar | Cerebral cortex |
| Dorsal root ganglion: sacral | Hippocampus |
| Kidney | Thalamus |
| Liver, distal | |
| Liver, proximal | |
| Spinal cord, cervical | |
| Spinal cord, thoracic cranial | |
| Spinal cord, thoracic caudal | |
| Spinal cord, lumbar | |
| Spleen | |

### 8.3.1.4. Bulk Sequencing NGS Library Preparation

Brain slices were dissected to isolate regions including, but not limited to cerebellum, forebrain (frontal and motor cortex), basal ganglia, temporal cortex, hippocampus, thalamus, substantia nigra, midbrain, brainstem. For all biodistribution and transduction analyses, total DNA was extracted from tissue samples using MagMAX DNA Multi-Sample Ultra 2.0 Kit (Thermofisher). Samples were first homogenized in the supplied lysis buffer, then incubated at 65° C. with Proteinase K before the lysates were added to a plate for automated extraction using the KingFisher Apex instrument (Thermofisher). RNA was extracted using the MagMAX™ mirVana™ Total RNA Isolation Kit (Thermofisher) according to the manufacturer's recommendations, and was further treated with DNase I-XT (New England Biolabs) to remove any vector DNA contamination in the RNA sample. Reverse transcription was done with Protoscript II Reverse Transcriptase (New England Biolabs) utilizing primers that were specific to the vector transgene and included unique molecular identifiers (UMIs). Control reactions lacking the reverse transcriptase enzyme (-RT control) were also prepared. Finally, samples were prepared for next-generation sequencing by amplifying the transgene barcode regions with primers compatible with Illumina NGS platform and sequenced with NextSeq 2000 (Illumina).

### 8.3.1.5. Bulk Tissue NGS Sample Parsing and Analysis

After sequencing, the barcode tags were extracted from reads with the expected amplicon structure, and the abundance (number of reads, number of UMIs, or number of unique id tags) of each barcode was recorded. Analyses were restricted to the set of barcodes that were present in the input virus sample, as measured by a separate sequencing assay of the input virus sample. To aggregate biodistribution samples, read counts from samples from the same tissue were summed. To aggregate transduction samples, UMIs from samples from the same tissue were summed.

Biodistribution and transduction of tissue were calculated by normalizing aggregated biodistribution or transduction counts with input virus abundance. The transduction and biodistribution rates were calculated as fold change relative to the wild-type (WT) AAV9. The measurements are reported as mean and standard deviation of barcode replicates (n=8-16).

### 8.3.1.6. Single-Nuclei RNA Sequencing and Analysis

Single-cell RNA sequencing has been previously demonstrated to allow characterization of cell-type specific tropism of barcoded rAAVs (Brown et al., Front. Immunol., 2021, 12:730825). An approach that combines single-nuclei RNA sequencing (snRNA-Seq) with targeted amplicon sequencing was developed to reliably detect cell-type specific transduction from up to 50-100 barcoded rAAVs with minimal sequencing depth was applied to the tissues collected from the experiment described in this example. To implement this approach: 1) protocols were developed for isolation of high quality single nuclei suspensions from flash frozen NHP brain tissues (cerebellum, motor cortex, and putamen), 2) flow cytometry was used to sort nuclei transduced with a rAAV (H2B-GFP reporter expressing), 3) the 10× Genomics Chromium X platform was used to encapsulate these nuclei and generate gene expression libraries for reliable identification of cell types, 4) barcoded viral transcripts were selectively amplified (for sequencing) that were captured using the 10× oligo dT capture probes, and 5) viral transcripts identified from the targeted libraries were computationally mapped to cells identified using the gene expression libraries. Using this approach, cell-type specific tropism of multiple rAAVs were investigated in Cynomolgus macaque cerebellum, motor cortex, and putamen. This snRNA-seq gene expression analysis identified all the major central nervous system (CNS) cell types including therapeutically relevant cells such as neurons, excitatory neurons, interneurons, oligodendrocytes, and astrocytes. Viral transduction events, as assessed from the targeted library sequencing, were detected in almost all clusters and successfully quantified differences in transduction rates between rAAVs and benchmarks within a cell type of interest. Overall, the ability to selectively enrich transduced nuclei from the primate CNS, perform snRNA-seq, and quantitate relative transduction between multiple rAAVs in major cell types of interest were demonstrated.

Details of the single nuclear experimental workflow that was applied to the medium throughput study described in this example are below:

TABLE 8
Materials used for the single nuclei RNA sequencing

EZ lysis buffer + 0.2 U/μl of murine RNAse inhibitor
1 × PBS + 20% BSA + 0.2 U/μl of murine RNase inhibitor
1 × PBS + 5% BSA + 0.2 U/μl of murine RNAse inhibitor/Wash
Eight 15 ml tubes pre-coated with 1 × PBS + 2% BSA + 0.2 U/μl
RNAse inhibitor
Two 50 ml tubes pre-coated with 1 × PBS + 2% BSA + 0.2 U/μl
RNAse inhibitor
Two polypropylene FACS tubes pre-coated with 1 × PBS + 2%
BSA + 0.2 U/μl RNAse inhibitor, one with 100 uL 1 × PBS + 5%
BSA + 0.2 U/μl RNAse inhibitor in the bottom for collection
Four 7 ml dounce homogenizer + Pestle A and Pestle B
2 × Dissection Scissors
2 × 0.4 μm filters
2 × 0.7 μm filters
1 ml wide bore pipette tips
Hemocytometer
Six 0.5 ml protein lobind tube with 15 μl of 1 × PBS + 5% BSA +
0.2 U/μl murine RNase inhibitor + 1:100 Propidium Iodide
Two 0.5 ml protein lobind tube with 1 uL Propidium Iodide
Sony MA900 Cell Sorter

### 8.3.1.6.1. Single Nuclei Dissociation from Cerebellum, Motor Cortex, and Putamen

Mincing: All CNS tissue pieces were cut into four ˜35 mg pieces on a cryostat and weighed. The 35 mg tissue pieces were placed in a tube on ice and 200 μl of EZ lysis buffer+RNAse inhibitor was added. The tissue was minced with a pair of microscissors for about 1 min and transferred to a 7 ml dounce homogenizer using a wide bore pipette. Each ˜35 mg tissue piece was homogenized in a separate 7 ml dounce.

Dounce homogenization: EZ lysis buffer+RNAse inhibitor was added to the sample in the dounce homogenizer for a total volume of 7 mls. A loose fitting pestle (Pestle A) was used to dounce the sample with steady strokes (about 1 stroke per second) followed by a tight fitting pestle (Pestle B). The number of dounces varies by tissue type and is indicated in Table 9 below.

TABLE 9
Number of dounces by tissue type

| Tissue | Dounce A | Dounce B |
|---|---|---|
| Motor Cortex | 10x | 5x, wait 20 seconds on ice, 5x |
| Cerebellum | 10x | 5x, wait 20 seconds on ice, 6x |
| Putamen | 10x | 5x, wait 20 seconds on ice, 5x |

Filtration and clean up: Post dounce, a 70 μm filter was stacked on top of a 40 μm filter over a 50 mL falcon pre-coated with 1×PBS+2% BSA+0.2 U/μl of murine RNAse inhibitor. Using a funnel, the sample (7 ml) was passed through the filters by pouring. Next, 7 ml of 1×PBS+20% BSA+0.2 U/μl of murine RNAse inhibitor was passed through the funnel and filters to rinse. Mincing, douncing and filtration was repeated with a second 35 mg tissue piece such that two pieces of tissue are combined during the filtering step. A small (10 μl) aliquot of the filtered sample was transferred to a 0.5 ml protein lobind tube with 1 μl Propidium Iodide for counting. The filtered sample was divided into 4 pre-coated 15 ml falcons with 7 ml sample each. The sample tubes were centrifuged at 200 RCF for 10 mins at 4° C. The supernatant was discarded and the pellet was resuspended in appropriate volume of 1×PBS+5% BSA+RNAse-Inhibitor to obtain ˜3 million nuclei/ml. The nuclei from all four 35 mg pieces were then combined. A small (5 μl) aliquot of the sample was transferred to a 0.5 ml protein lobind tube with 15 μl of 1×PBS+5% BSA+0.2 U/μl murine RNase inhibitor+1:100 Propidium Iodide for counting. The remaining nuclei were stained with 1:100 Draq7 for sorting.

FACS cleanup: The nuclei were sorted on a Sony MA900 cell sorter by gating for intact nuclei that were positively stained for Draq7 and discarding any doublets. The intact nuclei were further gated for GFP positivity and generous gates for sorting were implemented to maximize capture of even faintly GFP positive nuclei. The FACS cleaned nuclei were centrifuged at 200 RCF for 8-10 mins at 4° C. in a final concentration of 10% BSA. The pellet was resuspended in 1×PBS+2% BSA+RNAse-inhibitor and counted. Final nuclei concentration was adjusted as needed for 10× encapsulation.

10× Encapsulation and library preparation: The 10× Chromium X platform (10× Genomics) was used for single cell encapsulation as per the manufacturer's standard instructions. Reverse transcription was performed as per 10× protocols. cDNA amplification was performed using the 10× cDNA amplification kit which allows for amplification of oligo-dT captured transcripts. A viral transcript specific primer was added during cDNA amplification to enable early amplification and purification of viral transcripts. Post cDNA amplification a portion of the cDNA library was used to generate a gene expression library as per 10× standard protocol and the library was quality controlled and sequenced as per standard 10× protocols. A small portion of the cDNA library was used to generate targeted libraries by PCR amplifying the barcode region. For targeted amplification of viral transcripts captured by oligo-dT, primers binding to the TruSeq Handle in combination with a viral transcript specific primer were used. Once the targeted product was amplified pre-indexing and indexing PCRs and sequenced the libraries were performed using an Illumina NextSeq2000 sequencer.

### 8.3.1.6.2. Single Nuclei RNA Sequencing Data Analysis

Gene expression data processing: 10× gene expression libraries were sequenced at a depth of 5000 reads per nuclei. Gene expression sequencing data was demultiplexed using Illumina bcl-convert with default settings, then aligned to Macaca fascicularis reference genome (v6.0, assembly GCA_011100615.1) and quantified using the CellRanger pipeline v7.1.0 with intron mode activated. Doublet detection and filtering was performed using Scrublet package v0.2.3. Dimensionality reduction, batch effect removal, clustering, and identification of marker genes were carried out using Scanpy v1.9.3.

Cell type annotation: 10× gene expression RNA transcript data were plotted on a UMAP plot (Leiden clustering) to reveal the cell clusters. Identification of cell types was performed with an in-house algorithm that projects cell type labels from reference datasets, which was curated from published literature (Siletti et al., Science, 2023; Bakken et al., Nature, 2021; He et al., Curr Biol., 2021; Mortberg et al., Nucleic Acids Res. 2023, each of which is incorporated herein by reference in its entirety). This allowed annotation of major cell types in the CNS tissues, including neurons, oligodendrocytes, oligodendrocyte precursors, astrocytes, microglia, and vascular cells. To validate the annotations, curated cell type specific markers from the literature (Khrameeva et al., Genome Res. 2020; Han et al., Nature, 2022; He et al., Current Biology, 2021; Agarwal et al., Nature Comms., 2020, each of which is incorporated herein by reference in its entirety) were used and confirmed that these markers follow the expected expression patterns in data.

Targeted library data processing: Targeted libraries were processed using an in-house pipeline to obtain the identities of transducing variants and the 1OX feature barcode. To completely remove chimeric molecules, transcript per transcript (TPT) filtering (Dixit, bioRxiv 093237, 2021) was performed with a threshold of 0.5 and 0.02 for forward and reverse molecules, respectively. Targeted libraries were then filtered against gene expression libraries to associate cell type information and limit the analysis to valid cell barcodes. The data was further filtered with cut-offs of 10-1000 reads per molecule to remove any remaining sequencing artifacts. Finally, nuclei with more than 7 observed transduction events, which likely represent clumping artifacts, were excluded from downstream analysis.

Determination of transduction rates: To calculate the normalized transduction rate of variant ‘i’ in cell type ‘j’, the number of transduction events for variant ‘i’ observed in cell type ‘j’ was divided by the population count of cell type ‘j’. To generate the data shown in Table 12, this value was further normalized by the amount of vector genome (vg) dosed, which is defined as the fraction of reads (DNAseq) belonging to variant ‘i’ in the test article, multiplied by total vg dosed into the brain [transduction efficiency of variant ‘i’ in cell type ‘j’=(transduction events i/number of cells ‘j’)/#of dose vector genomes for variant ‘i’].

### 8.3.2. Results

The production efficiency values for V1 and V2 are summarized in Table 10.

TABLE 10
Production efficiency of variant virus particles relative to production
efficiency of WT AAV9 virus particles, in HEK293 cells in vitro.

| Variant | Fold Change Relative to wtAAV9 |
|---|---|
| V1 | 0.81 |
| V2 | 0.53 |

Biodistribution levels for V1 and V2 are summarized in Table 11A. Virus particles comprising V1 and V2 were associated with increased biodistribution levels in brain tissues as compared to virus particles comprising VAR-1. The biodistribution levels obtained with virus particles comprising V1 and V2 in non-brain tissues, especially liver, were lower than those comprising VAR-1.

TABLE 11A
Fold change in biodistribution of virus particles comprising
the indicated variant capsid polypeptide, relative to the
biodistribution of virus particles comprising “VAR-1”
as disclosed in WO2023060264A1 (amino acid sequence - SEQ ID
NO: 14; nucleotide sequence - SEQ ID NO: 15) for brain and
non-brain tissue. “CBh” indicates measurements from
virus particles comprising genomes with the cap gene under
the control of the CBh promoter. “Aggregated” in relation
to a tissue is an average across all measured areas in the specified organ.

| Promoter | Organ | Tissue | V1 | V2 |
|---|---|---|---|---|
| cbh | brain | aggregated | 1.61 | 2.70 |
| cbh | brain | basal ganglia | 2.08 | 3.29 |
| cbh | brain | brainstem | 1.59 | 2.72 |
| cbh | brain | cerebral cortex | 1.52 | 2.48 |
| cbh | brain | hippocampus | 1.30 | 2.37 |
| cbh | brain | thalamus | 1.57 | 2.82 |
| cbh | dorsal root ganglia | N/A | 0.42 | 0.73 |
| cbh | kidney | N/A | 0.74 | 0.71 |
| cbh | liver | N/A | 0.06 | 0.15 |
| cbh | spinal cord | N/A | 0.75 | 1.43 |
| cbh | spleen | N/A | 0.81 | 0.78 |

Transduction levels are summarized in Table 11B. Variant virus particles comprising V1 and V2 were associated with increased transduction levels in brain tissues as compared to VAR-1. In general, virus particles comprising V1 and V2 had lower levels of biodistribution in non-brain tissues (e.g., liver, kidney, and spleen) relative to those comprising VAR-1. Taken together these results indicate that variant virus particles comprising V1 and V2 display improved brain tissue specificity compared to those comprising VAR-1.

TABLE 11B
Fold change in transduction of virus particles comprising the
indicated variant capsid polypeptide, relative to the transduction
of virus particles comprising “VAR-1” as disclosed in
WO2023060264A1 (amino acid sequence - SEQ ID NO: 14; nucleotide
sequence - SEQ ID NO: 15) for brain and non-brain tissues. “CBh”
indicates measurements from virus particles comprising genomes
with the cap gene under the control of the CBh promoter. “Aggregated”
in relation to a tissue is an average across all measured areas
in the specified organ.

| Promoter | Organ | Tissue | V1 | V2 |
|---|---|---|---|---|
| cbh | brain | aggregated | 2.10 | 3.36 |
| cbh | brain | basal ganglia | 2.76 | 3.97 |
| cbh | brain | brainstem | 1.76 | 3.30 |
| cbh | brain | cerebral cortex | 2.42 | 3.39 |
| cbh | brain | hippocampus | 1.83 | 2.93 |
| cbh | brain | thalamus | 3.47 | 3.23 |
| cbh | dorsal root ganglia | aggregated | 0.57 | 0.88 |
| cbh | kidney | aggregated | 0.30 | 0.50 |
| cbh | liver | aggregated | 0.06 | 0.11 |
| cbh | spinal cord | aggregated | 1.39 | 3.46 |
| cbh | spleen | aggregated | 0.52 | 0.68 |

Results of the single-nuclei RNA sequencing and analysis showed that capsid polypeptide variants V1 and V2 resulted in viral particles with an improved transduction profile in various types of brain cells as shown in Table 12 below.

TABLE 12
Single-nuclei RNA sequencing data showing the fold change
in transduction of virus particles comprising the indicated
variant capsid polypeptide, relative to the transduction
of virus particles comprising “VAR-1” as disclosed
in WO2023060264A1 (amino acid sequence - SEQ ID NO: 14;
nucleotide sequence - SEQ ID NO: 15) for various brain cells.

| Brain Tissue | Cell Type | V1 | V2 |
|---|---|---|---|
| cerebellum | Astrocytes | 3.28 | 5.71 |
| cerebellum | Bergmann Glia | 4.14 | 6.15 |
| Motor cortex | Astrocytes | 1.37 | 3.29 |
| Motor cortex | Excitatory Neurons | 3.17 | 3.05 |
| Motor cortex | Interneurons | 5.85 | 5.54 |
| Motor cortex | Neurons | 3.40 | 3.27 |
| putamen | Astrocytes | 2.81 | 4.83 |
| putamen | Excitatory Neurons | 0.07 | 1.08 |
| putamen | Interneurons | 3.08 | 4.37 |
| putamen | Medium Spiny Neurons | 6.51 | 6.25 |
| putamen | Neurons | 1.86 | 2.77 |
| putamen | Oligodendrocytes | 6.51 | 1.24 |

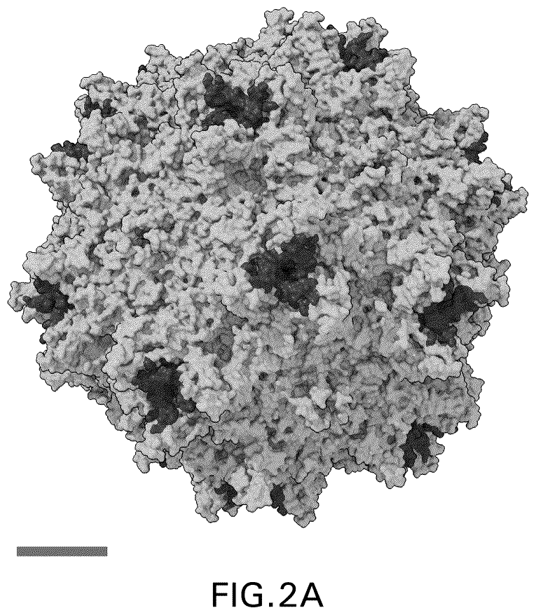

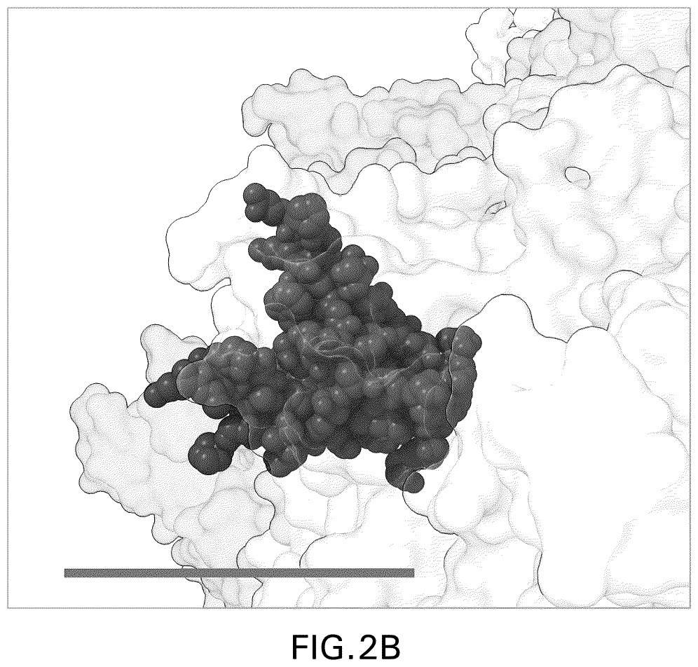

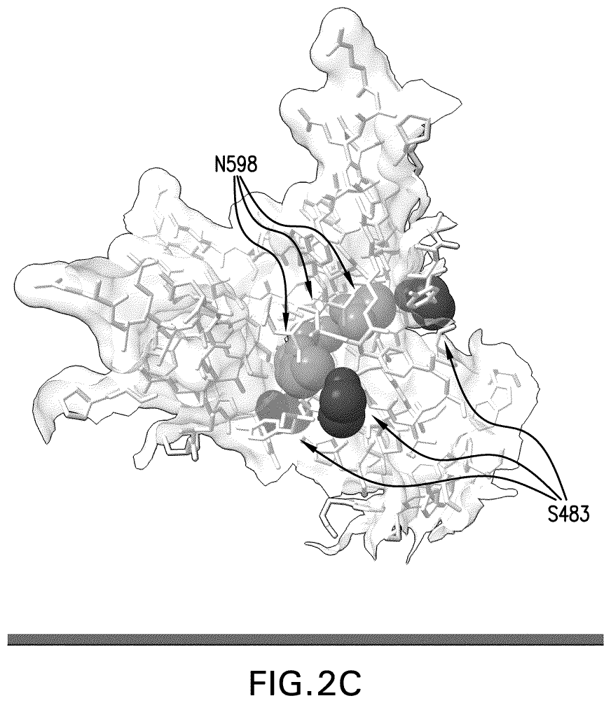

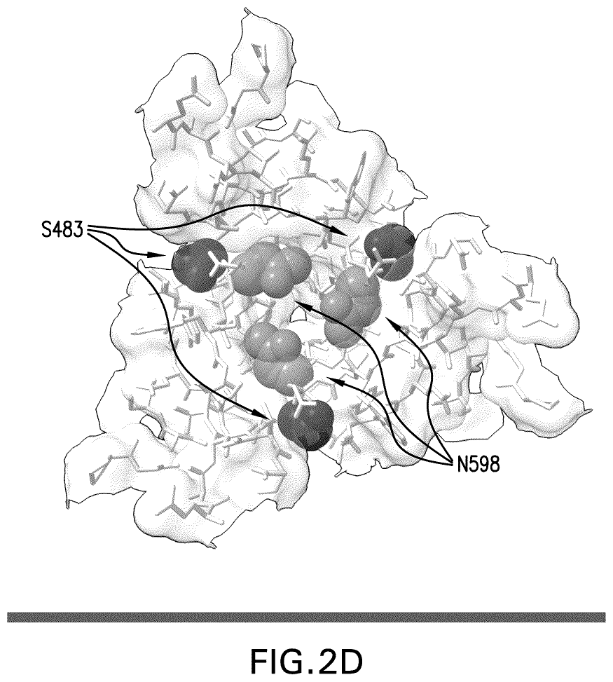

To evaluate the potential structural impact of the mutations present in V1-V8, the wt AAV9 capsid protein structure was visualized using UCSF ChimeraX version 1.8 with the pdb structure 7MT0. These structures are shown in FIGS. 2A-2D. FIG. 2A shows the full AAV9 capsid protein structure at pH 7.4. FIG. 2B highlights a luminal pocket directly under the 3-fold symmetry axis of the wt AAV9 capsid. This region includes amino acid positions 482, 483, 484, 504, 505, 507, 508, 576-583, 590-602, 607, and 629. FIGS. 2C and 2D highlight that residues S483 and N598, which are both mutated in V1 and V2, are in close structural proximity (<4 angstroms) at the 3-fold axis inward protrusion.

To investigate the potential cooperation of mutations at residues S483 and N598, biodistribution and transduction were also evaluated for V9, a variant having S483F, W595A, V596L, N598S, and I601A mutations (as in V1 and V2) with different mutations at the remaining positions (Q579, Q592, and T593). Biodistribution and transduction data are highlighted in Tables 13 and 14, respectively.

TABLE 13
Fold change in biodistribution of virus particles comprising
the indicated variant capsid polypeptide, relative to the biodistribution
of virus particles comprising a different variant, V10, that
is identical to V9 except that V10 lacks the S483F mutation
and has a serine at the S483 position. “CBh” indicates
measurements from virus particles comprising genomes with the
cap gene under the control of the CBh promoter. For each organ,
an average across all measured tissue areas is reported.

| Promoter | Organ | V9 |
|---|---|---|
| cbh | brain | 1.82 |
| cbh | liver | 0.23 |

TABLE 14
Fold change in transduction of virus particles comprising the
indicated variant capsid polypeptide, relative to the biodistribution
of virus particles comprising a different variant, V10, that
is identical to V9 except that V10 lacks the S483F mutation
and has a serine at the S483 position. “CBh” indicates
measurements from virus particles comprising genomes with the
cap gene under the control of the CBh promoter. For each organ,
an average across all measured tissue areas is reported.

| Promoter | Organ | V9 |
|---|---|---|
| cbh | brain | 1.86 |
| cbh | liver | 0.30 |

Virus particle comprising V9 were associated with increased biodistribution and transduction levels in brain tissues as compared to virus particles comprising V10. Virus particles comprising V9 also had significantly lower levels of biodistribution and transduction in liver tissues relative to those comprising V10. These results, taken together with the comparison of V2 relative to VAR-1 (where V2 shares the same mutations as VAR-1 with the addition of the S483F mutation) highlight the significant impact of the S483F mutation on increasing brain transduction and biodistribution as well as decreasing liver transduction and biodistribution.

### 8.4. Example 4—Treatment of Parkinson's Disease

Parkinson's Disease (“PD”) is generally characterized by neuronal inclusions of alpha-synuclein in neuronal cell bodies (Lewy bodies) and within neuronal cell processes (Lewy neurites). PD results in a loss of nerve cells, primarily within the substantia nigra of the midbrain, which are responsible for the production of dopamine. While PD is a multifaceted and complex neurodegenerative disorder, variants in the GBA1 gene, which encodes for the lysosomal hydrolase glucocerebrosidase (GCase), are considered a common genetic risk factor. GBA1 mutations can result in reduced lysosomal function and lead to abnormal cellular accumulation of specific proteins, including α-synuclein, contributing to disease pathogenesis of vulnerable neurons in PD. Studies indicate that replacement of mutated GBA1 with a functional copies of GBA1 via gene therapy can restore GBA1 activity in the affected neurons of the midbrain, prevent α-synuclein aggregation in the substantia nigra and striatum and α-synuclein-mediated degeneration, and slow or prevent disease progression. See, e.g., Rocha et al., 2015, Neurobiology of Disease 82:495-503 and Abeliovich et al., 2021, Journal of Parkinson's Disease, 11(s2):S183-S188.

Based on the results demonstrating increased transduction and biodistribution of variant virus particles comprising one of V1-V8 in a host of brain tissues (relative to wtAAV9), including high transgene transduction rates to the substantia nigra, a recombinant AAV virus particle is generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding a functional human GBA1 protein (e.g., a GBA1 polypeptide comprising the amino acid sequence identified by Uniprot accession no. P04062) operably linked to both a promoter that allows for expression of the GBA1 coding sequence (e.g., a CBH or a hSYN promoter) and a polyA sequence flanked by (ii) a pair ofAAV2 ITRs.

The recombinant AAV virus particle is administered systemically to pre-symptomatic patients at risk of PD (e.g., patients diagnosed with GBA1 mutations).

The recombinant AAV virus particle is also administered to patients diagnosed with PD, e.g., to patients exhibiting one or more symptoms of PD, for example motor symptoms such as akinesia, rigidity and/or tremor. The patients can be tested for GBA1 mutations prior to administration of the recombinant AAV virus particle.

### 8.5. Example 5—Treatment of Huntington's Disease

Huntington's Disease (“HD”) a fully neurodegenerative disease characterized by involuntary choreatic movements with cognitive and behavioral disturbances. HD is caused by a dominantly inherited CAG trinucleotide repeat expansion in the huntingtin gene on the short arm of chromosome 4p16.3 in the HTT gene, which encodes the huntingtin protein (e.g., Uniprot #P42858).

An increase in the size in the number of CAG repeats leads to the production of a mutant huntingtin protein with expanded glutamine repeats. The mutant huntingtin protein is more prone to aggregation and accumulation in neurons, resulting in neurotoxicity. Among the primary neurons affected by HTT toxicity are those of the cerebral cortex and of the striatum within the basal ganglia. Healthy individuals typically have 10 to 35 CAG repeats, individuals with 36 to 39 CAG repeats may or may not develop the signs and symptoms of HD, while people with 40 or more CAG repeats (e.g., 40 to 120 CAG repeats) almost always develop HD. The length of the CAG repeats correlates with the onset of the symptoms, with longer repeats resulting in earlier disease onset. See, e.g., McColgan and Tabrizi, 2018, European Journal of Neurology 25:24-34; Roos, 2010, Orphanet Journal of Rare Diseases 5:4; and Waldvogel et al., 2014, The Neuropathology of Huntington's Disease. In: Nguyen, H., Cenci, M. (eds) Behavioral Neurobiology of Huntington's Disease and Parkinson's Disease. Current Topics in Behavioral Neurosciences, vol 22. Springer, Berlin, Heidelberg. doi.org/10.1007/7854_2014_354.

As a result of the neurotoxic result of mutant huntingtin expression, a number of antisense approaches have been developed to downregulate protein production. Examples of antisense products targeting mutant huntingtin protein include tominersen, WVE-120101 and WVE-120102. See, e.g., Rook and Southwell, 2022, BioDrugs 36:105-119.

Based on the results demonstrating increased transduction and biodistribution of variant virus particles comprising one of V1-V8 in a host of brain tissues (relative to wtAAV9), including high transgene transduction rates to the basal ganglia and cortical regions of the brain, a recombinant AAV virus particle is generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding a huntingtin antisense molecule (e.g., a nucleic acid comprising the nucleotide sequence of tominersen, CUCAGTAACATTGACACCAC (SEQ ID NO:76) operably linked to both a promoter that allows for expression of the antisense molecule (e.g., a CBH or a hSYN promoter) and a polyA sequence flanked by (ii) a pair of AAV2 ITRs.

The recombinant AAV virus particle is administered systemically to pre-symptomatic patients at risk of HD (e.g., patients diagnosed with expanded HTT repeats).

The recombinant AAV virus particle is also administered systemically to patients diagnosed with expanded HTT repeats and having preclinical (prodromal) symptoms such as changes in cognition and behavior but no onset of motor systems.

The recombinant AAV virus particle is also administered to patients diagnosed with HD, e.g., to patients exhibiting one or more symptoms of PD, for example motor symptoms such as chorea. The patients can be tested for HTT mutations prior to administration of the recombinant AAV virus particle.

### 8.6. Example 6—Treatment of Stroke

Ischemic stroke is the consequence of occlusion of one or multiple arteries in the brain. Blockade of blood flow by an occlusive thrombus or embolus leads to rapid and irreversible damage of the non-perfused cerebral tissue. A mainstay of acute ischemic stroke treatment is to rapidly remove the occlusion to allow recanalization and tissue reperfusion to minimize tissue injury. See, e.g., Bacigaluppi et al., 2019, Journal of Cerebral Blood Flow & Metabolism 39(8):1433-1451.

Cross-linked fibrin proteins, as one of the main components of a thrombus, can be dissolved through the process of fibrinolysis. Vascular endothelial cells secrete tPA (tissue plasminogen activator), which has the ability to convert plasminogen to plasmin. Subsequently, plasmin enzymatically cleaves the fibrin skeleton, leading to the dissolution of the thrombus and the recanalization of the occluded vessel. See, e.g., Wang et al., 2024, Journal of the American Heart Association 13:e031692.

tPA and its analogs are of interest for the thrombolytic treatment of acute ischemic stroke. Endogenous tPA is a 70 kDa serine protease composed of 527 amino acid residues represented by SEQ ID NO:77 that are converted to a 2-chain form by hydrolysis of the R275-1276 peptide bond. The N-terminal region of tPA is composed of a fibronectin finger domain, an EGF domain, and 2 kringle domains, and a core region comprising residues 276-527 that constitute the serine protease with the active site composed of H322, D371, and S478. tPA is produced in endothelial cells that catalyzes the cleavage of plasminogen to plasmin and subsequent degradation of fibrin in thrombi as part of coagulation homeostasis. tPA analogs have been developed with altered pharmacokinetic properties or with altered functional properties, including binding to fibrin, fibrin-specific plasminogen activation, and prolonged half-life. See, e.g., Warach et al., 2020, Stroke 51:3440-3451, Llevadot et al., 2001, JAMA. 286:442-449, and references cited therein.

One analog of tPA is reteplase, a 39 kDa tPA analog with the finger, EGF domain, and kringle domains removed. The single-chain molecule consists of 355 amino acids, starting with S1 and ending with P527 of the original tPA sequence and lacking the amino acids V4 through E175. Reteplase can be converted to the double-chain form during fibrinolysis. Reteplase is represented by SEQ ID NO:78

Another analog of tPA is lanoteplase with a deletion of the fibronectin finger domain and EGF domain and an N117Q mutation. The deletion involves the removal of tPA residues C6 through 186. The N117Q mutation results in the elimination of an N-linked glycosylation site. This change was made based on previous findings that nonglycosylated wild-type tPA binds to fibrin better than glycosylated wild-type tPA. Lanoteplase is represented by SEQ ID NO:79

Another analog of tPA is tenecteplase with a variant amino acid sequence as compared to tPA. Tenecteplase includes two mutations the decrease plasma clearance: oT103Q, which adds a glycosylation site in the first kringle domain, and N117Q, which removes another glycosylation site in the first kringle domain. In addition, tenecteplase includes the mutations K296A, H297A, R298A, and R299A as compared to tPA. This tetra-alanine substitution confers enhanced fibrin specificity and resistance to inhibition by plasminogen activator inhibitor-1 (PAl-1). Tenecteplase is represented by SEQ ID NO:80

Based on the results demonstrating increased transduction and biodistribution of variant virus particles comprising one of V1-V8 in brain tissues (relative to wtAAV9), including high transgene transduction rates to various region of the brain, a recombinant AAV virus particle is generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding tPA or a tPA analog comprising an amino acid sequence having at least 90% or at least 95% sequence identity to amino acids 276 to 527 of tPA (e.g., a nucleic acid encoding tenecteplase (SEQ ID NO:81) operably linked to both a promoter that allows for expression of the antisense molecule (e.g., a CBH or a hSYN promoter) and a polyA sequence flanked by (ii) a pair of AAV2 ITRs.

The recombinant AAV virus particle is administered systemically to post-stroke patients, e.g., patients who have suffered an ischemic stroke, for example within 4.5 hours of an ischemic stroke.

### 8.7. Example 7—Treatment of Alzheimer's Disease with Aβ Antibodies

Alzheimer's disease (AD) is a chronic neurodegenerative disorder characterized by the progressive decline of cognitive and functional abilities. It is the most common form of dementia, accounting for up to 70% of all cases.

The underlying pathology of AD is characterized by the accumulation of amyloid-p (Ap) plaques and neurofibrillary tangles composed of hyperphosphorylated tau protein in the brain. These pathological hallmarks disrupt normal neuronal communication and ultimately lead to neuronal death and cognitive decline. Perneczky et al., 2024, Eur J Neurol. 31:e16049.

Targeting the accumulation of Aβ in the brain is currently the most prevalent approach to AD disease—modifying treatments, and recent evidence indicates that monoclonal antibodies aimed at amyloid-β have clinical benefits in the treatment of Aβ. Perneczky et al., 2024, Eur J Neurol. 31:e16049. Examples of such antibodies include bapineuzumab (having a VH represented by SEQ ID NO:81 and a VL represented by SEQ ID NO:82), which targets the N-terminus of Aβ; solanezumab (having a VH represented by SEQ ID NO:83 and a VL represented by SEQ ID NO:84), which targets the mid-region of Aβ; aducanumab (having a VH represented by SEQ ID NO:85 and a VL represented by SEQ ID NO:86), which targets aggregated forms of Aβ, specifically the Aβ fibrils that make up Aβ plaque; lecanemab (BAN2401) (having a VH represented by SEQ ID NO:87 and a VL represented by SEQ ID NO:88), which targets Aβ protofibrils; gantenerumab (having a VH represented by SEQ ID NO:89 and a VL represented by SEQ ID NO:90), which targets aggregated forms of Aβ at a different part of the Aβ protein than aducanumab; donanemab (having a VH represented by SEQ ID NO:91 and a VL represented by SEQ ID NO:92), which targets a specific conformation of Aβ that is present in the earliest stages of plaque formation; and trontinemab, an anti-Aβ/anti-transferrin receptor bispecific antibody (which has an anti-Aβ VH represented by SEQ ID NO:93, an anti-Aβ VL represented by SEQ ID NO:94, an anti-transferrin receptor VH represented by SEQ ID NO:95, and an anti-transferrin receptor VL represented by SEQ ID NO:96). The anti-transferrin component is included to facilitate transport across the blood-brain barrier (BBB).

Based on the results demonstrating increased transduction and biodistribution of variant virus particles comprising one of V1-V8 in a host of brain tissues (relative to wtAAV9), a first recombinant AAV virus particle is generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding an anti-Aβ antibody (e.g., an antibody comprising the VH and VL of gantenerumab) operably linked to both a promoter that allows for expression of the antisense molecule (e.g., a CBH or a hSYN promoter) and a polyA sequence flanked by (ii) a pair of AAV2 ITRs.

A second recombinant AAV virus particle is also generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding an anti-Aβ/anti-transferrin receptor bispecific antibody (e.g., a bispecific antibody which has the anti-Aβ and anti-transferrin receptor VH and VL sequences of trontinemab) operably linked to both a promoter that allows for expression of the antisense molecule (e.g., a CBH or a hSYN promoter) and a polyA sequence flanked by (ii) a pair of AAV2 ITRs.

A third recombinant AAV virus particle is also generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding an anti-Aβ antibody having the anti-Aβ VH and VL sequences of trontinemab operably linked to both a promoter that allows for expression of the antisense molecule (e.g., a CBH or a hSYN promoter) and a polyA sequence flanked by (ii) a pair of AAV2 ITRs.

The first, second or third recombinant AAV virus particle is administered systemically to pre-symptomatic patients at risk of AD (e.g., patients diagnosed with theApoE4 allele).

The first, second, or third recombinant AAV virus particle is also administered systemically to patients diagnosed with symptomatic AD, e.g., patients at early stages of AD.

### 8.8. Example 8—Treatment of Alzheimer's Disease with Tau Antibodies

Alzheimer's disease (AD) is a chronic neurodegenerative disorder characterized by the progressive decline of cognitive and functional abilities. It is the most common form of dementia, accounting for up to 70% of all cases.

The underlying pathology of AD is characterized by the accumulation of amyloid-β (Aβ) plaques and neurofibrillary tangles composed of hyperphosphorylated tau protein in the brain. These pathological hallmarks disrupt normal neuronal communication and ultimately lead to neuronal death and cognitive decline. Perneczky et al., 2024, Eur J Neurol. 31:e16049.

Several antibodies targeting tau have been developed. Bepranemab (RG6416/UCB0107) (having a VH represented by SEQ ID NO:97 and a VL represented by SEQ ID NO:98) binds to seed-capable tau oligomers. Zagotenemab (having a VH represented by SEQ ID NO:99 and a VL represented by SEQ ID NO:100) binds to soluble pathological tau aggregates. Semorinemab (having a VH represented by SEQ ID NO:101 and a VL represented by SEQ ID NO:102) binds the N-terminus of tau. JNJ-63733657 (having a VH represented by SEQ ID NO:103 and a VL represented by SEQ ID NO:104) targets the mid-region of tau and eliminate pathogenic tau seeds. RG7345 (R06926496, a humanized counterpart of rabbit monoclonal antibody MAb 086 binds to phosphorylated tau monomers and paired-helical filaments. MAb 086 comprises a VH represented by SEQ ID NO:115 and a VL represented by SEQ ID NO:116), and humanized counterparts thereof are disclosed in U.S. Pat. No. 10,465,000 B2.

Based on the results demonstrating increased transduction and biodistribution of variant virus particles comprising one of V1-V8 in a host of brain tissues (relative to wtAAV9), a recombinant AAV virus particle is generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding an anti-tau antibody (e.g., an antibody comprising the VH and VL of bepranemab) operably linked to both a promoter that allows for expression of the antisense molecule (e.g., a CBH or a hSYN promoter) and a polyA sequence flanked by (ii) a pair of AAV2 ITRs.

The recombinant AAV virus particle is administered systemically to pre-symptomatic patients at risk of AD (e.g., patients diagnosed with the ApoE4 allele).

The recombinant AAV virus particle is also administered systemically to patients diagnosed with symptomatic AD, e.g., patients at early stages of AD.

### 8.9. Example 9—Treatment of Multiple Sclerosis

Multiple sclerosis (MS) is a chronic inflammatory and demyelinating degenerative disease of the central nervous system. The disease manifests itself in a large number of possible combinations of deficits, including spinal cord, brainstem, cranial nerve, cerebellar, cerebral, and cognitive syndromes. McDonald et al., 2001, Ann Neurol 50:121-7. MS has four patterns of disease: relapsing-remitting MS (RRMS; 80%-85% of cases at onset), primary progressive MS (PPMS; 10%-15% at onset), progressive relapsing MS (PRMS; 5% at onset); and secondary progressive MS (SPMS). See Kremenchutzky et al., 1999, Brain 122 (Pt 10):1941-50 and Confavreux et al., 2000, N Engl J Med 343(20):1430-8). An estimated 50% of patients with RRMS will develop SPMS in 10 years, and up to 90% of RRMS patients will eventually develop SPMS. Weinshenker et al., 1989, Brain 112(Pt 1):133-46.

While historically thought to be a T-cell mediated disease, MS is now understood to involve interactions among different immune cell types, including B-cells, in both the CNS and periphery. Selective B-cell-depleting therapies (such as anti-CD20 monoclonal antibodies) have demonstrated strong efficacy and a good safety profile in the treatment of both relapsing and progressive MS patients with the identification of a new therapeutic target. See, e.g., Margoni et al., 2022, Journal of Neurology 269:1316-1334. A number of anti-CD20 antibodies have been successfully evaluated in MS patients, including rituximab (having a VH represented by SEQ ID NO:105 and a VL represented by SEQ ID NO:106), ocrelizumab (having a VH represented by SEQ ID NO:107 and a VL represented by SEQ ID NO:108), ofatumumab (having a VH represented by SEQ ID NO:109 and a VL represented by SEQ ID NO:110), and ublituximab (having a VH represented by SEQ ID NO:111 and a VL represented by SEQ ID NO:112). See, e.g., Frisch et al., 2021, Neurotherapeutics 18:1602-1622; de Seze et al., 2023, Front. Immunol. 14:1004795. doi: 10.3389/fimmu.2023.1004795; and Margoni et al., 2022, Journal of Neurology 269:1316-1334. Additionally, RG6035/R07121932, an anti-CD20/anti-transferrin receptor bispecific antibody (e.g., an antibody which has an anti-CD20 VH represented by SEQ ID NO:113, an anti-CD20 VL represented by SEQ ID NO:114, an anti-transferrin receptor VH represented by SEQ ID NO:95, and an anti-transferrin receptor VL represented by SEQ ID NO:96), is undergoing clinical trials. The anti-transferrin component is included to facilitate transport across the blood-brain barrier (BBB), and as a result RG6035/R07121932 is sometimes referred to as Brainshuttle (BS) CD20-Multiple Sclerosis.

Based on the results demonstrating increased transduction and biodistribution of variant virus particles comprising one of V1-V8 in a host of brain tissues (relative to wtAAV9), a first recombinant AAV virus particle is generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding an anti-CD20 antibody (e.g., an antibody comprising the VH and VL of ocrelizumab) operably linked to both a promoter that allows for expression of the antisense molecule (e.g., a CBH or a hSYN promoter) and a polyA sequence flanked by (ii) a pair of AAV2 ITRs.

A second recombinant AAV virus particle is also generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding an anti-CD20/anti-transferrin receptor bispecific antibody (e.g., a bispecific antibody which has the anti-CD20 and anti-transferrin receptor VH and VL sequences of RG6035) operably linked to both a promoter that allows for expression of the antisense molecule (e.g., a CBH or a hSYN promoter) and a polyA sequence flanked by (ii) a pair of AAV2 ITRs.

A third recombinant AAV virus particle is also generated comprising (a) capsid polypeptides having the mutation set of SEQ ID NO:12 (e.g., VP1 capsid polypeptides of SEQ ID NO:12 and VP2 and VP3 portions thereof), (b) a viral genome comprising (i) a nucleotide sequence encoding an anti-CD20 antibody having the anti-CD20 VH and VL sequences of RG6035 operably linked to both a promoter that allows for expression of the antisense molecule (e.g., a CBH or a hSYN promoter) and a polyA sequence flanked by (ii) a pair of AAV2 ITRs.

The first, second or third recombinant AAV virus particle is administered systemically to pre-symptomatic patients with RRMS.

The first, second, or third recombinant AAV virus particle is also administered systemically to patients with PPMS.

The first, second, or third recombinant AAV virus particle is also administered systemically to patients with SPMS.

### 9. SEQUENCE LISTING

Exemplary sequences of the present disclosure are provided in Table 15 below, where SEQ=SEQ ID NO.

TABLE 15

| Description | Sequence | SEQ |
|---|---|---|
| AAV9 VP1 | MAADGYLPDWLEDNLSEGIREWWALKPGAPQPKANQQHQDNARGLVLP | 1 |
| wild-type | GYKYLGPGNGLDKGEPVNAADAAALEHDKAYDQQLKAGDNPYLKYNHAD | |
| reference | AEFQERLKEDTSFGGNLGRAVFQAKKRLLEPLGLVEEAAKTAPGKKRPVE | |
| sequence | QSPQEPDSSAGIGKSGAQPAKKRLNFGQTGDTESVPDPQPIGEPPAAPSG | |
| (amino acid) | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMAVQGRNYIPGPSYRQQRVSTTVTQNN | |
| | NSEFAWPGASSWALNGRNSLMNPGPAMASHKEGEDRFFPLSGSLIFGKQ | |
| | GTGRDNVDADKVMITNEEEIKTTNPVATESYGQVATNHQSAQAQAQTGWV | |
| | QNQGILPGMVWQDRDVYLQGPIWAKIPHTDGNFHPSPLMGGFGMKHPPP | |
| | QILIKNTPVPADPPTAFNKDKLNSFITQYSTGQVSVEIEWELQKENSKRWNP | |
| | EIQYTSNYYKSNNVEFAVNTEGVYSEPRPIGTRYLTRNL | |
| AAV9 VP1 | ATGGCTGCCGATGGTTATCTTCCAGATTGGCTCGAGGACAACCTTAGTG | 2 |
| wild-type | AAGGTATTCGCGAGTGGTGGGCTTTGAAACCTGGAGCCCCTCAACCCA | |
| reference | AGGCAAATCAACAACATCAAGACAACGCTCGAGGTCTTGTGCTTCCGG | |
| sequence | GTTACAAATACCTTGGACCCGGCAACGGACTCGACAAGGGGGAGCCG | |
| (nucleotide) | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGGCTGTCCAGGGAAGAAACTACATACCTGGACCCAGCT | |
| | ACCGACAACAACGTGTCTCAACCACTGTGACTCAAAACAACAACAGCGA | |
| | ATTTGCTTGGCCTGGAGCTTCTTCTTGGGCTCTCAATGGACGTAATAGC | |
| | TTGATGAATCCTGGACCTGCTATGGCCAGCCACAAAGAAGGAGAGGAC | |
| | CGTTTCTTTCCTTTGTCTGGATCTTTAATTTTTGGCAAACAAGGAACTGG | |
| | AAGAGACAACGTGGATGCGGACAAAGTCATGATAACCAACGAAGAAGA | |
| | AATTAAAACTACTAACCCGGTAGCAACGGAGTCCTATGGACAAGTGGCC | |
| | ACAAACCACCAGAGTGCCCAAGCACAGGCGCAGACCGGCTGGGTTCAA | |
| | AACCAAGGAATACTTCCGGGTATGGTTTGGCAGGACAGAGATGTGTAC | |
| | CTGCAAGGACCCATTTGGGCCAAAATTCCTCACACGGACGGCAACTTTC | |
| | ACCCTTCTCCGCTGATGGGAGGGTTTGGAATGAAGCACCCGCCTCCTC | |
| | AGATCCTCATCAAAAACACACCTGTACCTGCGGATCCTCCAACGGCCTT | |
| | CAACAAGGACAAGCTGAACTCTTTCATCACCCAGTATTCTACTGGCCAA | |
| | GTCAGCGTGGAGATCGAGTGGGAGCTGCAGAAGGAAAACAGCAAGCG | |
| | CTGGAACCCGGAGATCCAGTACACTTCCAACTATTACAAGTCTAATAAT | |
| | GTTGAATTTGCTGTTAATACTGAAGGTGTATATAGTGAACCCCGCCCCA | |
| | TTGGCACCAGATACCTGACTCGTAATCTGTAA | |
| AAV2 VP1 | MAADGYLPDWLEDTLSEGIRQWWKLKPGPPPPKPAERHKDDSRGLVLPG | 3 |
| wild-type | YKYLGPFNGLDKGEPVNEADAAALEHDKAYDRQLDSGDNPYLKYNHADAE | |
| reference | FQERLKEDTSFGGNLGRAVFQAKKRVLEPLGLVEEPVKTAPGKKRPVEHS | |
| sequence | PVEPDSSSGTGKAGQQPARKRLNFGQTGDADSVPDPQPLGQPPAAPSGL | |
| (amino acid) | GTNTMATGSGAPMADNNEGADGVGNSSGNWHCDSTWMGDRVITTSTRT | |
| | WALPTYNNHLYKQISSQSGASNDNHYFGYSTPWGYFDFNRFHCHFSPRD | |
| | WQRLINNNWGFRPKRLNFKLFNIQVKEVTQNDGTTTIANNLTSTVQVFTDS | |
| | EYQLPYVLGSAHQGCLPPFPADVFMVPQYGYLTLNNGSQAVGRSSFYCLE | |
| | YFPSQMLRTGNNFTFSYTFEDVPFHSSYAHSQSLDRLMNPLIDQYLYYLSR | |
| | TNTPSGTTTQSRLQFSQAGASDIRDQSRNWLPGPCYRQQRVSKTSADNN | |
| | NSEYSWTGATKYHLNGRDSLVNPGPAMASHKDDEEKFFPQSGVLIFGKQ | |
| | GSEKTNVDIEKVMITDEEEIRTTNPVATEQYGSVSTNLQRGNRQAATADVN | |
| | TQGVLPGMVWQDRDVYLQGPIWAKIPHTDGHFHPSPLMGGFGLKHPPPQI | |
| | LIKNTPVPANPSTTFSAAKFASFITQYSTGQVSVEIEWELQKENSKRWNPEI | |
| | QYTSNYNKSVNVDFTVDTNGVYSEPRPIGTRYLTRNL | |
| AAV2 VP1 | ATGGCTGCCGATGGTTATCTTCCAGATTGGCTCGAGGACACTCTCTCTG | 4 |
| wild-type | AAGGAATAAGACAGTGGTGGAAGCTCAAACCTGGCCCACCACCACCAA | |
| reference | AGCCCGCAGAGCGGCATAAGGACGACAGCAGGGGTCTTGTGCTTCCT | |
| sequence | GGGTACAAGTACCTCGGACCCTTCAACGGACTCGACAAGGGAGAGCCG | |
| (nucleotide) | GTCAACGAGGCAGACGCCGCGGCCCTCGAGCACGACAAAGCCTACGA | |
| | CCGGCAGCTCGACAGCGGAGACAACCCGTACCTCAAGTACAACCACGC | |
| | CGACGCGGAGTTTCAGGAGCGCCTTAAAGAAGATACGTCTTTTGGGGG | |
| | CAACCTCGGACGAGCAGTCTTCCAGGCGAAAAAGAGGGTTCTTGAACC | |
| | TCTGGGCCTGGTTGAGGAACCTGTTAAGACGGCTCCGGGAAAAAAGAG | |
| | GCCGGTAGAGCACTCTCCTGTGGAGCCAGACTCCTCCTCGGGAACCG | |
| | GAAAGGCGGGCCAGCAGCCTGCAAGAAAAAGATTGAATTTTGGTCAGA | |
| | CTGGAGACGCAGACTCAGTACCTGACCCCCAGCCTCTCGGACAGCCAC | |
| | CAGCAGCCCCCTCTGGTCTGGGAACTAATACGATGGCTACAGGCAGTG | |
| | GCGCACCAATGGCAGACAATAACGAGGGCGCCGACGGAGTGGGTAAT | |
| | TCCTCGGGAAATTGGCATTGCGATTCCACATGGATGGGCGACAGAGTC | |
| | ATCACCACCAGCACCCGAACCTGGGCCCTGCCCACCTACAACAACCAC | |
| | CTCTACAAACAAATTTCCAGCCAATCAGGAGCCTCGAACGACAATCACT | |
| | ACTTTGGCTACAGCACCCCTTGGGGGTATTTTGACTTCAACAGATTCCA | |
| | CTGCCACTTTTCACCACGTGACTGGCAAAGACTCATCAACAACAACTGG | |
| | GGATTCCGACCCAAGAGACTCAACTTCAAGCTCTTTAACATTCAAGTCA | |
| | AAGAGGTCACGCAGAATGACGGTACGACGACGATTGCCAATAACCTTA | |
| | CCAGCACGGTTCAGGTGTTTACTGACTCGGAGTACCAGCTCCCGTACG | |
| | TCCTCGGCTCGGCGCATCAAGGATGCCTCCCGCCGTTCCCAGCAGACG | |
| | TCTTCATGGTGCCACAGTATGGATACCTCACCCTGAACAACGGGAGTCA | |
| | GGCAGTAGGACGCTCTTCATTTTACTGCCTGGAGTACTTTCCTTCTCAG | |
| | ATGCTGCGTACCGGAAACAACTTTACCTTCAGCTACACTTTTGAGGACG | |
| | TTCCTTTCCACAGCAGCTACGCTCACAGCCAGAGTCTGGACCGTCTCAT | |
| | GAATCCTCTCATCGACCAGTACCTGTATTACTTGAGCAGAACAAACACT | |
| | CCAAGTGGAACCACCACGCAGTCAAGGCTTCAGTTTTCTCAGGCCGGA | |
| | GCGAGTGACATTCGGGACCAGTCTAGGAACTGGCTTCCTGGACCCTGT | |
| | TACCGCCAGCAGCGAGTATCAAAGACATCTGCGGATAACAACAACAGT | |
| | GAATACTCGTGGACTGGAGCTACCAAGTACCACCTCAATGGCAGAGAC | |
| | TCTCTGGTGAATCCGGGCCCGGCCATGGCAAGCCACAAGGACGATGAA | |
| | GAAAAGTTTTTTCCTCAGAGCGGGGTTCTCATCTTTGGGAAGCAAGGCT | |
| | CAGAGAAAACAAATGTGGACATTGAAAAGGTCATGATTACAGACGAAGA | |
| | GGAAATCAGGACAACCAATCCCGTGGCTACGGAGCAGTATGGTTCTGT | |
| | ATCTACCAACCTCCAGAGAGGCAACAGACAAGCAGCTACCGCAGATGT | |
| | CAACACACAAGGCGTTCTTCCAGGCATGGTCTGGCAGGACAGAGATGT | |
| | GTACCTTCAGGGGCCCATCTGGGCAAAGATTCCACACACGGACGGACA | |
| | TTTTCACCCCTCTCCCCTCATGGGTGGATTCGGACTTAAACACCCTCCT | |
| | CCACAGATTCTCATCAAGAACACCCCGGTACCTGCGAATCCTTCGACCA | |
| | CCTTCAGTGCGGCAAAGTTTGCTTCCTTCATCACACAGTACTCCACGGG | |
| | ACAGGTCAGCGTGGAGATCGAGTGGGAGCTGCAGAAGGAAAACAGCA | |
| | AACGCTGGAATCCCGAAATTCAGTACACTTCCAACTACAACAAGTCTGT | |
| | TAATGTGGACTTTACTGTGGACACTAATGGCGTGTATTCAGAGCCTCGC | |
| | CCCATTGGCACCAGATACCTGACTCGTAATCTGTAA | |
| AAV5 VP1 | MSFVDHPPDWLEEVGEGLREFLGLEAGPPKPKPNQQHQDQARGLVLPGY | 5 |
| wild-type | NYLGPGNGLDRGEPVNRADEVAREHDISYNEQLEAGDNPYLKYNHADAEF | |
| reference | QEKLADDTSFGGNLGKAVFQAKKRVLEPFGLVEEGAKTAPTGKRIDDHFPK | |
| sequence | RKKARTEEDSKPSTSSDAEAGPSGSQQLQIPAQPASSLGADTMSAGGGG | |
| (amino acid) | PLGDNNQGADGVGNASGDWHCDSTWMGDRVVTKSTRTWVLPSYNNHQ | |
| | YREIKSGSVDGSNANAYFGYSTPWGYFDFNRFHSHWSPRDWQRLINNYW | |
| | GFRPRSLRVKIFNIQVKEVTVQDSTTTIANNLTSTVQVFTDDDYQLPYVVGN | |
| | GTEGCLPAFPPQVFTLPQYGYATLNRDNTENPTERSSFFCLEYFPSKMLRT | |
| | GNNFEFTYNFEEVPFHSSFAPSQNLFKLANPLVDQYLYRFVSTNNTGGVQ | |
| | FNKNLAGRYANTYKNWFPGPMGRTQGWNLGSGVNRASVSAFATTNRME | |
| | LEGASYQVPPQPNGMTNNLQGSNTYALENTMIFNSQPANPGTTATYLEGN | |
| | MLITSESETQPVNRVAYNVGGQMATNNQSSTTAPATGTYNLQEIVPGSVW | |
| | MERDVYLQGPIWAKIPETGAHFHPSPAMGGFGLKHPPPMMLIKNTPVPGNI | |
| | TSFSDVPVSSFITQYSTGQVTVEMEWELKKENSKRWNPEIQYTNNYNDPQ | |
| | FVDFAPDSTGEYRTTRPIGTRYLTRPL | |
| AAV5 VP1 | ATGTCTTTTGTTGATCACCCTCCAGATTGGTTGGAAGAAGTTGGTGAAG | 6 |
| wild-type | GTCTTCGCGAGTTTTTGGGCCTTGAAGCGGGCCCACCGAAACCAAAAC | |
| reference | CCAATCAGCAGCATCAAGATCAAGCCCGTGGTCTTGTGCTGCCTGGTTA | |
| sequence | TAACTATCTCGGACCCGGAAACGGGCTCGATCGAGGAGAGCCTGTCAA | |
| (nucleotide) | CAGGGCAGACGAGGTCGCGCGAGAGCACGACATCTCGTACAACGAGC | |
| | AGCTTGAGGCGGGAGACAACCCCTACCTCAAGTACAACCACGCGGACG | |
| | CCGAGTTTCAGGAGAAGCTCGCCGACGACACATCCTTCGGGGGAAACC | |
| | TCGGAAAGGCAGTCTTTCAGGCCAAGAAAAGGGTTCTCGAACCTTTTGG | |
| | CCTGGTTGAAGAGGGTGCTAAGACGGCCCCTACCGGAAAGCGGATAGA | |
| | CGACCACTTTCCAAAAAGAAAGAAGGCTCGGACCGAAGAGGACTCCAA | |
| | GCCTTCCACCTCGTCAGACGCCGAAGCTGGACCCAGCGGATCCCAGCA | |
| | GCTGCAAATCCCAGCCCAACCAGCCTCAAGTTTGGGAGCTGATACAAT | |
| | GTCTGCGGGAGGTGGCGGCCCATTGGGCGACAATAACCAAGGTGCCG | |
| | ATGGAGTGGGCAATGCCTCGGGAGATTGGCATTGCGATTCCACGTGGA | |
| | TGGGGGACAGAGTCGTCACCAAGTCCACCCGAACCTGGGTGCTGCCC | |
| | AGCTACAACAACCACCAGTACCGAGAGATCAAAAGCGGCTCCGTCGAC | |
| | GGAAGCAACGCCAACGCCTACTTTGGATACAGCACCCCCTGGGGGTAC | |
| | TTTGACTTTAACCGCTTCCACAGCCACTGGAGCCCCCGAGACTGGCAA | |
| | AGACTCATCAACAACTACTGGGGCTTCAGACCCCGGTCCCTCAGAGTC | |
| | AAAATCTTCAACATTCAAGTCAAAGAGGTCACGGTGCAGGACTCCACCA | |
| | CCACCATCGCCAACAACCTCACCTCCACCGTCCAAGTGTTTACGGACG | |
| | ACGACTACCAGCTGCCCTACGTCGTCGGCAACGGGACCGAGGGATGC | |
| | CTGCCGGCCTTCCCTCCGCAGGTCTTTACGCTGCCGCAGTACGGTTAC | |
| | GCGACGCTGAACCGCGACAACACAGAAAATCCCACCGAGAGGAGCAG | |
| | CTTCTTCTGCCTAGAGTACTTTCCCAGCAAGATGCTGAGAACGGGCAAC | |
| | AACTTTGAGTTTACCTACAACTTTGAGGAGGTGCCCTTCCACTCCAGCT | |
| | TCGCTCCCAGTCAGAACCTGTTCAAGCTGGCCAACCCGCTGGTGGACC | |
| | AGTACTTGTACCGCTTCGTGAGCACAAATAACACTGGCGGAGTCCAGTT | |
| | CAACAAGAACCTGGCCGGGAGATACGCCAACACCTACAAAAACTGGTT | |
| | CCCGGGGCCCATGGGCCGAACCCAGGGCTGGAACCTGGGCTCCGGG | |
| | GTCAACCGCGCCAGTGTCAGCGCCTTCGCCACGACCAATAGGATGGAG | |
| | CTCGAGGGCGCGAGTTACCAGGTGCCCCCGCAGCCGAACGGCATGAC | |
| | CAACAACCTCCAGGGCAGCAACACCTATGCCCTGGAGAACACTATGAT | |
| | CTTCAACAGCCAGCCGGCGAACCCGGGCACCACCGCCACGTACCTCG | |
| | AGGGCAACATGCTCATCACCAGCGAGAGCGAGACGCAGCCGGTGAAC | |
| | CGCGTGGCGTACAACGTCGGCGGGCAGATGGCCACCAACAACCAGAG | |
| | CTCCACCACTGCCCCCGCGACCGGCACGTACAACCTCCAGGAAATCGT | |
| | GCCCGGCAGCGTGTGGATGGAGAGGGACGTGTACCTCCAAGGACCCA | |
| | TCTGGGCCAAGATCCCAGAGACGGGGGCGCACTTTCACCCCTCTCCGG | |
| | CCATGGGCGGATTCGGACTCAAACACCCACCGCCCATGATGCTCATCA | |
| | AGAACACGCCTGTGCCCGGAAATATCACCAGCTTCTCGGACGTGCCCG | |
| | TCAGCAGCTTCATCACCCAGTACAGCACCGGGCAGGTCACCGTGGAGA | |
| | TGGAGTGGGAGCTCAAGAAGGAAAACTCCAAGAGGTGGAACCCAGAGA | |
| | TCCAGTACACAAACAACTACAACGACCCCCAGTTTGTGGACTTTGCCCC | |
| | GGACAGCACCGGGGAATACAGAACCACCAGACCTATCGGAACCCGATA | |
| | CCTTACCCGACCCCTTTAA | |
| AAV8 VP1 | MAADGYLPDWLEDNLSEGIREWWALKPGAPKPKANQQKQDDGRGLVLPG | 7 |
| wild-type | YKYLGPFNGLDKGEPVNAADAAALEHDKAYDQQLQAGDNPYLRYNHADA | |
| reference | EFQERLQEDTSFGGNLGRAVFQAKKRVLEPLGLVEEGAKTAPGKKRPVEP | |
| sequence | SPQRSPDSSTGIGKKGQQPARKRLNFGQTGDSESVPDPQPLGEPPAAPS | |
| (amino acid) | GVGPNTMAAGGGAPMADNNEGADGVGSSSGNWHCDSTWLGDRVITTST | |
| | RTWALPTYNNHLYKQISNGTSGGATNDNTYFGYSTPWGYFDFNRFHCHFS | |
| | PRDWQRLINNNWGFRPKRLSFKLFNIQVKEVTQNEGTKTIANNLTSTIQVFT | |
| | DSEYQLPYVLGSAHQGCLPPFPADVFMIPQYGYLTLNNGSQAVGRSSFYC | |
| | LEYFPSQMLRTGNNFQFTYTFEDVPFHSSYAHSQSLDRLMNPLIDQYLYYL | |
| | SRTQTTGGTANTQTLGFSQGGPNTMANQAKNWLPGPCYRQQRVSTTTG | |
| | QNNNSNFAWTAGTKYHLNGRNSLANPGIAMATHKDDEERFFPSNGILIFGK | |
| | QNAARDNADYSDVMLTSEEEIKTTNPVATEEYGIVADNLQQQNTAPQIGTV | |
| | NSQGALPGMVWQNRDVYLQGPIWAKIPHTDGNFHPSPLMGGFGLKHPPP | |
| | QILIKNTPVPADPPTTFNQSKLNSFITQYSTGQVSVEIEWELQKENSKRWNP | |
| | EIQYTSNYYKSTSVDFAVNTEGVYSEPRPIGTRYLTRNL | |
| AAV8 VP1 | ATGGCTGCCGATGGTTATCTTCCAGATTGGCTCGAGGACAACCTCTCTG | 8 |
| wild-type | AGGGCATTCGCGAGTGGTGGGCGCTGAAACCTGGAGCCCCGAAGCCC | |
| reference | AAAGCCAACCAGCAAAAGCAGGACGACGGCCGGGGTCTGGTGCTTCCT | |
| sequence | GGCTACAAGTACCTCGGACCCTTCAACGGACTCGACAAGGGGGAGCCC | |
| (nucleotide) | GTCAACGCGGCGGACGCAGCGGCCCTCGAGCACGACAAGGCCTACGA | |
| | CCAGCAGCTGCAGGCGGGTGACAATCCGTACCTGCGGTATAACCACGC | |
| | CGACGCCGAGTTTCAGGAGCGTCTGCAAGAAGATACGTCTTTTGGGGG | |
| | CAACCTCGGGCGAGCAGTCTTCCAGGCCAAGAAGCGGGTTCTCGAACC | |
| | TCTCGGTCTGGTTGAGGAAGGCGCTAAGACGGCTCCTGGAAAGAAGAG | |
| | ACCGGTAGAGCCATCACCCCAGCGTTCTCCAGACTCCTCTACGGGCAT | |
| | CGGCAAGAAAGGCCAACAGCCCGCCAGAAAAAGACTCAATTTTGGTCA | |
| | GACTGGCGACTCAGAGTCAGTTCCAGACCCTCAACCTCTCGGAGAACC | |
| | TCCAGCAGCGCCCTCTGGTGTGGGACCTAATACAATGGCTGCAGGCGG | |
| | TGGCGCACCAATGGCAGACAATAACGAAGGCGCCGACGGAGTGGGTA | |
| | GTTCCTCGGGAAATTGGCATTGCGATTCCACATGGCTGGGCGACAGAG | |
| | TCATCACCACCAGCACCCGAACCTGGGCCCTGCCCACCTACAACAACC | |
| | ACCTCTACAAGCAAATCTCCAACGGGACATCGGGAGGAGCCACCAACG | |
| | ACAACACCTACTTCGGCTACAGCACCCCCTGGGGGTATTTTGACTTTAA | |
| | CAGATTCCACTGCCACTTTTCACCACGTGACTGGCAGCGACTCATCAAC | |
| | AACAACTGGGGATTCCGGCCCAAGAGACTCAGCTTCAAGCTCTTCAACA | |
| | TCCAGGTCAAGGAGGTCACGCAGAATGAAGGCACCAAGACCATCGCCA | |
| | ATAACCTCACCAGCACCATCCAGGTGTTTACGGACTCGGAGTACCAGCT | |
| | GCCGTACGTTCTCGGCTCTGCCCACCAGGGCTGCCTGCCTCCGTTCCC | |
| | GGCGGACGTGTTCATGATTCCCCAGTACGGCTACCTAACACTCAACAAC | |
| | GGTAGTCAGGCCGTGGGACGCTCCTCCTTCTACTGCCTGGAATACTTT | |
| | CCTTCGCAGATGCTGAGAACCGGCAACAACTTCCAGTTTACTTACACCT | |
| | TCGAGGACGTGCCTTTCCACAGCAGCTACGCCCACAGCCAGAGCTTGG | |
| | ACCGGCTGATGAATCCTCTGATTGACCAGTACCTGTACTACTTGTCTCG | |
| | GACTCAAACAACAGGAGGCACGGCAAATACGCAGACTCTGGGCTTCAG | |
| | CCAAGGTGGGCCTAATACAATGGCCAATCAGGCAAAGAACTGGCTGCC | |
| | AGGACCCTGTTACCGCCAACAACGCGTCTCAACGACAACCGGGCAAAA | |
| | CAACAATAGCAACTTTGCCTGGACTGCTGGGACCAAATACCATCTGAAT | |
| | GGAAGAAATTCATTGGCTAATCCTGGCATCGCTATGGCAACACACAAAG | |
| | ACGACGAGGAGCGTTTTTTTCCCAGTAACGGGATCCTGATTTTTGGCAA | |
| | ACAAAATGCTGCCAGAGACAATGCGGATTACAGCGATGTCATGCTCACC | |
| | AGCGAGGAAGAAATCAAAACCACTAACCCTGTGGCTACAGAGGAATAC | |
| | GGTATCGTGGCAGATAACTTGCAGCAGCAAAACACGGCTCCTCAAATTG | |
| | GAACTGTCAACAGCCAGGGGGCCTTACCCGGTATGGTCTGGCAGAACC | |
| | GGGACGTGTACCTGCAGGGTCCCATCTGGGCCAAGATTCCTCACACGG | |
| | ACGGCAACTTCCACCCGTCTCCGCTGATGGGGGGCTTTGGCCTGAAAC | |
| | ATCCTCCGCCTCAGATCCTGATCAAGAACACGCCTGTACCTGCGGATC | |
| | CTCCGACCACCTTCAACCAGTCAAAGCTGAACTCTTTCATCACGCAATA | |
| | CAGCACCGGACAGGTCAGCGTGGAAATTGAATGGGAGCTGCAGAAGG | |
| | AAAACAGCAAGCGCTGGAACCCCGAGATCCAGTACACCTCCAACTACT | |
| | ACAAATCTACAAGTGTGGACTTTGCTGTTAATACAGAAGGCGTGTACTC | |
| | TGAACCCCGCCCCATTGGCACCCGTTACCTCACCCGTAATCTGTAA | |
| AAVrh74 | MAADGYLPDWLEDNLSEGIREWWDLKPGAPKPKANQQKQDNGRGLVLP | 9 |
| VP1 wild- | GYKYLGPFNGLDKGEPVNAADAAALEHDKAYDQQLQAGDNPYLRYNHAD | |
| type | AEFQERLQEDTSFGGNLGRAVFQAKKRVLEPLGLVESPVKTAPGKKRPVE | |
| reference | PSPQRSPDSSTGIGKKGQQPAKKRLNFGQTGDSESVPDPQPIGEPPAGPS | |
| sequence | GLGSGTMAAGGGAPMADNNEGADGVGSSSGNWHCDSTWLGDRVITTST | |
| (amino acid) | RTWALPTYNNHLYKQISNGTSGGSTNDNTYFGYSTPWGYFDFNRFHCHFS | |
| | PRDWQRLINNNWGFRPKRLNFKLFNIQVKEVTQNEGTKTIANNLTSTIQVFT | |
| | DSEYQLPYVLGSAHQGCLPPFPADVFMIPQYGYLTLNNGSQAVGRSSFYC | |
| | LEYFPSQMLRTGNNFEFSYNFEDVPFHSSYAHSQSLDRLMNPLIDQYLYYL | |
| | SRTQSTGGTAGTQQLLFSQAGPNNMSAQAKNWLPGPCYRQQRVSTTLSQ | |
| | NNNSNFAWTGATKYHLNGRDSLVNPGVAMATHKDDEERFFPSSGVLMFG | |
| | KQGAGKDNVDYSSVMLTSEEEIKTTNPVATEQYGVVADNLQQQNAAPIVG | |
| | AVNSQGALPGMVWQNRDVYLQGPIWAKIPHTDGNFHPSPLMGGFGLKHP | |
| | PPQILIKNTPVPADPPTTFNQAKLASFITQYSTGQVSVEIEWELQKENSKRW | |
| | NPEIQYTSNYYKSTNVDFAVNTEGTYSEPRPIGTRYLTRNL | |
| AAVrh74 | ATGGCTGCCGATGGTTATCTTCCAGATTGGCTCGAGGACAACCTCTCTG | 10 |
| VP1 wild- | AGGGCATTCGCGAGTGGTGGGACCTGAAACCTGGAGCCCCGAAACCC | |
| type | AAAGCCAACCAGCAAAAGCAGGACAACGGCCGGGGTCTGGTGCTTCCT | |
| reference | GGCTACAAGTACCTCGGACCCTTCAACGGACTCGACAAGGGGGAGCCC | |
| sequence | GTCAACGCGGCGGACGCAGCGGCCCTCGAGCACGACAAGGCCTACGA | |
| (nucleotide) | CCAGCAGCTCCAAGCGGGTGACAATCCGTACCTGCGGTATAATCACGC | |
| | CGACGCCGAGTTTCAGGAGCGTCTGCAAGAAGATACGTCTTTTGGGGG | |
| | CAACCTCGGGCGCGCAGTCTTCCAGGCCAAAAAGCGGGTTCTCGAACC | |
| | TCTGGGCCTGGTTGAATCGCCGGTTAAGACGGCTCCTGGAAAGAAGAG | |
| | GCCGGTAGAGCCATCACCCCAGCGCTCTCCAGACTCCTCTACGGGCAT | |
| | CGGCAAGAAAGGCCAGCAGCCCGCAAAAAAGAGACTCAATTTTGGGCA | |
| | GACTGGCGACTCAGAGTCAGTCCCCGACCCTCAACCAATCGGAGAACC | |
| | ACCAGCAGGCCCCTCTGGTCTGGGATCTGGTACAATGGCTGCAGGCG | |
| | GTGGCGCTCCAATGGCAGACAATAACGAAGGCGCCGACGGAGTGGGT | |
| | AGTTCCTCAGGAAATTGGCATTGCGATTCCACATGGCTGGGCGACAGA | |
| | GTCATCACCACCAGCACCCGCACCTGGGCCCTGCCCACCTACAACAAC | |
| | CACCTCTACAAGCAAATCTCCAACGGGACCTCGGGAGGAAGCACCAAC | |
| | GACAACACCTACTTCGGCTACAGCACCCCCTGGGGGTATTTTGACTTCA | |
| | ACAGATTCCACTGCCACTTTTCACCACGTGACTGGCAGCGACTCATCAA | |
| | CAACAACTGGGGATTCCGGCCCAAGAGGCTCAACTTCAAGCTCTTCAA | |
| | CATCCAAGTCAAGGAGGTCACGCAGAATGAAGGCACCAAGACCATCGC | |
| | CAATAACCTTACCAGCACGATTCAGGTCTTTACGGACTCGGAATACCAG | |
| | CTCCCGTACGTGCTCGGCTCGGCGCACCAGGGCTGCCTGCCTCCGTT | |
| | CCCGGCGGACGTCTTCATGATTCCTCAGTACGGGTACCTGACTCTGAA | |
| | CAATGGCAGTCAGGCTGTGGGCCGGTCGTCCTTCTACTGCCTGGAGTA | |
| | CTTTCCTTCTCAAATGCTGAGAACGGGCAACAACTTTGAATTCAGCTAC | |
| | AACTTCGAGGACGTGCCCTTCCACAGCAGCTACGCGCACAGCCAGAGC | |
| | CTGGACCGGCTGATGAACCCTCTCATCGACCAGTACTTGTACTACCTGT | |
| | CCCGGACTCAAAGCACGGGCGGTACTGCAGGAACTCAGCAGTTGCTAT | |
| | TTTCTCAGGCCGGGCCTAACAACATGTCGGCTCAGGCCAAGAACTGGC | |
| | TACCCGGTCCCTGCTACCGGCAGCAACGTGTCTCCACGACACTGTCGC | |
| | AGAACAACAACAGCAACTTTGCCTGGACGGGTGCCACCAAGTATCATCT | |
| | GAATGGCAGAGACTCTCTGGTGAATCCTGGCGTTGCCATGGCTACCCA | |
| | CAAGGACGACGAAGAGCGATTTTTTCCATCCAGCGGAGTCTTAATGTTT | |
| | GGGAAACAGGGAGCTGGAAAAGACAACGTGGACTATAGCAGCGTGATG | |
| | CTAACCAGCGAGGAAGAAATAAAGACCACCAACCCAGTGGCCACAGAA | |
| | CAGTACGGCGTGGTGGCCGATAACCTGCAACAGCAAAACGCCGCTCCT | |
| | ATTGTAGGGGCCGTCAATAGTCAAGGAGCCTTACCTGGCATGGTGTGG | |
| | CAGAACCGGGACGTGTACCTGCAGGGTCCCATCTGGGCCAAGATTCCT | |
| | CATACGGACGGCAACTTTCATCCCTCGCCGCTGATGGGAGGCTTTGGA | |
| | CTGAAGCATCCGCCTCCTCAGATCCTGATTAAAAACACACCTGTTCCCG | |
| | CGGATCCTCCGACCACCTTCAATCAGGCCAAGCTGGCTTCTTTCATCAC | |
| | GCAGTACAGTACCGGCCAGGTCAGCGTGGAGATCGAGTGGGAGCTGC | |
| | AGAAGGAGAACAGCAAACGCTGGAACCCAGAGATTCAGTACACTTCCA | |
| | ACTACTACAAATCTACAAATGTGGACTTTGCTGTCAATACTGAGGGTACT | |
| | TATTCCGAGCCTCGCCCCATTGGCACCCGTTACCTCACCCGTAATCTGT | |
| | AA | |
| AAVrh74 | MAADGYLPDWLEDNLSEGIREWWDLKPGAPKPKANQQKQDNGRGLVLP | 11 |
| VP1 wild- | GYKYLGPFNGLDKGEPVNAADAAALEHDKAYDQQLQAGDNPYLRYNHAD | |
| type | AEFQERLQEDTSFGGNLGRAVFQAKKRVLEPLGLVESPVKTAPGKKRPVE | |
| alternative | PSPQRSPDSSTGIGKKGQQPAKKRLNFGQTGDSESVPDPQPIGEPPAGPS | |
| reference | GLGSGTMAAGGGAPMADNNEGADGVGSSSGNWHCDSTWLGDRVITTST | |
| sequence | RTWALPTYNNHLYKQISNGTSGGSTNDNTYFGYSTPWGYFDFNRFHCHFS | |
| (amino acid) | PRDWQRLINNNWGFRPKRLNFKLFNIQVKEVTQNEGTKTIANNLTSTIQVFT | |
| | DSEYQLPYVLGSAHQGCLPPFPADVFMIPQYGYLTLNNGSQAVGRSSFYC | |
| | LEYFPSQMLRTGNNFEFSYNFEDVPFHSSYAHSQSLDRLMNPLIDQYLYYL | |
| | SRTQSTGGTAGTQQLLFSQAGPNNMSAQAKNWLPGPCYRQQRVSTTLSQ | |
| | NNNSNFAWTGATKYHLNGRDSLVNPGVAMATHKDDEERFFPSSGVLMFG | |
| | KQGAGKDNVDYSSVMLTSEEEIKTTNPVATEQYGVVADNLQQQNAAPIVG | |
| | AVNSQGALPGMVWQNRDVYLQGPIWAKIPHTDGNFHPSPLMGGFGLKHP | |
| | PPQILIKNTPVPADPPTTFTKAKLASFITQYSTGQVSVEIEWELQKENSKRW | |
| | NPEIQYTSNYYKSTNVDFAVNTEGTYSEPRPIGTRYLTRNL | |
| V1 (amino | MAADGYLPDWLEDNLSEGIREWWALKPGAPQPKANQQHQDNARGLVLP | 12 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMSAQGRNYIPGPFYRQQRVSTSVTQNN | |
| | NSEFAWPGASSWALNGRNSLMNPGPAMASHKEGEDRFFPLSGSLIFGKQ | |
| | GTGRDNVDADKVMITNEEEIKTTNPVATESYGVVATNHQSAQAQAIVGALQ | |
| | SQGALPGMVWQDRDVYLQGPIWAKIPHTDGNFHPSPLMGGFGMKHPPPQ | |
| | ILIKNTPVPADPPTAFNKDKLNSFITQYSTGQVSVEIEWELQKENSKRWNPE | |
| | IQYTSNYYKSNNVEFAVNTEGVYSEPRPIGTRYLTRNL | |
| V1 | ATGGCTGCCGATGGTTATCTTCCAGATTGGCTCGAGGACAACCTTAGTG | 13 |
| (nucleotide) | AAGGTATTCGCGAGTGGTGGGCTTTGAAACCTGGAGCCCCTCAACCCA | |
| | AGGCAAATCAACAACATCAAGACAACGCTCGAGGTCTTGTGCTTCCGG | |
| | GTTACAAATACCTTGGACCCGGCAACGGACTCGACAAGGGGGAGCCG | |
| | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGTCCGCTCAGGGAAGAAACTACATACCTGGACCCTTTTA | |
| | CCGACAACAACGTGTCTCAACCTCAGTGACTCAAAACAACAACAGCGAA | |
| | TTTGCTTGGCCTGGAGCTTCTTCTTGGGCTCTCAATGGACGTAATAGCT | |
| | TGATGAATCCTGGACCTGCTATGGCCAGCCACAAAGAAGGAGAGGACC | |
| | GTTTCTTTCCTTTGTCTGGATCTTTAATTTTTGGCAAACAAGGAACTGGA | |
| | AGAGACAACGTGGATGCGGACAAAGTCATGATAACCAACGAAGAAGAA | |
| | ATTAAAACTACTAACCCGGTAGCAACGGAGTCCTATGGAGTAGTGGCCA | |
| | CAAACCACCAGAGTGCCCAAGCACAGGCGATTGTTGGCGCTCTTCAAT | |
| | CTCAAGGAGCGCTTCCGGGTATGGTTTGGCAGGACAGAGATGTGTACC | |
| | TGCAAGGACCCATTTGGGCCAAAATTCCTCACACGGACGGCAACTTTCA | |
| | CCCTTCTCCGCTGATGGGAGGGTTTGGAATGAAGCACCCGCCTCCTCA | |
| | GATCCTCATCAAAAACACACCTGTACCTGCGGATCCTCCAACGGCCTTC | |
| | AACAAGGACAAGCTGAACTCTTTCATCACCCAGTATTCTACTGGCCAAG | |
| | TCAGCGTGGAGATCGAGTGGGAGCTGCAGAAGGAAAACAGCAAGCGC | |
| | TGGAACCCGGAGATCCAGTACACTTCCAACTATTACAAGTCTAATAATG | |
| | TTGAATTTGCTGTTAATACTGAAGGTGTATATAGTGAACCCCGCCCCATT | |
| | GGCACCAGATACCTGACTCGTAATCTGTAA | |
| “VAR-1” | MAADGYLPDWLEDNLSEGIREWWALKPGAPQPKANQQHQDNARGLVLP | 14 |
| disclosed in | GYKYLGPGNGLDKGEPVNAADAAALEHDKAYDQQLKAGDNPYLKYNHAD | |
| WO2023060 | AEFQERLKEDTSFGGNLGRAVFQAKKRLLEPLGLVEEAAKTAPGKKRPVE | |
| 264A1 | QSPQEPDSSAGIGKSGAQPAKKRLNFGQTGDTESVPDPQPIGEPPAAPSG | |
| (amino acid) | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMAVQGRNYIPGPSYRQQRVSTTVTQNN | |
| | NSEFAWPGASSWALNGRNSLMNPGPAMASHKEGEDRFFPLSGSLIFGKQ | |
| | GTGRDNVDADKVMITNEEEIKTTNPVATESYGVVATNHQSAQAQAIVGALQ | |
| | SQGALPGMVWQDRDVYLQGPIWAKIPHTDGNFHPSPLMGGFGMKHPPPQ | |
| | ILIKNTPVPADPPTAFNKDKLNSFITQYSTGQVSVEIEWELQKENSKRWNPE | |
| | IQYTSNYYKSNNVEFAVNTEGVYSEPRPIGTRYLTRNL | |
| “VAR-1” | ATGGCTGCCGATGGTTATCTTCCAGATTGGCTCGAGGACAACCTTAGTG | 15 |
| disclosed in | AAGGTATTCGCGAGTGGTGGGCTTTGAAACCTGGAGCCCCTCAACCCA | |
| WO2023060 | AGGCAAATCAACAACATCAAGACAACGCTCGAGGTCTTGTGCTTCCGG | |
| 264A1 | GTTACAAATACCTTGGACCCGGCAACGGACTCGACAAGGGGGAGCCG | |
| (nucleotide) | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGGCTGTCCAGGGAAGAAACTACATACCTGGACCCAGCT | |
| | ACCGACAACAACGTGTCTCAACCACTGTGACTCAAAACAACAACAGCGA | |
| | ATTTGCTTGGCCTGGAGCTTCTTCTTGGGCTCTCAATGGACGTAATAGC | |
| | TTGATGAATCCTGGACCTGCTATGGCCAGCCACAAAGAAGGAGAGGAC | |
| | CGTTTCTTTCCTTTGTCTGGATCTTTAATTTTTGGCAAACAAGGAACTGG | |
| | AAGAGACAACGTGGATGCGGACAAAGTCATGATAACCAACGAAGAAGA | |
| | AATTAAAACTACTAACCCGGTAGCAACGGAGTCCTATGGAGTAGTGGCC | |
| | ACAAACCACCAGAGTGCCCAAGCACAGGCGATTGTTGGCGCTCTTCAA | |
| | TCTCAAGGAGCGCTTCCGGGTATGGTTTGGCAGGACAGAGATGTGTAC | |
| | CTGCAAGGACCCATTTGGGCCAAAATTCCTCACACGGACGGCAACTTTC | |
| | ACCCTTCTCCGCTGATGGGAGGGTTTGGAATGAAGCACCCGCCTCCTC | |
| | AGATCCTCATCAAAAACACACCTGTACCTGCGGATCCTCCAACGGCCTT | |
| | CAACAAGGACAAGCTGAACTCTTTCATCACCCAGTATTCTACTGGCCAA | |
| | GTCAGCGTGGAGATCGAGTGGGAGCTGCAGAAGGAAAACAGCAAGCG | |
| | CTGGAACCCGGAGATCCAGTACACTTCCAACTATTACAAGTCTAATAAT | |
| | GTTGAATTTGCTGTTAATACTGAAGGTGTATATAGTGAACCCCGCCCCA | |
| | TTGGCACCAGATACCTGACTCGTAATCTGTAA | |
| Targeting | PLNGAVHLY | 16 |
| peptide | | |
| Targeting | IVMNSLK | 17 |
| peptide | | |
| Targeting | RDSPKGW | 18 |
| peptide | | |
| Targeting | YSTDVRM | 19 |
| peptide | | |
| Targeting | RESPRGL | 20 |
| peptide | | |
| Targeting | GNNTRSV | 21 |
| peptide | | |
| Targeting | GNNTRDT | 22 |
| peptide | | |
| Targeting | TNSTRPV | 23 |
| peptide | | |
| CBh | CCAACCTGAAAAAAAGTGATTTCAGGCAGGTGCTCCAGGTAATTAAACA | 24 |
| promoter | TTAATACCCCACCAACCAACCATCCCTTAAACCCTTACCTCTTGCTCAGC | |
| | TAATTACAGCCCGGAGGAGAAGGGCCGTCCCGCCCGCTCACCTGTGG | |
| | GAGTAACGCGGTCAGTCAGAGCCGGGGGGGGCGGCGCGAGGCGGCG | |
| | GCGGAGCGGGGCACGGGGCGAAGGCAGCGCGCAGCGACTCCCGCCC | |
| | GCCGCGCGCTTCGCTTTTTATAGGGCCGCCGCCGCCGCCGCCTCGCC | |
| | ATAAAAGGAAACTTTCGGAGCGCGCCGCTCTGATTGGCTGCCGCCGCA | |
| | CCTCTCCGCCTCGCCCCGCCCCGCCCCTCGCCCCGCCCCGCCCCGCC | |
| | TGGCGCGCGCCCCCCCCCCCCCCCCGCCCCCATCGCTGCACAAAATA | |
| | ATTAAAAAATAAATAAATACAAAATTGGGGGTGGGGAGGGGGGGGAGA | |
| | TGGGGAGAGTGAAGCAGAACGTGGGGCTCACCTCGACCATGGTAATAG | |
| | CGATGACTAATACGTAGATGTACTGCCAAGTAGGAAAGTCCCATAAGGT | |
| | CATGTACTGGGCACAATGCCAGGGGGGCCATTTACCGTCATTGACGTC | |
| | AATAGGGGGCGTACTTGGCATATGATACACTTGATGTACTGCCAAGTGG | |
| | GCAGTTTACCGTAAATACTCCACCCATTGACGTCAATGGAAAGTCCCTA | |
| | TTGGCGTTACTATTGACGTCAATGGGGGGGGGTCGTTGGGCGGTCAGC | |
| | CAGGCGGGCCATTTACCGTAAGTTATGTAACG | |
| hSYN | AGTGCAAGTGGGTTTTAGGACCAGGATGAGGCGGGGGGGGGTGCCT | 25 |
| promoter | ACCTGACGACCGACCCCGACCCACTGGACAAGCACCCAACCCCCATTC | |
| | CCCAAATTGCGCATCCCCTATCAGAGAGGGGGAGGGGAAACAGGATGC | |
| | GGCGAGGCGCGTGCGCACTGCCAGCTTCAGCACCGCGGACAGTGCCT | |
| | TCGCCCCCGCCTGGCGGCGCGCGCCACCGCCGCCTCAGCACTGAAGG | |
| | CGCGCTGACGTCACTCGCCGGTCCCCCGCAAACTCCCCTTCCCGGCCA | |
| | CCTTGGTCGCGTCCGCGCCGCCGCCGGCCCAGCCGGACCGCACCACG | |
| | CGAGGCGCGAGATAGGGGGGCACGGGCGCGACCATCTGCGCTGCGG | |
| | CGCCGGCGACTCAGCGCTGCCTCAGTCTGCGGTGGGCAGCGGAGGAG | |
| | TCGTGTCGTGCCTGAGAGCGCAG | |
| V2 (amino | MAADGYLPDWLEDNLSEGIREWWALKPGAPQPKANQQHQDNARGLVLP | 26 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMAVQGRNYIPGPFYRQQRVSTTVTQNN | |
| | NSEFAWPGASSWALNGRNSLMNPGPAMASHKEGEDRFFPLSGSLIFGKQ | |
| | GTGRDNVDADKVMITNEEEIKTTNPVATESYGVVATNHQSAQAQAIVGALQ | |
| | SQGALPGMVWQDRDVYLQGPIWAKIPHTDGNFHPSPLMGGFGMKHPPPQ | |
| | ILIKNTPVPADPPTAFNKDKLNSFITQYSTGQVSVEIEWELQKENSKRWNPE | |
| | IQYTSNYYKSNNVEFAVNTEGVYSEPRPIGTRYLTRNL | |
| V2 | ATGGCTGCCGATGGTTATCTTCCAGATTGGCTCGAGGACAACCTTAGTG | 27 |
| (nucleotide) | AAGGTATTCGCGAGTGGTGGGCTTTGAAACCTGGAGCCCCTCAACCCA | |
| | AGGCAAATCAACAACATCAAGACAACGCTCGAGGTCTTGTGCTTCCGG | |
| | GTTACAAATACCTTGGACCCGGCAACGGACTCGACAAGGGGGAGCCG | |
| | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGGCTGTCCAGGGAAGAAACTACATACCTGGACCCTTTTA | |
| | CCGACAACAACGTGTCTCAACCACTGTGACTCAAAACAACAACAGCGAA | |
| | TTTGCTTGGCCTGGAGCTTCTTCTTGGGCTCTCAATGGACGTAATAGCT | |
| | TGATGAATCCTGGACCTGCTATGGCCAGCCACAAAGAAGGAGAGGACC | |
| | GTTTCTTTCCTTTGTCTGGATCTTTAATTTTTGGCAAACAAGGAACTGGA | |
| | AGAGACAACGTGGATGCGGACAAAGTCATGATAACCAACGAAGAAGAA | |
| | ATTAAAACTACTAACCCGGTAGCAACGGAGTCCTATGGAGTAGTGGCCA | |
| | CAAACCACCAGAGTGCCCAAGCACAGGCGATTGTTGGCGCTCTTCAAT | |
| | CTCAAGGAGCGCTTCCGGGTATGGTTTGGCAGGACAGAGATGTGTACC | |
| | TGCAAGGACCCATTTGGGCCAAAATTCCTCACACGGACGGCAACTTTCA | |
| | CCCTTCTCCGCTGATGGGAGGGTTTGGAATGAAGCACCCGCCTCCTCA | |
| | GATCCTCATCAAAAACACACCTGTACCTGCGGATCCTCCAACGGCCTTC | |
| | AACAAGGACAAGCTGAACTCTTTCATCACCCAGTATTCTACTGGCCAAG | |
| | TCAGCGTGGAGATCGAGTGGGAGCTGCAGAAGGAAAACAGCAAGCGC | |
| | TGGAACCCGGAGATCCAGTACACTTCCAACTATTACAAGTCTAATAATG | |
| | TTGAATTTGCTGTTAATACTGAAGGTGTATATAGTGAACCCCGCCCCATT | |
| | GGCACCAGATACCTGACTCGTAATCTGTAA | |
| V3 (amino | MAADGYLPDWLEDNLSEGIREWWALKPGAPQPKANQQHQDNARGLVLP | 28 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMAAQGRNYIPGPSYRQQRVSTSVTQN | |
| | NNSEFAWPGASSWALNGRNSLMNPGPAMASHKEGEDRFFPLSGSLIFGK | |
| | QGTGRDNVDADKVMITNEEEIKTTNPVATESYGVVATNHQSAQAQAIVGAL | |
| | QSQGALPGMVWQDRDVYLQGPIWAKIPHTDGNFHPSPLMGGFGMKHPPP | |
| | QILIKNTPVPADPPTAFNKDKLNSFITQYSTGQVSVEIEWELQKENSKRWNP | |
| | EIQYTSNYYKSNNVEFAVNTEGVYSEPRPIGTRYLTRNL | |
| V3 | ATGGCTGCCGATGGTTATCTTCCAGATTGGCTCGAGGACAACCTTAGTG | 29 |
| (nucleotide) | AAGGTATTCGCGAGTGGTGGGCTTTGAAACCTGGAGCCCCTCAACCCA | |
| | AGGCAAATCAACAACATCAAGACAACGCTCGAGGTCTTGTGCTTCCGG | |
| | GTTACAAATACCTTGGACCCGGCAACGGACTCGACAAGGGGGAGCCG | |
| | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGGCTGCCCAGGGAAGAAACTACATACCTGGACCCAGCT | |
| | ACCGACAACAACGTGTCTCAACCTCCGTGACTCAAAACAACAACAGCGA | |
| | ATTTGCTTGGCCTGGAGCTTCTTCTTGGGCTCTCAATGGACGTAATAGC | |
| | TTGATGAATCCTGGACCTGCTATGGCCAGCCACAAAGAAGGAGAGGAC | |
| | CGTTTCTTTCCTTTGTCTGGATCTTTAATTTTTGGCAAACAAGGAACTGG | |
| | AAGAGACAACGTGGATGCGGACAAAGTCATGATAACCAACGAAGAAGA | |
| | AATTAAAACTACTAACCCGGTAGCAACGGAGTCCTATGGAGTAGTGGCC | |
| | ACAAACCACCAGAGTGCCCAAGCACAGGCGATTGTTGGCGCTCTTCAA | |
| | TCTCAAGGAGCGCTTCCGGGTATGGTTTGGCAGGACAGAGATGTGTAC | |
| | CTGCAAGGACCCATTTGGGCCAAAATTCCTCACACGGACGGCAACTTTC | |
| | ACCCTTCTCCGCTGATGGGAGGGTTTGGAATGAAGCACCCGCCTCCTC | |
| | AGATCCTCATCAAAAACACACCTGTACCTGCGGATCCTCCAACGGCCTT | |
| | CAACAAGGACAAGCTGAACTCTTTCATCACCCAGTATTCTACTGGCCAA | |
| | GTCAGCGTGGAGATCGAGTGGGAGCTGCAGAAGGAAAACAGCAAGCG | |
| | CTGGAACCCGGAGATCCAGTACACTTCCAACTATTACAAGTCTAATAAT | |
| | GTTGAATTTGCTGTTAATACTGAAGGTGTATATAGTGAACCCCGCCCCA | |
| | TTGGCACCAGATACCTGACTCGTAATCTGTAA | |
| V4 (amino | MAADGYLPDWLEDNLSEGIREWWALKPGAPQPKANQQHQDNARGLVLP | 30 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMAAQGRNYIPGPSYRQQRVSTTVTQNN | |
| | NSEFAWPGASSWALNGRNSLMNPGPAMASHKEGEDRFFPLSGSLIFGKQ | |
| | GTGRDNVDADKVMITNEEEIKTTNPVATESYGVVATNHQSAQAQAIVGALQ | |
| | SQGALPGMVWQDRDVYLQGPIWAKIPHTDGNFHPSPLMGGFGMKHPPPQ | |
| | ILIKNTPVPADPPTAFNKDKLNSFITQYSTGQVSVEIEWELQKENSKRWNPE | |
| | IQYTSNYYKSNNVEFAVNTEGVYSEPRPIGTRYLTRNL | |
| V4 | ATGGCTGCCGATGGTTATCTTCCAGATTGGCTCGAGGACAACCTTAGTG | 31 |
| (nucleotide) | AAGGTATTCGCGAGTGGTGGGCTTTGAAACCTGGAGCCCCTCAACCCA | |
| | AGGCAAATCAACAACATCAAGACAACGCTCGAGGTCTTGTGCTTCCGG | |
| | GTTACAAATACCTTGGACCCGGCAACGGACTCGACAAGGGGGAGCCG | |
| | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGGCTGCGCAGGGAAGAAACTACATACCTGGACCCAGCT | |
| | ACCGACAACAACGTGTCTCAACCACTGTGACTCAAAACAACAACAGCGA | |
| | ATTTGCTTGGCCTGGAGCTTCTTCTTGGGCTCTCAATGGACGTAATAGC | |
| | TTGATGAATCCTGGACCTGCTATGGCCAGCCACAAAGAAGGAGAGGAC | |
| | CGTTTCTTTCCTTTGTCTGGATCTTTAATTTTTGGCAAACAAGGAACTGG | |
| | AAGAGACAACGTGGATGCGGACAAAGTCATGATAACCAACGAAGAAGA | |
| | AATTAAAACTACTAACCCGGTAGCAACGGAGTCCTATGGAGTAGTGGCC | |
| | ACAAACCACCAGAGTGCCCAAGCACAGGCGATTGTTGGCGCTCTTCAA | |
| | TCTCAAGGAGCGCTTCCGGGTATGGTTTGGCAGGACAGAGATGTGTAC | |
| | CTGCAAGGACCCATTTGGGCCAAAATTCCTCACACGGACGGCAACTTTC | |
| | ACCCTTCTCCGCTGATGGGAGGGTTTGGAATGAAGCACCCGCCTCCTC | |
| | AGATCCTCATCAAAAACACACCTGTACCTGCGGATCCTCCAACGGCCTT | |
| | CAACAAGGACAAGCTGAACTCTTTCATCACCCAGTATTCTACTGGCCAA | |
| | GTCAGCGTGGAGATCGAGTGGGAGCTGCAGAAGGAAAACAGCAAGCG | |
| | CTGGAACCCGGAGATCCAGTACACTTCCAACTATTACAAGTCTAATAAT | |
| | GTTGAATTTGCTGTTAATACTGAAGGTGTATATAGTGAACCCCGCCCCA | |
| | TTGGCACCAGATACCTGACTCGTAATCTGTAA | |
| V5 (amino | MAADGYLPDWLEDNLSEGIREWWALKPGAPQPKANQQHQDNARGLVLP | 32 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMSAQGRNYIPGPSYRQQRVSTSVTQN | |
| | NNSEFAWPGASSWALNGRNSLMNPGPAMASHKEGEDRFFPLSGSLIFGK | |
| | QGTGRDNVDADKVMITNEEEIKTTNPVATESYGVVATNHQSAQAQAIVGAL | |
| | QSQGALPGMVWQDRDVYLQGPIWAKIPHTDGNFHPSPLMGGFGMKHPPP | |
| | QILIKNTPVPADPPTAFNKDKLNSFITQYSTGQVSVEIEWELQKENSKRWNP | |
| | EIQYTSNYYKSNNVEFAVNTEGVYSEPRPIGTRYLTRNL | |
| V5 | ATGGCTGCCGATGGTTATCTTCCAGATTGGCTCGAGGACAACCTTAGTG | 33 |
| (nucleotide) | AAGGTATTCGCGAGTGGTGGGCTTTGAAACCTGGAGCCCCTCAACCCA | |
| | AGGCAAATCAACAACATCAAGACAACGCTCGAGGTCTTGTGCTTCCGG | |
| | GTTACAAATACCTTGGACCCGGCAACGGACTCGACAAGGGGGAGCCG | |
| | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGTCAGCTCAGGGAAGAAACTACATACCTGGACCCAGCT | |
| | ACCGACAACAACGTGTCTCAACCTCAGTGACTCAAAACAACAACAGCGA | |
| | ATTTGCTTGGCCTGGAGCTTCTTCTTGGGCTCTCAATGGACGTAATAGC | |
| | TTGATGAATCCTGGACCTGCTATGGCCAGCCACAAAGAAGGAGAGGAC | |
| | CGTTTCTTTCCTTTGTCTGGATCTTTAATTTTTGGCAAACAAGGAACTGG | |
| | AAGAGACAACGTGGATGCGGACAAAGTCATGATAACCAACGAAGAAGA | |
| | AATTAAAACTACTAACCCGGTAGCAACGGAGTCCTATGGAGTAGTGGCC | |
| | ACAAACCACCAGAGTGCCCAAGCACAGGCGATTGTTGGCGCTCTTCAA | |
| | TCTCAAGGAGCGCTTCCGGGTATGGTTTGGCAGGACAGAGATGTGTAC | |
| | CTGCAAGGACCCATTTGGGCCAAAATTCCTCACACGGACGGCAACTTTC | |
| | ACCCTTCTCCGCTGATGGGAGGGTTTGGAATGAAGCACCCGCCTCCTC | |
| | AGATCCTCATCAAAAACACACCTGTACCTGCGGATCCTCCAACGGCCTT | |
| | CAACAAGGACAAGCTGAACTCTTTCATCACCCAGTATTCTACTGGCCAA | |
| | GTCAGCGTGGAGATCGAGTGGGAGCTGCAGAAGGAAAACAGCAAGCG | |
| | CTGGAACCCGGAGATCCAGTACACTTCCAACTATTACAAGTCTAATAAT | |
| | GTTGAATTTGCTGTTAATACTGAAGGTGTATATAGTGAACCCCGCCCCA | |
| | TTGGCACCAGATACCTGACTCGTAATCTGTAA | |
| V6 (amino | MAADGYLPDWLEDNLSEGIREWWALKPGAPQPKANQQHQDNARGLVLP | 34 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMSAQGRNYIPGPSYRQQRVSTTVTQNN | |
| | NSEFAWPGASSWALNGRNSLMNPGPAMASHKEGEDRFFPLSGSLIFGKQ | |
| | GTGRDNVDADKVMITNEEEIKTTNPVATESYGVVATNHQSAQAQAIVGALQ | |
| | SQGALPGMVWQDRDVYLQGPIWAKIPHTDGNFHPSPLMGGFGMKHPPPQ | |
| | ILIKNTPVPADPPTAFNKDKLNSFITQYSTGQVSVEIEWELQKENSKRWNPE | |
| | IQYTSNYYKSNNVEFAVNTEGVYSEPRPIGTRYLTRNL | |
| V6 | ATGGCTGCCGATGGTTATCTTCCAGATTGGCTCGAGGACAACCTTAGTG | 35 |
| (nucleotide) | AAGGTATTCGCGAGTGGTGGGCTTTGAAACCTGGAGCCCCTCAACCCA | |
| | AGGCAAATCAACAACATCAAGACAACGCTCGAGGTCTTGTGCTTCCGG | |
| | GTTACAAATACCTTGGACCCGGCAACGGACTCGACAAGGGGGAGCCG | |
| | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGTCTGCGCAGGGAAGAAACTACATACCTGGACCCAGCT | |
| | ACCGACAACAACGTGTCTCAACCACTGTGACTCAAAACAACAACAGCGA | |
| | ATTTGCTTGGCCTGGAGCTTCTTCTTGGGCTCTCAATGGACGTAATAGC | |
| | TTGATGAATCCTGGACCTGCTATGGCCAGCCACAAAGAAGGAGAGGAC | |
| | CGTTTCTTTCCTTTGTCTGGATCTTTAATTTTTGGCAAACAAGGAACTGG | |
| | AAGAGACAACGTGGATGCGGACAAAGTCATGATAACCAACGAAGAAGA | |
| | AATTAAAACTACTAACCCGGTAGCAACGGAGTCCTATGGAGTAGTGGCC | |
| | ACAAACCACCAGAGTGCCCAAGCACAGGCGATTGTTGGCGCTCTTCAA | |
| | TCTCAAGGAGCGCTTCCGGGTATGGTTTGGCAGGACAGAGATGTGTAC | |
| | CTGCAAGGACCCATTTGGGCCAAAATTCCTCACACGGACGGCAACTTTC | |
| | ACCCTTCTCCGCTGATGGGAGGGTTTGGAATGAAGCACCCGCCTCCTC | |
| | AGATCCTCATCAAAAACACACCTGTACCTGCGGATCCTCCAACGGCCTT | |
| | CAACAAGGACAAGCTGAACTCTTTCATCACCCAGTATTCTACTGGCCAA | |
| | GTCAGCGTGGAGATCGAGTGGGAGCTGCAGAAGGAAAACAGCAAGCG | |
| | CTGGAACCCGGAGATCCAGTACACTTCCAACTATTACAAGTCTAATAAT | |
| | GTTGAATTTGCTGTTAATACTGAAGGTGTATATAGTGAACCCCGCCCCA | |
| | TTGGCACCAGATACCTGACTCGTAATCTGTAA | |
| V7 (amino | MAADGYLPDWLEDNLSEGIREWWALKPGAPQPKANQQHQDNARGLVLP | 36 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMAVQGRNYIPGPSYRQQRVSTSVTQN | |
| | NNSEFAWPGASSWALNGRNSLMNPGPAMASHKEGEDRFFPLSGSLIFGK | |
| | QGTGRDNVDADKVMITNEEEIKTTNPVATESYGVVATNHQSAQAQAIVGAL | |
| | QSQGALPGMVWQDRDVYLQGPIWAKIPHTDGNFHPSPLMGGFGMKHPPP | |
| | QILIKNTPVPADPPTAFNKDKLNSFITQYSTGQVSVEIEWELQKENSKRWNP | |
| | EIQYTSNYYKSNNVEFAVNTEGVYSEPRPIGTRYLTRNL | |
| V7 | ATGGCTGCCGATGGTTATCTTCCAGATTGGCTCGAGGACAACCTTAGTG | 37 |
| (nucleotide) | AAGGTATTCGCGAGTGGTGGGCTTTGAAACCTGGAGCCCCTCAACCCA | |
| | AGGCAAATCAACAACATCAAGACAACGCTCGAGGTCTTGTGCTTCCGG | |
| | GTTACAAATACCTTGGACCCGGCAACGGACTCGACAAGGGGGAGCCG | |
| | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGGCTGTCCAGGGAAGAAACTACATACCTGGACCCAGCT | |
| | ACCGACAACAACGTGTCTCAACCTCGGTGACTCAAAACAACAACAGCGA | |
| | ATTTGCTTGGCCTGGAGCTTCTTCTTGGGCTCTCAATGGACGTAATAGC | |
| | TTGATGAATCCTGGACCTGCTATGGCCAGCCACAAAGAAGGAGAGGAC | |
| | CGTTTCTTTCCTTTGTCTGGATCTTTAATTTTTGGCAAACAAGGAACTGG | |
| | AAGAGACAACGTGGATGCGGACAAAGTCATGATAACCAACGAAGAAGA | |
| | AATTAAAACTACTAACCCGGTAGCAACGGAGTCCTATGGAGTAGTGGCC | |
| | ACAAACCACCAGAGTGCCCAAGCACAGGCGATTGTTGGCGCTCTTCAA | |
| | TCTCAAGGAGCGCTTCCGGGTATGGTTTGGCAGGACAGAGATGTGTAC | |
| | CTGCAAGGACCCATTTGGGCCAAAATTCCTCACACGGACGGCAACTTTC | |
| | ACCCTTCTCCGCTGATGGGAGGGTTTGGAATGAAGCACCCGCCTCCTC | |
| | AGATCCTCATCAAAAACACACCTGTACCTGCGGATCCTCCAACGGCCTT | |
| | CAACAAGGACAAGCTGAACTCTTTCATCACCCAGTATTCTACTGGCCAA | |
| | GTCAGCGTGGAGATCGAGTGGGAGCTGCAGAAGGAAAACAGCAAGCG | |
| | CTGGAACCCGGAGATCCAGTACACTTCCAACTATTACAAGTCTAATAAT | |
| | GTTGAATTTGCTGTTAATACTGAAGGTGTATATAGTGAACCCCGCCCCA | |
| | TTGGCACCAGATACCTGACTCGTAATCTGTAA | |
| V8 (amino | MAADGYLPDWLEDNLSEGIREWWALKPGAPQPKANQQHQDNARGLVLP | 38 |
| acid) | GYKYLGPGNGLDKGEPVNAADAAALEHDKAYDQQLKAGDNPYLKYNHAD | |
| | AEFQERLKEDTSFGGNLGRAVFQAKKRLLEPLGLVEEAAKTAPGKKRPVE | |
| | QSPQEPDSSAGIGKSGAQPAKKRLNFGQTGDTESVPDPQPIGEPPAAPSG | |
| | VGSLTMASGGGAPVADNNEGADGVGSSSGNWHCDSQWLGDRVITTSTR | |
| | TWALPTYNNHLYKQISNSTSGGSSNDNAYFGYSTPWGYFDFNRFHCHFSP | |
| | RDWQRLINNNWGFRPKRLNFKLFNIQVKEVTDNNGVKTIANNLTSTVQVFT | |
| | DSDYQLPYVLGSAHEGCLPPFPADVFMIPQYGYLTLNDGSQAVGRSSFYC | |
| | LEYFPSQMLRTGNNFQFSYEFENVPFHSSYAHSQSLDRLMNPLIDQYLYYL | |
| | SKTINGSGQNQQTLKFSVAGPSNMSVQGRNYIPGPSYRQQRVSTTVTQNN | |
| | NSEFAWPGASSWALNGRNSLMNPGPAMASHKEGEDRFFPLSGSLIFGKQ | |
| | GTGRDNVDADKVMITNEEEIKTTNPVATESYGVVATNHQSAQAQAIVGALQ | |
| | SQGALPGMVWQDRDVYLQGPIWAKIPHTDGNFHPSPLMGGFGMKHPPPQ | |
| | ILIKNTPVPADPPTAFNKDKLNSFITQYSTGQVSVEIEWELQKENSKRWNPE | |
| | IQYTSNYYKSNNVEFAVNTEGVYSEPRPIGTRYLTRNL | |
| V8 | ATGGCTGCCGATGGTTATCTTCCAGATTGGCTCGAGGACAACCTTAGTG | 39 |
| (nucleotide) | AAGGTATTCGCGAGTGGTGGGCTTTGAAACCTGGAGCCCCTCAACCCA | |
| | AGGCAAATCAACAACATCAAGACAACGCTCGAGGTCTTGTGCTTCCGG | |
| | GTTACAAATACCTTGGACCCGGCAACGGACTCGACAAGGGGGAGCCG | |
| | GTCAACGCAGCAGACGCGGCGGCCCTCGAGCACGACAAGGCCTACGA | |
| | CCAGCAGCTCAAGGCCGGAGACAACCCGTACCTCAAGTACAACCACGC | |
| | CGACGCCGAGTTCCAGGAGCGGCTCAAAGAAGATACGTCTTTTGGGGG | |
| | CAACCTCGGGCGAGCAGTCTTCCAGGCCAAAAAGAGGCTTCTTGAACC | |
| | TCTTGGTCTGGTTGAGGAAGCGGCTAAGACGGCTCCTGGAAAGAAGAG | |
| | GCCTGTAGAGCAGTCTCCTCAGGAACCGGACTCCTCCGCGGGTATTGG | |
| | CAAATCGGGTGCACAGCCCGCTAAAAAGAGACTCAATTTCGGTCAGACT | |
| | GGCGACACAGAGTCAGTCCCAGACCCTCAACCAATCGGAGAACCTCCC | |
| | GCAGCCCCCTCAGGTGTGGGATCTCTTACAATGGCTTCAGGTGGTGGC | |
| | GCACCAGTGGCAGACAATAACGAAGGTGCCGATGGAGTGGGTAGTTCC | |
| | TCGGGAAATTGGCATTGCGATTCCCAATGGCTGGGGGACAGAGTCATC | |
| | ACCACCAGCACCCGAACCTGGGCCCTGCCCACCTACAACAATCACCTC | |
| | TACAAGCAAATCTCCAACAGCACATCTGGAGGATCTTCAAATGACAACG | |
| | CCTACTTCGGCTACAGCACCCCCTGGGGGTATTTTGACTTCAACAGATT | |
| | CCACTGCCACTTCTCACCACGTGACTGGCAGCGACTCATCAACAACAAC | |
| | TGGGGATTCCGGCCTAAGCGACTCAACTTCAAGCTCTTCAACATTCAGG | |
| | TCAAAGAGGTTACGGACAACAATGGAGTCAAGACCATCGCCAATAACCT | |
| | TACCAGCACGGTCCAGGTCTTCACGGACTCAGACTATCAGCTCCCGTA | |
| | CGTGCTCGGGTCGGCTCACGAGGGCTGCCTCCCGCCGTTCCCAGCGG | |
| | ACGTTTTCATGATTCCTCAGTACGGGTATCTGACGCTTAATGATGGAAG | |
| | CCAGGCCGTGGGTCGTTCGTCCTTTTACTGCCTGGAATATTTCCCGTCG | |
| | CAAATGCTAAGAACGGGTAACAACTTCCAGTTCAGCTACGAGTTTGAGA | |
| | ACGTACCTTTCCATAGCAGCTACGCTCACAGCCAAAGCCTGGACCGAC | |
| | TAATGAATCCACTCATCGACCAATACTTGTACTATCTCTCAAAGACTATT | |
| | AACGGTTCTGGACAGAATCAACAAACGCTAAAATTCAGTGTGGCCGGAC | |
| | CCAGCAACATGTCCGTCCAGGGAAGAAACTACATACCTGGACCCAGCT | |
| | ACCGACAACAACGTGTCTCAACCACTGTGACTCAAAACAACAACAGCGA | |
| | ATTTGCTTGGCCTGGAGCTTCTTCTTGGGCTCTCAATGGACGTAATAGC | |
| | TTGATGAATCCTGGACCTGCTATGGCCAGCCACAAAGAAGGAGAGGAC | |
| | CGTTTCTTTCCTTTGTCTGGATCTTTAATTTTTGGCAAACAAGGAACTGG | |
| | AAGAGACAACGTGGATGCGGACAAAGTCATGATAACCAACGAAGAAGA | |
| | AATTAAAACTACTAACCCGGTAGCAACGGAGTCCTATGGAGTAGTGGCC | |
| | ACAAACCACCAGAGTGCCCAAGCACAGGCGATTGTTGGCGCTCTTCAA | |
| | TCTCAAGGAGCGCTTCCGGGTATGGTTTGGCAGGACAGAGATGTGTAC | |
| | CTGCAAGGACCCATTTGGGCCAAAATTCCTCACACGGACGGCAACTTTC | |
| | ACCCTTCTCCGCTGATGGGAGGGTTTGGAATGAAGCACCCGCCTCCTC | |
| | AGATCCTCATCAAAAACACACCTGTACCTGCGGATCCTCCAACGGCCTT | |
| | CAACAAGGACAAGCTGAACTCTTTCATCACCCAGTATTCTACTGGCCAA | |
| | GTCAGCGTGGAGATCGAGTGGGAGCTGCAGAAGGAAAACAGCAAGCG | |
| | CTGGAACCCGGAGATCCAGTACACTTCCAACTATTACAAGTCTAATAAT | |
| | GTTGAATTTGCTGTTAATACTGAAGGTGTATATAGTGAACCCCGCCCCA | |
| | TTGGCACCAGATACCTGACTCGTAATCTGTAA | |

### 10. CITATION OF REFERENCES

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 21, 2020. When one gene or protein references a plurality of sequence accession numbers, all of the sequence variants are encompassed.

1-36. (canceled)

37. A capsid polypeptide comprising an amino acid sequence having at least 95% sequence identity to SEQ ID NO:1 or a VP2 or VP3 portion thereof and comprising a phenylalanine at a position corresponding to S483 of the VP1 capsid polypeptide of SEQ ID NO:1.

38. A capsid polypeptide comprising an amino acid sequence having at least 95% sequence identity to SEQ ID NO:1 or a VP2 or VP3 portion thereof and comprising a phenylalanine at a position corresponding to S483 and a serine at a position corresponding to N598 of the VP1 capsid polypeptide of SEQ ID NO:1.

39. The capsid polypeptide of claim 37, further comprising:
(a) a serine at a position corresponding to A472 of the VP1 capsid polypeptide of SEQ ID NO:1;
(b) an alanine at a position corresponding to V473 of the VP1 capsid polypeptide of SEQ ID NO:1;
(c) a serine at a position corresponding to T492 of the VP1 capsid polypeptide of SEQ ID NO:1; or
(d) any combination of two or three of (a), (b), and (c).

40. The capsid polypeptide of claim 37, further comprising
(a) a serine at a position corresponding to A472 of the VP1 capsid polypeptide of SEQ ID NO:1;
(b) an alanine at a position corresponding to V473 of the VP1 capsid polypeptide of SEQ ID NO:1; and
(c) a serine at a position corresponding to T492 of the VP1 capsid polypeptide of SEQ ID NO:1.

41. The capsid polypeptide of claim 38, further comprising:
(a) a serine at a position corresponding to A472 of the VP1 capsid polypeptide of SEQ ID NO:1;
(b) an alanine at a position corresponding to V473 of the VP1 capsid polypeptide of SEQ ID NO:1;
(c) a serine at a position corresponding to T492 of the VP1 capsid polypeptide of SEQ ID NO:1; or
(d) any combination of two or three of (a), (b), and (c).

42. The capsid polypeptide of claim 37, further comprising:
(a) a valine at a position corresponding to Q579 of the VP1 capsid polypeptide of SEQ ID NO:1;
(b) an isoleucine at a position corresponding to Q592 of the VP1 capsid polypeptide of SEQ ID NO:1;
(c) a valine at a position corresponding to T593 of the VP1 capsid polypeptide of SEQ ID NO:1;
(d) an alanine at a position corresponding to W595 of the VP1 capsid polypeptide of SEQ ID NO:1;
(e) a leucine at a position corresponding to V596 of the VP1 capsid polypeptide of SEQ ID NO:1;
(f) a serine at a position corresponding to N598 of the VP1 capsid polypeptide of SEQ ID NO:1;
(g) an alanine at a position corresponding to I601 of the VP1 capsid polypeptide of SEQ ID NO:1; or
(h) any combination of two, three, four, five, six, or all seven of (a), (b), (c), (d), (e), (f), and (g).

43. The capsid polypeptide of claim 38, further comprising:
(a) a valine at a position corresponding to Q579 of the VP1 capsid polypeptide of SEQ ID NO:1;
(b) an isoleucine at a position corresponding to Q592 of the VP1 capsid polypeptide of SEQ ID NO:1;
(c) a valine at a position corresponding to T593 of the VP1 capsid polypeptide of SEQ ID NO:1;
(d) an alanine at a position corresponding to W595 of the VP1 capsid polypeptide of SEQ ID NO:1;
(e) a leucine at a position corresponding to V596 of the VP1 capsid polypeptide of SEQ ID NO:1;
(f) an alanine at a position corresponding to I601 of the VP1 capsid polypeptide of SEQ ID NO:1; or
(g) any combination of two, three, four, five, or all six of (a), (b), (c), (d), (e), and (f).

44. The capsid polypeptide of claim 37, further comprising:
(a) a valine at a position corresponding to Q579 of the VP1 capsid polypeptide of SEQ ID NO:1;
(b) an isoleucine at a position corresponding to Q592 of the VP1 capsid polypeptide of SEQ ID NO:1;
(c) a valine at a position corresponding to T593 of the VP1 capsid polypeptide of SEQ ID NO:1;
(d) an alanine at a position corresponding to W595 of the VP1 capsid polypeptide of SEQ ID NO:1;
(e) a leucine at a position corresponding to V596 of the VP1 capsid polypeptide of SEQ ID NO:1;
(f) a serine at a position corresponding to N598 of the VP1 capsid polypeptide of SEQ ID NO:1; and
(g) an alanine at a position corresponding to I601 of the VP1 capsid polypeptide of SEQ ID NO:1.

45. The capsid polypeptide of claim 37, further comprising:
(a) a serine at a position corresponding to A472 of the VP1 capsid polypeptide of SEQ ID NO:1;
(b) an alanine at a position corresponding to V473 of the VP1 capsid polypeptide of SEQ ID NO:1;
(c) a serine at a position corresponding to T492 of the VP1 capsid polypeptide of SEQ ID NO:1;
(d) a valine at a position corresponding to Q579 of the VP1 capsid polypeptide of SEQ ID NO:1;
(e) an isoleucine at a position corresponding to Q592 of the VP1 capsid polypeptide of SEQ ID NO:1;
(f) a valine at a position corresponding to T593 of the VP1 capsid polypeptide of SEQ ID NO:1;
(g) an alanine at a position corresponding to W595 of the VP1 capsid polypeptide of SEQ ID NO:1;
(h) a leucine at a position corresponding to V596 of the VP1 capsid polypeptide of SEQ ID NO:1;
(i) a serine at a position corresponding to N598 of the VP1 capsid polypeptide of SEQ ID NO:1;
(j) an alanine at a position corresponding to I601 of the VP1 capsid polypeptide of SEQ ID NO:1; or
(k) any combination of two, three, four, five, six, or all seven of (a), (b), (c), (d), (e), (f), and (g).

46. The capsid polypeptide of claim 37, wherein the amino acid sequence has at least 96% sequence identity to SEQ ID NO:1.

47. The capsid polypeptide of claim 37, wherein the amino acid sequence has at least 97% sequence identity to SEQ ID NO:1.

48. The capsid polypeptide of claim 37, whose sequence has an edit distance of 13 or lower to a VP1 capsid polypeptide of SEQ ID NO:1 or the VP2 or VP3 portion thereof.

49. The capsid polypeptide of claim 37, whose sequence has an edit distance of 12 or lower to a VP1 capsid polypeptide of SEQ ID NO:1 or the VP2 or VP3 portion thereof.

50. The capsid polypeptide of claim 37, whose sequence has an edit distance of 11 or lower to a VP1 capsid polypeptide of SEQ ID NO:1 or the VP2 or VP3 portion thereof.

51. The capsid polypeptide of claim 37, which comprises the amino acid sequence of the VP1 capsid polypeptide of SEQ ID NO:12 or a VP2 or VP3 portion thereof.

52. The capsid polypeptide of claim 37, which is a VP1 capsid polypeptide.

53. The capsid polypeptide of claim 37, which is a VP2 capsid polypeptide.

54. The capsid polypeptide of claim 37, which is a VP3 capsid polypeptide.

55. A nucleic acid comprising a nucleotide sequence encoding the capsid polypeptide of claim 37.

56. A virus particle comprising the capsid polypeptide of claim 37.

57. The virus particle of claim 56, further comprising a nucleic acid comprising a payload and a promoter.

58. An isolated host cell comprising the capsid polypeptide of claim 37.

59. A method of producing a virus particle comprising a capsid polypeptide, the method comprising introducing a nucleic acid molecule according to claim 37 into a cell and harvesting virus particles therefrom.

60. A method of producing a virus particle comprising a capsid polypeptide, comprising culturing a cell engineered to express a capsid polypeptide of claim 37 and harvesting virus particles therefrom.

61. A method of delivering a payload to a cell, comprising contacting the cell with a virus particle comprising the capsid polypeptide of claim 37 and the payload.

62. A method of delivering a payload to a subject, comprising administering to the subject a virus particle comprising the capsid polypeptide of claim 37 and the payload.

63. A method of delivering a payload to the CNS of a subject, comprising administering to the subject the virus particle of any one of claim 56.

64. A method of treating a disease or condition in a subject, comprising administering to the subject in an amount effective to treat the disease or condition a virus particle comprising the capsid polypeptide of claim 37 and a heterologous nucleic acid sequence encoding a therapeutic product suitable for treating the disease or condition.

65. The method of claim 64, wherein the disease or condition is a disease or condition of the CNS.


SEQUENCE LISTING (116 sequences)

SEQ ID NO: 1 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - DNA, 2211 bp, Adeno-associated virus
  source 1..2211: mol_type = genomic DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 3 - protein, 735 aa, Adeno-associated virus 2 [sequence omitted]

SEQ ID NO: 4 - DNA, 2208 bp, Adeno-associated virus 2
  source 1..2208: mol_type = genomic DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 5 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - DNA, 2175 bp, Adeno-associated virus 5
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 7 - protein, 738 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 8 - DNA, 2217 bp, Adeno-associated virus
  source 1..2217: mol_type = genomic DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 9 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 10 - DNA, 2217 bp, Adeno-associated virus
  source 1..2217: mol_type = genomic DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagaggccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgtgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 11 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 12 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMSAQGRNYIPGPFYRQQRVSTSVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 13 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatgtccgctcagggaagaaactacatacctggacccttttaccgacaacaacgtgtctcaacctcagtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 14 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 16 - protein, 9 aa, synthetic construct
PLNGAVHLY

SEQ ID NO: 17 - protein, 7 aa, synthetic construct
IVMNSLK

SEQ ID NO: 18 - protein, 7 aa, synthetic construct
RDSPKGW

SEQ ID NO: 19 - protein, 7 aa, synthetic construct
YSTDVRM

SEQ ID NO: 20 - protein, 7 aa, synthetic construct
RESPRGL

SEQ ID NO: 21 - protein, 7 aa, synthetic construct
GNNTRSV

SEQ ID NO: 22 - protein, 7 aa, synthetic construct
GNNTRDT

SEQ ID NO: 23 - protein, 7 aa, synthetic construct
TNSTRPV

SEQ ID NO: 24 - DNA, 798 bp, synthetic construct
  source 1..798: mol_type = other DNA
ccaacctgaaaaaaagtgatttcaggcaggtgctccaggtaattaaacattaataccccaccaaccaaccatcccttaaacccttacctcttgctcagctaattacagcccggaggagaagggccgtcccgcccgctcacctgtgggagtaacgcggtcagtcagagccggggcgggcggcgcgaggcggcggcggagcggggcacggggcgaaggcagcgcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgaccatggtaatagcgatgactaatacgtagatgtactgccaagtaggaaagtcccataaggtcatgtactgggcacaatgccaggcgggccatttaccgtcattgacgtcaatagggggcgtacttggcatatgatacacttgatgtactgccaagtgggcagtttaccgtaaatactccacccattgacgtcaatggaaagtccctattggcgttactattgacgtcaatgggcgggggtcgttgggcggtcagccaggcgggccatttaccgtaagttatgtaacg

SEQ ID NO: 25 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 26 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPFYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccttttaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 28 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAAQGRNYIPGPSYRQQRVSTSVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgcccagggaagaaactacatacctggacccagctaccgacaacaacgtgtctcaacctccgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 30 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAAQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgcgcagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 32 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMSAQGRNYIPGPSYRQQRVSTSVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 33 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatgtcagctcagggaagaaactacatacctggacccagctaccgacaacaacgtgtctcaacctcagtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 34 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMSAQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatgtctgcgcagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 36 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTSVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 37 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaacctcggtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 38 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMSVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGVVATNHQSAQAQAIVGALQSQGALPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 39 - DNA, 2211 bp, synthetic construct
  source 1..2211: mol_type = other DNA
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatgtccgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggagtagtggccacaaaccaccagagtgcccaagcacaggcgattgttggcgctcttcaatctcaaggagcgcttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 40 - protein, 289 aa, synthetic construct
MRHIICHGGVITEEMAASLLDQLIEEVLADNLPPPSHFEPPTLHELYDLDVTAPEDPNEEAVSQIFPDSVMLAVQEGIDLLTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKMAPAILRRPTSPVSRECNSSTDSCDSGPSNTPPEIHPVVPLCPIKPVAVRVGGRRQAVECIEDLLNEPGQPLDLSCKRPRP

SEQ ID NO: 41 - protein, 176 aa, synthetic construct
MEAWECLEDFSAVRNLLEQSSNSTSWFWRFLWGSSQAKLVCRIKEDYKWEFEELLKSCGELFDSLNLGHQALFQEKVIKTLDFSTPGRAAAAVAFLSFIKDKWSEETHLSGGYLLDFLAMHLWRAVVRHKNRLLLLSSVRPAIIPTEEQQQQQEEARRRRQEQSPWNPRAGLDPRE

SEQ ID NO: 42 - protein, 496 aa, synthetic construct
MERRNPSERGVPAGFSGHASVESGCETQESPATVVFRPPGDNTDGGAAAAAGGSQAAAAGAEPMEPESRPGPSGMNVVQVAELYPELRRILTITEDGQGLKGVKRERGACEATEEARNLAFSLMTRHRPECITFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 43 - protein, 107 aa, synthetic construct
MLSGEAEQLRLKHLVHCRRHKCFARDSGEFCYFELPEDHIEGPAHGVRLTAQGELARSLIREFTQRPLLVERDRGPCVLTVICNCPNLGLHQDLCCHLCAEYNKYRN

SEQ ID NO: 44 - protein, 63 aa, synthetic construct
MNNSSNSTGYSNSGFSRIGVGVILCLVILFILILTLLCLRLAACCVHICIYCQLFKRWGRHPR

SEQ ID NO: 45 - protein, 160 aa, synthetic construct
MIRYIILGLLTLASAHGTTQKVDFKEPACNVTFAAEANECTTLIKCTTEHEKLLIRHKNKIGKYAVYAIWQPGDTTEYNVTVFQGKSHKTFMYTFPFYEMCDITMYMSKQYKLWPPQNCVENTGTFCCTAMLITVLALVCTLLYIKYKSRRSFIEEKKMP

SEQ ID NO: 46 - protein, 93 aa, synthetic construct
MTNTTNAAAATGLTSTTNTPQVSAFVNNWDNLGMWWFSIALMFVCLIIMWLICCLKRKRARPPIYSPIIVLHPNNDGIHRLDGLKHMFFSLTV

SEQ ID NO: 47 - protein, 91 aa, synthetic construct
MIPRVFILLTLVALFCACSTLAAVSHIEVDCIPAFTVYLLYGFVTLTLICSLITVVIAFIQCIDWVCVRFAYLRHHPQYRDRTIAELLRIL

SEQ ID NO: 48 - protein, 132 aa, synthetic construct
MKFTVTFLLIICTLSAFCSPTSKPQRHISCRFTRIWNIPSCYNEKSDLSEAWLYAIISVMVFCSTILALAIYPYLDIGWKRIDAMNHPTFPAPAMLPLQQVVAGGFVPANQPRPTSPTPTEISYFNLTGGDD

SEQ ID NO: 49 - protein, 128 aa, synthetic construct
MTDTLDLEMDGIITEQRLLERRRAAAEQQRMNQELQDMVNLHQCKRGIFCLVKQAKVTYDSNTTGHRLSYKLPTKRQKLVVMVGEKPITITQHSVETEGCIHSPCQGPEDLCTLIKTLCGLKDLIPFN

SEQ ID NO: 50 - protein, 150 aa, synthetic construct
MTTSGVPFGMTLRPTRSRLSRRTPYSRDRLPPFETETRATILEDHPLLPECNTLTMHNAWTSPSPPVKQPQVGQQPVAQQLDSDMNLSELPGEFINITDERLARQETVWNITPKNMSVTHDMMLFKASRGERTVYSVCWEGGGRLNTRVL

SEQ ID NO: 51 - protein, 294 aa, synthetic construct
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM

SEQ ID NO: 52 - protein, 114 aa, synthetic construct
MVLPALPAPPVCDSQNECVGWLGVAYSAVVDVIRAAAHEGVYIEPEARGRLDALREWIYYNYYTERSKRRDRRRRSVCHARTWFCFRKYDYVRRSIWHDTTTNTISVVSAHSVQ

SEQ ID NO: 53 - protein, 116 aa, synthetic construct
MIRCLRLKVEGALEQIFTMAGLNIRDLLRDILRRWRDENYLGMVEGAGMFIEEIHPEGFSLYVHLDVRAVCLLEAIVQHLTNAIICSLAVEFDHATGGERVHLIDLHFEVLDNLLE

SEQ ID NO: 54 - protein, 130 aa, synthetic construct
MFERKMVSFSVVVPELTCLYLHEHDYDVLSFLREALPDFLSSTLHFISPPMQQAYIGATLVSIAPSMRVIISVGSFVMVPGGEVAALVRADLHDYVQLALRRDLRDRGIFVNVPLLNLIQVCEEPEFLQS

SEQ ID NO: 55 - protein, 128 aa, synthetic construct
MAAAVEALYVVLEREGAILPRQEGFSGVYVFFSPINFVIPPMGAVMLSLRLRVCIPPGYFGRFLALTDVNQPDVFTESYIMTPDMTEELSVVLFNHGDQFFYGHAGMAVVRLMLIRVVFPVVRQASNV

SEQ ID NO: 56 - protein, 289 aa, synthetic construct
MRHIICHGGVITEEMAASLLDQLIEEVLADNLPPPSHFEPPTLHELYDLDVTAPEDPNEEAVSQIFPESVMLAVQEGIDLFTFPPAPGSPEPPHLSRQPEQPEQRALGPVSMPNLVPEVIDLTCHEAGFPPSDDEDEEGEEFVLDYVEHPGHGCRSCHYHRRNTGDPDIMCSLCYMRTCGMFVYSPVSEPEPEPEPEPEPARPTRRPKLVPAILRRPTSPVSRECNSSTDSCDSGPSNTPPEIHPVVPLCPIKPVAVRVGGRRQAVECIEDLLNESGQPLDLSCKRPRP

SEQ ID NO: 57 - protein, 175 aa, synthetic construct
MEAWECLEDFSAVRNLLEQSSNSTSWFWRFLWGSSQAKLVCRIKEDYKWEFEELLKSCGELFDSLNLGHQALFQEKVIKTLDFSTPGRAAAAVAFLSFIKDKWSEETHLSGGYLLDFLAMHLWRAVVRHKNRLLLLSSVRPAIIPTEEQQQEEARRRRRQEQSPWNPRAGLDPRE

SEQ ID NO: 58 - protein, 495 aa, synthetic construct
MERRNPSERGVPAGFSGHASVESGGETQESPATVVFRPPGNNTDGGATAGGSQAAAAAGAEPMEPESRPGPSGMNVVQVAELFPELRRILTINEDGQGLKGVKRERGASEATEEARNLTFSLMTRHRPECVTFQQIKDNCANELDLLAQKYSIEQLTTYWLQPGDDFEEAIRVYAKVALRPDCKYKISKLVNIRNCCYISGNGAEVEIDTEDRVAFRCSMINMWPGVLGMDGVVIMNVRFTGPNFSGTVFLANTNLILHGVSFYGFNNTCVEAWTDVRVRGCAFYCCWKGVVCRPKSRASIKKCLFERCTLGILSEGNSRVRHNVASDCGCFMLVKSVAVIKHNMVCGNCEDRASQMLTCSDGNCHLLKTIHVASHSRKAWPVFEHNILTRCSLHLGNRRGVFLPYQCNLSHTKILLEPESMSKVNLNGVFDMTMKIWKVLRYDETRTRCRPCECGGKHIRNQPVMLDVTEELRPDHLVLACTRAEFGSSDEDTD

SEQ ID NO: 59 - protein, 107 aa, synthetic construct
MTSGEAERLRLTHLDHCRRHKCFARGSGEFCYFELPEEHIEGPAHGVRLTTQVELTRSLIREFTKRPLLVERERGPCVLTVVCNCPNPGLHQDLCCHLCAEYNKYRN

SEQ ID NO: 60 - protein, 61 aa, synthetic construct
MSNSSNSTSLSNFSGIGVGVILTLVILFILILALLCLRVAACCTHVCTYCQLFKRWGQHPR

SEQ ID NO: 61 - protein, 159 aa, synthetic construct
MRYMILGLLALAAVCSAAKKVEFKEPACNVTFKSEANECTTLIKCTTEHEKLIIRHKDKIGKYAVYAIWQPGDTNDYNVTVFQGENRKTFMYKFPFYEMCDITMYMSKQYKLWPPQKCLENTGTFCSTALLITALALVCTLLYLKYKSRRSFIDEKKMP

SEQ ID NO: 62 - protein, 101 aa, synthetic construct
MTGSTIAPTTDYRNTTATGLTSALNLPQVHAFVNDWASLDMWWFSIALMFVCLIIMWLICCLKRRRARPPIYRPIIVLNPHNEKIHRLDGLKPCSLLLQYD

SEQ ID NO: 63 - protein, 91 aa, synthetic construct
MIPRVLILLTLVALFCACSTLAAVAHIEVDCIPPFTVYLLYGFVTLILICSLVTVVIAFIQFIDWVCVRIAYLRHHPQYRDRTIADLLRIL

SEQ ID NO: 64 - protein, 130 aa, synthetic construct
MKRSVIFVLLIFCALPVLCSQTSAPPKRHISCRFTQIWNIPSCYNKQSDLSEAWLYAIISVMVFCSTIFALAIYPYLDIGWNAIDAMNHPTFPVPAVIPLQQVIAPINQPRPPSPTPTEISYFNLTGGDD

SEQ ID NO: 65 - protein, 128 aa, synthetic construct
MTESLDLELDGINTEQRLLERRKAASERERLKQEVEDMVNLHQCKRGIFCVVKQAKLTYEKTTTGNRLSYKLPTQRQKLVLMVGEKPITVTQHSAETEGCLHFPYQGPEDLCTLIKTMCGIRDLIPFN

SEQ ID NO: 66 - protein, 150 aa, synthetic construct
MTTSGVPFGMTLRPTRSRLSRRTPYSRDRLPPFETETRATILEDHPLLPECNTLTMHNAWTSPSPPVEQPQVGQQPVAQQLDSDMNLSELPGEFINITDERLARQETVWNITPKNMSVTHDMMLFKASRGERTVYSVCWEGGGRLNTRVL

SEQ ID NO: 67 - protein, 294 aa, synthetic construct; identical to SEQ ID NO: 51

SEQ ID NO: 68 - protein, 114 aa, synthetic construct
MVLPALPAPPVCDSQNECVGWLGVAYSAVVDVIRAAAHEGVYIEPEARGRLDALREWIYYNYYTERAKRRDRRRRSVCHARTWFCFRKYDYVRRSIWHDTTTNTISVVSAHSVQ

SEQ ID NO: 69 - protein, 116 aa, synthetic construct
MIRCLRLKVEGALEQIFTMAGLNIRDLLRDILIRWRDENYLGMVEGAGMFIEEIHPEGFSLYVHLDVRAVCLLEAIVQHLTNAIICSLAVEFDHATGGERVHLIDLHFEVLDNLLE

SEQ ID NO: 70 - protein, 130 aa, synthetic construct
MFERKMVSFSVVVPELTCLYLHEHDYDVLAFLREALPDFLSSTLHFISPPMQQAYIGATLVSIAPSMRVIISVGSFVMVPGGEVAALVRADLHDYVQLALRRDLRDRGIFVNVPLLNLIQVCEEPEFLQS

SEQ ID NO: 71 - protein, 128 aa, synthetic construct; identical to SEQ ID NO: 55

SEQ ID NO: 72 - protein, 621 aa, synthetic construct
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 73 - protein, 536 aa, synthetic construct
MPGFYEIVIKVPSDLDGHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 74 - protein, 397 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 75 - protein, 312 aa, synthetic construct
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 76 - DNA, 20 bp, synthetic construct
  source 1..20: mol_type = other DNA
  modified_base 2: mod_base = m5u
ctcagtaacattgacaccac

SEQ ID NO: 77 - protein, 527 aa, synthetic construct
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 78 - protein, 355 aa, synthetic construct
MDAHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTL

SEQ ID NO: 79 - protein, 449 aa, synthetic construct
GARSYQVIDTRATCYEDQGISYRGTWSTAESGAECTNWQSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHMLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 80 - protein, 527 aa, synthetic construct
SYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGNWSTAESGAECTNWQSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAAAAASPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 81 - protein, 119 aa, synthetic construct [sequence omitted]

SEQ ID NO: 82 - protein, 112 aa, synthetic construct [sequence omitted]

SEQ ID NO: 83 - protein, 112 aa, synthetic construct
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSS

SEQ ID NO: 84 - protein, 112 aa, synthetic construct
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIK

SEQ ID NO: 85 - protein, 124 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSS

SEQ ID NO: 86 - protein, 107 aa, synthetic construct [sequence omitted]

SEQ ID NO: 87 - protein, 124 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSS

SEQ ID NO: 88 - protein, 112 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIK

SEQ ID NO: 89 - protein, 126 aa, synthetic construct [sequence omitted]

SEQ ID NO: 90 - protein, 108 aa, synthetic construct [sequence omitted]

SEQ ID NO: 91 - protein, 115 aa, synthetic construct
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 92 - protein, 112 aa, synthetic construct
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 93 - protein, 126 aa, synthetic construct [sequence omitted]

SEQ ID NO: 94 - protein, 108 aa, synthetic construct [sequence omitted]

SEQ ID NO: 95 - protein, 122 aa, synthetic construct
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSS

SEQ ID NO: 96 - protein, 110 aa, synthetic construct
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIK

SEQ ID NO: 97 - protein, 118 aa, synthetic construct
EVQLQESGPGLVKPSETLSLTCTVSGFSLTSNDIAWIRQPPGKGLEWMGTIWTDGSTNYNTAVQSRVTISVDTSKNQFSLKLSSVTAADTAVYYCARHRLYYGAFDYWGQGTMVTVSS

SEQ ID NO: 98 - protein, 112 aa, synthetic construct
DIVMTQTPLSLSVTPGQPASISCRSSQSLEYSDGYTYLEWYLQKPGQSPQLLIYEVSNRFSGVPDRFSGSGSGTDFTLKISRVEAEDVGVYYCFQATHNPYTFGQGTKLEIK

SEQ ID NO: 99 - protein, 116 aa, synthetic construct
EVQLVQSGAEVKKPGESLKISCKGSGYTFSNYWIEWVRQMPGKGLEWMGEILPGSDSIKYEKNFKGQVTISADKSISTAYLQWSSLKASDTAMYYCARRGNYVDDWGQGTLVTVSS

SEQ ID NO: 100 - protein, 112 aa, synthetic construct
EIVLTQSPGTLSLSPGERATLSCRSSQSLVHSNQNTYLHWYQQKPGQAPRLLIYKVDNRFSGIPDRFSGSGSGTDFTLTISRLEPEDFAVYYCSQSTLVPLTFGGGTKVEIK

SEQ ID NO: 101 - protein, 117 aa, synthetic construct
EVQLVESGGGLVQPGGSLRLSCAASGLIFRSYGMSWVRQAPGKGLEWVATINSGGTYTYYPDSVKGRFTISRDNSKNTLYLQMNSLRAEDTAVYYCANSYSGAMDYWGQGTLVTVSS

SEQ ID NO: 102 - protein, 112 aa, synthetic construct
DDVLTQTPLSLPVTPGQPASISCRSSQSIVHSNGNTYLEWYLQKPGQSPQLLIYKVSNRFSGVPDRFSGSGSGTDFTLKISRVEAEDVGVYYCFQGSLVPWTFGQGTKVEIK

SEQ ID NO: 103 - protein, 118 aa, synthetic construct
QVQLVESGGGVVQPGRSLRLSCAASGFTFSSYAMSWVRQAPGKGLEWVASISKGGNTYYADSVKGRFTISRDNSKNTLYLQMNSLRAEDTAVYYCARGWGDYGWFAYWGQVTLVTVSS

SEQ ID NO: 104 - protein, 107 aa, synthetic construct
DIQMTQSPSSLSASVGDRVTITCKASQDINRYLNWFQQKPGKAPKSLIYRANRLLDGVPSRFSGSGSGTDFTLTISSLQPEDFATYYCLQYDEFPLTFGQGTKLEIK

SEQ ID NO: 105 - protein, 121 aa, synthetic construct [sequence omitted]

SEQ ID NO: 106 - protein, 106 aa, synthetic construct [sequence omitted]

SEQ ID NO: 107 - protein, 122 aa, synthetic construct [sequence omitted]

SEQ ID NO: 108 - protein, 106 aa, synthetic construct [sequence omitted]

SEQ ID NO: 109 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 110 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 111 - protein, 118 aa, synthetic construct
QAYLQQSGAELVRPGASVKMSCKASGYTFTSYNMHWVKQTPRQGLEWIGGIYPGNGDTSYNQKFKGKATLTVGKSSSTAYMQLSSLTSEDSAVYFCARYDYNYAMDYWGQGTSVTVSS

SEQ ID NO: 112 - protein, 106 aa, synthetic construct
QIVLSQSPAILSASPGEKVTMTCRASSSVSYMHWYQQKPGSSPKPWIYATSNLASGVPARFSGSGSGTSYSFTISRVEAEDAATYYCQQWTFNPPTFGGGTRLEIK

SEQ ID NO: 113 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 114 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 115 - protein, 108 aa, synthetic construct
QSVEESGGRLVTPGTPLTLTCTVSGFSLSSNAINWVRQAPGKGLEWIGYIAVSGNTYYASWAKGRFTISKASTTVDLKMTSPTAEDTGTYFCGKSNIWGPGTLVTVSL

SEQ ID NO: 116 - protein, 111 aa, synthetic construct
QVLTQTTSPVSAAVGSTVTISCQSSQSVRTNKLAWFQQKPGQPPKRLIYSASTLDFGVPSRFSASGSGTQFTLTISDVQCDDAATYYCLGYFDCSIADCVAFGGGTEVVVK